(19) United States
(12) Patent Application Publication
(10) Pub. No.: US 2026/0022357 A1
(43) Pub. Date: Jan. 22, 2026

(54) RECOMBINANT PROTEINS FOR GENE DELIVERY AND INSERTION

(71) Applicant: Averna Therapeutics Ltd, Ness Ziona (IL)

(72) Inventors: Yaron BEN SHUSHAN-GOLECZKI, Tel Aviv-Jaffa (IL); Noga KOWALSMAN, Nessa Ziona (IL); Devin TRUDEAU, Rehovot (IL)

(21) Appl. No.: 19/224,551

(22) Filed: May 30, 2025

Related U.S. Application Data
(63) Continuation of application No. PCT/IB2023/062154, filed on Dec. 2, 2023.
(60) Provisional application No. 63/429,955, filed on Dec. 2, 2022.

(51) Int. Cl. C12N 9/22; A61K 38/00; C07K 14/195; C07K 14/47; C12N 9/12; C12N 15/85; C12N 15/88

(52) U.S. Cl. CPC C12N 9/22; C07K 14/195; C07K 14/4702; C12N 9/1252; C12N 9/1276; C12N 15/85; C12N 15/88; A61K 38/00; C07K 2319/80; C12N 2800/22; C12Y 207/07007; C12Y 207/07049; C12Y 207/13003; C12Y 301/26004

(57) ABSTRACT

The present disclosure provides compositions and methods for delivering a gene of interest to a subject. Aspects of the application relate to nucleic acids encoding modified retroelement-derived polypeptides and gene delivery constructs that can direct integration of a nucleic acid sequence into a target nucleic acid (e.g., a genome of a subject).

### CROSS-REFERENCE TO RELATED APPLICATION

This application is a continuation application of International Application No. PCT/IB2023/062154 filed Dec. 2, 2023, which claims priority to U.S. Provisional Application No. 63/429,955, filed on Dec. 2, 2022. This application is hereby incorporated by reference in their entirety.

### REFERENCE TO AN ELECTRONIC SEQUENCE LISTING

The contents of the electronic sequence listing (AVRT_001_01 US_SeqList_ST26.xml; Size: 828,842 bytes; and Date of Creation: May 29, 2025) are herein incorporated by reference in its entirety.

### BACKGROUND OF INVENTION

Gene therapy can be used to treat diseases or conditions associated with genetic defects by delivering one or more therapeutic genes or cells with corrected defects to a subject. For example, therapeutic genes can be delivered in vectors that promote integration into the host genome of a subject.

Despite recent activity in the field of retrotransposon delivery and genome insertion, there remains a need for nucleic acids and proteins to carry out these methods with improved, inter alia, efficiency, specificity, accuracy, fidelity and processivity of integration. Provided herein are methods and compositions that address the same.

### SUMMARY OF THE INVENTION

In one aspect, provided herein is a nucleic acid encoding an engineered protein comprising a retroelement-derived polypeptide and at least one heterologous polypeptide; wherein the retroelement-derived polypeptide is derived from a non-long terminal repeat (non-LTR) retrotransposon; wherein the at least one heterologous polypeptide comprises an RNA/DNA processing polypeptide or domain thereof, an RNA/DNA repair polypeptide or domain thereof, a nucleic acid binding polypeptide or domain thereof, or a nucleosome binding polypeptide or domain thereof; and wherein the engineered protein exhibits at least one improved integration characteristic, as compared to a retroelement-derived polypeptide not fused to the at least one heterologous polypeptide.

In another aspect, provided herein is a nucleic acid encoding an engineered protein comprising a retroelement-derived polypeptide variant having at least one amino acid modification when compared to a naturally occurring retroelement-derived polypeptide; wherein the retroelement-derived polypeptide variant is derived of a non-long terminal repeat (non-LTR) retrotransposon; and wherein the retroelement-derived polypeptide variant exhibits at least one improved integration characteristic, as compared to the naturally occurring retroelement-derived polypeptide without the at least one amino acid modification.

In another aspect provided herein is a nucleic acid encoding a retroelement-derived reverse transcriptase domain having at least one amino acid modification that stabilizes the reverse transcriptase domain and/or stabilizes its association with RNA and/or DNA relative to an unsubstituted reverse transcriptase domain, wherein the reverse transcriptase domain is an amino acid variant of a reverse transcriptase domain of a non-LTR retrotransposon.

In another aspect provided herein is a nucleic acid encoding a retroelement-derived endonuclease domain comprising at least one amino acid modification that promotes association of the retroelement-derived endonuclease domain with DNA relative to an unmodified endonuclease domain.

In another aspect provided herein are methods of modifying polynucleotides, e.g. in a cell or a subject, comprising using any one or more of the nucleic acids encoding the engineered proteins described herein.

### BRIEF DESCRIPTION OF DRAWINGS

The figures and figure descriptions provided herein are intended to illustrate embodiments by way of example only.

FIGS. 1A-1E illustrate non-limiting examples of gene delivery constructs that are useful for promoting insertion of a transgene (e.g., a heterologous nucleic acid comprising a gene of interest) into a target nucleic acid. FIG. 1A illustrates a non-limiting example of a gene delivery construct comprising a transgene (a “gene of interest”) flanked by two terminal regions that can interact with a retroelement-derived polypeptide and promote integration of the transgene into a target nucleic acid. FIG. 1B illustrates a non-limiting example of two separate nucleic acid molecules, wherein the first nucleic acid is a gene delivery construct comprising a gene of interest flanked by two terminal regions (as shown in FIG. 1A), and the second nucleic acid is a driver construct encoding a driver comprising an engineered protein (e.g., comprising a retroelement-derived polypeptide) are in trans configuration. FIG. 1C illustrates a non-limiting example of a single nucleic acid comprising both a gene of interest ad a sequence encoding an engineered protein (e.g., comprising a retroelement-derived polypeptide) flanked by two terminal regions, with the gene of interest and the sequence encoding an engineered protein being in a cis configuration (i.e., in the same nucleic acid). FIG. 1D illustrates a non-limiting example of gene integration into a target nucleic acid (e.g., genomic DNA) promoted by two separate nucleic acid molecules (a gene delivery construct a gene of interest flanked by two terminal regions, and a driver construct comprising a coding sequence for an engineered retroelement-derived polypeptide) in a trans configuration. FIG. 1E illustrates a non-limiting example of gene integration into a target nucleic acid (e.g., genomic DNA) promoted by a single nucleic acid comprising both a gene of interest and an engineered protein coding sequence flanked by two terminal regions.

FIGS. 2A-2L illustrate non-limiting examples of different configurations of an engineered protein comprising a retroelement-derived polypeptide fused to one or more heterologous polypeptides that can promote integration of a transgene into a target nucleic acid.

FIG. 2A illustrates a non-limiting example of an engineered protein comprising a retroelement-derived polypeptide fused to a heterologous polypeptide at its N-terminus. FIG. 2B illustrates a non-limiting example of an engineered protein comprising a retroelement-derived polypeptide fused to a heterologous polypeptide at its C-terminus. FIG. 2C illustrates a non-limiting example of an engineered protein comprising a retroelement-derived polypeptide that has a first heterologous polypeptide fused to its N-terminus and a second heterologous polypeptide fused to its C-terminus. FIG. 2D illustrates a non-limiting example of a heterologous polypeptide comprising a first domain and a second domain fused to the N-terminus of a retroelement-derived polypeptide. FIG. 2E illustrates a non-limiting example of a heterologous polypeptide comprising an optional linker, a domain, and an optional nuclear localization sequence (NLS) fused to the N-terminus of a retroelement-derived polypeptide. FIG. 2F illustrates a non-limiting example of a heterologous polypeptide comprising an optional linker, a domain, and an optional NLS fused to the C-terminus of a retroelement-derived polypeptide. FIG. 2G illustrates a non-limiting example of a first heterologous polypeptide comprising a first optional linker, a first domain, and a first optional NLS at the N-terminus of a retroelement-derived polypeptide, and a second heterologous polypeptide comprising a second optional linker, a second domain, and a second optional NLS at the C-terminus of the retroelement-derived polypeptide. FIG. 2H illustrates a non-limiting example of a heterologous polypeptide comprising an optional NLS, a first domain, a first optional linker, a second domain, and a second optional linker fused to the N-terminus of a retroelement-derived polypeptide. FIG. 2I illustrates a non-limiting example of a heterologous polypeptide comprising a first optional linker, a first domain, a second optional linker, a second domain, and an optional NLS fused to the C-terminus of a retroelement-derived polypeptide. FIG. 2J illustrates a non-limiting example of a first heterologous polypeptide comprising an optional NLS, a first domain, a first optional linker, a second domain, and a second optional linker fused to the N-terminus of a retroelement-derived polypeptide, and a second heterologous polypeptide comprising a third optional linker, a third domain, a fourth optional linker, a fourth domain, and a second optional NLS fused to the C-terminus of the retroelement-derived polypeptide. FIG. 2K illustrates a non-limiting example of a first heterologous polypeptide comprising an optional NLS, a first domain, a first optional linker, a second domain, and a second optional linker fused to the N-terminus of a retroelement-derived polypeptide, and a second heterologous polypeptide comprising a third optional linker, a third domain, and a second optional NLS fused to the C-terminus of the retroelement-derived polypeptide. FIG. 2L illustrates a non-limiting example of a first heterologous polypeptide comprising an optional NLS, a first domain, and a first optional linker fused to the N-terminus of a retroelement-derived polypeptide, and a second heterologous polypeptide comprising a second optional linker, a second domain, a third optional linker, a third domain, and a second optional NLS fused to the C-terminus of the retroelement-derived polypeptide.

FIG. 3 illustrates the results of integration assays using retroelement-derived polypeptides comprising HDR and chromatin opening domains along with p53 inhibition.

FIGS. 4A-4B illustrate the results of integration assays using retroelement-derived polypeptides comprising point mutations.

FIG. 5 illustrates the results of integration assays using drivers with combinations of domain fusions and point mutations. The different retrotransposable element (driver) constructs were used in the trans configuration (driver and GFP reporter encoded by different RNA). A common gene delivery construct encoding the GFP reporter (EX2107; SEQ ID NO: 319) was used for all driver constructs tested.

FIGS. 6A-6I show integration assays results using Vingi-1_Acar drivers with combinations of domain fusions and point mutations. In these experiment, different retrotransposable element (driver) constructs were used in the trans configuration (driver and GFP reporter encoded by different RNA). A common gene delivery construct encoding the GFP reporter (EX2988; SEQ ID NO: 328) was used for all driver constructs tested. Aside from the mutations and fusions listed, all constructs were identical in sequence to EX2985 (WT, marked with pattern).

FIG. 7A shows results of integration assays using Vingi-1 drivers with point mutations. In this experiment, different retrotransposable element (driver) constructs were used in the trans configuration (driver and GFP reporter encoded by different RNA). A common gene delivery construct encoding the GFP reporter (EX2988; SEQ ID NO: 328) was used for all driver constructs tested. The following mutations in Vingi-1 were tested in this experiment: Q634L (SEQ ID NO: 71), F238Y+M16I (SEQ ID NO:376), I45L (SEQ ID NO: 77), G833I (SEQ ID NO: 84), K703R (SEQ ID NO: 119), K480Q (SEQ ID NO: 120), K675R (SEQ ID NO: 121), P808K (SEQ ID NO: 125), M570L (SEQ ID NO: 151), L590F (SEQ ID NO: 153), M735E (SEQ ID NO: 156), K966R (SEQ ID NO: 168), A901H (SEQ ID NO: 83), L493R (SEQ ID NO: 102). The wild-type Vingi-1_Acar driver is shown in grey (SEQ ID NO:327).

FIG. 7B shows results of integration assays using Vingi-1 drivers with point mutations. In this experiment, different retrotransposable element (driver) constructs were used in the trans configuration (driver and GFP reporter encoded by different RNA). A common gene delivery construct encoding the GFP reporter (EX2988; SEQ ID NO: 328) was used for all driver constructs tested. The following mutations in Vingi-1 were tested in this experiment: D191A (SEQ ID NO: 174), A684S (SEQ ID NO: 72), Y313F (SEQ ID NO: 93), Q215D (SEQ ID NO: 96), K966R (SEQ ID NO: 168), K675R (SEQ ID NO: 121), G116N (SEQ ID NO: 111), N695R (SEQ ID NO: 178), R696H (SEQ ID NO: 179), S754T (SEQ ID NO: 243), P808T (SEQ ID NO: 185), N-terminal fusion of PaRecT (SEQ ID NO: 275), Codon-optimized Vingi-1 driver mRNA (SEQ ID NO: 397).

### DETAILED DESCRIPTION OF INVENTION

In some aspects, the application relates to engineered retroelement-derived polypeptides, nucleic acids (e.g., RNA and/or DNA) encoding the engineered retroelement-derived polypeptides, and their use to promote integration of a transgene (e.g., a heterologous nucleic acid encoding a gene of interest) into a target nucleic acid.

### Definitions

The term “about,” as used herein, is intended to qualify the numerical values which it modifies, denoting such a value as variable within a margin of ±10%.

The terms “protein”, “peptide” and “polypeptide” may be used interchangeably herein and refer to a polymer of amino acid residues linked together by peptide bonds, which may be canonical amide bonds or other types of bonds linking amino acids. Typically, a protein, peptide or polypeptide will be at least three amino acids long, however, any size is contemplated. In some embodiments, a protein, peptide or polypeptide may have at least one domain, which is a structure defined by a portion or portions of the amino acid sequence, which provides a functional property. In some embodiments, the domain can fold independently to produce the functional structure. The function property may be an enzymatic “activity” (nuclease activity, DNA transcriptase activity, integrase activity). For example, an amino acid sequence of a natural or synthetic polypeptide or protein having a reverse transcriptase (RT) activity comprises an RT domain. In some embodiments, the domain may be a motif, which confers a given characteristic or property that is not an enzymatic activity, such as stabilization, binding specificity, interaction specificity, or recruiting specificity. A protein, peptide or polypeptide may comprise several different domains.

A protein, peptide or polypeptide may be natural or synthetic and may optionally include one or more mutations or modifications (e.g. amino acid substitutions, amino acid deletions, amino acid additions, or amino acid truncations) thereby generating a “variant”, i.e., a variant protein, variant peptide, or variant polypeptide that has a different amino acid sequence compared to the naturally occurring protein, peptide or polypeptide. In non-limiting examples, an amino acid mutation, is an amino acid substitution that improves packing of hydrophobic residues in the core of the active domain, stabilizes a loop region, and/or alters electrostatic charge, H-bond stability, or S-bond stability. In other non-limiting examples, the substitution and/or addition that stabilizes a loop region is a proline substitution. Without wishing to be bound to theory, certain substitutions may alter an electrostatic charge, for example an amino acid having a positive charge may increase affinity towards DNA or RNA or to increase specificity by altering the H-bond network with a polar substitution. Hydrophobic mutations may increase secondary and tertiary structure and helical propensity and size mutations can lead to improved stability. For example, by mutation to lysine or arginine, or mutation from aspartate or glutamate to a non-charged residue such as alanine. A “variant” polypeptide may have from about 70%, to about 99%, or 80%, 85%, 90%, 95%, 96%, 97%, 98%, or 99% identity to the wild-type or reference polypeptide and having the same or substantially the same function as the wild-type or reference polypeptide. The percent identity between two such polypeptides can be determined by manual inspection of the two optimally aligned polypeptide sequences or by using software programs or algorithms (e.g., CLUSTAL, MUSCLE, MAFFT) using standard parameters, familiar to one with skill in the art.

A “fusion” protein, polypeptide, or peptide refers to a protein, polypeptide, or peptide that has been modified by adding (fusing) at least one polypeptide, which may be a domain, from a different (i.e., heterologous) protein, polypeptide, or peptide. The heterologous polypeptide may be located at the amino-terminal (N-terminal) portion of the fusion protein thus forming an “amino-terminal” (or “N-terminus”) fusion protein. Alternatively, the heterologous polypeptide may be located at the carboxy-terminal (C-terminal) protein thus forming a “carboxy-terminal” (or “C-terminus”) fusion protein. As described herein, a heterologous polypeptide may be fused at the N-terminus, C-terminus, or internally.

An “engineered” protein, peptide or polypeptide refers to a protein, peptide or polypeptide that comprises (1) at least one amino acid modification to create a variant and/or (2) at least one domain from a heterologous polypeptide, which may be a domain. An engineered protein, peptide or polypeptide may comprise a plurality (e.g., 2, 3, 4, 5, 6, 7, or more) of heterologous domains and/or a plurality (e.g., 2, 3, 4, 5, 6, 7, or more) of amino acid modifications.

In some embodiments, an engineered protein, peptide or polypeptide described herein is encoded by a nucleic acid (e.g., an RNA molecule or a DNA molecule). The nucleic acids and polypeptides disclosed herein may be produced by methods known in the art. For example, the nucleic acids disclosed herein may be prepared synthetically or via in-vitro transcription (IVT) methods known in the art. Likewise, the polypeptides described herein may be produced via recombinant protein expression and purification, which is well suited for fusion proteins.

Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The terms “polynucleotide,” “nucleic acid” and “NA” may be used interchangeably and refer to a polymer of nucleotides. In some embodiments, the polynucleotide comprises one or more chemical and/or sequence modifications. The polymer may include natural nucleosides (i.e., adenosine, thymidine, guanosine, cytidine, uridine, deoxyadenosine, deoxythymidine, deoxyguanosine and deoxycytidine), and nucleoside analogs having modified bases, modified sugars (e.g., 2′-fluororibose, 2′-methoxy), or modified phosphate groups (e.g., phosphorothioates, 2′-5′ linkage). In some embodiments, a nucleic acid comprises one or more chemical and/or sequence modifications. In some embodiments, the modification is an RNA CAP, a modified polyA length (e.g., relative to a natural polyA), a chemically modified nucleotide, a 5′ UTR (untranslated region) modification, a 3′ UTR modification, a modified Sirloin (SINE-derived nuclear RNA localization, Lubelsky, 2018) sequence, a modified (e.g., truncated) stem loop, an RNA stabilization motif, a 5-methoxyuridine (5-MO-U) modification, a 5-methylcytidine (5mC) modification, or one or more additional and/or modified microsatellites. In some embodiments, a nucleic acid encoding an engineered protein and/or other gene (e.g., a therapeutic RNA or protein) is codon optimized (e.g., codon optimized for expression in human cells). In some embodiments, codon optimization is implemented for RNA optimization. In some embodiments, RNA optimization comprises one or more of the following modifications compared to the wild-type RNA molecule: In some embodiments, RNA optimization comprises reducing the uracil (U) load of an RNA molecule. In some embodiments, RNA optimization comprises reducing the GC % content of an RNA molecule. In some embodiments, RNA optimization comprises reducing the length and/or number of intron sequences of an RNA molecule. In some embodiments, RNA optimization comprises reducing RNA binding motifs or sites within an RNA molecule. In some embodiments, RNA optimization comprises lowering AG (free energy) of an RNA molecule. In some embodiments, RNA optimization comprises reducing the nucleotide repeats found in a sequence of an RNA molecule. In some embodiments, RNA optimization comprises increased human or tissue tRNA frequency usage (AKA codon-usage) of an RNA molecule. In some embodiments, RNA optimization comprises reducing the number of palindromic sequences in an RNA molecule. In some embodiments, RNA optimization comprises maximizing pairing of bases of an in an RNA molecule. In some embodiments, RNA optimization comprises removing splicing site sequences from an RNA molecule. In some embodiments, RNA optimization comprises removing rare codons or other slowly translated codons. A person with skill in the art will be able to generate a polynucleotide encoding any one of the polypeptides disclosed herein, based on the polypeptide sequence, as provided herein.

In some embodiments, a nucleic acid encoding an engineered enzyme (e.g. a driver) and/or a gene delivery construct comprises a promoter. In some embodiments, the promoter is a naturally occurring promoter. In some embodiments, the promoter is a recombinant promoter. In some embodiments, the promoter is a constitutive, inducible, and/or tissue or cell specific promoter.

A “nuclear localization sequence” or “NLS” refers to an amino acid sequence that promotes import of a non-nuclear polypeptide into the cell nucleus. Nuclear localization sequences and methods for assessing an NLS peptide's ability to direct a polypeptide to the nucleus are known to those with skill in the art, and examples are provided herein.

In some embodiments, two molecules or components (i.e., nucleic acid to nucleic acid, polypeptide to polypeptide, etc.) may be linked together via a linker. The linker can be an amino acid sequence (about 2 to about 100 amino acids or about 2 to about 50 amino acids) in the case of a linker joining two polypeptides. For example, a retroelement-derived polypeptide may be fused to a heterologous polypeptide by an amino acid linker known to those with skill in the art, and examples are provided herein.

The terms “retrotransposon”, “retrotransposable element”, “RTE” or “retroelement” may be used interchangeably herein. Without wishing to be bound to theory, retrotransposons are genetic elements that are ubiquitous components of the genomic DNA of many vertebrate and non-vertebrate organisms and can amplify themselves via an RNA intermediate.

The term “retroelement-derived polypeptide” refers to a polypeptide that is based on one or more naturally occurring proteins encoded by a naturally occurring retroelement or a portion thereof. In some embodiments, retroelement-derived polypeptide may comprise one or more amino acid modifications (e.g. amino acid substitutions, amino acid deletions, amino acid additions, or amino acid truncations) thereby generating a variant that has a different amino acid compared to the one or more naturally occurring proteins.

A “DNA polymerase” refers to a polymerase that can catalyze synthesis of a nucleic acid strand. The DNA polymerase activity of a DNA polymerase domain in a retroelement-derived polypeptide may be a DNA-dependent DNA polymerase, which may be referred to herein as a “DNA pol” that catalyzes synthesis of a DNA strand based on a DNA template strand. The DNA polymerase may be an RNA-dependent DNA polymerase, which may also be referred to here as a reverse transcriptase or “RT” that catalyzes synthesis of a DNA strand based on an RNA template strand. In some embodiments, retroelement derived polypeptide comprises a DNA pol domain. In some embodiments, the DNA polymerase activity is provided by an RT domain.

The nuclease activity of a nuclease domain in a retroelement-derived polypeptide may be referred to herein as “cutting” or “cleaving”. Suitable nucleases will be apparent to those of skill in the art based on this disclosure.

An “Apurinic/apyrimidinic endonuclease” or “APE” refers to polypeptide domain that recognizes and cleaves the sugar-phosphate backbone of DNA at abasic sites when found in the context of duplex DNA. It can recognize and cleave not only true abasic sites but other substrates including tetrayhydrofuran moieties which lack an oxygen atom on the 1′ carbon of the sugar ring.

An “APE-type retroelement” refers to a non-LTR retroelement that contains an Apurinic/apyrimidinic endonuclease sequence.

A “gene delivery construct” (or “gene delivery nucleic acid”) comprises a sequence of interest, which may be referred to as a transgene, that encodes a RNA or protein (e.g. a therapeutic RNA or protein). In some embodiments, a gene delivery construct may comprise a plurality of transgenes. The gene delivery construct may further one or more one or more 5′ regulatory nucleic acid sequence elements and/or one or more 3′ nucleic acid sequence elements. In some embodiments, the gene delivery construct does not include any 5′ or 3′ regulatory nucleic acid sequences. In some embodiments the gene delivery construct includes one or more 5′ and/or 3′ regulatory nucleic acid sequences. In some embodiments the regulatory nucleic acid sequences are derived from a non-LTR retroelement. In some embodiments, the gene delivery construct includes a promoter. In some embodiments, the gene delivery construct includes an open reading frame encoding a RNA or protein. In some embodiments, the gene delivery construct includes untranslated regions (UTRs) that stabilize the RNA transcript. In some embodiments, the gene delivery construct includes a polyadenylation signal. In some embodiments, the gene delivery construct includes homology arms that direct integration to one or more genomic sites of interest. In some embodiments, the gene delivery construct has sequence elements that interact with the driver. In such cases, a given gene delivery construct may be referred to by the retroelement from which the retroelement-derived polypeptide comprised in the driver was derived. For example, a gene delivery construct having sequence elements that interact with a driver comprising a retroelement-derived polypeptide derived from the ZFL2-2 retroelement may be referred to herein as a “ZFL2-2 gene delivery construct”. As another example, a gene delivery construct having sequence elements that interact with a driver comprising a retroelement-derived polypeptide derived from the Vingi-1_Acar retroelement may be referred to herein as a “Vingi-1 gene delivery construct”.

A “driver nucleic acid” (or “driver construct”) encodes a “driver” or “driver polypeptide” which includes an engineered protein comprising a retroelement-derived polypeptide. The driver may be referred to by the retroelement from which the retroelement-derived polypeptide was derived. For example, a driver comprising a retroelement-derived polypeptide derived from the ZFL2-2 retroelement (optionally with heterologous domains and/or optionally with amino acid modifications) may be referred to herein as a ““ZFL2-2 driver”. An another example, a driver comprising a retroelement-derived polypeptide derived from the Vingi-1_Acar retroelement (optionally with heterologous domains and/or optionally with amino acid modifications) may be referred to herein as a ““Vingi-1_Acar driver” or “Vingi-1 driver”).

The retroelement-derived polypeptide may have endonuclease activity and/or polymerase activity. In some embodiments, the driver has endonuclease activity. In some embodiments, the driver has polymerase activity. In some embodiments, polymerase activity comprises reverse transcriptase activity. In some embodiments, the driver has endonuclease activity and polymerase activity. In some embodiments, the driver has endonuclease activity and reverse transcriptase activity. In some embodiments, the driver RNA construct expressing the driver/driver polypeptide includes untranslated regions (UTRs) that stabilize the RNA transcript.

In some embodiments, the gene delivery nucleic acid and the driver nucleic acid are separate nucleic acids, i.e., in trans. In some embodiments, the gene delivery nucleic acid and the driver nucleic acid are present on a single nucleic acid, i.e., in cis. For clarity, in a trans configuration, the gene delivery nucleic acid includes a transgene which may be flanked 5′ and 3′, independently, with one or more regulatory elements. In a trans configuration, in some embodiments the gene delivery nucleic acid includes a transgene which is flanked 5′ and 3′, independently, with one or more regulatory elements. In a cis configuration, the gene delivery nucleic acid includes an adjacent driver nucleic acid and may further include one or more regulatory elements at the termini of the nucleic acid. In a cis configuration, the gene delivery nucleic acid includes an adjacent driver nucleic acid and further includes one or more regulatory elements at the termini of the nucleic acid. These embodiments are further depicted in the drawings.

The term “efficiency” with respect to gene delivery as used herein refers to the percent of total gene insertions/integrations at the target site. Efficiency can be measured by amplicon sequencing and comparing the number of insertions to non-insertions at a target site and characterizing as a percentage.

The term “specificity” with respect to gene delivery as used herein refers to the fidelity of insertion at a specific target site. An engineered protein with high specificity would exhibit few or no off-target insertions/integrations compared to insertions into a safe harbor site. Specificity may be measured by amplicon sequencing of known or predicted off-target sites.

The term “accuracy” with respect to gene delivery as used herein refers to the percentage of correct insertions/integrations (e.g., full sequence insertions/integrations) at the target site and can be measured by sequencing the target site and comparing correct insertions to total insertions at the site of interest.

The term “fidelity” with respect to gene delivery as used herein refers to the error rate as measured at a per nucleotide basis of the inserted/integrated sequence compared to the desired sequence.

The term “processivity” with respect to gene delivery as used herein is a measure of the length of insert that may be incorporated in its entirety. In some embodiments, large insertions are desired. A large insertion may be insertion of a nucleic acid sequence of about 20 to about 10,000 bases or more. For example, about 20 bases, about 50 bases, about 100 bases, about 200 bases, about 300 bases, about 400 bases, about 500 bases, about 200 bases, about 300 bases, about 400 bases, about 500 bases, about 750 bases, about 1000 bases, about 1250 bases, about 1500 bases, about 2,000 bases, about 3,000 bases, about 4,000 bases, about 5,000 bases, about 6,000 bases, about 7,000 bases, about 8,000 bases, about 9,000 bases, about 10,000 bases or more.

Accordingly, some aspects of the application relate to methods and compositions for delivering a transgene (e.g., a heterologous nucleic acid comprising a gene of interest) to a cell by promoting integration of the transgene into a target nucleic acid in the cell (e.g., into a cellular nucleic acid, for example into the genome of the cell). In some aspects, a gene delivery construct (or gene delivery nucleic acid) comprises a transgene (e.g., a heterologous nucleic acid comprising a gene of interest, for example encoding a therapeutic RNA or protein) optionally independently flanked by one or two terminal regions from a retroelement (e.g., 5′ and 3′ regulatory regions of a non-LTR element). In some embodiments, a gene delivery construct (or gene delivery nucleic acid) comprises a transgene flanked by one or two terminal regions from a retroelement (e.g., 5′ and 3′ regulatory regions of a non-LTR element).

In some embodiments, integration of the transgene from the gene delivery construct into a target nucleic acid is mediated, at least in part, by an engineered protein comprising a retroelement-derived polypeptide which may be referred to herein as “driver” or “driver protein”, and which may be encoded by a “driver nucleic acid”. In some embodiments, the retroelement-derived polypeptide is derived from a non-LTR retroelement. In some embodiments, the engineered protein comprise a retroelement-derived polypeptide fused to a heterologous polypeptide that promotes integration of the transgene into the target nucleic acid (e.g., by increasing integration efficiency, specificity, accuracy, fidelity and/or processivity and/or by redirecting integration to a different location in the target nucleic acid relative to an unmodified retroelement polypeptide). In some embodiments, the engineered protein is encoded by a nucleic acids (e.g., RNA or DNA) that is provided along with a gene delivery construct.

The nucleic acid encoding the engineered protein may be included on the gene delivery nucleic acid in cis, or alternatively provided on one or more separate nucleic acids (e.g. as a driver nucleic acid) in trans. In some embodiments, the engineered protein is provided as polypeptide along with the gene delivery construct.

Accordingly, in some embodiments a nucleic acid (e.g., a DNA or RNA) may comprise a sequence of interest (e.g., a transgene that encodes a therapeutic RNA or protein), and in some embodiments a nucleic acid (e.g., a DNA or RNA) may comprise a sequence that encodes an engineered protein. In some embodiments, a DNA that encodes a protein may be transcribed to produce an RNA having a nucleotide sequence that codes for (e.g., can be translated into) a protein (e.g., a therapeutic protein or an engineered protein). In some embodiments, DNA may be transcribed to produce an RNA that itself is functional, for example a regulatory RNA. In some embodiments, the RNA may be a therapeutic RNA.

### Retroelement Polypeptides

In some embodiments, an engineered protein encoded by a driver construct may comprise a retroelement-derived polypeptide that is a naturally occurring retroelement protein, or variant thereof. In some embodiments, the retroelement-derived polypeptide comprises a nuclease activity. In some embodiments, the retroelement-derived polypeptide comprises a reverse transcriptase activity. In some embodiments, the retroelement-derived polypeptide comprises a nuclease domain. In some embodiments, the retroelement-derived polypeptide comprises a full length, naturally occurring retroelement-derived protein. In some embodiments, the retroelement-derived polypeptide includes one or more amino acid modification relative to a naturally occurring sequence.

In some embodiments, a retroelement-derived polypeptide is based on a naturally occurring retroelement protein or portion thereof, for example derived from a non-LTR retrotransposon.

In some embodiments, a retroelement-derived polypeptide disclosed herein is mutated or modified compared to a non-LTR retrotransposons.

### Gene Delivery Constructs

In some embodiments, an RNA molecule encoded in a gene delivery construct and encoding a transgene contains homology arms having homology to nucleic acid sequences of one or more genomic sites of interest in the human genome, to direct integration to the one or more genomic sites of interest. In some embodiments, each homology arm is independently selected and is from about 4 to about 200 nucleotides in length or more, for example about 4, 10, 15, 20, 25, 30, 35, 45, 50, 55, 60, 65, 70, 75, 80, 85, 90, 95, 100, 200 or more nucleotides in length. In some embodiments, the homology arms correspond to a sequence in the 28S rDNA locus in the human genome. In some embodiments, the homology arms correspond to a sequence in the AAVS1 locus in the human genome. In some embodiments, the nucleic acid sequences of the homology arms are in a reading frame that is different than the open reading frame of the transgene. In some embodiments, the nucleic acid sequences of the homology arms are in the same reading frame as the transgene.

Accordingly, some aspects of the application relate to methods and compositions for delivering a transgene (e.g., a heterologous nucleic acid comprising a gene of interest) to a cell by promoting integration of the transgene into a target nucleic acid in the cell (e.g., into a cellular nucleic acid, for example into the genome of the cell). In some aspects, a gene delivery construct comprises a transgene (e.g., a heterologous nucleic acid comprising a gene of interest, for example encoding a therapeutic RNA or protein) flanked by one or two terminal regions from a retroelement (e.g., terminal repeats of a long terminal repeat (LTR) element, or 5′ and 3′ regulatory regions of a non-LTR element). In some embodiments, integration of the transgene into a target nucleic acid is mediated, at least in part, by a engineered protein comprising a retroelement-derived polypeptide (e.g., a polypeptide derived from a non-LTR retroelement or from an LTR retroelement). In some embodiments, the engineered protein comprises a retroelement-derived polypeptide fused to a heterologous polypeptide that promotes integration of the transgene into the target nucleic acid (e.g., by increasing integration efficiency and/or by redirecting integration to a different location in the target nucleic acid relative to an unmodified retroelement-derived polypeptide). In some embodiments, the engineered protein can be encoded by one or more nucleic acids (e.g., RNA or DNA) that are provided along with the gene delivery construct (e.g., included on the gene delivery nucleic acid in cis or provided on one or more separate nucleic acids in trans). In some embodiments, the engineered protein can be provided as a polypeptide along with the gene delivery construct.

Accordingly, in some embodiments, a nucleic acid (e.g., a DNA or RNA) may comprise a sequence of interest (e.g., a transgene that encodes a therapeutic RNA or protein), and in some embodiments a nucleic acid (e.g., a DNA or RNA) may comprise a sequence that encodes an engineered protein. In some embodiments, a DNA that encodes a protein may be transcribed to produce an RNA having a nucleotide sequence that codes for (e.g., can be translated into) a protein (e.g., a therapeutic protein or an engineered protein). In some embodiments, a DNA may be transcribed to produce an RNA that itself is functional, for example a regulatory RNA. In some embodiments, the RNA may be a therapeutic RNA.

In some aspects, an engineered protein comprises a retroelement-derived polypeptide (e.g., containing a reverse transcriptase) of a naturally occurring retroelement protein or an amino acid sequence variant thereof. In some embodiments, the retroelement-derived polypeptide comprises a full length, naturally occurring retroelement-derived protein. In some embodiments, the retroelement-derived polypeptide includes one or more amino acid modification relative to a naturally occurring sequence.

In some embodiments, an engineered protein comprises a retroelement-derived polypeptide fused to one or more heterologous polypeptides or domains thereof (e.g., at the N-terminus of the retroelement-derived polypeptide, at the C-terminus of the retroelement-derived polypeptide, internally within the retroelement-derived polypeptide, or any combination thereof).

In some embodiments, a heterologous polypeptide or domain thereof comprises one or more RNA/DNA processing polypeptides (e.g., RNase H), one or more RNA/DNA repair polypeptides (e.g., Rad51), one or more nucleic acid binding polypeptides, one or more nucleosome binding polypeptides, or any combination thereof. In some embodiments, a heterologous polypeptide or domain thereof may comprise a localization signal, e.g., a nuclear localization sequence (NLS) or a nucleolar localization sequence (NoLS). In some embodiments, a heterologous polypeptide or domain thereof further comprises one or more linkers (e.g., at the N- or C-terminus of the heterologous polypeptide and/or within the heterologous polypeptide, for example between different domains or sequences within the heterologous polypeptide). In some embodiments, an engineered protein comprises a heterologous polypeptide or domain thereof having one or more amino acid substitutions relative to a naturally occurring counterpart. In some embodiments, the engineered protein (e.g., comprising one or more fusions and/or one or more amino acid substitutions) redirects and/or increases the efficiency of integration of a transgene into a target nucleic acid relative to a naturally occurring retroelement protein.

In some embodiments a gene-delivery construct may include as a transgene (or one transgene out of a plurality of transgenes), a detection peptide to detect integration of the transgene by a given driver. Alternatively, in a cis configuration, a cis driver/transgene construct may comprise, as a transgene (or one transgene out of a plurality of transgenes), the detection peptide. In some embodiments, a detection peptide is a human influenza hemagglutinin (HA), FLAG, green fluorescent protein (GFP or variants such as EGFP), or mCherry peptide.

As used herein, an RNA/DNA processing polypeptide is an enzyme that directly causes chemical changes to RNA and/or DNA, for example by promoting RNA degradation. As used herein, an RNA/DNA repair polypeptide is a polypeptide that is or interacts with a host repair protein that acts on RNA and/or DNA. As used herein, a host repair protein is a host processing enzyme. As used herein, a nucleic acid binding polypeptide binds to RNA and/or DNA. As used herein, a nucleosome binding polypeptide binds to nucleosomes.

A “retroelement-derived polypeptide” refers to a polypeptide that is based on one or more naturally occurring proteins encoded by a naturally occurring retroelement or a portion thereof. In some embodiments, the retroelement-derived polypeptide comprises one or more proteins or domains thereof that are found in a retroelement, optionally a wild type or naturally occurring retroelement. In some embodiments, the retroelement-derived polypeptide may be a full length naturally occurring and/or wild type retroelement, or a portion thereof. In some embodiments, a retroelement-derived polypeptide includes at least one of reverse transcriptase domain and endonuclease domain. In some embodiments, retroelement-derived polypeptide may comprise one or more amino acid modifications (e.g. amino acid substitutions, amino acid deletions, amino acid additions, or amino acid truncations) thereby generating a variant that has a different amino acid compared to the one or more naturally occurring proteins. In some embodiments, the naturally occurring retroelement may be a non-LTR retroelement. In some embodiments, the non-LTR retrotransposon is a long-interspersed element polypeptide (LINE) or a short interspersed element (SINE). LINEs (Long INterspersed Elements) and SINEs (Short INterspersed Elements) are non-LTR retrotransposons that are found in almost all eukaryotes, and are among the most common retrotransposons in the human genome. Wild-type LINEs typically encode a reverse transcriptase and an endonuclease. SINEs do not encode reverse transcriptase or endonuclease, and depend on reverse transcriptase and endonuclease encoded by partner LINEs. In some embodiments, the LINE is a LINE-1, a LINE-2, or a LINE-3. In some embodiments, the LINE is from a clade selected from: CRE, R4, Hero, NeSL, R2, RandI, Proto1, L1, Tx1, RTEPT, Proto2, RTEX, RTE, Outcast, Ingi (which includes sub-clade Vingi), I, Nimb, Tad1, Loa, R1, Jockey, Rex1, CR1, L2, L2A, L2B, Daphne, or Crack. In some embodiments, the engineered protein comprises a naturally occurring polypeptide sequence including a reverse transcriptase domain. In some embodiment, a naturally occurring polypeptide including a reverse transcriptase domain is from a group II intron, or a retron.

In some embodiments, an engineered protein is encoded by a first nucleic acid (e.g., an RNA molecule or a DNA molecule), that can be provided, in trans or in cis, with second a nucleic acid encoding a gene of interest (e.g., flanked by terminal regions). In some embodiments, the first nucleic acid and/or the second nucleic acid comprises one or more chemical and/or sequence modifications. In some embodiments, the modification is an RNA CAP, a modified polyA length (e.g., relative to a natural polyA), a chemical modification (e.g., a pseudouridine and/or a methylpseudouridine), a 5′ UTR modification, a 3′ UTR modification, a modified Kozak sequence, a modified (e.g., truncated) stem loop, an RNA stabilization motif, a 5-methoxyuridine (5-MO-U) modification, a 5-methylcytidine (5mC) modification, or one or more additional and/or modified microsatellites. In some embodiments, a nucleic acid encoding an engineered protein and/or a transgene (e.g., encoding a therapeutic RNA or protein) is codon optimized (e.g., codon optimized for expression in human cells). In some embodiments, codon optimization is for RNA optimization. In some embodiments, RNA optimization comprises reducing the Uracil (U) load of an RNA molecule.

In some embodiments, a nucleic acid encoding an engineered protein and/or other gene comprises a promoter. In some embodiments, the promoter is a naturally occurring promoter. In some embodiments, the promoter is a recombinant promoter. In some embodiments, the promoter is a constitutive, inducible, and/or tissue or cell specific promoter.

In some aspects, methods and compositions described in this application are useful for delivering one or more transgenes (e.g., a heterologous nucleic acid comprising a gene of interest) to a host cell and promoting integration of the heterologous nucleic acid into a target nucleic acid (e.g., a genomic nucleic acid) in the host cell, for example for therapeutic purposes (e.g., to provide or supplement expression of an RNA and/or polypeptide that provides a therapeutic benefit to a subject, for example a human subject having a disease or disorder associated with a loss of normal gene function).

In some aspects, methods and compositions of the application are useful for high efficiency integration of a transgene into a target region (e.g., a sequence specific target) within a target nucleic acid (e.g., within a host genome). In some embodiments, methods and compositions of the application are useful to redirect integration of a transgene to a particular target region (e.g., relative to integration mediated by a naturally occurring protein).

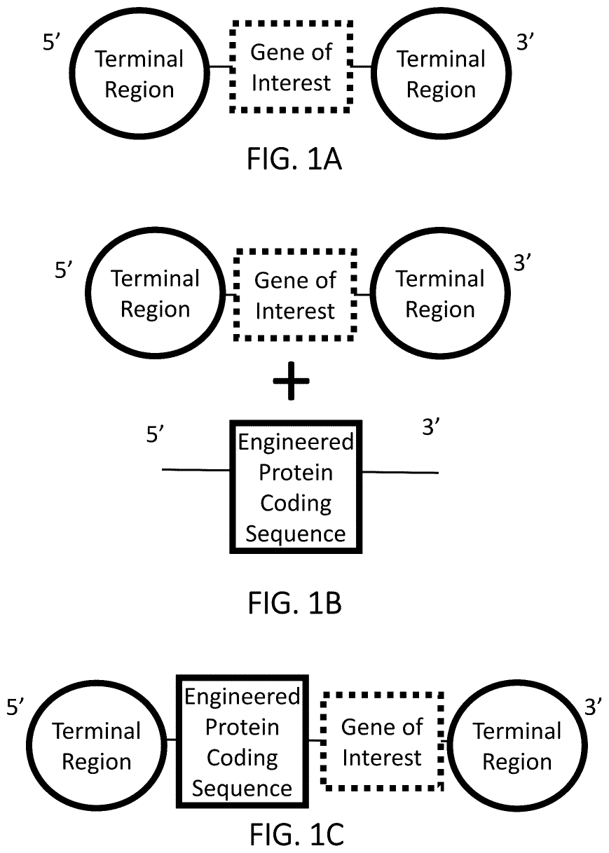

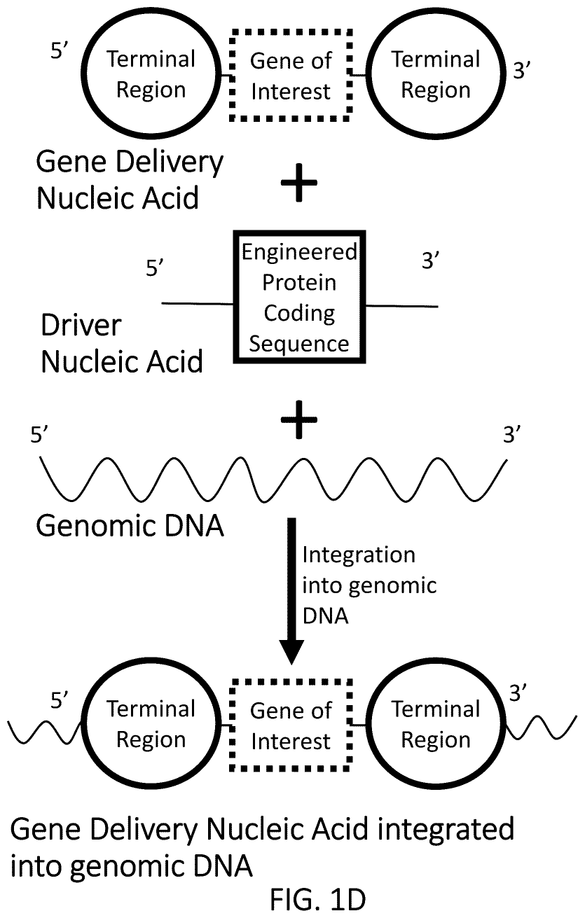

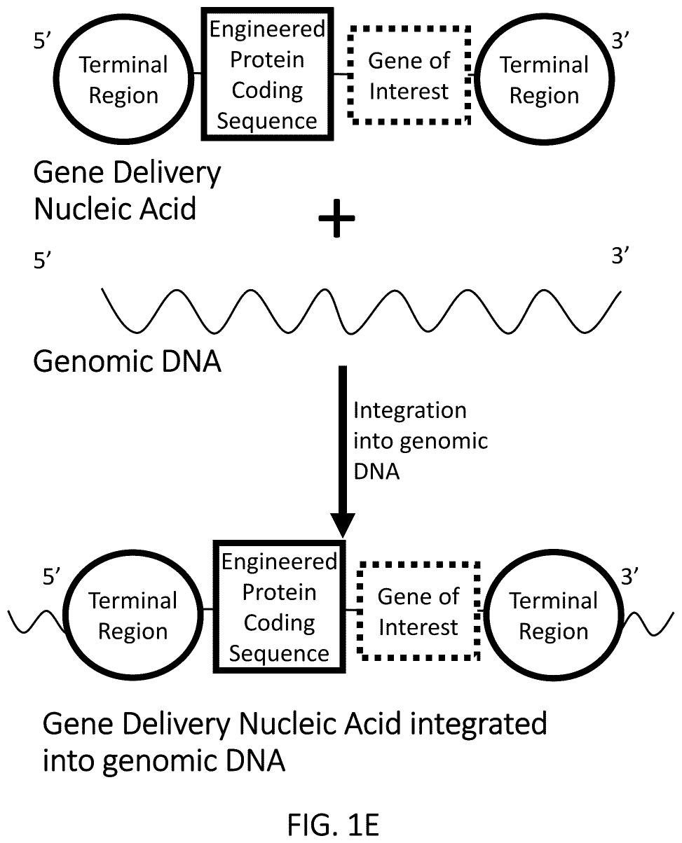

FIGS. 1A-1E illustrate a non-limiting example of a gene delivery construct. In some embodiments, a gene delivery construct is a nucleic acid (e.g., a DNA or an RNA) comprising a transgene (e.g., a heterologous nucleic acid comprising a gene of interest) flanked by two terminal regions as illustrated in FIG. 1A. In some embodiments, the terminal regions contain sequences recognized by retroelement-derived polypeptides that promote integration of the transgene into a target nucleic acid. In some embodiments, the terminal regions are distinct regions. In some embodiments, the terminal regions are terminal repeat regions. In some embodiments, a terminal region is a 5′ UTR, a 3′ UTR, or a portion thereof (e.g., from a non-LTR retroelement). In some embodiments, a terminal region is a long terminal repeat (LTR) or a portion thereof (e.g., from an LTR retroelement).

In some embodiments, the one or more engineered proteins (e.g., comprising a retroelement-derived polypeptide, or a retroelement-derived polypeptide fused to a heterologous polypeptide) are encoded on one or more nucleic acids that are distinct and separate from the nucleic acid that comprises the gene of interest (e.g., in trans). A non-limiting example of a trans configuration is illustrated in FIG. 1B. FIG. 1B shows a non-limiting example of a configuration where the gene of interest is flanked by terminal regions on a first nucleic acid and the engineered protein is encoded by a separate second nucleic acid that is distinct from the first nucleic acid. In some embodiments, the second nucleic acid does not contain terminal regions such that only the gene of interest is integrated into the genome of a host cell and the sequences encoding the engineered protein and any other genes that are on the second nucleic acid are not integrated into the host genome. The configuration illustrated in FIG. 1B shows only the engineered protein coding sequence encoded by the second nucleic acid. In some embodiments one or more additional genes also may be included on the first and/or second nucleic acid. As used herein, an engineered protein coding sequence and an engineered protein-encoding nucleic acid are used interchangeably to refer to a nucleic acid (e.g., an RNA or a DNA) comprising a sequence that codes for the engineered protein.

In some embodiments, one or more engineered proteins (e.g., comprising a retroelement-derived polypeptide, or a retroelement-derived polypeptide fused to a heterologous polypeptide) are encoded on the same nucleic acid that comprises the gene of interest (e.g., in cis). A non-limiting example of a cis configuration is illustrated in FIG. 1C. FIG. 1C shows a configuration with an engineered protein encoding nucleic acid upstream of a gene of interest.

However, other configurations can be provided, for example including an engineered protein encoding nucleic acid that is downstream from a gene of interest, and/or wherein the engineered protein coding sequence is outside of the terminal repeats (e.g., it is either upstream or downstream from the gene of interest flanked by the terminal repeats).

A non-limiting example of a trans configuration, in which the gene of interest is integrated into genomic DNA is illustrated in FIG. 1D. FIG. 1D shows a configuration where the gene of interest is flanked by terminal regions on a first nucleic acid (gene delivery construct) and the engineered protein (driver) coding sequence is encoded by a separate second nucleic acid (driver construct) that is distinct from the first nucleic acid. The driver nucleic acid encodes an engineered retroelement-derived polypeptide that promotes integration of the gene of interest into genomic DNA (FIG. 1D). In some embodiments, the gene delivery nucleic acid is DNA. In some embodiments, the gene delivery nucleic acid is RNA. In some embodiments, the driver nucleic acid is DNA. In some embodiments, the driver nucleic acid is RNA. In some embodiments one or more additional genes also may be included on the gene delivery nucleic acid and/or the driver nucleic acid.

A non-limiting example of a cis configuration, in which the gene of interest and the engineered protein coding sequence are integrated into genomic DNA is illustrated in FIG. 1E.

The engineered protein coding sequence encodes an engineered retroelement-derived polypeptide that promotes integration of the gene of interest into genomic DNA. Other configurations are envisioned and can be provided, for example including an engineered protein coding sequence that is downstream from the gene of interest, and/or wherein the gene of interest is flanked by terminal repeats but the engineered protein coding sequence is not flanked by the terminal repeats (e.g., it is either upstream or downstream from the gene of interest flanked by the terminal repeats). In some embodiments, the nucleic acid is DNA. In some embodiments, the nucleic acid is RNA.

### Engineered Proteins and Nucleic Acids Encoding the Engineered Proteins

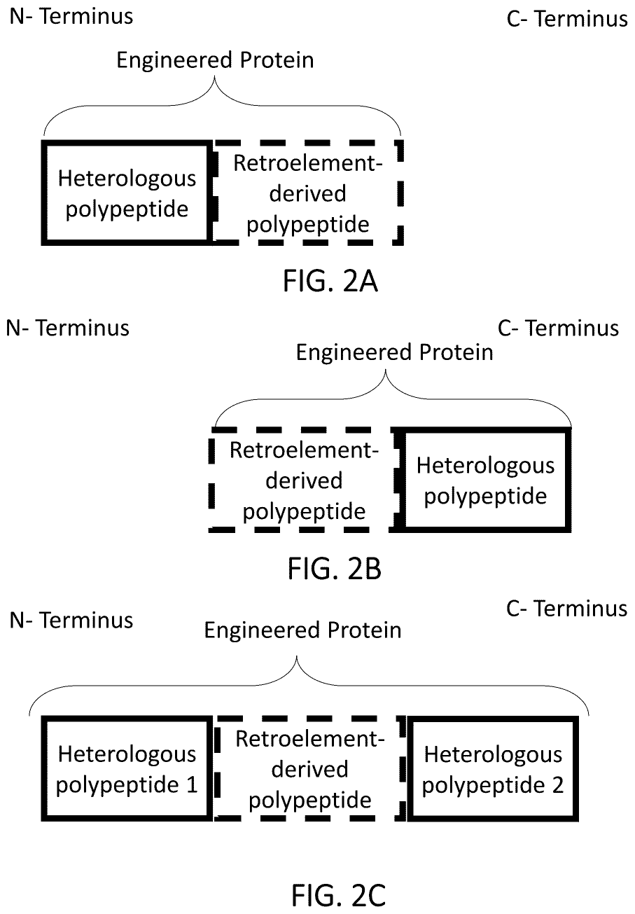

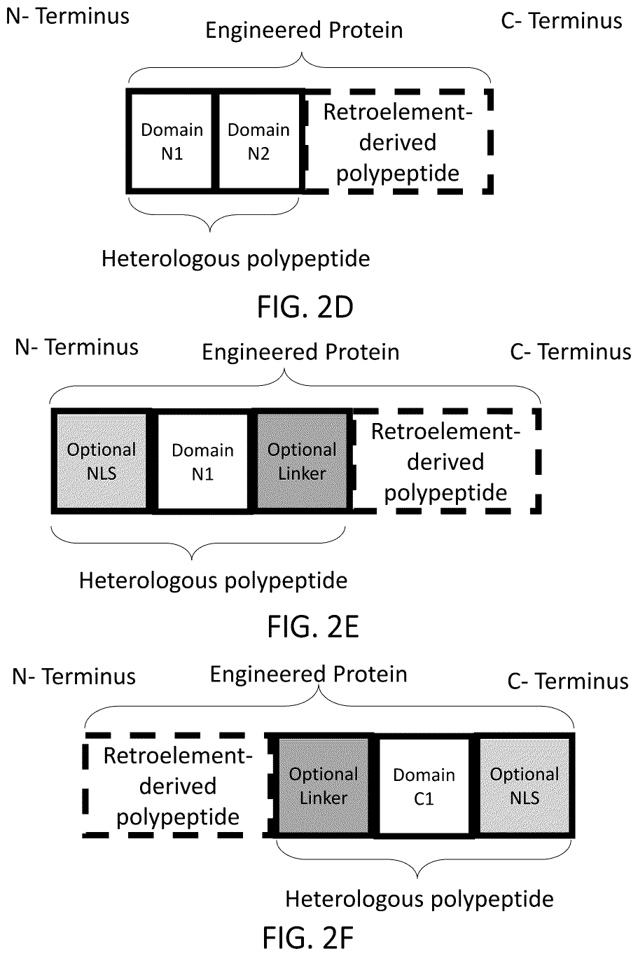

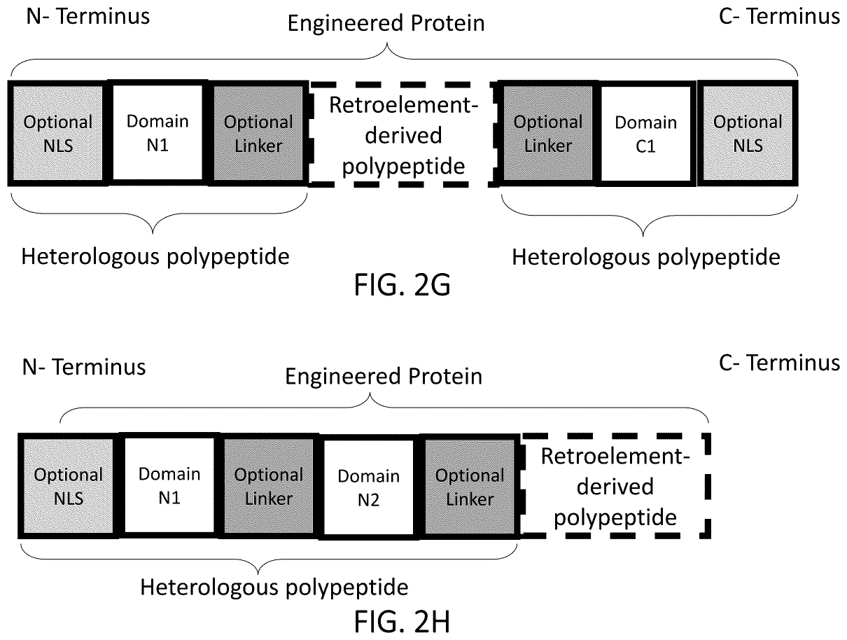

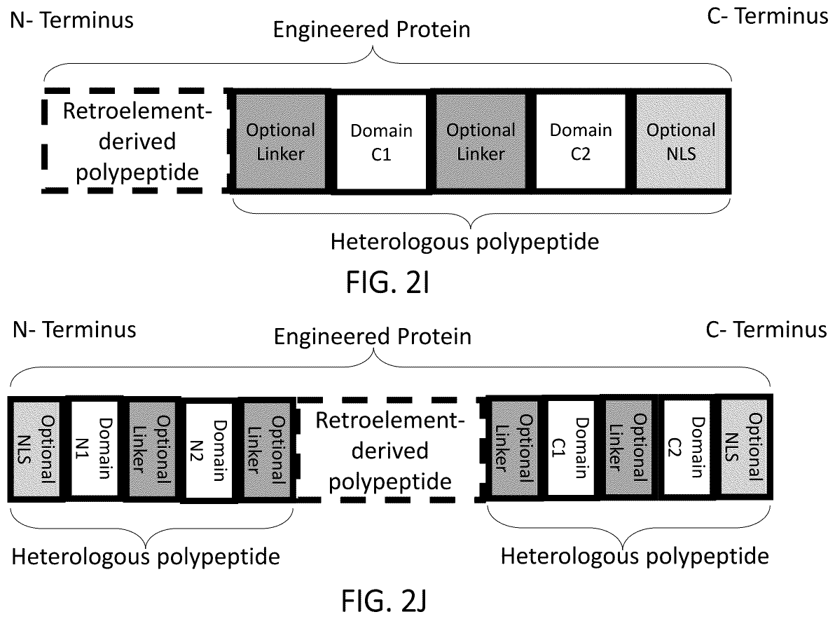

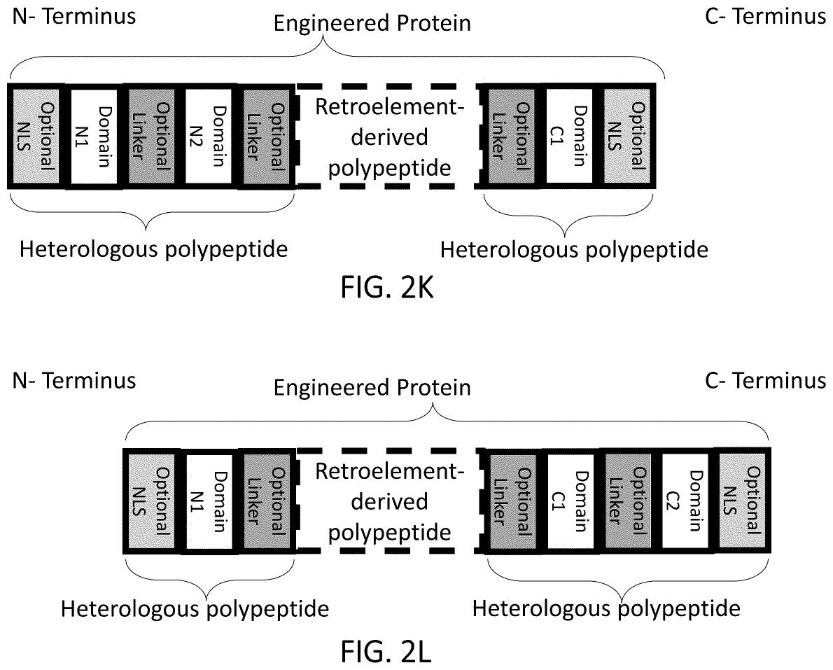

In some embodiments, a nucleic acid encodes an engineered protein. In some embodiments, the nucleic acid is DNA. In some embodiments, the nucleic acid is RNA. Non-limiting examples of different engineered protein configurations that can be encoded by a nucleic acid are illustrated in FIGS. 2A-2L.

FIGS. 2A-2L illustrate non-limiting examples of different configurations of an engineered protein comprising a retroelement-derived polypeptide and one or more heterologous polypeptides that can promote and/or redirect integration of a transgene into a target nucleic acid (e.g., into the genome of a cell). In some embodiments, an engineered protein comprises at least one heterologous polypeptide (e.g., comprising one or more RNA/DNA processing polypeptides, RNA/DNA repair polypeptides, nucleic acid binding polypeptides, and/or nucleosome binding polypeptides) fused to the N-terminus of a retroelement-derived polypeptide, the C-terminus of a retroelement-derived polypeptide, and/or internally (e.g., between two domains of a retroelement-derived polypeptide).

FIG. 2A illustrates a non-limiting example of an engineered protein comprising a retroelement-derived polypeptide that has a heterologous polypeptide fused to its N-terminus.

FIG. 2B. illustrates a non-limiting example of an engineered protein comprising a retroelement-derived polypeptide that has a heterologous polypeptide fused to its C-terminus.

FIG. 2C illustrates a non-limiting example of an engineered protein comprising a retroelement-derived polypeptide that has a first heterologous polypeptide fused to its N-terminus and a second heterologous polypeptide fused to its C-terminus.

In some embodiments, each heterologous polypeptide can itself independently comprise one or more (e.g., two, three, four, or more) different polypeptides (for example one or more of an RNA/DNA processing polypeptide, RNA/DNA repair polypeptide, nucleic acid binding polypeptide, and/or nucleosome binding polypeptide), optionally along with one or more linkers and/or localization sequences.

FIG. 2D illustrates a non-limiting embodiment of an N-terminal heterologous polypeptide comprising a first domain and a second domain (domain N1 and domain N2). FIG. 2E illustrates a non-limiting embodiment of an N-terminal heterologous polypeptide comprising a linker, a domain, and a nuclear localization sequence (NLS). FIG. 2F illustrates a non-limiting embodiment of a C-terminal heterologous polypeptide comprising a linker, a domain, and an NLS. FIG. 2G illustrates a non-limiting example of an N-terminal heterologous polypeptide comprising a first linker, a first domain, and a first NLS; and a C-terminal heterologous polypeptide comprising a second linker, a second domain, and a second NLS. FIG. 2H illustrates a non-limiting example of an N-terminal heterologous polypeptide comprising a first linker, a first domain, a second linker, a second domain, and an NLS. FIG. 2I illustrates a non-limiting example of a C-terminal heterologous polypeptide comprising a first linker, a first domain, a second linker, a second domain, and an NLS. FIG. 2J illustrates a non-limiting example of an N-terminal heterologous polypeptide comprising a first linker, a first domain, a second linker, a second domain, and a first NLS; and a C-terminal heterologous polypeptide comprising a third linker, a third domain, a fourth linker, a fourth domain, and a second NLS.

FIG. 2K illustrates a non-limiting example of an N-terminal heterologous polypeptide comprising a first linker, a first domain, a second linker, a second domain, and a first NLS; and a C-terminal heterologous polypeptide comprising a third linker, a third domain, and a second NLS. FIG. 2L illustrates a non-limiting example of an N-terminal heterologous polypeptide comprising a first linker, a first domain, and a first NLS; and a C-terminal heterologous polypeptide comprising a second linker, a second domain, a third linker, a third domain, and a second NLS. However, other configurations can include additional and/or different elements within a heterologous polypeptide that is fused to the N-terminus of a retroelement-derived polypeptide, the C-terminus of the retroelement-derived polypeptide, and/or internally with the retroelement-derived polypeptide.

In some embodiments, each domain independently comprises an RNA/DNA processing polypeptide, an RNA/DNA repair polypeptide, a nucleic acid binding polypeptide, or a nucleosome binding polypeptide. A linker is optional. In some embodiments, a linker can independently be present or absent in the N-terminal, C-terminal, and/or internal heterologous polypeptide. An NLS is optional. In some embodiments, the NLS is present or absent in the N-terminal, C-terminal, and/or internal heterologous polypeptide. In some embodiments, the relative position of the NLS and/or one or more of the domains and or linkers can be different.

In some embodiments, the NLS can be fused to the other elements of a heterologous polypeptide via a further optional linker. In some embodiments, a heterologous polypeptide includes an NoLS (e.g., in addition to the NLS or instead of the NLS). In some embodiments, an engineered protein comprises a reverse transcriptase domain, e.g., fused to one or more heterologous polypeptides. In some embodiments, an engineered protein comprises an integrase domain, e.g., fused to one or more heterologous polypeptides.

In some embodiments, a heterologous polypeptide (e.g., an N-terminal, a C-terminal, and/or an internal heterologous polypeptide) can include more than two domains and/or linkers.

In some embodiments, an engineered protein comprises one or more domains illustrated in the examples. In some embodiments, an engineered protein has an amino acid sequence of any of SEQ ID NOs: 1-22, 27, 29, 31, 33, 35, 37, 39, 41, 43, 45, 47, 49, 51, 70-246, 248-259, 263-277, 293-307, or 341-368, or an amino acid sequence that is at least 70% identical (e.g., at least 75% identical, at least 80% identical, at least 85% identical, at least 90% identical, at least 95% identical, 99% identical, or 100% identical) to a sequence of any of SEQ ID NOs: 1-22, 27, 29, 31, 33, 35, 37, 39, 41, 43, 45, 47, 49, 51, 70-246, 248-259, 263-277, 293-307, or 341-368.

In some embodiments, an engineered protein has an amino acid sequence of SEQ ID NO 1. In some embodiments, an engineered protein has an amino acid sequence of SEQ ID NO 2. In some embodiments, an engineered protein has an amino acid sequence of SEQ ID NO 3.

In some embodiments, an engineered protein has an amino acid sequence of SEQ ID NO 4. In some embodiments, an engineered protein has an amino acid sequence of SEQ ID NO 5. In some embodiments, an engineered protein has an amino acid sequence of SEQ ID NO 6. In some embodiments, an engineered protein has an amino acid sequence of SEQ ID NO 7. In some embodiments, an engineered protein has an amino acid sequence of SEQ ID NO 8. In some embodiments, an engineered protein has an amino acid sequence of SEQ ID NO 9. In some embodiments, an engineered protein has an amino acid sequence of SEQ ID NO 10. In some embodiments, an engineered protein has an amino acid sequence of SEQ ID NO 11. In some embodiments, an engineered protein has an amino acid sequence of SEQ ID NO 12. In some embodiments, an engineered protein has an amino acid sequence of SEQ ID NO 13. In some embodiments, an engineered protein has an amino acid sequence of SEQ ID NO 14. In some embodiments, an engineered protein has an amino acid sequence of SEQ ID NO 15. In some embodiments, an engineered protein has an amino acid sequence of SEQ ID NO 16. In some embodiments, an engineered protein has an amino acid sequence of SEQ ID NO 17. In some embodiments, an engineered protein has an amino acid sequence of SEQ ID NO 18. In some embodiments, an engineered protein has an amino acid sequence of SEQ ID NO 19. In some embodiments, an engineered protein has an amino acid sequence of SEQ ID NO 20. In some embodiments, an engineered protein has an amino acid sequence of SEQ ID NO 21. In some embodiments, an engineered protein has an amino acid sequence of SEQ ID NO 22. In some embodiments, an engineered protein has an amino acid sequence of SEQ ID NO 27. In some embodiments, an engineered protein has an amino acid sequence of SEQ ID NO 29. In some embodiments, an engineered protein has an amino acid sequence of SEQ ID NO 31. In some embodiments, an engineered protein has an amino acid sequence of SEQ ID NO 33. In some embodiments, an engineered protein has an amino acid sequence of SEQ ID NO 35. In some embodiments, an engineered protein has an amino acid sequence of SEQ ID NO 37. In some embodiments, an engineered protein has an amino acid sequence of SEQ ID NO 39. In some embodiments, an engineered protein has an amino acid sequence of SEQ ID NO 41. In some embodiments, an engineered protein has an amino acid sequence of SEQ ID NO 43. In some embodiments, an engineered protein has an amino acid sequence of SEQ ID NO 45. In some embodiments, an engineered protein has an amino acid sequence of SEQ ID NO 47. In some embodiments, an engineered protein has an amino acid sequence of SEQ ID NO 49. In some embodiments, an engineered protein has an amino acid sequence of SEQ ID NO 51. In some embodiments, an engineered protein has an amino acid sequence of any one of SEQ ID NOs 70-246, 248-259, 263-277, 293-307, or 341-368. In some embodiments, an engineered protein is at least 70% identical (e.g., at least 75% identical, at least 80% identical, at least 85% identical, at least 90% identical, at least 95% identical, 99% identical, or 100% identical) to any one of the aforementioned amino acid sequences.

In some embodiments, an engineered protein is encoded by a nucleic acid. In some embodiments, the nucleic acid encoding an engineered protein is a DNA. In some embodiments, the nucleic acid encoding an engineered protein is an RNA. In some embodiments, a nucleic acid encoding an engineered protein comprises a nucleic acid encoding a heterologous polypeptide. In some embodiments, the nucleic acid has a sequence that is at least 70% identical (e.g., at least 75% identical, at least 80% identical, at least 85% identical, at least 90% identical, at least 95% identical, 99% identical, or 100% identical) to a sequence of any of SEQ ID NOs: 28, 30, 32, 34, 36, 38, 40, 42, 44, 46, 48, 50, 52, 247, 319-326, 328, 397-398 or a fragment of any one thereof that encodes a polypeptide domain.

In some embodiments, a nucleic acid has the sequence of SEQ ID NO 28. In some embodiments, a nucleic acid has the sequence of SEQ ID NO 30. In some embodiments, a nucleic acid has the sequence of SEQ ID NO 32. In some embodiments, a nucleic acid has the sequence of SEQ ID NO 34. In some embodiments, a nucleic acid has the sequence of SEQ ID NO 36. In some embodiments, a nucleic acid has the sequence of SEQ ID NO 38. In some embodiments, a nucleic acid has the sequence of SEQ ID NO 40. In some embodiments, a nucleic acid has the sequence of SEQ ID NO 42. In some embodiments, a nucleic acid has the sequence of SEQ ID NO 44. In some embodiments, a nucleic acid has the sequence of SEQ ID NO 46. In some embodiments, a nucleic acid has the sequence of SEQ ID NO 48. In some embodiments, a nucleic acid has the sequence of SEQ ID NO 40. In some embodiments, a nucleic acid is at least 70% identical (e.g., at least 75% identical, at least 80% identical, at least 85% identical, at least 90% identical, at least 95% identical, 99% identical, or 100% identical) to any one of the aforementioned nucleic acid sequences.

### Retroelement-Derived Polypeptides and Nucleic Acids Encoding the Retroelement-Derived Polypeptides

In some embodiments, a nucleic acid encoding an engineered protein comprises a sequence that encodes a retroelement-derived polypeptide. In some embodiments, the nucleic acid is DNA. In some embodiments, the nucleic acid is RNA. Non-limiting examples of different engineered retroelement-derived polypeptides are described herein and are illustrated in the Examples.

In some embodiments, an engineered protein comprises a retroelement-derived polypeptide. In some embodiments, an engineered protein comprises a modified retroelement-derived polypeptide, for example a retroelement-derived polypeptide that includes one or more amino acid modifications relative to a naturally occurring counterpart. In some embodiments, the retroelement-derived polypeptide, which may be a modified retroelement-derived polypeptide, is fused to one or more heterologous polypeptides (e.g., N-terminally, C-terminally, and/or internally).

In some embodiments, a retroelement-derived polypeptide comprises a reverse transcriptase domain of a retroelement. Non-LTR and LTR retroelements typically encode multi-domain proteins having several enzymatic activities, for example including a reverse transcriptase domain, e.g., from a POL protein of an LTR-RE or an ERV (endogenous retrovirus). In some embodiments, a reverse transcriptase domain is modified to include one or more amino acid modifications relative to a naturally occurring reverse transcriptase domain. In some embodiments, a reverse transcriptase domain is fused to one or more heterologous polypeptides that can promote integration of a transgene flanked by terminal regions (e.g., terminal repeat regions or regulatory sequences), and/or redirect integration of the transgene to a different target sequence. In some embodiments, the retroelement-derived polypeptide is an integrase domain (e.g., from a protein encoded by a retroelement). In some embodiments, an integrase domain is modified to include one or more amino acid modifications relative to a naturally occurring integrase domain. In some embodiments, an integrase domain is fused to one or more polypeptides that can promote and/or redirect integration of a transgene.

In some embodiments, an engineered protein comprises a retroelement-derived polypeptide together with one or more additional domains from a naturally occurring protein (e.g., one or more other domains of a POL protein or an ORF protein) fused to a heterologous polypeptide.

In some embodiments, a retroelement-derived polypeptide is a full-length or essentially full-length protein comprising a retroelement enzyme domain (e.g., a full-length or essentially full-length POL or ORF protein). In some embodiments, an ORF protein is an ORF2 protein (e.g., of LINE-2 retroelement). In some embodiments, a retroelement-derived polypeptide comprises a reverse transcriptase domain, e.g., from a murine leukemia virus.

In some embodiments, the retroelement-derived polypeptide (e.g., reverse transcriptase domain and/or integrase domain) comprises a sequence from an LTR retrotransposon or an ERV. In some embodiments, the reverse transcriptase domain comprises a sequence from a non-LTR retrotransposon. In some embodiments, the reverse transcriptase domain comprises a sequence from a LINE-1, a LINE-2, or a LINE-3 retrotransposon. In some embodiments, the reverse transcriptase domain comprises a sequence from a retrotransposon of clade CRE, R4, Hero, NeSL, R2, RandI, Proto1, L1, Tx1, RTEPT, Proto2, RTEX, RTE, Outcast, Ingi (which includes sub-clade Vingi), I, Nimb, Tad1, Loa, R1, Jockey, Rex1, CR1, L2, L2A, L2B, Daphne, or Crack. In some embodiments, the reverse transcriptase domain comprises a sequence from a LINE 2-2 (L2-2) retrotransposon from clade L2. In some embodiments, the LINE 2-2 retrotransposon is a zebrafish LINE 2-2 (ZFL2-2) retrotransposon.

In some embodiments, a LINE-1, LINE-2, LINE-3, CRE, R4, Hero, NeSL, R2, RandI, Proto1, L1, Tx1, RTEPT, Proto2, RTEX, RTE, Outcast, Ingi (optionally sub-clade Vingi), I, Nimb, Tad1, Loa, R1, Jockey, Rex1, CR1, L2, L2A, L2B, Daphne, or Crack retrotransposon contains a 5′ UTR, an ORF1, an ORF2, a 3′ UTR, or a combination thereof. In some embodiments, the ORF2 contains a reverse transcriptase and an endonuclease domain. In some embodiments, an ORF2 has an apurinic endonuclease (APE) and/or a restriction enzyme like endonuclease (RLE), for example at the N-terminus or at the C-terminus.

In some embodiments, a reverse transcriptase domain is a variant reverse transcriptase domain. In some embodiments, a variant reverse transcriptase domain comprises at least one amino acid substitution that improves at least one of stability, interaction with RNA, or interaction with DNA relative to an unsubstituted reverse transcriptase domain. In some embodiments, the variant reverse transcriptase domain comprises at least one amino acid substitution that stabilizes its association with RNA or DNA relative to an unsubstituted reverse transcriptase domain. In some embodiments, the amino acid substitution adds a positive charge (e.g., via the addition of lysine or arginine), removes a negative charge (e.g., via the removal of an aspartate or glutamate), alters at least one H-bond forming residue, or alters at least one S-bond forming residue. In some embodiments, the amino acid substitution corresponds to a substitution of certain amino acids in the amino acid sequence of a LINE 2-2 reverse transcriptase. In some embodiments, the amino acid substitution corresponds to a substitution selected from the group consisting of D550T, D770H, K815R, S883R, K952R, K542R, N546R, H569R, S577R, H463K, Q478K, K566R, and K815R relative to SEQ ID NO: 51, which is an amino acid sequence of a Line 2-2 retroelement protein.

Accordingly, in some embodiments, a recombinant reverse transcriptase domain is a variant of a reverse transcriptase domain from an LTR retrotransposon. In some embodiments, the recombinant reverse transcriptase domain is a variant of a reverse transcriptase domain from a non-LTR retrotransposon.

In some embodiments, a reverse transcriptase domain comprises a reverse transcriptase sequence from an LTR-RE or an ERV. In some embodiments, a recombinant reverse transcriptase domain comprises a reverse transcriptase domain sequence from a non-LTR element, for example from a LINE-1 or LINE-2 retrotransposon, having at least one stabilizing amino acid substitution. In some embodiments, a recombinant reverse transcriptase domain comprises a reverse transcriptase domain sequence from a LINE 2-2 retrotransposon having at least one stabilizing amino acid substitution. In some embodiments, the LINE 2-2 retrotransposon is from a zebrafish. In some embodiments, the amino acid substitution corresponds to a substitution of a LINE 2-2 reverse transcriptase. In some embodiments, the amino acid substitution corresponds to a substitution selected from the group consisting of I625L, H521P, S737P, P705A, M558L, M733L, M760S, M750L, A757P, H717A, H717K, D497S, I625H, L825G, D278S, L837I, A464P, K762R, A948T, P675S, H698P, L742P, E541K, Q547R, S814P, S672P, N560P, H853P, L514P, L524P, Q449P, H650P, G674P, S800P, I896P, S474P, and D520P relative to SEQ ID NO: 51, which is an amino acid sequence of a Line 2-2 retroelement protein.

In some embodiments, a stabilizing amino acid substitution (e.g., in a reverse transcriptase domain or an integrase domain) is an amino acid substitution that improves packing of hydrophobic residues in the core of the domain, stabilizes a loop region, and/or alters electrostatic, H-bond stability, or S-bond stability. In some embodiments, the substitution and/or addition that stabilizes a loop region is a proline substitution. In some embodiments, the substitution that alters electrostatic, H-bond stability, or S-bond stability adds a positive charge, for example by mutation to lysine or arginine, or mutation from aspartate or glutamate to a non-charged residue such as alanine.

In some embodiments, a retroelement-derived polypeptide (e.g., a reverse transcriptase and/or integrase domain) is fused to an endonuclease domain. In some embodiments, the endonuclease domain is derived from the same protein (e.g., a LINE-1 or a LINE-2 ORF2) as the reverse transcriptase domain. In some embodiments, the endonuclease domain is heterologous to the reverse transcriptase domain (e.g., derived from a different protein). In some embodiments, a heterologous endonuclease includes but is not limited to a Cas nuclease (e.g., a Cas9 nuclease), a Cas9 nickase (e.g., SpCas9 with a H840 mutation), a homing endonuclease, or a FokI nuclease.

In some embodiments, a recombinant endonuclease domain comprises at least one amino acid substitution that improves its association with DNA relative to an unsubstituted endonuclease domain. In some embodiments, the amino acid substitution corresponds to a substitution of a LINE 2-2 endonuclease domain. In some embodiments, the amino acid substitution corresponds to a substitution selected from the group consisting of Y139K and D64K relative to SEQ ID NO: 51.

In some embodiments a retroelement-derived polypeptide comprises a polypeptide encoded by a ZFL2-2 retrotransposon. In some embodiments, the ZFL2-2 polypeptide is a modified ZFL2-2 polypeptide. In some embodiments, a modified ZFL2-2 comprises one or more amino acid modifications relative to a naturally occurring ZFL2-2. In some embodiments, a modified ZFL2-2 has an RNA binding mutation. In some embodiments, a modified ZFL2-2 has a mutation that stabilizes the ZFL2-2 protein. In some embodiments, a modified ZFL2-2 has a mutation that inhibits the endonuclease activity of the ZFL2-2 protein.

In some embodiments a retroelement-derived polypeptide comprises a polypeptide encoded by Vingi-1 retrotransposon. In some embodiments, the Vingi-1 polypeptide is a modified Vingi-1 polypeptide. In some embodiments, a modified Vingi-1 comprises one or more amino acid substitutions relative to a naturally occurring Vingi-1. In some embodiments, a modified Vingi-1 has an RNA binding mutation. In some embodiments, a modified Vingi-1 has a mutation that stabilizes the Vingi-1 protein. In some embodiments, a modified Vingi-1 has a mutation that inhibits the endonuclease activity of the Vingi-1 protein.

In some embodiments, a retroelement-derived polypeptide has a modification that inactivates its enzymatic activity. In some embodiments, the modification comprises a deletion. In some embodiments, the modification comprises one or more mutations (e.g., point mutations). In some embodiments, the modification is in the endonuclease domain. In some embodiments, the modification is in the integrase domain. In some embodiments, the modification is in the reverse transcriptase domain. In some embodiments, the modification is in the PCNA-interaction peptide (PIP) motif. For example, in some embodiments a heterologous endonuclease domain (e.g., a Cas domain) is fused to a retroelement-derived polypeptide (e.g., a LINE 2-2 polypeptide) that comprises a modification in its endonuclease domain. For example, in some embodiments a UL12 polypeptide is fused to a retroelement-derived polypeptide (e.g., a LINE 2-2 polypeptide) that comprises a modification in its PIP motif.

### Heterologous Polypeptides and Nucleic Acids Encoding the Heterologous Polypeptides

In some embodiments, a nucleic acid encoding an engineered protein comprises a sequence that encodes a heterologous polypeptide fused to a retroelement-derived polypeptide. In some embodiments, the nucleic acid is DNA. In some embodiments, the nucleic acid is RNA. Non-limiting examples of different heterologous polypeptides are described herein and are illustrated in the Examples.

In some embodiments, a retroelement-derived polypeptide (e.g., a reverse transcriptase or integrase domain) is fused to one or more heterologous polypeptides that can promote and/or redirect integration of a transgene (e.g., a heterologous nucleic acid comprising a gene of interest). Non-limiting examples of heterologous polypeptides that can promote and/or redirect integration of a transgene include RNA/DNA processing polypeptides, RNA/DNA repair polypeptides, nucleic acid binding polypeptides, and/or nucleosome binding polypeptides. In some embodiments, an engineered protein comprising a retroelement-derived polypeptide fused to a heterologous polypeptide further comprises a localization signal (e.g., a nuclear localization sequence or a nucleolar localization sequence). In some embodiments, an engineered protein comprising a retroelement-derived polypeptide fused to a heterologous polypeptide further comprises one or more linkers (e.g., at the N- or C-terminus of the heterologous polypeptide and/or within the heterologous polypeptide, for example between different domains within the heterologous polypeptide).

In some embodiments, a retroelement-derived polypeptide (e.g., a reverse transcriptase or integrase domain) is fused to one or more RNA/DNA processing polypeptides (e.g., N-terminally, C-terminally, and/or internally).

In some embodiments, an RNA/DNA processing polypeptide comprises an enzyme that directly causes chemical changes to RNA and/or DNA molecules, for example by promoting degradation of RNA. In addition to interacting with host cell repair and DNA-damage response proteins, proteins that directly process and/or repair RNA/DNA intermediates involved in retrotransposition also may improve retrotransposition efficiency. In some embodiments, an RNA/DNA processing polypeptide improves retrotransposition efficiency and/or redirects retrotransposition to a different target location (e.g., within the genome of a cell).

In some embodiments, the RNA/DNA processing polypeptide is an RNase H domain, or the catalytic region thereof. In some embodiments, an RNase H domain is a prokaryotic RNase H1 domain (e.g., an E. coli RNase H1 domain) or a eukaryotic RNase H1 domain (e.g., a human RNase H1 domain). In some embodiments, the RNA/DNA processing polypeptide is an E. coli RNase H1 domain. Reverse transcription of an RNA template containing a transgene generates an RNA/DNA intermediate which requires processing by cellular RNase H to remove the RNA.

In some embodiments, the RNA/DNA processing polypeptide is a DNA polymerase, or the catalytic region thereof, or an accessory subunit thereof. In some embodiments, the DNA polymerase is a DNA polymerase associated with DNA damage repair. In some embodiments, the RNA/DNA processing polypeptide is PolD3.

In some embodiments, the RNA/DNA processing polypeptide is a RAD51 protein domain. RAD51 is a protein involved in the homology directed repair (HDR) pathway.

In some embodiments, a retroelement-derived polypeptide (e.g., a reverse transcriptase or integrase domain) is fused to one or more RNA/DNA repair polypeptides (e.g., N-terminally, C-terminally, and/or internally).

In some embodiments, an RNA/DNA repair polypeptide is a protein that interacts with host repair proteins (e.g., a repair protein in the host cell). In some embodiments, a host repair protein is a host processing enzyme (e.g., a host DNA repair protein). In some embodiments, host repair proteins are non-homologous end joining (NHEJ) pathway proteins, mismatch repair (MMR), microhomology-mediated end-joining (MMEJ), or homology directed repair (HDR) pathway proteins, or other DNA damage response proteins. In some embodiments, an RNA/DNA repair polypeptide promotes homology directed repair (HDR). In some embodiments, the RNA/DNA repair polypeptide is a CtIP-derived polypeptide, a RecT-derived polypeptide, an HSV-1 alkaline nuclease-derived polypeptide, a BRCA2-derived polypeptide, a DSS1-derived polypeptide, a nanog-derived polypeptide, an NBN-derived polypeptide, a RAD17-derived polypeptide, an ANKRD28-derived polypeptide a PCNA interaction motif polypeptide, a MDC1-derived polypeptide, a MSH4-derived polypeptide, a SCML1-derived polypeptide, a CDKN2A-derived polypeptide, a 53BP1 inhibitor, or a p53 inhibitor.

A CtIP-derived polypeptide is capable of recruiting cellular HDR factors. A RecT-derived polypeptide is capable of promoting ssDNA strand invasion. In some embodiments, a RecT-derived polypeptide is derived from a Pseudomonas aeruginosa RecT. An HSV-1 alkaline nuclease-derived polypeptide (e.g., a UL12 polypeptide) is capable of recruiting the MRN complex and promoting HDR and MMEJ. A BRCA2-derived polypeptide is capable of modulating RAD51 and promoting HDR. A DSS1-derived polypeptide is capable of recruiting RAD52. A Nanog-derived polypeptide can inhibit Rad51, which is important for HDR, thereby inducing repression of HDR. An NBN (Nibrin) polypeptide is capable of interacting with and recruiting MRE11 to form the MRN complex, which is involved in both HDR and MMEJ. A RAD17 polypeptide is capable of interacting with and recruiting factors involved in double-strand break repair.

In some embodiments, the DNA/RNA repair polypeptide is a PCNA interaction motif (PIP motif) is a peptide believed to recruit the cellular PCNA protein which may act as a processivity factor and may be involved in DNA repair and synthesis. RTEs may also include native PIP motifs. In some embodiments, the native RTE PIP motif is replaced with a PIP motif from another protein (such as p21, FEN1 or CHAF1A) which may improve PCNA recruitment.

In some embodiments, PIP motifs may be added as a polypeptide in the C-terminal or N-terminal of RTE protein.

In some embodiments, the RNA/DNA repair polypeptide is an inhibitor of p53. Without wishing to be bound to theory, cellular DNA damage response pathways may act to broadly inhibit genome editing, for example p53 can cause senescence of edited cells.

In some embodiments, the p53 inhibitor is a MDM2-derived peptide, or a peptide 14-derived peptide. MDM2 interacts with and represses p53. A synthetic polypeptide that inhibits p53 can be used to enhance local repression of p53 when delivered together (e.g., in trans) with a retroelement-derived polypeptide.

In some embodiments, the RNA/DNA repair polypeptide is an inhibitor of 53BP1 (p53 binding protein 1). In some embodiments, the 53BP1 inhibitor is an i53 peptide (an engineered ubiquitin variant that has a high binding affinity to 53BP1), or a synthetic peptide. Without wishing to be bound to theory, cellular DNA damage repair may follow the Non-homologous End Joining (NHEJ). 53BP1 is a key regulator of DSB repair pathway in eukaryotic cells and suppresses end resection, thus favoring NHEJ over HDR.

In some embodiments, the heterologous polypeptide is a host factor interaction peptide. Without being bound by theory, a host factor interaction peptide inhibits host defense against retroelements. In some embodiments, the host defense is based around APOBEC3 deaminases.

Without wishing to be bound by theory, APOBEC3-catalyzed deamination of RNA/DNA intermediates can inhibit retrotransposition. In some embodiments a host factor interaction peptide inhibits APOBEC3 deamination. In some embodiments, the host factor interaction peptide is derived from HIV Viral Infectivity Factor (VIF).

The precise class of host cell proteins will depend on the mechanism of integration (e.g., depending on the retroelement-derived polypeptide that is used along with the terminal regions that flank the transgene). In a non-limiting example, retroelements that integrate into a precise location in the genome may rely on an HDR-based integration mechanism, while retroelements that integrate into a random location in the genome may rely on NHEJ or MMEJ-based mechanisms. Activating the corresponding repair pathway while suppressing the alternative repair pathways may increase the efficiency of desired retrotransposition.

In some embodiments, a retroelement-derived polypeptide (e.g., a reverse transcriptase or integrase domain) is fused to one or more nucleic acid binding polypeptides (e.g., N-terminally, C-terminally, and/or internally).

In some embodiments, such nucleic acid binding polypeptide binds RNA and/or DNA. In some embodiments, the nucleic acid binding polypeptide comprises a non-sequence specific DNA binding domain. In some embodiments, the nucleic acid binding polypeptide comprises a sequence specific DNA binding domain. In some embodiments, the non-sequence specific DNA binding domain is a Sto7d DNA binding domain or a Sso7d DNA binding domain. In some embodiments the non-sequence specific DNA binding domain is the T4 phage sliding clamp GP45. In some embodiments, sequence specific DNA binding domains include CRISPR proteins, zing finger proteins, TALES, and the like. Exemplary sequence specific DNA binding domains include, but are not limited to, Cas9 (e.g., dCas9, a SpCas9 with mutations D10A and H840A), a Zinc finger DNA binding domain, a zinc finger targeting AAVS1, and a Transcription activator-like effector (TALE) DNA binding domain. In some embodiments, a sequence-specific endonuclease also may be used to replace a native endonuclease domain of a retroelement-derived polypeptide.

In some embodiments, site-specific endonuclease domains also may be used to replace the native endonuclease of retroelements, thus redirecting the native endonuclease activity to another site. Such domains may retarget integration to a site of interest. Non-limiting examples of an endonuclease fusion/replacement include a site-specific homing endonuclease targeting the a gene fused to retroelement-derived polypeptide deficient in endonuclease activity, either through an inactivating mutation in the nuclease domain (e.g., by a D237A, H238 substitution, and/or D216A in an L2-2 domain or a corresponding mutation in an alternative domain), or through a deletion of the entire predicted endonuclease domain. Similarly, a homing endonuclease nickase variant may be used to introduce a single-strand break instead of a double-strand break. Other non-limiting examples include a Cas9 nuclease or nickase fused to an endonuclease-deficient retroelement-derived polypeptide.

In some embodiments, a retroelement-derived polypeptide (e.g., a reverse transcriptase or integrase domain) is fused to one or more nucleosome binding polypeptides (e.g., N-terminally, C-terminally, and/or internally).

In some embodiments, a nucleosome binding polypeptide binds to nucleosomes. In some embodiments, a nucleosome binding polypeptide is a chromatin modulating polypeptide.

In some embodiments, a nucleosome binding polypeptide alters chromatin accessibility. In some embodiments, a nucleosome binding polypeptide alters the activity of genome editing proteins.

In some embodiments, a nucleosome binding polypeptide comprises an HMGN1 polypeptide, an HMGB1 polypeptide, or a StkC DNA binding domain. In some embodiments, a nucleosome binding polypeptide comprises an HMGN1 polypeptide. In some embodiments, a nucleosome binding polypeptide comprises an HMGB1 polypeptide. In some embodiments, a nucleosome binding polypeptide comprises a StkC DNA binding domain.

In some embodiments, a heterologous polypeptide further comprises a localization signal (e.g., a nuclear localization sequence or a nucleolar localization sequence). In some embodiments, a heterologous polypeptide further comprises one or more linkers (e.g., at the N- or C-terminus of the heterologous polypeptide and/or within the heterologous polypeptide, for example between different domains within the heterologous polypeptide).

In some embodiments, a nuclear localization sequence (NLS) or a nucleolar-localization sequence (NoLS) is included in an engineered protein (or is encoded by a nucleic acid that encodes the engineered protein). In some embodiments, an NLS comprises an SV40 sequence (e.g., PKKKRKV SEQ ID NO: 54), a nucleoplasmin sequence (e.g., KRPAATKKAGQAKKKK SEQ ID NO: 55), or a bipartite SV40 sequence (e.g., KRTADGSEFESPKKKRKV SEQ ID NO: 56). In some embodiments, a NoLS comprises a PNRC sequence (e.g., PKKRRKKK SEQ ID NO: 57), a poly R sequence (e.g., RRRRRRR SEQ ID NO: 58), or a H2B sequence (e.g., KKRKRSRK SEQ ID NO: 59) or TOPBP1 sequence (e.g., KKKSKK SEQ ID NO: 269), or PARP1 sequence (e.g., RQRKRHK SEQ ID NO: 277), or Mdm2sequence (e.g., PSQQKRK SEQ ID NO: 268). The charge and length of NLS and NoLS linkers can affect their ability to mediate localization.

In some embodiments a retroelement-derived polypeptide (e.g., reverse transcriptase and/or integrase domain) is fused to a heterologous polypeptide via a linker. In some embodiments, the linker is a rigid linker. In some embodiments, the linker is a flexible linker. In some embodiments, the linker is a cleavable linker. Flexible linkers are generally made up of small, non-polar (e.g., Gly) or polar (e.g., Ser or Thr) amino acids. Alternating Gly and Ser residues provides flexibility. Solubility of the linker and associated sequences may be enhanced by the inclusion of charged residues, e.g., two positively charged residues (e.g., Lys) and one negatively charged residue (e.g., Glu). In some embodiments, the linker is from 2 to 35 amino acids long.

### Transgenes

In some aspects, one or more engineered proteins and/or nucleic acids encoding the engineered protein(s) are provided along with a gene delivery construct (e.g., a DNA or RNA molecule) comprising a transgene (e.g., a heterologous nucleic acid comprising a gene of interest) to be delivered to a cell or a subject (e.g., to be integrated into a target locus, for example within the genome of the cell).

In some embodiments, a gene delivery construct comprises a heterologous nucleic acid sequence flanked by one or more terminal repeat regions from a non-LTR or an LTR retroelement (e.g., an LTR-RE or an ERV). In some embodiments, a heterologous nucleic acid is a nucleic acid that is not naturally flanked by the terminal repeat regions.

In some embodiments, the heterologous nucleic acid encodes a gene of interest. In some embodiments, the gene of interest encodes an RNA of interest (e.g., a therapeutic RNA, a regulatory RNA, or an RNA enzyme). In some embodiments, the RNA is a messenger RNA (mRNA), antisense RNA (asRNA), RNA interference (RNAi), or an RNA aptamer. In some embodiments, the RNA is an mRNA that encodes a therapeutic protein.

Accordingly, in some embodiments, a gene of interest encodes a therapeutic RNA, a regulatory RNA, an mRNA, an RNA enzyme, or other RNA. A regulatory RNA can be an siRNA, an miRNA, an antisense RNA, or other regulatory RNA. In some embodiments, an encoded RNA is an aptamer. An mRNA can be an RNA that encodes a protein of interest (for example a protein that has therapeutic, diagnostic, and/or other properties). Non limiting examples of proteins of interest include antibodies, regulatory proteins, hormones, cytokines, structural proteins, enzymes, membrane proteins, and other useful therapeutic or diagnostic proteins. Such proteins can be naturally occurring proteins or modified proteins (e.g., containing one or more amino acid substitutions relative to a naturally occurring counterpart protein). In some embodiments, a heterologous nucleic acid can encode one or more genes of interest (e.g., one or more regulatory RNAs and/or proteins of interest).

In some embodiments, non-limiting examples of a gene of interest include the genes that encode Factor VIII, Factor IX, Phenylalanine hydroxylase, ATP7B, alpha glucosidase, argininosuccinate synthetase, galactose-1-phosphate uridyltransferase, ornithine transcarbamylase, or and the like.

In some embodiments, the heterologous nucleic acid encodes two or more genes of interest.

### Terminal Regions

In some embodiments, a gene delivery construct is a nucleic acid (e.g., a DNA or an RNA) comprising a heterologous nucleic acid (e.g., encoding one or more genes of interest) flanked by two terminal regions. These terminal regions may be different when the gene delivery constructs are in trans, i.e. the retrotransposon protein (driver) and transgene (reporter) are encoded by different mRNA.

In some embodiments, the terminal regions are from a non-LTR retrotransposon (e.g., 5′ and/or 3′ UTRs from a non-LTR retrotransposon). In some embodiments, the terminal regions are sequences from a LINE-1, LINE-2, LINE-3/CR-1, CRE, R4, Hero, NeSL, R2, RandI, Proto1, L1, Tx1, RTEPT, Proto2, RTEX, RTE, Outcast, Ingi (optionally sub-clade Vingi), I, Nimb, Tad1, Loa, R1, Jockey, Rex1, CR1, L2, L2A, L2B, Daphne, or Crack retrotransposon. In some embodiments, the terminal regions are sequences from a LINE 2-2 retrotransposon (e.g., a fish LINE 2-2 retrotransposon). In some embodiments, the terminal regions are sequences from a Vingi-1 retrotransposon (e.g., a lizard Vingi-1 retrotransposon).

In some embodiments, a 5′ UTR is a human globin 5′ UTR and/or comprises a Kozak sequence. In some embodiments a 5′UTR is from a zebrafish LINE 2-2 (ZFL2-2), zebrafish LINE 2-1 (ZFL2-1), UnaL2, or Vingi-1. In some embodiments, the ZFL2-2 5′ UTR sequence is AGAGATATCCCTAGCTAGTTCACCGCGGCAGCGGTCGCGGCAGCCTCGTGTGAAGA CCGACGAGGGTAAAGACCATCGACTCTACCTGCGCGACTCCACCGAGCAAAGACAC CGACAAAGCACTTGAGTACTTTACTTTATTGTTTTACTTTACACTTATTTTTTGTTGTC AGTGCACTTTTATT (SEQ ID NO: 64). In some embodiments, the ZFL2-1 5′ UTR sequence is GCGGCCGCTCGAGCATCCGCCTGTTGTTTGTAGCTTTAGCCTGCTAGCGCCGCTGGT CAGCTAAAGCTACCGACCTCTTTAACCATACACTTACTGGCTTTGCTCTTTACCCCGT AAA (SEQ ID NO: 65). In some embodiments, the UnaL2 3′ UTR sequence is TCGACCCACTACCAGGGGAGTCAGGAGAGGTGCAGACGTGGCATCAGTGTGCATCT GATTGTGTCGTCGCTTCTGCCGTCCCCCGCGATTCAGATAAGCGTATCTTAACTTGA TTTGTCTCTGCTGTTGCTAGTTAGAGAACATAGTTGTGCGTAATTTAGATAATCTTTT TTAAACGTGTCTTTACTGTTGCTAGTCAGCGAACTTAGTTGTGCGTTAGCTGAGAAT CTCTGTAGTTGACTCACTGTTGTTAGTTAGTTAAACGCGTTAGTGAAACTGTGTGTG GGGGTTGGTGTTTAACTGCCCGGTATTGTTGAGCTAATTTCAAGTAGCTTCACCTGG TGCTTATCTGCCTTAATGAAGGTGATGCAAGCACGTAATTGTCACCCGGTATTTATA GCTCCAGCGGAGGCTGCCATaGGCAGCCTCGTCGTCAGTTTGTG (SEQ ID NO: 66). In some embodiments the Vingi-1 5′UTR is: GGGGGACACGGAAAGAGCCTCCCCGAAGATTGAGTgAATTCAGTCGGGCGTCCCCT GGGCAACGTTTCTTGTAAGCGGCCGATCTTTCCAcCCCAAAAGCATTGGATGa (SEQ ID NO:67)

In some embodiments, a 3′ UTR is from a zebrafish LINE 2-2 (ZFL2-2), zebrafish LINE 2-1 (ZFL2-1), or UnaL2. In some embodiments, the ZFL2-2 3′ UTR sequence is TGAAACTTGCCTTTAGTACTTATTCATTGTTGCTCTTAGTTGTGTAAATTGCTTCCTT GTCCTCATTTGTAAGTCGCTTTGGATAAAAGCGTCTGCTAAATGACTAAATGTAAAT GTAAATGTAAA (SEQ ID NO: 60). In some embodiments, the ZFL2-1 3′ UTR sequence is GGATCCTGACCATTTATGTGAAGCTGCTTTGACACAATCTACATTGTAAAAGCGCTA TACAAATAAAGCTGAATTGAATTGAATTGAAT (SEQ ID NO: 61).

In some embodiments, the UnaL2 3′ UTR sequence is:
(SEQ ID NO: 62)
CACTTGTATTTGTCTTTGTCCTAATACTGTAGCTTACTCTTCTGCCTAG
TTGGCTTTGCACAGGTTAGGTTAGAATAGTGTTCACTGTGTGAACTGTG
TTCTTAGCTAGAAATAGCTGTACAAAATAAGTATTATACCTTTCTGAAC
TTGTGTTCAGCAGATGCCTACGACCATGATATGCACTTTTGTACGTCGC
TTTGGATAAAAGCGTCTGCGAAATAAATGTAATGTAATGTAATGTAA.
In some embodiments the Vingi-1 3′UTR sequence is:
(SEQ ID NO: 67)
TTGCTTGTGATTTCTTTTCTTTTtTaTTTTATTTCCATTATTTGAAATG
TATTTGcTGTAcCAATGCTTTTGACACGAAATAAATAAA.
In some embodiments the 3′UTR is from human beta
globin, which may have the sequence of:
(SEQ ID NO: 68)
GCTCGCTTTCTTGCTGTCCAATTTCTATTAAAGGTTCCTTTGTTCCCTA
AGTCCAACTACTAAACTGGGGGATATTATGAAGGGCCTTGAGCATCTGG
ATTCTGCCTAATAAAAAACATTTATTTTCATTGCAA.
In some embodiments the 3′UTR is from human alpha
globin, which may have the sequence of:
(SEQ ID NO: 69)
GCTGGAGCCTCGGTGGCCATGCTTCTTGCCCCTTGGGCCTCCCCCCAGC
CCCTCCTCCCCTTCCTGCACCCGTACCCCCGTGGTCTTTGAATAAAGTC
TGAGTGGGCGGCA.

In some embodiments, a gene delivery construct is a nucleic acid (e.g., a DNA or an RNA) comprising a transgene (e.g., heterologous nucleic acid encoding one or more genes of interest) flanked by terminal regions from a non-LTR retrotransposon. In some embodiments, the terminal regions comprise one or more UTRs (e.g., a 5′ UTR and a 3′ UTR). In some embodiments, the terminal regions include one or more regions from a 5′ UTR and/or a 3′ UTR (e.g., a portion of one or both UTRs) from a non-LTR retroelement.

In some embodiments, a terminal region of a gene delivery construct comprises a regulator region of a non-LTR retroelement, for example, one or more 5′ UTR and/or 3′ UTR terminal regions (e.g., from a LINE-1, LINE-2, LINE-3/CR-1, CRE, R4, Hero, NeSL, R2, RandI, Proto1, L1, Tx1, RTEPT, Proto2, RTEX, RTE, Outcast, Ingi (optionally sub-clade Vingi), I, Nimb, Tad1, Loa, R1, Jockey, Rex1, CR1, L2, L2A, L2B, Daphne, or Crack retrotransposon). In some embodiments, the regulatory region comprises a full or partial 5′ UTR or 3′ UTR. For example, in some embodiments the 3′ UTR of a LINE-2-2 comprises a conserved Stem Loop (SL) region and a variable number of a microsatellite repeats (e.g., a minimal 3′ UTR required for efficient retrotransposition).

Accordingly, in some embodiments the terminal regions flanking a gene of interest are not identical sequences. In some embodiments, 3′ UTR terminal regions are approximately 200-600 nucleotides long (e.g., fish LINE-2-2 retrotransposons). In some embodiments, 3′ UTR terminal regions of a gene delivery construct are from natural non-LTR retroelements (non-LTR-REs). In some embodiments, 3′ UTR terminal regions are selected from non-LTR-REs regions found in plants, fungi, insects, and vertebrates (e.g., found in eukaryotes, for example in vertebrates, for example in mammals, for example in humans). Non-limiting examples of non-LTR-REs from which 3′ UTR terminal regions can be used include elements from clades CRE, R4, Hero, NeSL, R2, RandI, Proto1, L1, Tx1, RTEPT, Proto2, RTEX, RTE, Outcast, Ingi (which includes sub-clade Vingi), I, Nimb, Tad1, Loa, R1, Jockey, Rex1, CR1, L2, L2A, L2B, Daphne, and Crack.

In some embodiments, the terminal regions comprise one or more LTR regions. In some embodiments, the terminal repeat regions include one or more LTR regions (e.g., one or more U5, R, and/or U3 regions) from an LTR-RE and/or an ERV.

In some embodiments, a terminal region of a gene delivery construct comprises one or more long terminal repeat (LTR) regions (e.g., from an LTR-RE or an ERV). In some embodiments, the terminal repeat region comprises one or more of a U3, R, and/or U5 regions. For example, in some embodiments a terminal repeat region comprises a U3 region, an R region, a U5 region, a U3 and an R region, an R and a U5 region, or a U3 and an R and a U5 region (e.g., a complete LTR from an LTR-RE or an ERV).

Accordingly, in some embodiments, the first and second terminal regions of a gene delivery construct have identical sequences (e.g., both having a U3-R-U5 configuration). In some embodiments, the first and second terminal regions are approximately 200-1,500 nucleotides long (e.g., 250-1,400 nucleotides long). In some embodiments, the terminal regions of a gene delivery construct are selected from natural LTR-RE or ERV terminal repeat regions.

In some embodiments, the terminal regions are selected from LTR-RE regions found in plants, fungi, insects, and vertebrates, and/or ERV regions (e.g., found in eukaryotes, for example in vertebrates, for example in mammals, for example in humans). Non-limiting examples of LTR-REs and/or ERVs from which terminal regions can be used include Copia, Gypsy, Bel, and Dirs, ERV class I (ERV1), ERV class II (ERV2), EVR class III (ERV3), retroviral-like Intracisternal A Particle (IAP), MusD/Early Transposon (ETn), and ERV mammalian apparent LRT-RE (ERV MaLR). ERV1 regions include gammaretroviral and epsilon retroviral regions. ERV2 regions include betaretroviral regions. ERV3 regions include spumaretroviral regions. In some embodiments, regions from ERVs such as errant-like or errantiviruses can be used. In some embodiments, regions from a human ERV (HERV) can be used.

### Gene Delivery Compositions

In some embodiments, a gene delivery construct (e.g., a DNA and/or RNA molecule) is provided along with (either in cis or in trans) a second nucleic acid that encodes an engineered protein and/or with the engineered protein itself. In some embodiments, the second nucleic acid that encodes the engineered protein can include one or more sequences that encode one or more other proteins (e.g., one or more other LTR or non-LTR retroelement proteins). In some embodiments, the one or more other proteins include a GAG, PRO, and/or ENV protein of an LTR element.

The nucleic acids provided in the cis or trans configurations can be DNA or RNA molecules as described in more detail in this application. In some embodiments, nucleic acids provided in a trans configuration can be DNA, RNA, or a combination of DNA and RNA molecules. For example, the gene delivery construct comprising the transgene (e.g., gene of interest) can be provided as a DNA molecule along with a second nucleic acid that is an RNA molecule (e.g., encoding an engineered protein). However, in some embodiments, the gene delivery construct comprising the transgene (e.g., gene of interest) can be provided as an RNA molecule along with a second nucleic acid that is a DNA molecule (e.g., encoding an engineered protein).

The nucleic acids described in this application (e.g., the gene delivery construct and/or a nucleic acid that encodes one or more proteins that promote genomic integration of the transgene) also can include different regulatory sequences that act as promoters, transcriptional regulators, polyadenylation signals, translational sequences (e.g., ribosome binding sites, etc.).

In some embodiments, such regulatory sequences can be the regulatory sequences that are naturally associated with the genes and/or terminal regions. However, in some embodiments, one or more heterologous regulatory sequences can be added or substituted for the natural sequences, e.g., to provide different levels and/or patterns of expression (for example, higher expression levels than the natural sequences, lower expression levels than the natural sequences, inducible expression, tissue-specific expression, or other patterns of expression). In some embodiments, one or more of the regulatory sequences (e.g., promoters) are constitutive, inducible, and/or tissue specific.

Accordingly, in some embodiments, a gene delivery construct and/or a nucleic acid that encodes an engineered protein comprises one or more naturally occurring promoters, polyadenylation signal sequences, and/or other regulatory sequences. In some embodiments, the naturally occurring promoters, polyadenylation signal sequences, and/or other regulatory sequences can be heterologous sequences (e.g., a CMV promoter, EFIa promoter, MNDU promoter, SFFV promoter, and/or an SV40 polyadenylation sequence). Also, in some embodiments, one or more modified (e.g., having a sequence that differs from a wild-type sequence) promoter, polyadenylation signal sequence, and/or other regulatory sequences are used. In some embodiments, a sequence alteration changes the activity (e.g., increases or decreases the effectiveness, changes the cell or tissue specificity, or otherwise changes the activity) of one or more of these sequences.

In some embodiments, one or more of the naturally occurring promoter and/or transcription regulatory and/or transcription enhancer elements within a terminal region are deleted and/or mutated to increase or decrease transcription from the terminal region. In some embodiments, a nucleic acid may include naturally occurring transcription elements (e.g., promoter, transcription regulatory, and/or transcription enhancer elements) within a terminal region of a gene delivery construct along with additional transcription elements located in a polynucleotide flanking the gene delivery construct (e.g., upstream from the first terminal region on a DNA molecule).

In some embodiments, a promoter that is located within a polynucleotide flanking a gene delivery construct is an inducible promoter. In some embodiments, a promoter that is located within a polynucleotide flanking a gene delivery construct is a tissue-specific promoter.

In some embodiments, a gene delivery construct comprises one or more sequences that are homologous to a target sequence (e.g., a target sequence in a host genome). Non-limiting examples of target sequences include safe harbor genomic targets. In some embodiments, a safe harbor genomic target is a AAVS1, a hROSA26, a CCR5, a SHS231, or a PCSK9 safe harbor.

Accordingly, in some embodiments, a gene delivery construct may include a 5′ terminal region (e.g., a 5′ UTR), a 3′ terminal region (e.g., a 3′ UTR), a polyA sequence, a sequence that is recognized by a retrotransposable element (e.g., a retroelement-derived polypeptide or domain thereof comprised in a driver) for binding, reverse transcription, and/or integration into a target nucleic acid (e.g., into a target genome), and a transgene (e.g., a sequence comprising a gene of interest). In some embodiments, a transgene comprises a promoter that is active (e.g., selectively active) in target cells of interest (e.g., an EF1a, CMV, A1AT, Albumin, or ApoE promoter), and a polyadenylation sequence in addition to a sequence encoding a protein of interest. In some embodiments, a gene delivery construct may comprise one or more RNA nuclear localization sequences (e.g., a SAFB motif) and/or one or more stabilization motifs (e.g., a WPRE motif). In some embodiments, a construct also may comprise flanking regions homologous to a target sequence in a genome.

In some embodiments, a gene delivery construct is provided along with a driver nucleic acid that provides an engineered protein that promotes integration of the gene delivery construct into a target nucleic acid (e.g., a genomic nucleic acid of a host cell). In some embodiments, the gene delivery construct and/or the driver nucleic acid encoding the engineered protein is an RNA molecule. In some embodiments, the gene delivery construct and/or the driver nucleic acid encoding the engineered protein is a DNA molecule (e.g., a single-stranded or a double-stranded DNA molecule). In some embodiments, the DNA and/or RNA molecules further comprise additional flanking nucleic acids. In some embodiments, the additional flanking nucleic acids are Adeno-associated viruses (AAV) or lentiviral nucleic acids. In some embodiments, the additional flanking nucleic acids are AAV inverted terminal repeats (ITRs).

In some embodiments, one or more nucleic acids (e.g., a gene delivery construct and a nucleic acid encoding one or more engineered proteins, for example in cis or in trans) and/or proteins described in this application are provided in a composition for delivery to a cell. In some embodiments, the cell is a mammalian cell. In some embodiments, one or more nucleic acids and/or proteins described in this application are provided in a composition for delivery to a subject. In some embodiments, the subject is a mammalian subject (e.g., a human subject).

In some embodiments, a composition comprises one or more nucleic acids and/or one or more proteins.

In some embodiments, the composition comprises a lipid nanoparticle (LNP). In some embodiments, the average size of an LNP is between 10 to 1000 nm in diameter. Any technique known in the art may be used to determine the size of the LNP. For example, LNP size could be measured using dynamic light scattering (DLS).

In some embodiments, the LNP is comprised of an ionizable lipid, a PEGylated lipid a phospholipid, a cholesterol, a sterol, a non-cationic lipid, or any combination thereof.

In some embodiments, a composition comprises one or more nucleic acids and an LNP.

In some embodiments, a composition comprises one or more proteins and an LNP. In some embodiments, a composition comprises one or more nucleic acids, one or more proteins, and an LNP.

In some embodiments, a composition further comprises a pharmaceutically acceptable carrier, adjuvant, diluent, or excipient.

In some embodiments, one or more nucleic acid(s) and/or proteins are provided in a composition for delivery to a cell, or to a subject. In some embodiments, the subject is a mammal. In some embodiments, the mammal is human.

Methods of administration include, but are not limited to intravenous, intraperitoneal, intramuscular, subcutaneous, intrathecal, and intradermal administration. In some embodiments, administration is via injection or intravenous infusion. In some embodiments, the injection is intramuscular, intraperitoneal, intravascular, or subcutaneous. In some embodiments, two or more compositions (e.g., different compositions, for example comprising different nucleic acids) can be administered together or simultaneously. In some embodiments, two or more compositions (e.g., different compositions, comprising different nucleic acids) can be administered separately (e.g., sequentially).

### EXEMPLARY EMBODIMENTS

The following embodiments are provided as exemplary.

### Set I

Embodiment I-1. A nucleic acid encoding an engineered protein comprising a retroelement-derived polypeptide fused to at least one heterologous polypeptide, wherein the heterologous polypeptide comprises an RNA/DNA processing polypeptide, an RNA/DNA repair polypeptide, a nucleic acid binding polypeptide, a nucleosome binding polypeptide, or any combination thereof.

Embodiment I-2. The nucleic acid of embodiment I-1, wherein the encoded retroelement-derived polypeptide comprises a reverse transcriptase domain, an endonuclease domain, an RNA binding domain, and/or an integrase domain.

Embodiment I-3. The nucleic acid of embodiment I-1 or I-2, encoding a heterologous polypeptide fused to the N-terminus of the retroelement-derived polypeptide.

Embodiment I-4. The nucleic acid of embodiment I-1 or I-2, encoding a heterologous polypeptide fused to the C-terminus of the retroelement-derived polypeptide.

Embodiment I-5. The nucleic acid of embodiment I-1 or I-2, encoding an N-terminal heterologous polypeptide fused to the N-terminus of the retroelement-derived polypeptide, and a C-terminal heterologous polypeptide fused to the C-terminus of the retroelement-derived polypeptide.

Embodiment I-6. The nucleic acid of any prior embodiment, wherein the at least one heterologous polypeptide is inserted within the retroelement-derived polypeptide.

Embodiment I-7. The nucleic acid of embodiment I-6, wherein the at least one heterologous polypeptide is fused a) at its N-terminus to a first domain of the retroelement-derived polypeptide and b) at its C-terminus to a second domain of the retroelement-derived polypeptide.

Embodiment I-8. The nucleic acid of any prior embodiment, wherein at least one heterologous polypeptide comprises an RNA/DNA processing polypeptide.

Embodiment I-9. The nucleic acid of embodiment I-8, wherein the RNA/DNA processing polypeptide is an RNase H polypeptide.

Embodiment I-10. The nucleic acid of any prior embodiment, wherein at least one heterologous polypeptide comprises an RNA/DNA repair polypeptide.

Embodiment I-11. The nucleic acid of embodiment I-10, wherein the RNA/DNA repair polypeptide is a Rad51 polypeptide, a CtlP polypeptide, a HSV-1 alkaline nuclease polypeptide, a BRCA2 polypeptide, a DSS1 polypeptide, a UL12 polypeptide, a Nanog polypeptide, a NBN polypeptide, a p53 inhibitor, an MDM2 polypeptide, or a Peptide 14 polypeptide.

Embodiment I-12. The nucleic acid of any prior embodiment, wherein at least one heterologous polypeptide comprises a nucleic acid binding polypeptide.

Embodiment I-13. The nucleic acid of embodiment I-12, wherein the nucleic acid binding polypeptide is a non-sequence specific DNA binding polypeptide.

Embodiment I-14. The nucleic acid of embodiment I-13, wherein the non-sequence specific DNA binding polypeptide is a Sto7d DNA binding domain or an Sso7d DNA binding domain.

Embodiment I-15. The nucleic acid of embodiment I-12, wherein the nucleic acid binding polypeptide is a sequence specific DNA binding polypeptide.

Embodiment I-16. The nucleic acid of embodiment I-15, wherein the sequence specific DNA binding polypeptide is a dead Cas nuclease, SpCas9 having D10A and/or H840A amino acid substitutions, a Zinc finger DNA binding domain, or a Transcription activator-like effector (TALE) DNA binding domain.

Embodiment I-17. The nucleic acid of any prior embodiment, wherein at least one heterologous polypeptide comprises a nucleosome binding polypeptide.

Embodiment I-18. The nucleic acid of embodiment I-17, wherein the nucleosome binding polypeptide is an HMGN1 polypeptide, a HMGB1 polypeptide, or an StkC DNA binding domain.

Embodiment I-19. The nucleic acid of any prior embodiment, wherein at least one heterologous polypeptide further comprises a localization signal.

Embodiment I-20. The nucleic acid of embodiment I-19, wherein the localization signal is a nuclear localization signal (NLS).

Embodiment I-21. The nucleic acid of embodiment I-20, wherein the NLS is an SV40 (e.g., PKKKRKV), nucleoplasmin (e.g., KRPAATKKAGQAKKKK), or bipartite SV40 (e.g., KRTADGSEFESPKKKRKV) sequence.

Embodiment I-22. The nucleic acid of embodiment I-19, wherein the localization signal is a nucleolar localization signal (NoLS).

Embodiment I-23. The nucleic acid of embodiment I-22, wherein the NoLS is a PNRC (e.g., PKKRRKKK), poly R (e.g., RRRRRRR), or H2B (e.g., KKRKRSRK) sequence.

Embodiment I-24. The nucleic acid of any prior embodiment, wherein at least one heterologous polypeptide comprises at least one linker.

Embodiment I-25. The nucleic acid of embodiment I-24, wherein the linker is at the C-terminus of an N-terminal heterologous polypeptide.

Embodiment I-26. The nucleic acid of embodiment I-24, wherein the linker is at the N-terminus of a C-terminal heterologous polypeptide.

Embodiment I-27. The nucleic acid of any one of embodiments I-24 to I-26, wherein the linker is a rigid linker.

Embodiment I-28. The nucleic acid of any one of embodiments I-24 to I-26, wherein the linker is a flexible linker.

Embodiment I-29. The nucleic acid of embodiment I-24, wherein the linker is a glycine-serine based linker or a XTEN peptide linker.

Embodiment I-30. The nucleic acid of any one of embodiments I-24 to I-29, wherein the linker is 2-35 amino acids long.

Embodiment I-31. The nucleic acid of any prior embodiment, wherein at least one heterologous polypeptide further comprises a viral infectivity factor (VIF).

Embodiment I-32. The nucleic acid of any prior embodiment, wherein the retroelement-derived polypeptide comprises a reverse transcriptase domain.

Embodiment I-33. The nucleic acid of embodiment I-32, wherein the reverse transcriptase domain is from an LTR retrotransposon.

Embodiment I-34. The nucleic acid of embodiment I-32, wherein the reverse transcriptase domain is from a non-LTR retrotransposon.

Embodiment I-35. The nucleic acid of embodiment I-32, wherein the reverse transcriptase domain is from a LINE-1, a LINE-2, or a LINE-3/CR-1 retrotransposon.

Embodiment I-36. The nucleic acid of embodiment I-32, wherein the reverse transcriptase domain is from a LINE 2-2 retrotransposon.

Embodiment I-37. The nucleic acid of embodiment I-36, wherein the LINE-2-2 retrotransposon is a zebrafish LINE 2-2 retrotransposon.

Embodiment I-38. The nucleic acid of embodiment I-32, wherein the reverse transcriptase domain is from murine leukemia virus.

Embodiment I-39. The nucleic acid of embodiment I-32, wherein the retroelement-derived polypeptide further comprises an endonuclease domain.

Embodiment I-40. The nucleic acid of any prior embodiment, wherein the retroelement-derived polypeptide is a POL protein of an LTR retroelement, or an ORF protein of a non-LTR retroelement.

Embodiment I-41. The nucleic acid of embodiment I-40, wherein the retroelement-derived polypeptide is a ZFL2-2 protein.

Embodiment I-42. The nucleic acid of any prior embodiment, encoding at least one heterologous polypeptide that comprises a heterologous endonuclease domain.

Embodiment I-43. The nucleic acid of embodiment I-42, wherein the heterologous endonuclease domain is a Cas9 nuclease, a Cas9 nickase, a SpCas9 with H840A mutation, a homing endonuclease, or a FokI nuclease.

Embodiment I-44. The nucleic acid of any prior embodiment, wherein the nucleic acid encodes an amino acid sequence that is at least 80% identical to any of SEQ ID NOs: 1-22, 27, 29, 31, 33, 35, 37, 39, 41, 43, 45, 47, or 49.

Embodiment I-45. The nucleic acid of any prior embodiment, wherein the nucleic acid encodes an amino acid sequence that is at least 85% identical to any of SEQ ID NOs: 1-22, 27, 29, 31, 33, 35, 37, 39, 41, 43, 45, 47, or 49.

Embodiment I-46. The nucleic acid of any prior embodiment, wherein the nucleic acid encodes an amino acid sequence that is at least 90% identical to any of SEQ ID NOs: 1-22, 27, 29, 31, 33, 35, 37, 39, 41, 43, 45, 47, or 49.

Embodiment I-47. The nucleic acid of any prior embodiment, wherein the nucleic acid encodes an amino acid sequence that is at least 95% identical to any of SEQ ID NOs: 1-22, 27, 29, 31, 33, 35, 37, 39, 41, 43, 45, 47, or 49.

Embodiment I-48. The nucleic acid of any prior embodiment, wherein the nucleic acid encodes an amino acid sequence that is at least 99% identical to any of SEQ ID NOs: 1-22, 27, 29, 31, 33, 35, 37, 39, 41, 43, 45, 47, or 49.

Embodiment I-49. The nucleic acid of any prior embodiment, wherein the reverse transcriptase fusion protein consists of a polypeptide having an amino acid sequence of any of SEQ ID NOs: 1-22, 27, 29, 31, 33, 35, 37, 39, 41, 43, 45, 47, or 49.

Embodiment I-50. The nucleic acid of any prior embodiment, encoding a retroelement-derived reverse transcriptase domain having at least one amino acid substitution that stabilizes the reverse transcriptase domain and/or its association with RNA and/or DNA relative to an unsubstituted reverse transcriptase domain.

Embodiment I-51. The nucleic acid of any prior embodiment, encoding a retroelement-derived endonuclease domain comprising at least one amino acid substitution that promotes its association with DNA relative to an unsubstituted endonuclease domain.

Embodiment I-52. A nucleic acid encoding an engineered protein comprising a retroelement-derived reverse transcriptase domain having at least one amino acid substitution that stabilizes the reverse transcriptase domain and/or its association with RNA and/or DNA relative to an unsubstituted reverse transcriptase domain.

Embodiment I-53. The nucleic acid of embodiment I-52, wherein the reverse transcriptase domain is an amino acid variant of a reverse transcriptase domain of an LTR retrotransposon.

Embodiment I-54. The nucleic acid of embodiment I-52, wherein the reverse transcriptase domain is an amino acid variant of a reverse transcriptase domain of a non-LTR retrotransposon.

Embodiment I-55. The nucleic acid of embodiment I-54, wherein non-LTR retrotransposon is a LINE-1, LINE-2, or a LINE-3/CR-1 retrotransposon.

Embodiment I-56. The nucleic acid of embodiment I-54, wherein non-LTR retrotransposon is a LINE 2-2 retrotransposon.

Embodiment I-57. The nucleic acid of embodiment I-56, wherein the LINE-2-2 retrotransposon is a zebrafish LINE 2-2 retrotransposon.

Embodiment I-58. The nucleic acid of any one of embodiments I-52 to I-57, wherein the at least one amino acid substitution stabilizes the reverse transcriptase domain relative to an unsubstituted reverse transcriptase domain.

Embodiment I-59. The nucleic acid of embodiment I-58, wherein the at least one amino acid substitution a) improves packing of hydrophobic residues in the core of the reverse transcriptase domain, b) stabilizes a loop region of the reverse transcriptase domain, c) alters electrostatic or H-bond stability within the reverse transcriptase domain, d) reduces the size of the active site of the reverse transcriptase domain relative to an unsubstituted reverse transcriptase domain, and/or e) increases the size of an amino acid side chain in the active site of the reverse transcriptase domain relative to an unsubstituted reverse transcriptase domain.

Embodiment I-60. The nucleic acid of embodiment I-58, wherein
a) the at least one amino acid substitution that stabilizes a loop region is a proline substitution and/or addition,
b) the at least one amino acid substitution that alters electrostatic or H-bond stability substitutes a charge or H-bond acceptor/donor preference, or
c) the at least one amino acid substitution that reduces the size of the active site and/or increases the size of an amino acid side chain in the active site of the reverse transcriptase domain comprises at least one amino substitution corresponding to a substitution selected from the group consisting of: A688V, A688I relative to SEQ ID NO: 51.

Embodiment I-61. The nucleic acid of embodiment I-60, wherein the amino acid substitution that substitutes a charge or H-bond acceptor/donor preference
a) substitutes a non-charged amino acid (e.g., alanine) with a positively charged amino acid (e.g., arginine or lysine) or a hydrogen bond donor (e.g., histidine),
b) substitutes a hydrogen bond donor (e.g., asparagine) with a charged amino acid (e.g., lysine), or
c) substitutes a negatively charged amino acid (e.g., aspartate) with a non-charged amino acid (e.g., proline).

Embodiment I-62. The nucleic acid of any one of embodiments I-58 to I-61, wherein the reverse transcriptase domain comprises at least one amino substitution corresponding to a substitution selected from the group consisting of:
a) I625L, H521P, S737P, P705A, M558L, M733L, M760S, M750L, A757P, H717A, H717K, D497S, I625H, L825G, D278S, L837I, A464P, K762R, A948T, P675S, H698P, L742P, E541K, Q547R, S814P, S672P, N560P, H853P, L514P, L524P, Q449P, H650P, G674P, S800P, I896P, S474P, and D520P, relative to SEQ ID NO: 51, and
b) N647K, H717K, I625H, and D520P, relative to SEQ ID NO: 51.

Embodiment I-63. The nucleic acid of any one of embodiments I-52 to I-57, wherein the at least one amino acid substitution
a) stabilizes the association of the reverse transcriptase domain with RNA and/or DNA relative to an unsubstituted reverse transcriptase domain.
b) stabilizes the association of the RNA binding domain of the reverse transcriptase with its cognate RNA relative to an unsubstituted RNA binding domain.

Embodiment I-64. The nucleic acid of embodiment I-63, wherein the at least one amino substitution a) adds a positive charge, b) removes a negative charge, or c) alters at least one H-bond forming residue.

Embodiment I-65. The nucleic acid of embodiment I-63 or I-64, wherein the reverse transcriptase domain comprises at least one amino substitution corresponding to a substitution selected from the group consisting of:
a) D550T, D770H, K815R, S883R, K952R, K542R, N546R, H569R, S577R, H463K, Q478K, K566R, K815R, and S960R, relative to SEQ ID NO: 51, and
b) I343K, L354N, Q357K, and E366N, relative to SEQ ID NO: 51.

Embodiment I-66. A nucleic acid encoding an engineered protein comprising a retroelement-derived endonuclease domain comprising at least one amino acid substitution that promotes its association with DNA relative to an unsubstituted endonuclease domain.

Embodiment I-67. The nucleic acid of embodiment I-66, wherein the endonuclease domain comprises at least one amino acid substitution corresponding to a substitution selected from the group consisting of: Y139K, and D64K relative to SEQ ID NO: 51.

Embodiment I-68. The nucleic acid of any prior embodiment, wherein the nucleic acid is an RNA molecule.

Embodiment I-69. The nucleic acid of any prior embodiment, wherein the nucleic acid is a DNA molecule.

Embodiment I-70. The nucleic acid of embodiment I-69, wherein the nucleic acid comprises a T7 promoter.

Embodiment I-71. The nucleic acid of any of embodiments I-1 to I-69, wherein the nucleic acid comprises a heterologous promoter.

Embodiment I-72. The nucleic acid of embodiment I-71, wherein the promoter is a constitutive promoter.

Embodiment I-73. The nucleic acid of embodiment I-71, wherein the promoter is an inducible promoter.

Embodiment I-74. The nucleic acid of embodiment I-71, wherein the promoter is a tissue specific promoter.

Embodiment I-75. The nucleic acid of embodiment I-71, wherein the promoter is selected from the group consisting of an EF1a promoter, a CMV promoter, an A1AT promoter, an Albumin gene promoter, an MNDU promoter, an SFFV promoter, and an ApoE promoter.

Embodiment I-76. The nucleic acid of any prior embodiment, wherein the nucleic acid comprises one or more chemical or sequence modifications.

Embodiment I-77. The nucleic acid of embodiment I-76, wherein the one or more chemical or sequence modifications are selected from the group consisting of an RNA CAP, a modified polyA length, a chemical modification (e.g., a pseudouridine and/or a methylpseudouridine), a 5′ UTR modification, a 3′ UTR modification, a modified Kozak sequence, a modified (e.g., truncated) stem loop, an RNA stabilization motif, a 5-methoxyuridine (5-MO-U) modification, a 5-methylcytidine (5mC) modification, and one or more additional and/or modified microsatellites.

Embodiment I-78. The nucleic acid of any prior embodiment comprising a codon optimized sequence.

Embodiment I-79. The nucleic acid of embodiment I-78, wherein the codon optimized sequence is optimized for expression in human cells.

Embodiment I-80. The nucleic acid of embodiment I-78, wherein the codon optimized sequence has a reduced Uracil (U) load relative to a corresponding naturally occurring sequence.

Embodiment I-81. The nucleic acid of embodiment I-77, wherein the RNA stabilization motif is a WPRE motif.

Embodiment I-82. An engineered protein encoded by any one of the nucleic acids of any one of embodiments I-1 to I-81.

Embodiment I-83. A composition comprising:
a) a first nucleic acid of any one of embodiments I-1 to I-81; and
b) a second nucleic acid comprising a polynucleotide encoding a gene of interest.

Embodiment I-84. The composition of embodiment I-83, wherein the first and second nucleic acids are separate DNA molecules.

Embodiment I-85. The composition of embodiment I-83, wherein the first and second nucleic acids are separate RNA molecules.

Embodiment I-86. The composition of embodiment I-83, wherein the one of the first and second nucleic acids is a DNA molecule and one of the first and second nucleic acids is an RNA molecule.

Embodiment I-87. The composition of any one of embodiments I-83 to I-86, wherein the first polynucleotide is operably linked to a first heterologous promoter.

Embodiment I-88. The composition of any one of embodiments I-83 to I-87, wherein the second polynucleotide is operably linked to a second heterologous promoter.

Embodiment I-89. The composition of embodiment I-87 or I-88, wherein at least one of the first and second heterologous promoters is a constitutive promoter.

Embodiment I-90. The composition of embodiment I-87 or I-88, wherein at least one of the first and second heterologous promoters is an inducible promoter.

Embodiment I-91. The composition of embodiment I-87 or I-88, wherein at least one of the first and second heterologous promoters is a constitutive promoter.

Embodiment I-92. The composition of embodiment I-87 or I-88, wherein the first and second heterologous promoters are independently an EF1a promoter, a CMV promoter, an A1AT promoter, an Albumin gene promoter, MNDU promoter, SFFV promoter, or an ApoE promoter.

Embodiment I-93. The composition of any one of embodiments I-83 to I-92, wherein one or both of the first and second nucleic acids further comprise one or more of the following modifications: an RNA CAP, a modified polyA length, a chemical modification (e.g., a pseudouridine and/or a methylpseudouridine), a 5′ UTR modification, a 3′ UTR modification, a modified Kozak sequence, a modified (e.g., truncated) stem loop, an RNA stabilization motif, a 5-methoxyuridine (5-MO-U) modification, a 5-methylcytidine (5mC) modification, or one or more additional and/or modified microsatellites.

Embodiment I-94. The composition of any one of embodiments I-83 to I-93, wherein one or both of the first and second nucleic acids comprises a codon optimized sequence.

Embodiment I-95. The composition of embodiment I-94, wherein the codon optimized sequence is optimized for expression in human cells.

Embodiment I-96. The composition of embodiment I-94, wherein the codon optimized sequence has a reduced Uracil (U) load relative to a corresponding naturally occurring sequence.

Embodiment I-97. The composition of embodiment I-93, wherein the RNA stabilization motif is a WPRE motif.

Embodiment I-98. The composition of any one of embodiments I-83 to I-97, wherein one or both of the first and second nucleic acids further comprise an RNA nuclear localization sequence.

Embodiment I-99. The composition of embodiment I-98, wherein the RNA nuclear localization sequence is an SAFB motif.

Embodiment I-100. The composition of any one of embodiments I-83 to I-99, wherein the second polynucleotide is flanked by a first terminal region and a second terminal region.

Embodiment I-101. The composition of embodiment I-100, wherein the first and second terminal regions are LTRs.

Embodiment I-102. The composition of embodiment I-100, wherein the first terminal region is the 5′ UTR of a LINE, and the second terminal region is the 3′ UTR of a LINE.

Embodiment I-103. The composition of embodiment I-102, wherein the LINE 3′UTR region comprises a truncated stem loop relative to a wild-type stem loop.

Embodiment I-104. The composition of any one of embodiments I-83 to I-103, wherein the second nucleic acid further comprises a 5′ UTR, a 3′UTR, a polyA sequence, a sequence that is recognized, by the engineered protein encoded by the first nucleic acid, for binding, reverse transcription, and integration of the gene of interest into a target nucleic acid.

Embodiment I-105. The composition of embodiment I-104, wherein the target nucleic acid is a genome of a target cell.

Embodiment I-106. The composition of any one of embodiments I-83 to I-105, wherein the second nucleic acid comprises i) a promoter operably linked to the second polynucleotide encoding the gene of interest, and ii) a polyadenylation sequence, and wherein the promoter is selectively active in one or more target cell types.

Embodiment I-107. The composition of any one of embodiments I-83 to I-106, wherein the gene of interest encodes a therapeutic RNA and/or a therapeutic protein.

Embodiment I-108. The composition of embodiment I-107, wherein the therapeutic RNA is an antisense RNA (asRNA), small interfering RNA (siRNA), microRNA (miRNA), or RNA aptamer.

Embodiment I-109. The composition of embodiment I-107, wherein the therapeutic protein is an antibody, regulatory protein, hormone, cytokine, structural protein, enzyme, or membrane protein.

Embodiment I-110. The composition of embodiment I-107, wherein the therapeutic protein is Factor VIII, Factor IX, Phenylalanine hydroxylase, ATP7B, alpha glucosidase, argininosuccinate synthetase, galactose-1-phosphate uridyltransferase, or ornithine transcarbamylase.

Embodiment I-111. The composition of any one of embodiments I-83 to I-110, wherein the second nucleic acid comprises flanking regions homologous to target sites in a genome of a target cell.

Embodiment I-112. The composition of any one of embodiments I-83 to I-111, wherein the first and second nucleic acids are comprised within a plurality of LNP particles.

Embodiment I-113. A method comprising administering a nucleic acid of any one of embodiments I-1 to I-81, a composition of any one of embodiments I-83 to I-112, or an engineered protein embodiment I-82 to a subject.

Embodiment I-114. The method of embodiment I-113, wherein the subject is a human.

### Set II

Embodiment II-1. A nucleic acid encoding an engineered protein comprising a retroelement-derived polypeptide and at least one heterologous polypeptide; wherein the retroelement-derived polypeptide is derived from a non-long terminal repeat (non-LTR) retrotransposon; wherein the at least one heterologous polypeptide comprises an RNA/DNA processing polypeptide or domain thereof, an RNA/DNA repair polypeptide or domain thereof, a nucleic acid binding polypeptide or domain thereof, or a nucleosome binding polypeptide or domain thereof; and wherein the engineered protein exhibits at least one improved integration characteristic, as compared to a retroelement-derived polypeptide not fused to the at least one heterologous polypeptide.

Embodiment II-2. The nucleic acid of embodiment II-1, wherein the at least one improved integration characteristic is one or more of improved efficiency of integration, accuracy of integration, fidelity of integration, and processivity of integration.

Embodiment II-3. The nucleic acid of any one of embodiments II-1 to II-2, wherein the at least one heterologous polypeptide is capable of one or more of: promoting homology directed repair, promoting chromatin binding, promoting chromatin accessibility, promoting DNA binding, and promoting RNA binding.

Embodiment II-4. The nucleic acid of any one of embodiments II-1 to II-3, wherein the at least one heterologous polypeptide comprises an RNA/DNA processing polypeptide or domain thereof.

Embodiment II-5. The nucleic acid of embodiment II-4, wherein the at least one heterologous polypeptide comprising an RNA/DNA processing polypeptide or domain thereof is capable of: RNAseH activity, DNA polymerase activity, inhibiting ApoBec3 deaminase, and/or strand invasion of single-stranded DNA.

Embodiment II-6. The nucleic acid of embodiment II-4, wherein the RNA/DNA processing polypeptide is a Rad51 polypeptide, an RNAseH domain, a DNA polymerase.

Embodiment II-7. The nucleic acid of embodiment II-6, wherein the RNA/DNA processing polypeptide is a Rad51 polypeptide having an amino acid sequence that is at least 70% identical to the sequence as set forth in SEQ ID NO: 311.

Embodiment II-8. The nucleic acid of any one of embodiments II-1 to II-7, wherein the at least one heterologous polypeptide comprises an RNA/DNA repair polypeptide or domain thereof.

Embodiment II-9. The nucleic acid of embodiment II-8, wherein the at least one heterologous polypeptide comprising an RNA/DNA repair polypeptide or domain thereof is capable of: recruiting proteins involved in homologous recombination, recruiting DNA damage/signaling/repair factors, recruiting PCNA, inhibiting p53, and/or inhibiting 53BP1.

Embodiment II-10. The nucleic acid of embodiment II-8, wherein the RNA/DNA repair polypeptide is a CtIP-derived polypeptide, a RecT-derived polypeptide, an HSV-1 alkaline nuclease-derived polypeptide, a BRCA2-derived polypeptide, a DSS1-derived polypeptide, a nanog-derived polypeptide, an NBN-derived polypeptide, a RAD17-derived polypeptide, an ANKRD28-derived polypeptide, a PCNA interaction motif polypeptide, a MDC1-derived polypeptide, a MSH4-derived polypeptide, a SCML1-derived polypeptide, a CDKN2A-derived polypeptide, a 53BP1 inhibitor, or a p53 inhibitor.

Embodiment II-11. The nucleic acid of embodiment II-10, wherein the RNA/DNA repair polypeptide is a Rad17 polypeptide having an amino acid sequence that is at least 70% identical to the sequence as set forth in SEQ ID NO: 386.

Embodiment II-12. The nucleic acid of embodiment II-10, wherein the RNA/DNA repair polypeptide is an ANKRD28 polypeptide having an amino acid sequence that is at least 70% identical to the sequence as set forth in SEQ ID NO: 396

Embodiment II-13. The nucleic acid of embodiment II-10, wherein the RNA/DNA repair polypeptide is an HSV-1 alkaline nuclease (UL12) polypeptide having an amino acid sequence that is at least 70% identical to the sequence as set forth in SEQ ID NO: 25.

Embodiment II-14. The nucleic acid of embodiment II-10, wherein the RNA/DNA repair polypeptide is a BRCA2-derived polypeptide having an amino acid sequence that is at least 70% identical to the sequence as set forth in SEQ ID NO: 308.

Embodiment II-15. The nucleic acid of embodiment II-10, wherein the RNA/DNA repair polypeptide is a PCNA interaction motif having an amino acid sequence that is at least 70% identical to the sequence as set forth in any one of SEQ ID NOS: 250, 251, 391, 394, and 395.

Embodiment II-16. The nucleic acid of embodiment II-10, wherein the RNA/DNA repair polypeptide is a MDC1 polypeptide having an amino acid sequence that is at least 70% identical to the sequence as set forth in SEQ ID NO: 388.

Embodiment II-17. The nucleic acid of embodiment II-10, wherein the RNA/DNA repair polypeptide is a MSH4 polypeptide having an amino acid sequence that is at least 70% identical to the sequence as set forth in SEQ ID NO: 392.

Embodiment II-18. The nucleic acid of embodiment II-10, wherein the RNA/DNA repair polypeptide is a SCML1 polypeptide having an amino acid sequence that is at least 70% identical to the sequence as set forth in SEQ ID NO: 387.

Embodiment II-19. The nucleic acid of embodiment II-10, wherein the RNA/DNA repair polypeptide is a CDKN2A polypeptide having an amino acid sequence that is at least 70% identical to the sequence as set forth in SEQ ID NO: 389.

Embodiment II-20. The nucleic acid of embodiment II-10, wherein the RNA/DNA repair polypeptide is a p53 inhibitor.

Embodiment II-21. The nucleic acid of embodiment II-20, wherein the p53 inhibitor is a MDM2-derived peptide having an amino acid sequence that is at least 70% identical to the sequence as set forth in SEQ ID NO: 313, or a peptide 14-derived peptide having an amino acid sequence that is at least 70% identical to the sequence as set forth in SEQ ID NO: 314.

Embodiment II-22. The nucleic acid of any one of embodiments II-1 to II-21, wherein the at least one heterologous polypeptide comprises a nucleic acid binding polypeptide or domain thereof.

Embodiment II-23. The nucleic acid of embodiment II-22, wherein the nucleic acid binding polypeptide comprises a non-sequence specific DNA binding polypeptide or domain thereof.

Embodiment II-24. The nucleic acid of embodiment II-23, wherein the non-sequence specific DNA binding polypeptide comprises a Sto7d DNA binding domain having an amino acid sequence that is at least 70% identical to the sequence as set forth in SEQ ID NO: 26, or an Sso7d DNA binding domain having an amino acid sequence that is at least 70% identical to the sequence as set forth in SEQ ID NO: 377.

Embodiment II-25. The nucleic acid of embodiment II-22, wherein the nucleic acid binding polypeptide comprises a sequence specific DNA binding polypeptide or domain thereof.

Embodiment II-26. The nucleic acid of embodiment II-25, wherein the sequence specific DNA binding polypeptide is a Cas9 nuclease, dead Cas nuclease, SpCas9 having D10A and/or H840A amino acid substitutions, a Zinc finger DNA binding domain, or a transcription activator-like effector (TALE) DNA binding domain, having an amino acid sequence that is at least 70% identical to the sequence as set forth in any one of SEQ ID NOS: 259, 330, 318, 333, 400, and 401.

Embodiment II-27. The nucleic acid of any one of embodiments II-1 to II-26, wherein at least one heterologous polypeptide comprises a nucleosome binding polypeptide or domain thereof.

Embodiment II-28. The nucleic acid of embodiment II-27, wherein the nucleosome binding polypeptide comprises an HMGN1 polypeptide having an amino acid sequence that is at least 70% identical to the sequence as set forth in SEQ ID NO:23.

Embodiment II-29. The nucleic acid of embodiment II-27, wherein the nucleosome binding polypeptide comprises an HMGB1 polypeptide having an amino acid sequence that is at least 70% identical to the sequence as set forth in SEQ ID NO:24.

Embodiment II-30. The nucleic acid of embodiment II-27, wherein the nucleosome binding polypeptide comprises an StkC DNA binding domain having an amino acid sequence that is at least 70% identical to the sequence as set forth in SEQ ID NO:402.

Embodiment II-31. The nucleic acid of any one of embodiments II-1 to II-30, wherein the engineered protein comprises the at least one heterologous polypeptide fused to the N-terminus of the retroelement-derived polypeptide, to the C-terminus of the retroelement-derived polypeptide and/or internally within the retroelement-derived polypeptide.

Embodiment II-32. The nucleic acid of embodiment II-31, wherein the engineered protein comprises the at least one heterologous polypeptide fused to the C-terminus of the retroelement-derived polypeptide.

Embodiment II-33. The nucleic acid of embodiment II-31 or II-32, wherein the engineered protein comprises the at least one heterologous polypeptide fused to the N-terminus of the retroelement-derived polypeptide.

Embodiment II-34. The nucleic acid of any one of embodiments II-31 to II-33, wherein the engineered protein comprises the at least one heterologous polypeptide fused internally within the retroelement-derived polypeptide.

Embodiment II-35. The nucleic acid of any one of embodiments II-31 to II-34, wherein the engineered protein comprises a first heterologous polypeptide fused to the N-terminus of the retroelement-derived polypeptide and a second heterologous polypeptide fused to the C-terminus of the retroelement-derived polypeptide.

Embodiment II-36. The nucleic acid of any one of embodiments II-1 to II-35, wherein engineered protein comprises a plurality of heterologous polypeptides.

Embodiment II-37. The nucleic acid of any one of embodiment II-1 to II-36 wherein engineered protein comprises at least one localization signal.

Embodiment II-38. The nucleic acid of embodiment II-37, wherein the at least one localization signal is a nuclear localization signal (NLS).

Embodiment II-39. The nucleic acid of embodiment II-38, wherein the NLS comprises an amino acid sequence that is at least 80% identical, at least 90% identical, or 100% identical to a sequence as set forth in any one of SEQ ID NOs:54-56, 58, 59, 382, 384, or 390.

Embodiment II-40. The nucleic acid of embodiment II-37, wherein the at least one localization signal is a nucleolar localization signal (NoLS).

Embodiment II-41. The nucleic acid of embodiment II-40, wherein the NoLS. comprises an amino acid sequence that is at least 80% identical, at least 90% identical, or 100% identical to a sequence as set forth in SEQ ID NO:57.

Embodiment II-42. The nucleic acid of any one of embodiments II-1 to II-41, wherein the engineered protein comprises at least one linker.

Embodiment II-43. The nucleic acid of embodiment II-42, wherein the linker is at the C-terminus of a heterologous polypeptide located at the N-terminus of the engineered protein.

Embodiment II-44. The nucleic acid of embodiment II-42, wherein the linker is at the N-terminus of a heterologous polypeptide located at the C-terminus of the engineered protein.

Embodiment II-45. The nucleic acid of any one of embodiments II-42 to II-44, wherein the linker is a rigid linker.

Embodiment II-46. The nucleic acid of any one of embodiments II-42 to II-44, wherein the linker is a flexible linker.

Embodiment II-47. The nucleic acid of embodiment II-42, wherein the linker is a glycine-serine based linker.

Embodiment II-48. The nucleic acid of embodiment II-42, wherein the linker isa XTEN peptide linker.

Embodiment II-49. The nucleic acid of any one of embodiments II-42 to II-47, wherein the linker is 2-35 amino acids long.

Embodiment II-50. The nucleic acid of any one of embodiments II-42 to II-49, wherein the linker is selected from any one of SEQ ID NOs:334-340.

Embodiment II-51. The nucleic acid of any one of embodiments II-1 to II-49, wherein the engineered protein comprises a viral infectivity factor (VIF), optionally wherein the VIF is selected from any one of SEQ ID NOs:378-380.

Embodiment II-52. The nucleic acid of any one of embodiments II-1 to II-51, wherein the non-LTR retrotransposon is an apurinic/apyrimidinic endonucleases (APE)-type retrotransposon.

Embodiment II-53. The nucleic acid of embodiment II-52, wherein the APE-type retrotransposon is a ZFL2-2 retrotransposon, a Vingi-1_Acar retrotransposon, a Vingi-2_Acar retrotransposon, a L2-18_Acar retrotransposon, or a CR1-1_Acar retrotransposon.

Embodiment II-54. The nucleic acid of any one of embodiments II-1 to II-53, wherein the retroelement-derived polypeptide is a wild type retroelement-derived polypeptide, or at least one domain thereof.

Embodiment II-55. The nucleic acid of any one of embodiments II-1 to II-53, wherein the retroelement-derived polypeptide is a retroelement-derived polypeptide variant comprising an amino acid substitution, an amino acid deletion, an amino acid truncation, or a combination thereof, when compared to a wild type retroelement-derived polypeptide.

Embodiment II-56. The nucleic acid of any one of embodiments II-1 to II-55, wherein the retroelement-derived polypeptide comprises a reverse transcriptase domain, an endonuclease domain, integrase domain, and/or an RNA binding domain.

Embodiment II-57. The nucleic acid of embodiment II-56, wherein the retroelement-derived polypeptide comprises a reverse transcriptase domain.

Embodiment II-58. The nucleic acid of embodiment II-57, wherein the reverse transcriptase domain is from an APE-type retrotransposon.

Embodiment II-59. The nucleic acid of embodiment II-57, wherein the reverse transcriptase domain is from a CRE, R4, Hero, NeSL, R2, RandI, Proto1, L1, Tx1, RTEPT, Proto2, RTEX, RTE, Outcast, Ingi, I, Nimb, Tad1, Loa, R1, Jockey, Rex1, CR1, L2, L2A, L2B, Daphne, or Crack retrotransposon.

Embodiment II-60. The nucleic acid of embodiment II-57, wherein the reverse transcriptase domain is from a LINE 2-2 retrotransposon.

Embodiment II-61. The nucleic acid of embodiment II-60, wherein the LINE-2-2 retrotransposon is a zebrafish LINE 2-2 retrotransposon.

Embodiment II-62. The nucleic acid of embodiment II-57, wherein the reverse transcriptase domain is from murine leukemia virus.

Embodiment II-63. The nucleic acid of embodiment II-57, wherein the retroelement-derived polypeptide further comprises an endonuclease domain.

Embodiment II-64. The nucleic acid of any one of embodiments II-1 to II-63, wherein the retroelement-derived polypeptide is an ORF protein of a non-LTR retrotransposon.

Embodiment II-65. The nucleic acid of embodiment II-64, wherein the retroelement-derived polypeptide is a ZFL2-2 protein.

Embodiment II-66. The nucleic acid of embodiment II-64, wherein the retroelement-derived polypeptide is a Vingi-1 protein.

Embodiment II-67. The nucleic acid of embodiment II-1, wherein the heterologous endonuclease domain comprises a Cas9 nuclease having an amino acid sequence as set forth in any one of SEQ ID NOs:22, 293, 294, 296 or 663, or having an amino acid sequence that is at least 70% identical to the sequence as set forth in any one of SEQ ID NOs:22, 293, 294, 296 or 663.

Embodiment II-68. The nucleic acid of any one of embodiments II-1 to II-67, wherein the nucleic acid encodes an amino acid sequence that is at least 80% identical to any one of SEQ ID NOs: 1-22, 27, 29, 31, 33, 35, 37, 39, 41, 43, 45, 47, 49, 51, 70-246, 248-259, 263-277, 293-307, or 341-368.

Embodiment II-69. The nucleic acid of any one of embodiments II-1 to II-67, wherein the nucleic acid encodes an amino acid sequence that is at least 85% identical to any one of SEQ ID NOs: 1-22, 27, 29, 31, 33, 35, 37, 39, 41, 43, 45, 47, 49, 51, 70-246, 248-259, 263-277, 293-307, or 341-368.

Embodiment II-70. The nucleic acid of any one of embodiments II-1 to II-67, wherein the nucleic acid encodes an amino acid sequence that is at least 90% identical to any one of SEQ ID NOs: 1-22, 27, 29, 31, 33, 35, 37, 39, 41, 43, 45, 47, 49, 51, 70-246, 248-259, 263-277, 293-307, or 341-368.

Embodiment II-71. The nucleic acid of any one of embodiments II-1 to II-67, wherein the nucleic acid encodes an amino acid sequence that is at least 95% identical to any one of SEQ ID NOs: 1-22, 27, 29, 31, 33, 35, 37, 39, 41, 43, 45, 47, 49, 51, 70-246, 248-259, 263-277, 293-307, or 341-368.

Embodiment II-72. The nucleic acid of any one of embodiments II-1 to II-67, wherein the nucleic acid encodes an amino acid sequence that is at least 99% identical to any of SEQ ID NOs: 1-22, 27, 29, 31, 33, 35, 37, 39, 41, 43, 45, 47, 49, 51, 70-246, 248-259, 263-277, 293-307, or 341-368.

Embodiment II-73. The nucleic acid of any one of embodiments II-1 to II-67, wherein the reverse transcriptase fusion protein consists of a polypeptide having an amino acid sequence of any of SEQ ID NOs: 1-22, 27, 29, 31, 33, 35, 37, 39, 41, 43, 45, 47, 49, 51, 70-246, 248-259, 263-277, 293-307, or 341-368.

Embodiment II-74. The nucleic acid of any one of embodiments II-1 to II-73, encoding a retroelement-derived reverse transcriptase domain having at least one amino acid substitution that stabilizes the reverse transcriptase domain and/or its association with RNA and/or DNA relative to an unsubstituted reverse transcriptase domain.

Embodiment II-75. The nucleic acid of any one of embodiments II-1 to II-73, encoding a retroelement-derived endonuclease domain comprising at least one amino acid substitution that promotes its association with DNA relative to an unsubstituted endonuclease domain.

Embodiment II-76. A nucleic acid encoding an engineered protein comprising a retroelement-derived polypeptide variant having at least one amino acid modification when compared to a naturally occurring retroelement-derived polypeptide;
wherein the retroelement-derived polypeptide variant is derived from a non-long terminal repeat (non-LTR) retrotransposon; and
wherein the engineered protein exhibits at least one improved characteristic, as compared to the naturally occurring retroelement-derived polypeptide without the at least one amino acid modification.

Embodiment II-77. The nucleic acid of embodiment II-76, wherein the amino acid modification is an amino acid substitution, an amino acid deletion, an amino acid addition, an amino acid truncation, or a combination thereof.

Embodiment II-78. The nucleic acid of embodiment II-76 or II-77, wherein the retroelement-derived polypeptide variant comprises an amino acid sequence that is at least 99% identical to any one of SEQ ID NOs: 70-246, 298-306, or 341-368.

Embodiment II-79. The nucleic acid of embodiment II-76 or II-77, wherein the non-LTR retrotransposon is a LINE2-2 retrotransposon.

Embodiment II-80. The nucleic acid of embodiment II-79, wherein the LINE2-2 retrotransposon is a zebra fish LINE2-2 (ZFL2-2) retrotransposon.

Embodiment II-81. The nucleic acid of embodiment II-79, wherein the retroelement-derived polypeptide variant comprises an amino acid sequence that is at least 95% identical, at least 97% identical, at least 99% identical, or 100% identical, to any one of SEQ ID NOs: 341-368.

Embodiment II-82. The nucleic acid of any one of embodiments II-79 to II-81, wherein the retroelement-derived polypeptide variant comprises at least one amino substitution corresponding to a substitution selected from the group consisting of: I343K, Q372K, D588A, N647K, H521P, S737P, P705A, M750L, A757P, and H717A relative to the wild-type ZFL2-2 retrotransposon having the sequence SEQ ID NO:49.

Embodiment II-83. The nucleic acid of embodiment II-82, wherein the retroelement-derived polypeptide variant comprises at least one amino substitution corresponding to a substitution selected from the group consisting of: N647K, H521P, S737P, and M750L relative to the wild-type ZFL2-2 retrotransposon having the sequence SEQ ID NO:49.

Embodiment II-84. The nucleic acid of embodiment II-76 or II-77, wherein the non-LTR retrotransposon is a Vingi-1 retrotransposon.

Embodiment II-85. The nucleic acid of embodiment II-76 or II-77, wherein the Vingi-1 retrotransposon is an Anolis carolinensis Vingi-1 retrotransposon.

Embodiment II-86. The nucleic acid of embodiment II-85, wherein the retroelement-derived polypeptide variant comprises an amino acid sequence that is at least 95% identical, at least 97% identical, at least 99% identical, or 100% identical to any one of SEQ ID NOs: 70-246 or 298-306.

Embodiment II-87. The nucleic acid of any one of embodiments II-83 to II-86, wherein the retroelement-derived polypeptide variant comprises at least one amino substitution corresponding to a substitution selected from the group consisting of: Q634L, F238Y+M16I, I45L, G833I, K703R, K480Q, K675R, P808K, M570L, L590F, M735E, K966R, A901H, and L493R relative to the wild-type Anolis carolinensis Vingi-1 retrotransposon having the sequence SEQ ID NO:327.

Embodiment II-88. The nucleic acid of any one of embodiments II-83 to II-86, wherein the retroelement-derived polypeptide variant comprises at least one amino substitution corresponding to a substitution selected from the group consisting of: M570L, K966R, and A901H. relative to the wild-type Anolis carolinensis Vingi-1 retrotransposon having the sequence SEQ ID NO:327.

Embodiment II-89. The nucleic acid of any one of embodiments II-76 to II-88, wherein the engineered protein further comprises at least one heterologous polypeptide.

Embodiment II-90. The nucleic acid of embodiment II-89, wherein the at least one heterologous polypeptide is capable of one or more of: promoting homology directed repair, promoting chromatin binding, promoting chromatin accessibility, promoting DNA binding, and promoting RNA binding.

Embodiment II-91. The nucleic acid of embodiment II-89 or II-90, wherein the at least one heterologous polypeptide comprises an RNA/DNA processing polypeptide or domain thereof.

Embodiment II-92. The nucleic acid of embodiment II-91, wherein the at least one heterologous polypeptide comprising an RNA/DNA processing polypeptide or domain thereof is capable of: RNAseH activity, DNA polymerase activity, inhibiting ApoBec3 deaminase, and/or strand invasion of single-stranded DNA.

Embodiment II-93. The nucleic acid of embodiment II-91, wherein the RNA/DNA processing polypeptide is a Rad51 polypeptide, a RAD17 polypeptide, or a RAD6 polypeptide.

Embodiment II-94. The nucleic acid of any one of embodiments II-89 to II-93, wherein the at least one heterologous polypeptide comprises an RNA/DNA repair polypeptide or domain thereof.

Embodiment II-95. The nucleic acid of embodiment II-94, wherein the at least one heterologous polypeptide comprising an RNA/DNA repair polypeptide or domain thereof is capable of: recruiting proteins involved in homologous recombination, recruiting DNA damage/signaling/repair factors, recruiting PCNA, inhibiting p53, and/or inhibiting 53BP1.

Embodiment II-96. The nucleic acid of embodiment II-94, wherein the RNA/DNA repair polypeptide is a CtIP-derived polypeptide, a RecT-derived polypeptide, an HSV-1 alkaline nuclease-derived polypeptide, a BRCA2-derived polypeptide, a DSS1-derived polypeptide, a nanog-derived polypeptide, an NBN-derived polypeptide, a RAD17-derived polypeptide, an ANKRD28-derived polypeptide, a PCNA interaction motif polypeptide, a MDC1-derived polypeptide, a MSH4-derived polypeptide, a SCML1-derived polypeptide, a CDKN2A-derived polypeptide, a 53BP1 inhibitor, or a p53 inhibitor.

Embodiment II-97. The nucleic acid of any one of embodiments II-89 to II-96, wherein the at least one heterologous polypeptide comprises a nucleic acid binding polypeptide or domain thereof.

Embodiment II-98. The nucleic acid of embodiment II-97, wherein the nucleic acid binding polypeptide comprises a non-sequence specific DNA binding polypeptide or domain thereof.

Embodiment II-99. The nucleic acid of embodiment II-98, wherein the non-sequence specific DNA binding polypeptide comprises a Sto7d DNA binding domain.

Embodiment II-100. The nucleic acid of embodiment II-97, wherein the nucleic acid binding polypeptide comprises a sequence specific DNA binding polypeptide or domain thereof.

Embodiment II-101. The nucleic acid of embodiment II-100, wherein the sequence specific DNA binding polypeptide is a Cas9 nuclease, dead Cas nuclease, SpCas9 having D10A and/or H840A amino acid substitutions, a Zinc finger DNA binding domain, or a transcription activator-like effector (TALE) DNA binding domain.

Embodiment II-102. A nucleic acid encoding a retroelement-derived reverse transcriptase domain comprising at least one amino acid modification that stabilizes the reverse transcriptase domain and/or stabilizes its association with RNA and/or DNA relative to an unsubstituted reverse transcriptase domain, wherein the reverse transcriptase domain is an amino acid variant of a reverse transcriptase domain of a non-LTR retrotransposon.

Embodiment II-103. The nucleic acid of embodiment II-102, wherein the non-LTR retrotransposon is a LINE-1, LINE-2, or a LINE-3/CR-1 retrotransposon.

Embodiment II-104. The nucleic acid of embodiment II-102, wherein the non-LTR retrotransposon is a retrotransposon from a clade selected from the group consisting of: CRE, R4, Hero, NeSL, R2, RandI, Proto1, L1, Tx1, RTEPT, Proto2, RTEX, RTE, Outcast, Ingi, I, Nimb, Tad1, Loa, R1, Jockey, Rex1, CR1, L2, L2A, L2B, Daphne, and Crack.

Embodiment II-105. The nucleic acid of embodiment II-102, wherein non-LTR retrotransposon is a LINE 2-2 retrotransposon.

Embodiment II-106. The nucleic acid of embodiment II-105, wherein the LINE-2-2 retrotransposon is a zebrafish LINE 2-2 retrotransposon.

Embodiment II-107. The nucleic acid of any one of embodiments II-102 to II-106, wherein the at least one amino acid modification stabilizes the reverse transcriptase domain relative to an unmodified reverse transcriptase domain.

Embodiment II-108. The nucleic acid of embodiment II-107, wherein the at least one amino acid modification a) improves packing of hydrophobic residues in the core of the reverse transcriptase domain, b) stabilizes a loop region of the reverse transcriptase domain, c) alters electrostatic or H-bond stability within the reverse transcriptase domain, d) reduces the size of the active site of the reverse transcriptase domain relative to an unsubstituted reverse transcriptase domain, and/or e) increases the size of an amino acid side chain in the active site of the reverse transcriptase domain relative to an unsubstituted reverse transcriptase domain.

Embodiment II-109. The nucleic acid of embodiment II-108, wherein
a) the at least one amino acid modification that stabilizes a loop region is a proline substitution and/or addition,
b) the at least one amino acid modification that alters electrostatic or H-bond stability substitutes a charge or H-bond acceptor/donor preference, or
c) the at least one amino acid modification that reduces the size of the active site and/or increases the size of an amino acid side chain in the active site of the reverse transcriptase domain comprises at least one amino substitution corresponding to a substitution selected from the group consisting of: A688V, A688I relative to SEQ ID NO: 51.

Embodiment II-110. The nucleic acid of embodiment II-109, wherein the amino acid modification that substitutes a charge or H-bond acceptor/donor preference:
a) substitutes a non-charged amino acid with a positively charged amino acid or a hydrogen bond donor (e.g., histidine),
b) substitutes a hydrogen bond donor with a charged amino acid, or
c) substitutes a negatively charged amino acid with a non-charged amino acid.

Embodiment II-111. The nucleic acid of any one of embodiments II-107 to II-110, wherein the reverse transcriptase domain comprises at least one amino modification corresponding to a substitution selected from the group consisting of:
a) I625L, H521P, S737P, P705A, M558L, M733L, M760S, M750L, A757P, H717A, H717K, D497S, I625H, L825G, D278S, L837I, A464P, K762R, A948T, P675S, H698P, L742P, E541K, Q547R, S814P, S672P, N560P, H853P, L514P, L524P, Q449P, H650P, G674P, S800P, I896P, S474P, and D520P, relative to SEQ ID NO: 51, and
b) N647K, H717K, I625H, and D520P, relative to SEQ ID NO: 51.

Embodiment II-112. The nucleic acid of any one of embodiments II-76 to II-106, wherein the at least one amino acid modification:
a) stabilizes the association of the reverse transcriptase domain with RNA and/or DNA relative to an unmodified reverse transcriptase domain; or
b) stabilizes the association of the RNA binding domain of the reverse transcriptase with its cognate RNA relative to an unmodified RNA binding domain.

Embodiment II-113. The nucleic acid of embodiment II-112, wherein the at least one amino acid modification a) adds a positive charge, b) removes a negative charge, or c) alters at least one H-bond forming residue.

Embodiment II-114. The nucleic acid of embodiment II-112 or II-113, wherein the reverse transcriptase domain comprises at least one amino modification corresponding to a substitution selected from the group consisting of:
a) D550T, D770H, K815R, S883R, K952R, K542R, N546R, H569R, S577R, H463K, Q478K, K566R, K815R, and S960R, relative to SEQ ID NO: 51, and
b) I343K, L354N, Q357K, and E366N, relative to SEQ ID NO: 51.

Embodiment II-115. A nucleic acid encoding a retroelement-derived endonuclease domain comprising at least one amino acid modification that promotes association of the retroelement-derived endonuclease domain with DNA relative to an unmodified endonuclease domain.

Embodiment II-116. The nucleic acid of embodiment II-115, wherein the at least one amino acid modification corresponds to a substitution selected from the group consisting of: Y139K, and D64K relative to SEQ ID NO: 51.

Embodiment II-117. The nucleic acid of any one of embodiments II-1 to II-116, wherein the nucleic acid is an RNA molecule.

Embodiment II-118. The nucleic acid of any one of embodiments II-1 to II-116, wherein the nucleic acid is a DNA molecule.

Embodiment II-119. The nucleic acid of embodiment II-118, wherein the nucleic acid comprises a T7 promoter.

Embodiment II-120. The nucleic acid of any one of embodiments II-1 to II-118, wherein the nucleic acid comprises a heterologous promoter.

Embodiment II-121. The nucleic acid of embodiment II-120, wherein the promoter is a constitutive promoter.

Embodiment II-122. The nucleic acid of embodiment II-120, wherein the promoter is an inducible promoter.

Embodiment II-123. The nucleic acid of embodiment II-120, wherein the promoter is a tissue specific promoter.

Embodiment II-124. The nucleic acid of embodiment II-120, wherein the promoter is selected from the group consisting of an EF1a promoter, a CMV promoter, an A1AT promoter, an Albumin gene promoter, an MNDU promoter, an SFFV promoter, and an ApoE promoter.

Embodiment II-125. The nucleic acid of any one of embodiments II-1 to II-124, wherein the nucleic acid comprises one or more chemical or sequence modifications.

Embodiment II-126. The nucleic acid of embodiment II-125, wherein the one or more chemical or sequence modifications are selected from the group consisting of an RNA CAP, a modified polyA length, a chemical modification (e.g., a pseudouridine and/or a methylpseudouridine), a 5′ UTR modification, a 3′ UTR modification, a modified Kozak sequence, a modified stem loop, an RNA stabilization motif, a 5-methoxyuridine (5-MO-U) modification, a 5-methylcytidine (5mC) modification, and one or more additional and/or modified microsatellites.

Embodiment II-127. The nucleic acid of any one of embodiments II-1 to II-126 comprising a codon optimized sequence.

Embodiment II-128. The nucleic acid of embodiment II-127, wherein the codon optimized sequence is optimized for expression in human cells.

Embodiment II-129. The nucleic acid of embodiment II-127, wherein the codon optimized sequence has a reduced Uracil (U) load relative to a corresponding naturally occurring sequence.

Embodiment II-130. The nucleic acid of embodiment II-126, wherein the RNA stabilization motif is a WPRE motif.

Embodiment II-131. An engineered protein encoded by any one of the nucleic acids of any one of embodiments II-1 to II-130.

Embodiment II-132. A composition comprising:
a) a first nucleic acid of any one of embodiments II-1 to I1-130; and
b) a second nucleic acid comprising a polynucleotide encoding a gene of interest.

Embodiment II-133. The composition of embodiment II-132, wherein the first and second nucleic acids are separate DNA molecules.

Embodiment II-134. The composition of embodiment II-132, wherein the first and second nucleic acids are separate RNA molecules.

Embodiment II-135. The composition of embodiment II-132, wherein the one of the first and second nucleic acids is a DNA molecule and one of the first and second nucleic acids is an RNA molecule.

Embodiment II-136. The composition of any one of embodiments II-132 to II-135, wherein the first polynucleotide is operably linked to a first heterologous promoter.

Embodiment II-137. The composition of any one of embodiments I1-132 to I1-136, wherein the second polynucleotide is operably linked to a second heterologous promoter.

Embodiment II-138. The composition of embodiment II-136 or II-137, wherein at least one of the first and second heterologous promoters is a constitutive promoter.

Embodiment II-139. The composition of embodiment II-136 or II-137, wherein at least one of the first and second heterologous promoters is an inducible promoter.

Embodiment II-140. The composition of embodiment II-136 or II-137, wherein at least one of the first and second heterologous promoters is a constitutive promoter.

Embodiment II-141. The composition of embodiment II-136 or II-137, wherein the first and second heterologous promoters are independently an EF1a promoter, a CMV promoter, an A1AT promoter, an Albumin gene promoter, MNDU promoter, SFFV promoter, or an ApoE promoter.

Embodiment II-142. The composition of any one of embodiments II-132 to II-141, wherein one or both of the first and second nucleic acids further comprise one or more of the following modifications: an RNA CAP, a modified polyA length, a chemical modification, a 5′ UTR modification, a 3′ UTR modification, a modified Kozak sequence, a modified stem loop, an RNA stabilization motif, a 5-methoxyuridine (5-MO-U) modification, a 5-methylcytidine (5mC) modification, or one or more additional and/or modified microsatellites.

Embodiment II-143. The composition of any one of embodiments II-132 to II-142, wherein one or both of the first and second nucleic acids comprises a codon optimized sequence.

Embodiment II-144. The composition of embodiment II-143, wherein the codon optimized sequence is optimized for expression in human cells.

Embodiment II-145. The composition of embodiment II-143, wherein the codon optimized sequence has a reduced Uracil (U) load relative to a corresponding naturally occurring sequence.

Embodiment II-146. The composition of embodiment II-142, wherein the RNA stabilization motif is a WPRE motif.

Embodiment II-147. The composition of any one of embodiments II-132 to II-146, wherein one or both of the first and second nucleic acids further comprise an RNA nuclear localization sequence.

Embodiment II-148. The composition of embodiment II-147, wherein the RNA nuclear localization sequence is an SAFB motif.

Embodiment II-149. The composition of any one of embodiments I1-132 to I1-148, wherein the second polynucleotide is flanked by a first terminal region and a second terminal region.

Embodiment II-150. The composition of embodiment II-149, wherein the first and second terminal regions are LTRs.

Embodiment II-151. The composition of embodiment II-149, wherein the first terminal region is the 5′ UTR of a LINE, and the second terminal region is the 3′ UTR of a LINE.

Embodiment II-152. The composition of embodiment II-151, wherein the LINE 3′UTR region comprises a truncated stem loop relative to a wild-type stem loop.

Embodiment II-153. The composition of any one of embodiments I1-132 to I1-152, wherein the second nucleic acid further comprises a 5′ UTR, a 3′UTR, a polyA sequence, a sequence that is recognized, by the engineered protein encoded by the first nucleic acid, for binding, reverse transcription, and integration of the gene of interest into a target nucleic acid.

Embodiment II-154. The composition of embodiment II-153, wherein the target nucleic acid is a genome of a target cell.

Embodiment II-155. The composition of any one of embodiments II-132 to II-154, wherein the second nucleic acid comprises i) a promoter operably linked to the second polynucleotide encoding the gene of interest, and ii) a polyadenylation sequence, and wherein the promoter is selectively active in one or more target cell types.

Embodiment II-156. The composition of any one of embodiments II-132 to II-155, wherein the gene of interest encodes a therapeutic RNA and/or a therapeutic protein.

Embodiment II-157. The composition of embodiment II-156, wherein the therapeutic RNA is an antisense RNA (asRNA), small interfering RNA (siRNA), microRNA (miRNA), or RNA aptamer.

Embodiment II-158. The composition of embodiment II-156, wherein the therapeutic protein is an antibody, regulatory protein, hormone, cytokine, structural protein, enzyme, or membrane protein.

Embodiment II-159. The composition of embodiment II-156, wherein the therapeutic protein is Factor VIII, Factor IX, Phenylalanine hydroxylase, ATP7B, alpha glucosidase, argininosuccinate synthetase, galactose-1-phosphate uridyltransferase, or ornithine transcarbamylase.

Embodiment II-160. The composition of any one of embodiments II-132 to II-159, wherein the second nucleic acid comprises flanking regions homologous to target sites in a genome of a target cell.

Embodiment II-161. The composition of any one of embodiments II-132 to II-160, wherein the first and second nucleic acids are comprised within a plurality of LNP particles.

Embodiment II-162. A method of modifying a polynucleotide, comprising contacting a polynucleotide with a nucleic acid of any one of embodiments II-1 to II-130, a composition of any one of embodiments II-132 to II-161, or an engineered protein embodiment II-131 to a subject.

Embodiment II-163. The method of embodiment II-162, wherein the polynucleotide is in a cell.

Embodiment II-164. A method of treating a subject in need thereof, comprising administering to the subject a nucleic acid of any one of embodiments II-1 to II-130, a composition of any one of embodiments II-132 to II-161, or an engineered protein embodiment 1I-131 to a subject.

Embodiment II-165. Use of a nucleic acid of any one of embodiments II-1 to II-130, an engineered protein of embodiment II-131 or a composition of any one of embodiments II-132 to II-161 to treat a subject in need thereof.

Embodiment II-166. The method of embodiment II-162, or use of embodiment II-165, wherein the subject is a human.

Embodiment II-167. A nucleic acid encoding an engineered protein comprising a retroelement-derived polypeptide variant comprising an amino acid modification when compared to a wild type retroelement-derived polypeptide;
wherein the retroelement-derived polypeptide variant is derived from a non-long terminal repeat (non-LTR) retrotransposon; and
wherein the engineered protein exhibits at least one improved characteristic, as compared to the wild-type retroelement-derived polypeptide without the at least one amino acid modification.

These and other aspects are illustrated by the following non-limiting examples.

### EXAMPLES

### Example 1

Non-limiting examples of engineered proteins that can be used (e.g., directly or encoded on an RNA and/or DNA molecule) to promote integration of a heterologous gene into a target nucleic acid in a host cell include protein fusions comprising at least one domain of a retroelement-derived protein fused to at least one heterologous polypeptide that redirects and/or enhances insertion of the heterologous gene. In some embodiments, each of the at least one heterologous polypeptide comprises one or more of an RNA/DNA processing polypeptide, an RNA/DNA repair polypeptide, a nucleic acid binding polypeptide, or a nucleosome binding polypeptide. The at least one heterologous polypeptide can be fused to the N-terminus and/or C-terminus of a retroelement-derived protein or protein domain, and/or internally within a retroelement-derived protein (e.g., between two domains of the protein). Nucleic acids (e.g., RNA and/or DNA) encoding one or more engineered proteins can be used to promote insertion of a transgene (e.g., a heterologous nucleic acid comprising a gene of interest) flanked by retroelement-derived terminal regions (e.g., by 5′ and 3′ terminal regions) into a target nucleic acid. In some embodiments, a first nucleic acid that encodes an engineered protein is provided (e.g., administered to a subject) along with a separate second nucleic acid that includes a transgene flanked by the retroelement-derived terminal regions. In some embodiments, the nucleic acid that encodes the engineered protein and the transgene that is flanked by retroelement-derived terminal regions are provided (e.g., administered to a subject) on the same nucleic acid molecule.

a) Examples of Different Non-Limiting Protein Configurations that Include a Retroelement Derived RT-EN Protein or Domain Fused to One or More Heterologous Polypeptides (HPs)

The following schemes represent non-limiting alternative configurations of one or more heterologous polypeptides fused to a retroelement-derived polypeptide that includes an endonuclease domain (EN) and/or a reverse transcriptase domain (RT). A heterologous polypeptide fused to an N-terminus of the retroelement-derived polypeptide is referred to herein as an nHP. A heterologous polypeptide fused to a C-terminus of the retroelement-derived polypeptide is referred to herein as a cHP. An internally fused heterologous polypeptide is referred to as an iHP. “N-” indicates the N-terminus of the fusion protein, and “—C” indicates the C-terminus of the fusion protein. In some embodiments, a retroelement-derived polypeptide is modified to remove the naturally occurring endonuclease domain (e.g., to remove a restriction-like endonuclease (RLE) domain). In some embodiments, a heterologous endonuclease domain (hEN) is fused to the RT domain of the retroelement-derived polypeptide (e.g., to replace the naturally occurring endonuclease domain). In some embodiments, a heterologous polypeptide comprises one or more linkers and/or a localization polypeptide (e.g., NLS or NoLS). For example, a linker may be located at the N-terminus and/or the C-terminus of a heterologous polypeptide to connect the heterologous polypeptide to a retroelement-derived polypeptide. In some embodiments, the heterologous polypeptide may include one or more internal linkers, for example between different domains (e.g., different enzymatic domains) of the heterologous polypeptide.

### Fusions to a Retroelement-Derived Polypeptide:

i) N-nHP-RT-EN-C
ii) N-RT-EN-cHP-C
iii) N-nHP-RT-EN-cHP-C
iv) N-nHP-RT-iHP-EN-C
v) N-RT-iHP-EN-cHP-C
vi) N-nHP-RT-iHP-EN-cHP-C
   Fusions to a Retroelement-Derived Polypeptide Comprising a Heterologous Endonuclease Domain (hEN):
i) N-nHP-RT-hEN-C
ii) N-RT-hEN-cHP-C
iii) N-nHP-RT-hEN-cHP-C
iv) N-nHP-RT-iHP-hEN-C
v) N-RT-iHP-hEN-cHP-C
vi) N-nHP-RT-iHP-hEN-cHP-C

### Domain Fusions:

i) N-nHP-RT-C
ii) N-RT-cHP-C
iii) N-nHP-EN-C
iv) N-EN-cHP-C
   b) Examples of Different Non-Limiting Protein Configurations that Include a Retroelement Derived EN-RT Protein or Domain (e.g., from an L1, RTE, I, or Jockey Type Retroelement) Fused to One or More Heterologous Polypeptides (HPs)

The following schemes represent non-limiting alternative configurations of one or more heterologous polypeptides fused to a retroelement-derived polypeptide that include an endonuclease domain (EN) and/or a reverse transcriptase domain (RT). A heterologous polypeptide fused to an N-terminus of the retroelement-derived polypeptide is referred to herein as an nHP. A heterologous polypeptide fused to a C-terminus of the retroelement-derived polypeptide is referred to herein as a cHP. An internally fused heterologous polypeptide is referred to as an iHP. “N-” indicates the N-terminus of the fusion protein, and “-C” indicates the C-terminus of the fusion protein. In some embodiments, a retroelement-derived polypeptide is modified to remove the naturally occurring endonuclease domain (e.g., to remove an apurinic-apyrimidinic endonuclease (APE) domain). In some embodiments, a heterologous endonuclease domain (hEN) is fused to the RT domain (e.g., to replace the naturally occurring endonuclease domain). In some embodiments, a heterologous polypeptide comprises one or more linkers and/or a localization polypeptide (e.g., NLS or NoLS). For example, a linker may be located at the N-terminus and/or the C-terminus of a heterologous polypeptide to connect the heterologous polypeptide to a retroelement-derived polypeptide. In some embodiments, the heterologous polypeptide may include one or more internal linkers, for example between different domains (e.g., different enzymatic domains) of the heterologous polypeptide.

### Fusions to a Retroelement-Derived Polypeptide:

i) N-nHP-EN-RT-C
ii) N-EN-RT-cHP-C
iii) N-nHP-EN-RT-cHP-C
iv) N-nHP-EN-iHP-RT-C
v) N-EN-iHP-RT-cHP-C
vi) N-nHP-EN-iHP-RT-cHP-C
   Fusions to a Retroelement-Derived Polypeptide Comprising a Heterologous Endonuclease Domain (hEN):
i) N-nHP-hEN-RT-C
ii) N-hEN-RT-cHP-C
iii) N-nHP-hEN-RT-cHP-C
iv) N-nHP-hEN-iHP-RT-C
v) N-hEN-iHP-RT-cHP-C
vi) N-nHP-hEN-iHP-RT-cHP-C

### Domain Fusions:

i) N-nHP-RT-C
ii) N-RT-cHP-C
iii) N-nHP-EN-C
iv) N-EN-cHP-C

One or more nucleic acids (e.g., RNA and/or DNA) encoding at least one of these engineered proteins can be provided, in trans or in cis, to target cells (e.g., ex vivo or in vivo) along with one or more nucleic acids (e.g., RNA and/or DNA) encoding a transgene (e.g., flanked by terminal regions) to promote integration of the transgene into a nucleic acid (e.g., a genomic nucleic acid) of the target cells.

### Example 2

Non-limiting examples of engineered proteins that can be used (e.g., directly or encoded on an RNA and/or DNA molecule) as drivers to promote insertion of a transgene into a target nucleic acid (e.g., a host genome) are provided. One or more of the following engineered proteins can be provided alone or in combination with other engineered proteins. One or more of the following engineered proteins that are illustrated as N-terminal fusions (a heterologous polypeptide having one or more enzyme domains, e.g., 1, 2, 3, or more domains, fused to the N-terminus of a retroelement-derived polypeptide) also could be provided as C-terminal fusions (the heterologous polypeptide fused to the C-terminus of the retroelement-derived polypeptide) or as internal fusions. Similarly, one or more of the following engineered proteins that are illustrated as C-terminal fusions also could be provided as N-terminal fusions, and/or as internal fusions In some embodiments, alternative linker sequences and/or lengths could be included in the heterologous polypeptide. In some embodiments, no linkers are used between domains. In some embodiments, a nuclear and/or nucleolar localization sequence could be included.

In some embodiments, the engineered protein (for use, e.g., as a driver) may comprise a retroelement-derived polypeptide comprising a wild-type zebrafish LINE 2-2 (ZFL2-2) retroelement (SM002), the retroelement-derived polypeptide having the following sequence:

SM002 amino acid sequence:
(SEQ ID NO: 51)
MCFLIPVVINTRKTREVRCKRNPHNLRSIHVSTISQLSLSVGLWNCQSAVNKADFITSIATYSD
YNLMALTETWLRPEDTATHATLSANFSFSHTPRQTGRGGGTGLLISKEWKFTLIPSLPTISSFE
FHAVTIIHPFYINVVVIYRPPGKLGHFLDELDVLLSSFSNFDTPLLVLGDFNIYVDKPQAADFQ
TLLASFDLKRAPTSATHKSGNQLDLIYTRHCFTDQTIVTPLQISDHFLLSLNIHITPEPPHTPT
LVTFRRNLRSLSPNRLSTIVSDSLPPSRKLTALDSNSATNTLCSTLASCLDRLCPLASRPARAS
PPAPWLSDALREHRSKLRAAERIWRKTKNPAHLLTYQTLLSSFSAEVTSAKQTYYRLKINNAIN
PRLLFKTFSSLLYPPPPPASSTLTTDDFATFFCTKTAKISAQFAAPTINTQDTTPTPHILISFS
QLSESEVSKLVLSSHATTCPLDPIPSHLLQAISPAVIPTLTHIINTSLDSGLFPTTFKQARVTP
LLKKPNLDHTLLENYRPVSLLPFMAKILEKVVFNQVLDFLTQNNLMDNKQSGFKKGHSTETALL
SVVEDLRLAKADSKSSVLILLDLSAAFDTVNHQILLSTLESLGVAGTVIQWFRSYLSDRSFRVS
WRGEVSNLQHLNTGVPQGSVLGPLLFSIYTSSLGPVIQRHGFSYHCYADDTQLYLSFHPDDPSV
PARISACLLDISHWMKDHHLQLNLAKTEMLVVSANPTLHHNFSIQMDGATITASKMVKSLGVTI
DDQLNFSDHISRTARSCRFALYNIRKIRPFLSEHAAQLLVQALVLSKLDYCNSLLALLPANSIK
PLQLLQNAAARVVFNEPKRAHVTPLLVRLHWLPVAARIKFKTLMFAYKVTSGLAPSYLHSLLQI
YVPSRNLRSVNERRLVVPSQRGKKSLSRTLTLNLPSWWNELPNCIRTAESLAIFKKRLKTQLFS
LHFTS

In some examples, the following CtIP N-terminal fragment (SEQ ID:310) was incorporated into an engineered protein (e.g. as an N-terminal fusion with the N-terminus of a retroelement-derived polypeptide) for use, e.g., as a driver:

(SEQ ID: 310)
NISGSSCGSPNSADTSSDFKDLWTKLKECHDREVQGLQVKVTKLK
QERILDAQRLEEFFTKNQQLREQQKVLHETIKVLEDRLRAGLCDR
CAVTEEHMRKKQQEFENIRQQNLKLITELMNERNTLQEENKKLSE
QLQQKIENDQQHQAAELECEEDVIPDSPITAFSESGVNRLRRKEN
PHVRYIEQTHTKLEHSVCANEMRKVSKSSTHPQHNPNENEILVAD
TYDQSQSPMAKAHGTSSYTPDKSSFNLATVVAETLGLGVQEESET
QGPMSPLGDELYHCLEGNHKKQPFES

In some examples, the following RAD51-derived protein (SEQ ID NO: 311) was incorporated into an engineered protein (e.g., as an N-terminal fusion with the N-terminus of a retroelement-derived polypeptide) for use, e.g., as a driver:

(SEQ ID NO: 311)
GIHGVPAAAMQMQLEANADTSVEEESFGPQPISRLEQCGINANDV
KKLEEAGFHTVEAVAYAPKKELINIKGISEAKADKILAEAAKLVP
MGFTTETEFHQRRSEIIQITTGSKELDKLLQGGIETGSITEMFGE
FRTGKTQICHTLAVTCQLPIDRGGGEGKAMYIDTEGTFRPERLLA
VAERYGLSGSDVLDNVAYARAFNTDHQTQLLYQASAMMVESRYAL
LIVDSATALYRTDYSGRGELSARQMHLARFLRMLLRLADEFGVAV
VITNQVVAQVDGAAMFAADPKKPIGGNIIAHASTTRLYLRKGRGE
TRICKIYDSPCLPEAEAMFAINADGVGDAKD

In some examples, the following UL12 protein (SEQ ID NO: 25) was incorporated into an engineered protein (e.g., as an N-terminal fusion with the N-terminus of a retroelement-derived polypeptide) for use, e.g., as a driver:

(SEQ ID NO: 25)
ESTVGPACPPGRTVTKRPWALAEDTPRGPDSPPKRPRPNSLPLTT
TFRPLPPPPQTTSAVDPSSHSPVNPPRDQHATDTADEKPRAASPA
LSDASGPPTPDIPLSPGGTHARDPDADPDSPDLDS

In some examples, the following BRCA2-derived protein (SEQ ID NO: 308) was incorporated into an engineered protein (e.g., as an N-terminal fusion with the N-terminus of a retroelement-derived polypeptide) for use, e.g., as a driver:

(SEQ ID NO: 308)
PTLLGFHTASGKKVKIAKESLDKVKNLFDEKEQ

In some examples, the following DSS1-derived protein (SEQ ID NO: 309) was incorporated into an engineered protein (e.g., as an N-terminal fusion with the N-terminus of a retroelement-derived polypeptide) for use, e.g., as a driver:

(SEQ ID NO: 309)
SEKKQPVDLGLLEEDDEFEEFPAEDWAGLDEDEDAHVWEDNWDDD
NVEDDFSNQLRAELEKHGYKMETS

In some examples, the following HMGN1 polypeptide (SEQ ID NO: 23) was incorporated into an engineered protein (e.g., as an N-terminal fusion with the N-terminus of a retroelement-derived polypeptide) for use, e.g., as a driver:

(SEQ ID NO: 23)
MPKRKVSSAEGAAKEEPKRRSARLSAKPPAKVEAKPKKAAAKDKS
SDKKVQTKGKRGAKGKQAEVANQETKEDLPAENGETKTEESPASD
EAGEKEAKSD

In some examples, the following HMGB1 polypeptide (SEQ ID NO: 24) was incorporated into an engineered protein (e.g., as C-terminal fusion with the C-terminus of a retroelement-derived polypeptide) for use, e.g., as a driver:

(SEQ ID NO: 24)
GKGDPKKPRGKMSSYAFFVQTCREEHKKKHPDASVNFSEFSKKCS
ERWKTMSAKEKGKFEDMAKADKARYEREMKTYIPPKGE

In some examples, the following Sto7d polypeptide (SEQ ID NO: 26) was incorporated into an engineered protein (e.g., as a C-terminal fusion) for use, e.g., as a driver:

(SEQ ID NO: 26)
VTVKFKYKGEEKEVDISKIKKVWRVGKMISFTYDDNGKTGRGAVS
EKDAPKELLQMLEK

In some examples the following Nibrin derived MRE11 recruitment peptide (SEQ ID NO: 312) was incorporated into an engineered protein (e.g., as a C-terminal fusion) for use, e.g., as a driver:

(SEQ ID NO: 312)
KNSTSRNPSGINDDYGQLKNFKKFKKVTYGS

In some examples the following MDM2 derived p53 inhibitory peptide (SEQ ID NO: 313) was incorporated into an engineered protein (e.g., as a C-terminal fusion) for use, e.g., as a driver:

(SEQ ID NO: 313)
CNTNMSVPTDGAVTTSQIPASEQETLVRPKPLLLKLLKSVGAQKD
TYTMKEVLFYLGQYIMTKRLYDEKQQHIVYCSNDLLGDLFGVPSF
SVKEHRKIYTMIYRNLVVVNQQESSDSGTSVSENGS

In some examples the following p53 inhibitory peptide (SEQ ID NO: 314) was incorporated into an engineered protein (e.g., as a C-terminal fusion) for use, e.g., as a driver:

(SEQ ID NO: 314)
YGFRLGFLHSGTAKSVTCTYGS

In some examples the following Nango-derived peptide (SEQ ID NO: 315) was incorporated into an engineered protein (e.g., as a C-terminal fusion) for use, e.g., as a driver:

(SEQ ID NO: 315)
LQSCMQFQPNSPASDLEAALEAAGEGLNVIQQTTRYFSTPQTMDL
FLNYSMNMQPEDVGS

In some examples the following E. coli RnaseH1 domain (SEQ ID NO: 316) was incorporated into an engineered protein (e.g., as a C-terminal fusion) for use, e.g., as a driver:

(SEQ ID NO: 316)
LKQVEIFTDGSCLGNPGPGGYGAILRYRGREKTFSAGYTRTTNNR
MELMAAIVALEALKEHCEVILSTDSQYVRQGITQWIHNWKKRGWK
TADKKPVKNVDLWQRLDAALGQHQIKWEWVKGHAGHPENERCDEL
ARAAAMNPTLEDTGYQVEVGS

In some examples the following human RNase H1 catalytic domain (SEQ ID NO: 317) was incorporated into an engineered protein (e.g., as a C-terminal fusion) for use, e.g., as a driver:

(SEQ ID NO: 317)
GDFVVVYTDGCCSSNGRRRPRAGIGVYWGPGHPLNVGIRLPGRQT
NQRAEIHAACKAIEQAKTQNINKLVLYTDSMFTINGITNWVQGWK
KNGWKTSAGKEVINKEDFVALERLTQGMDIQWMHVPGHSGFIGNE
EADRLAREGAKQSEDGS

In some examples the following zinc finger AAVS1 DNA-binding domain (SEQ ID NO: 318) was incorporated into an engineered protein (e.g., as an N-terminal fusion) for use, e.g., as a driver:

(SEQ ID NO: 318)
GIHGVPAAMAERPFQCRICMRNFSYNWHLQRHIRTHTGEKPFACD
ICGRKFARSDHLTTHTKIHTGSQKPFQCRICMRNFSHNYARDCHI
RTHTGEKPFACDICGRKFAQNSTRIGHTKIHLRGSSGSETPGTSE
SATPEGIHGVPAAMAERPFQCRICMRNFSQSSNLARHIRTHTGEK
PFACDICGRKFARTDYLVDHTKIHTGSQKPFQCRICMRNFSYNTH
LTRHIRTHTGEKPFACDICGRKFAQGYNLAGHTKIHLRGS

In some examples, the following dead SpCas9 (containing mutations D10A and H840A; SEQ ID NO: 330) was incorporated into an engineered protein (e.g., as an N-terminal fusion) for use, e.g., as a driver:

(SEQ ID NO: 330)
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDAIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD

In some examples, the following PCSK9 homing endonuclease SEQ ID NO: 331) was incorporated into an engineered protein (e.g., as an N-terminal fusion) for use, e.g., as a driver:

(SEQ ID NO: 331)
NTKYNKEFLLYLAGFVDGDGSIFARIKPSQRSKFKHKLHLVFAVY
QKTQRRWFLDKLVDEIGVGYVLDSGSVSFYSLSEIKPLHNFLTQL
QPFLKLKQKQANLVLKIIEQLPSAKESPDKFLEVCTWVDQIAALN
DSKTRKTTSETVRAVLDSLPGSVGGLSPSQASSAASSASSSPGSG
ISEALRAGAGSGTGYNKEFLLYLAGFVDGDGSIYARIKPVQRAKF
KHELVLGFDVTQKTQRRWFLDKLVDEIGVGYVYDKGSVSAYRLSQ
IKPLHNFLTQLQPFLKLKQKQANLVLKIIEQLPSAKESPDKFLEV
CTWVDQIAALNDSKTRKTTSETVRAVLDSLSEKKKSSP

In some examples, the following PCSK9 homing nickase (Q47E substitution; SEQ ID NO: 332) was incorporated into an engineered protein (e.g., as an N-terminal fusion) for use, e.g., as a driver:

(SEQ ID NO: 332)
NTKYNKEFLLYLAGFVDGDGSIFARIKPSQRSKFKHKLHLVFAVY
EKTQRRWFLDKLVDEIGVGYVLDSGSVSFYSLSEIKPLHNFLTQL
QPFLKLKQKQANLVLKIIEQLPSAKESPDKFLEVCTWVDQIAALN
DSKTRKTTSETVRAVLDSLPGSVGGLSPSQASSAASSASSSPGSG
ISEALRAGAGSGTGYNKEFLLYLAGFVDGDGSIYARIKPVQRAKF
KHELVLGFDVTQKTQRRWFLDKLVDEIGVGYVYDKGSVSAYRLSQ
IKPLHNFLTQLQPFLKLKQKQANLVLKIIEQLPSAKESPDKFLEV
CTWVDQIAALNDSKTRKTTSETVRAVLDSLSEKKKSSP

In some examples, the following Cas9 nickase (H840A substitution; SEQ ID NO: 333) was incorporated into an engineered protein (e.g., as an N-terminal fusion) for use, e.g., as a driver:

(SEQ ID NO: 333)
MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDAIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD

In addition, in some examples one or more nuclear or nucleolar localization sequences were included into an engineered protein (e.g., as a N or C-terminal fusion) for use, e.g., as a driver:

SV40 NLS:
(SEQ ID NO: 54)
PKKKRKV
Nucleoplasmin NLS:
(SEQ ID NO: 55)
KRPAATKKAGQAKKKK
Bipartite SV40 NLS:
(SEQ ID NO: 56)
KRTADGSEFESPKKKRKV
PNRC NLS:
(SEQ ID NO: 57)
PKKRRKKK
PolyR NLS:
(SEQ ID NO: 58)
RRRRRRR
H2B NLS:
(SEQ ID NO: 59)
KKRKRSRK

In addition, in some examples, one or more peptide linkers were included between peptides or protein domains:

Rigid linker:
(SEQ ID NO: 334)
SGSETPGTSESATPEGS
GS linker 1:
(SEQ ID NO: 335)
GS
GS linker 2:
(SEQ ID NO: 336)
GGSG
GS linker 3:
(SEQ ID NO: 337)
GSGS
GS linker 4:
(SEQ ID NO: 338)
GGGS
GS linker 5:
(SEQ ID NO: 339)
GGSGGG
GS linker 6:
(SEQ ID NO: 340)
GSGSGSGS

SEQ ID NO: 1 illustrates an N-terminal CtIP fragment L2-2 fusion. The CtIP fragment is shown near the N-terminus of the engineered protein, fused via a XTEN linker (shown in bold italics text) to a C-terminal L2-2 protein (shown in underlined text). A nuclear localization sequence (shown in bold underlined lowercase text) is shown fused at the N-terminus of the CtIP fragment.

(SEQ ID NO: 1)
MAkkkrkvNISGSSCGSNSADTSSDFKDLWTKLKECHDREVQGLQ
VKVTKLKQERILDAQRLEEFFTKNQQLREQQKVLHETIKVLEDRL
RAGLCDRCAVTEEHMRKKQQEFENIRQQNLKLITELMNERNTLQE
ENKKLSEQLQQKIENDQQHQAAELECEEDVIDSITAFSFSGVNRL
RRKENHVRYIEQTHTKLEHSVCANEMRKVSKSSTHQHNNENEILV
ADTYDQSQSMAKAHGTSSYTDKSSFNLATVVAETLGLGVQEESET
QGMSLGDELYHCLEGNHKKQFESGSETGTSESATEGSCFLIVVIN
TRKTREVRCKRNHNLRSIHVSTISQLSLSVGLWNCQSAVNKADFI
TSIATYSDYNLMALTETWLREDTATHATLSANFSFSHTRQTGRGG
GTGLLISKEWKFTLISLTISSFEFHAVTIIHFYINVVVIYRGKLG
HFLDELDVLLSSFSNFDTLLVLGDENIYVDKQAADFQTLLASFDL
KRATSATHKSGNQLDLIYTRHCFTDQTIVTLQISDHFLLSLNIHI
TEHTTLVTFRRNLRSLSNRLSTIVSDSLSRKLTALDSNSATNTLC
STLASCLDRLCLASRARASAWLSDALREHRSKLRAAERIWRKTKN
AHLLTYQTLLSSFSAEVTSAKQTYYRLKINNAINRLLFKTFSSLL
YASSTLTTDDFATFFCTKTAKISAQFAATTNTQDTTTHTLTSFSQ
LSESEVSKLVLSSHATTCLDISHLLQAISAVITLTHIINTSLDSG
LFTTFKQARVTLLKKNLDHTLLENYRVSLLFMAKILEKVVENQVL
DELTQNNLMDNKQSGFKKGHSTETALLSVVEDLRLAKADSKSSVL
ILLDLSAAFDTVNHQILLSTLESLGVAGTVIQWERSYLSDRSFRV
SWRGEVSNLQHLNTGVQGSVLGLLFSIYTSSLGVIQRHGFSYHCY
ADDTQLYLSFHDDSVARISACLLDISHWMKDHHLQLNLAKTEMLV
VSANTLHHNFSIQMDGATITASKMVKSLGVTIDDQLNFSDHISRT
ARSCRFALYNIRKIRFLSEHAAQLLVQALVLSKLDYCNSLLALLA
NSIKLQLLQNAAARVVFNEKRAHVTLLVRLHWLVAARIKFKTLMF
AYKVTSGLASYLHSLLQIYVSRNLRSVNERRLVVSQRGKKSLSRT
LTLNLSWWNELNCIRTAESLAIFKKRLKTQLESLHFTS

SEQ ID NO: 2 illustrates an N-terminal RAD51 L2-2 fusion. The RAD51-derived protein is shown near the N-terminus of the engineered protein, fused via a linker (shown in bold italics text) to a C-terminal L2-2 protein (shown in underlined text). A nuclear localization sequence (shown in bold underlined lowercase text) is shown at the N-terminus of the engineered protein, fused to the RAD51-derived protein.

(SEQ ID NO: 2)
MAkkkrkvGIHGVAAAMQMQLEANADTSVEEESFGQISRLEQCGI
NANDVKKLEEAGFHTVEAVAYAKKELINIKGISEAKADKILAEAA
KLVMGETTETEFHQRRSEIIQITTGSKELDKLLQGGIETGSITEM
FGEFRTGKTQICHTLAVTCQLIDRGGGEGKAMYIDTEGTFRERLL
AVAERYGLSGSDVLDNVAYARAFNTDHQTQLLYQASAMMVESRYA
LLIVDSATALYRTDYSGRGELSARQMHLARFLRMLLRLADEFGVA
VVITNQVVAQVDGAAMFAADKKIGGNIIAHASTTRLYLRKGRGET
RICKIYDSCLEAEAMFAINADGVGDAKDGGSGSETGTSESATESG
GSGCFLIVVTNTRKTREVRCKRNHNLRSIHVSTISQLSLSVGLWN
CQSAVNKADFITSIATYSDYNLMALTETWLREDTATHATLSANFS
ESHTRQTGRGGGTGLLISKEWKFTLISLTISSFEFHAVTIIHFYI
NVVVIYRGKLGHFLDELDVLLSSFSNEDTLLVLGDFNIYVDKQAA
DFQTLLASFDLKRATSATHKSGNQLDLIYTRHCFTDQTIVTLQIS
DHFLLSLNIHITEHTTLVTERRNLRSLSNRLSTIVSDSLSRKLTA
LDSNSAINTLCSTLASCLDRLCLASRARASAWLSDALREHRSKLR
AAERIWRKTKNAHLLTYQTLLSSFSAEVTSAKQTYYRLKINNATN
RLLEKTFSSLLYASSTLTTDDFATFFCTKTAKISAQFAATTNTQD
TTTHTLTSFSQLSESEVSKLVLSSHATTCLDISHLLQAISAVITL
THIINTSLDSGLFTTFKQARVTLLKKNLDHTLLENYRVSLLFMAK
ILEKVVFNQVLDELTQNNLMDNKQSGFKKGHSTETALLSVVEDLR
LAKADSKSSVLILLDLSAAFDTVNHQILLSTLESLGVAGTVIQWE
RSYLSDRSFRVSWRGEVSNLQHLNTGVQGSVLGLLFSIYTSSLGV
IQRHGFSYHCYADDTQLYLSFHDDSVARISACLLDISHWMKDHHL
QLNLAKTEMLVVSANTLHHNESIQMDGATITASKMVKSLGVTIDD
QLNFSDHISRTARSCRFALYNIRKIRFLSEHAAQLLVQALVLSKL
DYCNSLLALLANSIKLQLLQNAAARVVFNEKRAHVTLLVRLHWLV
AARIKFKTLMFAYKVTSGLASYLHSLLQIYVSRNLRSVNERRLVV
SQRGKKSLSRTLTLNLSWWNELNCIRTAESLAIFKKRLKTQLFSL
HFTS

SEQ ID NO: 3 illustrates an N-terminal UL12 L2-2 fusion. The UL12 polypeptide is shown near the N-terminus of the engineered protein, fused via a linker (shown in bold italics text) to a C-terminal L2-2 protein (shown in underlined text). A nuclear localization sequence (shown in bold underlined lowercase text) is shown at the N-terminus of the engineered protein, fused to the UL12 polypeptide.

(SEQ ID NO: 3)
MAkkkrkvESTVGACGRTVTKRWALAEDTRGDSKRRNSLLITTFR
LQTTSAVDSSHSVNRDQHATDTADEKRAASALSDASGTDILSGGT
HARDDADDSDLDSGSETGTSESATESCFLIVVTNTRKTREVRCKR
NHNLRSIHVSTISQLSLSVGLWNCQSAVNKADFITSIATYSDYNL
MALTETWLREDTATHATLSANFSFSHTRQTGRGGGTGLLISKEWK
FTLISLTISSFEFHAVTIIHFYINVVVIYRGKLGHFLDELDVLLS
SFSNEDTLLVLGDFNIYVDKQAADFQTLLASFDLKRATSATHKSG
NQLDLIYTRHCFTDQTIVTLQISDHFLLSLNIHITEHTTLVTERR
NLRSLSNRLSTIVSDSLSRKLTALDSNSAINTLCSTLASCLDRLC
LASRARASAWLSDALREHRSKLRAAERIWRKTKNAHLLTYQTLLS
SFSAEVTSAKQTYYRLKINNATNRLLFKTFSSLLYASSTLTTDDF
ATFFCTKTAKISAQFAATTNTQDTTTHTLTSFSQLSESEVSKLVL
SSHATTCLDISHLLQAISAVITLTHIINTSLDSGLFTTFKQARVT
LLKKNLDHTLLENYRVSLLFMAKILEKVVENQVLDELTQNNLMDN
KQSGFKKGHSTETALLSVVEDLRLAKADSKSSVLILLDLSAAFDT
VNHQILLSTLESLGVAGTVIQWFRSYLSDRSFRVSWRGEVSNLQH
LNTGVQGSVLGLLFSIYTSSLGVIQRHGFSYHCYADDTQLYLSFH
DDSVARISACLLDISHWMKDHHLQLNLAKTEMLVVSANTLHHNFS
IQMDGATITASKMVKSLGVTIDDQLNFSDHISRTARSCRFALYNI
RKIRFLSEHAAQLLVQALVLSKLDYCNSLLALLANSIKLQLLQNA
AARVVFNEKRAHVTLLVRLHWLVAARIKFKTLMFAYKVTSGLASY
LHSLLQIYVSRNLRSVNERRLVVSQRGKKSLSRTLTLNLSWWNEL
NCIRTAESLAIFKKRLKTQLFSLHFTS

SEQ ID NO: 4 illustrates an N-terminal BRCA2-derived peptide L2-2 fusion. The BRCA2-derived peptide is shown near the N-terminus of the engineered protein, fused via a linker (shown in bold italics text) to a C-terminal L2-2 protein (shown in underlined text). A nuclear localization sequence (shown in bold underlined lowercase text) is shown at the N-terminus of the engineered protein, fused to the BRCA2-derived peptide.

(SEQ ID NO: 4)
MAkkkrkvTLLGFHTASGKKVKIAKESLDKVKNLFDEKEQGGSGG
GCFLIVVTNTRKTREVRCKRNHNLRSIHVSTISQLSLSVGLWNCQ
SAVNKADFITSIATYSDYNLMALTETWLREDTATHATLSANFSFS
HTRQTGRGGGTGLLISKEWKFTLISLTISSFEFHAVTIIHFYINV
VVIYRGKLGHFLDELDVLLSSFSNFDTLLVLGDFNIYVDKQAADF
QTLLASFDLKRATSATHKSGNQLDLIYTRHCFTDQTIVTLQISDH
FLLSLNIHITEHTTLVTERRNLRSLSNRLSTIVSDSLSRKLTALD
SNSATNTLCSTLASCLDRLCLASRARASAWLSDALREHRSKLRAA
ERIWRKTKNAHLLTYQTLLSSFSAEVTSAKQTYYRLKINNATNRL
LFKTESSLLYASSTLTTDDFATFFCTKTAKISAQFAATTNTQDTT
THTLTSFSQLSESEVSKLVLSSHATTCLDISHLLQAISAVITLTH
IINTSLDSGLFTTFKQARVTLLKKNLDHTLLENYRVSLLFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVQGSVLGLLFSIYTSSLGVIQ
RHGFSYHCYADDTQLYLSFHDDSVARISACLLDISHWMKDHHLQL
NLAKTEMLVVSANTLHHNFSIQMDGATITASKMVKSLGVTIDDQL
NFSDHISRTARSCRFALYNIRKIRFLSEHAAQLLVQALVLSKLDY
CNSLLALLANSIKLQLLQNAAARVVENEKRAHVTLLVRLHWLVAA
RIKFKTLMFAYKVTSGLASYLHSLLQIYVSRNLRSVNERRLVVSQ
RGKKSLSRTLTLNLSWWNELNCIRTAESLAIFKKRLKTQLFSLHF
TS

SEQ ID NO: 5 illustrates an N-terminal DSS1-derived peptide L2-2 fusion. The DSS1-derived peptide is shown near the N-terminus of the engineered protein, fused via a linker (shown in bold italics text) to a C-terminal L2-2 protein (shown in underlined text). A nuclear localization sequence (shown in bold underlined lowercase text) is shown at the N-terminus of the engineered protein, fused to the N-terminus of the DSS1-derived peptide.

(SEQ ID NO: 5)
MAkkkrkvSEKKQVDLGLLEEDDEFEEFAEDWAGLDEDEDAHVWE
DNWDDDNVEDDFSNQLRAELEKHGYKMETSGGSGGGSGCFLIVVT
NTRKTREVRCKRNHNLRSIHVSTISQLSLSVGLWNCQSAVNKADF
ITSIATYSDYNLMALTETWLREDTATHATLSANFSFSHTRQTGRG
GGTGLLISKEWKFTLISLTISSFEFHAVTIIHFYINVVVIYRGKL
GHELDELDVLLSSFSNEDTLLVLGDFNIYVDKQAADFQTLLASFD
LKRATSATHKSGNQLDLIYTRHCFTDQTIVTLQISDHFLLSLNIH
ITEHTTLVTFRRNLRSLSNRLSTIVSDSLSRKLTALDSNSATNTL
CSTLASCLDRLCLASRARASAWLSDALREHRSKLRAAERIWRKTK
NAHLLTYQTLLSSFSAEVTSAKQTYYRLKINNATNRLLFKTESSL
LYASSTLTTDDFATFFCTKTAKISAQFAATTNTQDTTTHTLTSFS
QLSESEVSKLVLSSHATTCLDISHLLQAISAVITLTHIINTSLDS
GLFTTFKQARVTLLKKNLDHTLLENYRVSLLFMAKILEKVVFNQV
LDELTQNNLMDNKQSGFKKGHSTETALLSVVEDLRLAKADSKSSV
LILLDLSAAFDTVNHQILLSTLESLGVAGTVIQWERSYLSDRSFR
VSWRGEVSNLQHLNTGVQGSVLGLLFSIYTSSLGVIQRHGFSYHC
YADDTQLYLSFHDDSVARISACLLDISHWMKDHHLQLNLAKTEML
VVSANTLHHNESIQMDGATITASKMVKSLGVTIDDQLNFSDHISR
TARSCRFALYNIRKIRFLSEHAAQLLVQALVLSKLDYCNSLLALL
ANSIKLQLLQNAAARVVFNEKRAHVTLLVRLHWLVAARIKFKTLM
FAYKVTSGLASYLHSLLQIYVSRNLRSVNERRLVVSQRGKKSLSR
TLTLNLSWWNELNCIRTAESLAIFKKRLKTQLFSLHFTS

SEQ ID NO: 6 illustrates an N-terminal HMGN1 L2-2 fusion. A HMGN1 polypeptide is shown at the N-terminus of the engineered protein, fused via a linker (shown in bold italics text) to the N-terminus of the L2-2 protein (shown in underlined text).

(SEQ ID NO: 6)
MKRKVSSAEGAAKEEKRRSARLSAKAKVEAKKKAAAKDKSSDKKV
QTKGKRGAKGKQAEVANQETKEDLAENGETKTEESASDEAGEKEA
KSDSGSETGTSESATESCFLIVVTNTRKTREVRCKRNHNLRSIHV
STISQLSLSVGLWNCQSAVNKADFITSIATYSDYNLMALTETWLR
EDTATHATLSANFSFSHTRQTGRGGGTGLLISKEWKFTLISLTIS
SFEFHAVTIIHFYINVVVIYRGKLGHFLDELDVLLSSFSNFDTLL
VLGDFNIYVDKQAADFQTLLASFDLKRATSATHKSGNQLDLIYTR
HCFTDQTIVTLQISDHFLLSLNIHITEHTTLVTFRRNLRSLSNRL
STIVSDSLSRKLTALDSNSATNTLCSTLASCLDRLCLASRARASA
WLSDALREHRSKLRAAERIWRKTKNAHLLTYQTLLSSFSAEVTSA
KQTYYRLKINNATNRLLFKTFSSLLYASSTLTTDDFATFFCTKTA
KISAQFAATINTQDTTTHTLTSFSQLSESEVSKLVLSSHATTCLD
ISHLLQAISAVITLTHIINTSLDSGLFTTFKQARVTLLKKNLDHT
LLENYRVSLLFMAKILEKVVFNQVLDELTQNNLMDNKQSGFKKGH
STETALLSVVEDLRLAKADSKSSVLILLDLSAAFDTVNHQILLST
LESLGVAGTVIQWERSYLSDRSFRVSWRGEVSNLQHLNTGVQGSV
LGLLFSIYTSSLGVIQRHGFSYHCYADDTQLYLSFHDDSVARISA
CLLDISHWMKDHHLQLNLAKTEMLVVSANTLHHNFSIQMDGATIT
ASKMVKSLGVTIDDQLNFSDHISRTARSCRFALYNIRKIRFLSEH
AAQLLVQALVLSKLDYCNSLLALLANSIKLQLLQNAAARVVFNEK
RAHVTLLVRLHWLVAARIKFKTLMFAYKVTSGLASYLHSLLQIYV
SRNLRSVNERRLVVSQRGKKSLSRTLTLNLSWWNELNCIRTAESL
AIFKKRLKTQLFSLHFTS

SEQ ID NO: 7 illustrates a C-terminal HMGB1 L2-2 fusion. An HMGB1 polypeptide is shown near the C-terminus of the engineered protein, fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the HMGB1 polypeptide.

(SEQ ID NO: 7)
MCFLIVVTNTRKTREVRCKRNHNLRSIHVSTISQLSLSVGLWNCQ
SAVNKADFITSIATYSDYNLMALTETWLREDTATHATLSANFSFS
HTRQTGRGGGTGLLISKEWKFTLISLTISSFEFHAVTIIHFYINV
VVIYRGKLGHELDELDVLLSSFSNEDTLLVLGDFNIYVDKQAADF
QTLLASFDLKRATSATHKSGNQLDLIYTRHCFTDQTIVTLQISDH
FLLSLNIHITEHTTLVTFRRNLRSLSNRLSTIVSDSLSRKLTALD
SNSATNTLCSTLASCLDRLCLASRARASAWLSDALREHRSKLRAA
ERIWRKTKNAHLLTYQTLLSSFSAEVTSAKQTYYRLKINNAINRL
LFKTFSSLLYASSTLTTDDFATFFCTKTAKISAQFAATTNTQDTT
THTLTSFSQLSESEVSKLVLSSHATTCLDISHLLQAISAVITLTH
IINTSLDSGLFTTEKQARVTLLKKNLDHTLLENYRVSLLFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVQGSVLGLLFSIYTSSLGVIQ
RHGFSYHCYADDTQLYLSFHDDSVARISACLLDISHWMKDHHLQL
NLAKTEMLVVSANTLHHNFSIQMDGATITASKMVKSLGVTIDDQL
NFSDHISRTARSCRFALYNIRKIRFLSEHAAQLLVQALVLSKLDY
CNSLLALLANSIKLQLLQNAAARVVFNEKRAHVTLLVRLHWLVAA
RIKFKTLMFAYKVTSGLASYLHSLLQIYVSRNLRSVNERRLVVSQ
RGKKSLSRTLTLNLSWWNELNCIRTAESLAIFKKRLKTQLESLHF
TSSGSETGTSESATEGKGDKKRGKMSSYAFFVQTCREEHKKKHDA
SVNFSEFSKKCSERWKTMSAKEKGKFEDMAKADKARYEREMKTYI
KGEGGSGkrtadgsefeskkkrkv

SEQ ID NO: 8 illustrates a C-terminal Sto7d L2-2 fusion. A Sto7d DNA binding domain is shown near the C-terminus of the engineered protein, fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the Sto7d DNA binding domain.

(SEQ ID NO: 8)
MCFLIVVTNTRKTREVRCKRNHNLRSIHVSTISQLSLSVGLWNCQ
SAVNKADFITSIATYSDYNLMALTETWLREDTATHATLSANFSFS
HTRQTGRGGGTGLLISKEWKFTLISLTISSFEFHAVTIIHFYINV
VVIYRGKLGHFLDELDVLLSSFSNEDTLLVLGDENIYVDKQAADF
QTLLASFDLKRATSATHKSGNQLDLIYTRHCFTDQTIVTLQISDH
FLLSLNIHITEHTTLVTFRRNLRSLSNRLSTIVSDSLSRKLTALD
SNSATNTLCSTLASCLDRLCLASRARASAWLSDALREHRSKLRAA
ERIWRKTKNAHLLTYQTLLSSFSAEVTSAKQTYYRLKINNAINRL
LFKTFSSLLYASSTLTTDDFATFFCTKTAKISAQFAATTNTQDTT
THTLTSFSQLSESEVSKLVLSSHATTCLDISHLLQAISAVITLTH
IINTSLDSGLFTTFKQARVTLLKKNLDHTLLENYRVSLLFMAKIL
EKVVENQVLDELTQNNLMDNKQSGFKKGHSTETALLSVVEDLRLA
KADSKSSVLILLDLSAAFDTVNHQILLSTLESLGVAGTVIQWFRS
YLSDRSFRVSWRGEVSNLQHLNTGVQGSVLGLLFSIYTSSLGVIQ
RHGFSYHCYADDTQLYLSFHDDSVARISACLLDISHWMKDHHLQL
NLAKTEMLVVSANTLHHNFSIQMDGATITASKMVKSLGVTIDDQL
NFSDHISRTARSCRFALYNIRKIRFLSEHAAQLLVQALVLSKLDY
CNSLLALLANSIKLQLLQNAAARVVFNEKRAHVTLLVRLHWLVAA
RIKFKTLMFAYKVTSGLASYLHSLLQIYVSRNLRSVNERRLVVSQ
RGKKSLSRTLTLNLSWWNELNCIRTAESLAIFKKRLKTQLESLHF
TSGGSGVTVKFKYKGEEKEVDISKIKKVWRVGKMISFTYDDNGKT
GRGAVSEKDAKELLQMLEKGGSGkrtadgsefeskkkrkv

SEQ ID NO: 9 illustrates a C-terminal Nibrin MRE11 recruitment peptide L2-2 fusion. The Nibrin MRE11 recruitment peptide is shown near the C-terminus of the engineered protein, fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the Nibrin MRE11 recruitment peptide.

(SEQ ID NO: 9)
MCFLIVVTNTRKTREVRCKRNHNLRSIHVSTISQLSLSVGLWNCQ
SAVNKADFITSIATYSDYNLMALTETWLREDTATHATLSANFSFS
HTRQTGRGGGTGLLISKEWKFTLISLTISSFEFHAVTIIHFYINV
VVIYRGKLGHELDELDVLLSSFSNEDTLLVLGDFNIYVDKQAADF
QTLLASFDLKRATSATHKSGNQLDLIYTRHCFTDQTIVTLQISDH
FLLSLNIHITEHTTLVTFRRNLRSLSNRLSTIVSDSLSRKLTALD
SNSATNTLCSTLASCLDRLCLASRARASAWLSDALREHRSKLRAA
ERIWRKTKNAHLLTYQTLLSSFSAEVTSAKQTYYRLKINNAINRL
LFKTFSSLLYASSTLTTDDFATFFCTKTAKISAQFAATTNTQDTT
THTLTSFSQLSESEVSKLVLSSHATTCLDISHLLQAISAVITLTH
IINTSLDSGLFTTEKQARVTLLKKNLDHTLLENYRVSLLFMAKIL
EKVVENQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVQGSVLGLLFSIYTSSLGVIQ
RHGFSYHCYADDTQLYLSFHDDSVARISACLLDISHWMKDHHLQL
NLAKTEMLVVSANTLHHNFSIQMDGATITASKMVKSLGVTIDDQL
NFSDHISRTARSCRFALYNIRKIRFLSEHAAQLLVQALVLSKLDY
CNSLLALLANSIKLQLLQNAAARVVFNEKRAHVTLLVRLHWLVAA
RIKFKTLMFAYKVTSGLASYLHSLLQIYVSRNLRSVNERRLVVSQ
RGKKSLSRTLTLNLSWWNELNCIRTAESLAIFKKRLKTQLFSLHF
TSGSGSGSGSKNSTSRNSGINDDYGQLKNFKKFKKVTYGSkkkrk
v

SEQ ID NO: 10 illustrates a C-terminal MDM2 p53 inhibitory peptide L2-2 fusion. The MDM2 p53 inhibitory peptide is shown near the C-terminus of the engineered protein, fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the MDM2 p53 inhibitory peptide.

(SEQ ID NO: 10)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNATNPRLLFKTESSLLYPPPPPASS
TLTTDDFATFFCTKTAKISAQFAAPTTNTQDTTPTPHTLISESQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSGSGSGSGSCNTNMSVPTDGAVTTSQ
IPASEQETLVRPKPLLLKLLKSVGAQKDTYTMKEVLFYLGQYIMT
KRLYDEKQQHIVYCSNDLLGDLFGVPSFSVKEHRKIYTMIYRNLV
VVNQQESSDSGTSVSENGSpkkkrkv

SEQ ID NO: 11 illustrates a p53-inhibiting peptide L2-2 fusion. The p53-inhibiting peptide (YGFRLGFLHSGTAKSVTCTY; SEQ ID NO: 314) is shown near the C-terminus fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the p53-inhibiting peptide.

(SEQ ID NO: 11)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNATNPRLLFKTFSSLLYPPPPPASS
TLTTDDFATFFCTKTAKISAQFAAPTTNTQDTTPTPHILISFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDFLTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSGSGSGSGSGSYGFRLGFLHSGTAKS
VTCTYGSpkkkrkv

SEQ ID NO: 12 illustrates a Nanog-derived peptide L2-2 fusion. The Nanog-derived peptide is shown near the C-terminus fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the Nanog-derived peptide.

(SEQ ID NO: 12)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDEN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNATNPRLLFKTFSSLLYPPPPPASS
TLTTDDFATFFCTKTAKISAQFAAPTTNTQDTTPTPHTLISESQL
SESEVSKLVLSSHATTCPLDPIPSHLLQAISPAVIPTLTHIINTS
LDSGLFPTTEKQARVTPLLKKPNLDHTLLENYRPVSLLPFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKEKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSGSGSGSGSSLQSCMQFQPNSPASDL
EAALEAAGEGLNVIQQTTRYFSTPQTMDLFLNYSMNMQPEDVGSp
kkkrkv

SEQ ID NO: 13 illustrates an E. coli RNase H1 domain L2-2 fusion. The E. coli RNase H1 domain is shown near the C-terminus fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the E. coli RNase H1 domain.

(SEQ ID NO: 13)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNATNPRLLFKTFSSLLYPPPPPASS
TLTTDDFATFFCTKTAKISAQFAAPTTNTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVENQVLDELTQNNLMDNKQSGFKKGHSTETALLSVVEDLRLA
KADSKSSVLILLDLSAAFDTVNHQILLSTLESLGVAGTVIQWFRS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSGSGSGSGSLKQVEIFTDGSCLGNPG
PGGYGAILRYRGREKTFSAGYTRTTNNRMELMAAIVALEALKEHC
EVILSTDSQYVRQGITQWIHNWKKRGWKTADKKPVKNVDLWQRLD
AALGQHQIKWEWVKGHAGHPENERCDELARAAAMNPTLEDTGYQV
EVGSpkkkrkv

SEQ ID NO: 14 illustrates a human RNase H1 catalytic domain L2-2 fusion. The human RNaseH1 catalytic domain is shown near the C-terminus fused via a linker (shown in bold italics text) to a N-terminal L2-2 protein (shown in underlined text). A nuclear localization sequence (shown in bold underlined lowercase text) is shown at the C-terminus fused via a linker (shown in bold italics text) to the human RNase H1 catalytic domain.

(SEQ ID NO: 14)
MCFLIPVVINTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNATNPRLLFKTFSSLLYPPPPPASS
TLTTDDFATFFCTKTAKISAQFAAPTINTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDFLTQNNLMDNKQSGFKKGHSTETALLSVVEDLRLA
KADSKSSVLILLDLSAAFDTVNHQILLSTLESLGVAGTVIQWFRS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLFSLHFTSGSGSGSGSGSGDFVVVYTDGCCSSN
GRRRPRAGIGVYWGPGHPLNVGIRLPGRQTNQRAEIHAACKAIEQ
AKTQNINKLVLYTDSMFTINGITNWVQGWKKNGWKTSAGKEVINK
EDFVALERLTQGMDIQWMHVPGHSGFIGNEEADRLAREGAKQSED
GSpkkkrkv

SEQ ID NO: 15 illustrates a Zinc finger targeting AAVS1 safe harbor L2-2 fusion. The Zinc finger targeting AAVS1 safe harbor is shown near the N-terminus of the engineered protein fused to a C-terminal L2-2 protein (shown in underlined text). A nuclear localization sequence (shown in bold underlined lowercase text) is shown at the N-terminus of the engineered protein fused to the Zinc finger targeting AAVS1 safe harbor.

(SEQ ID NO: 15)
MApkkkrkvGIHGVPAAMAERPFQCRICMRNFSYNWHLQRHIRTH
TGEKPFACDICGRKFARSDHLTTHTKIHTGSQKPFQCRICMRNFS
HNYARDCHIRTHTGEKPFACDICGRKFAQNSTRIGHTKIHLRGSS
GSETPGTSESATPEGIHGVPAAMAERPFQCRICMRNFSQSSNLAR
HIRTHTGEKPFACDICGRKFARTDYLVDHTKIHTGSQKPFQCRIC
MRNFSYNTHLTRHIRTHTGEKPFACDICGRKFAQGYNLAGHTKIH
LRGSSGSETPGTSESATPECFLIPVVINTRKTREVRCKRNPHNLR
SIHVSTISQLSLSVGLWNCQSAVNKADFITSIATYSDYNLMALTE
TWLRPEDTATHATLSANFSFSHTPRQTGRGGGTGLLISKEWKFTL
IPSLPTISSFEFHAVTIIHPFYINVVVIYRPPGKLGHFLDELDVL
LSSFSNFDTPLLVLGDFNIYVDKPQAADFQTLLASFDLKRAPTSA
THKSGNQLDLIYTRHCFTDQTIVTPLQISDHFLLSLNIHITPEPP
HTPTLVTERRNLRSLSPNRLSTIVSDSLPPSRKLTALDSNSATNT
LCSTLASCLDRLCPLASRPARASPPAPWLSDALREHRSKLRAAER
IWRKTKNPAHLLTYQTLLSSFSAEVTSAKQTYYRLKINNATNPRL
LFKTFSSLLYPPPPPASSTLTTDDFATFFCTKTAKISAQFAAPTT
NTQDTTPTPHTLTSFSQLSESEVSKLVLSSHATTCPLDPIPSHLL
QAISPAVIPTLTHIINTSLDSGLFPTTFKQARVTPLLKKPNLDHT
LLENYRPVSLLPFMAKILEKVVFNQVLDFLTQNNLMDNKQSGFKK
GHSTETALLSVVEDLRLAKADSKSSVLILLDLSAAFDTVNHQILL
STLESLGVAGTVIQWERSYLSDRSFRVSWRGEVSNLQHLNTGVPQ
GSVLGPLLFSIYTSSLGPVIQRHGFSYHCYADDTQLYLSFHPDDP
SVPARISACLLDISHWMKDHHLQLNLAKTEMLVVSANPTLHHNFS
IQMDGATITASKMVKSLGVTIDDQLNFSDHISRTARSCRFALYNI
RKIRPFLSEHAAQLLVQALVLSKLDYCNSLLALLPANSIKPLQLL
QNAAARVVFNEPKRAHVTPLLVRLHWLPVAARIKFKTLMFAYKVT
SGLAPSYLHSLLQIYVPSRNLRSVNERRLVVPSQRGKKSLSRTLT
LNLPSWWNELPNCIRTAESLAIFKKRLKTQLFSLHFTS

SEQ ID NO: 16 illustrates a dead Cas9 L2-2 fusion. The Cas9 portion is shown at the N-terminus of the engineered protein fused via a linker (shown in bold italics text) to a C-terminal L2-2 protein (shown in underlined text). A nuclear localization sequence (shown in bold underlined lowercase text) is shown at the N-terminus of the engineered protein fused to the Cas9 portion.

(SEQ ID NO: 16)
MApkkkrkvGRGMDKKYSIGLAIGTNSVGWAVITDEYKVPSKKFK
VLGNTDRHSIKKNLIGALLFDSGETAEATRLKRTARRRYTRRKNR
ICYLQEIFSNEMAKVDDSFFHRLEESFLVEEDKKHERHPIFGNIV
DEVAYHEKYPTIYHLRKKLVDSTDKADLRLIYLALAHMIKERGHF
LIEGDLNPDNSDVDKLFIQLVQTYNQLFEENPINASGVDAKAILS
ARLSKSRRLENLIAQLPGEKKNGLFGNLIALSLGLTPNFKSNEDL
AEDAKLQLSKDTYDDDLDNLLAQIGDQYADLFLAAKNLSDAILLS
DILRVNTEITKAPLSASMIKRYDEHHQDLTLLKALVRQQLPEKYK
EIFFDQSKNGYAGYIDGGASQEEFYKFIKPILEKMDGTEELLVKL
NREDLLRKQRTFDNGSIPHQIHLGELHAILRRQEDFYPFLKDNRE
KIEKILTFRIPYYVGPLARGNSRFAWMTRKSEETITPWNFEEVVD
KGASAQSFIERMINEDKNLPNEKVLPKHSLLYEYFTVYNELTKVK
YVTEGMRKPAFLSGEQKKAIVDLLEKTNRKVTVKQLKEDYFKKIE
CFDSVEISGVEDRFNASLGTYHDLLKIIKDKDFLDNEENEDILED
IVLTLTLFEDREMIEERLKTYAHLFDDKVMKQLKRRRYTGWGRLS
RKLINGIRDKQSGKTILDELKSDGFANRNFMQLIHDDSLTFKEDI
QKAQVSGQGDSLHEHIANLAGSPAIKKGILQTVKVVDELVKVMGR
HKPENIVIEMARENQTTQKGQKNSRERMKRIEEGIKELGSQILKE
HPVENTQLQNEKLYLYYLQNGRDMYVDQELDINRLSDYDVDAIVP
QSFLKDDSIDNKVLTRSDKNRGKSDNVPSEEVVKKMKNYWRQLLN
AKLITQRKFDNLTKAERGGLSELDKAGFIKRQLVETRQITKHVAQ
ILDSRMNTKYDENDKLIREVKVITLKSKLVSDFRKDFQFYKVREI
NNYHHAHDAYLNAVVGTALIKKYPKLESEFVYGDYKVYDVRKMIA
KSEQEIGKATAKYFFYSNIMNFFKTEITLANGEIRKRPLIEINGE
TGEIVWDKGRDFATVRKVLSMPQVNIVKKTEVQTGGFSKESILPK
RNSDKLIARKKDWDPKKYGGFDSPTVAYSVLVVAKVEKGKSKKLK
SVKELLGITIMERSSFEKNPIDFLEAKGYKEVKKDLIIKLPKYSL
FELENGRKRMLASAGELQKGNELALPSKYVNFLYLASHYEKLKGS
PEDNEQKQLFVEQHKHYLDEIIEQISEFSKRVILADANLDKVLSA
YNKHRDKPIREQAENIIHLFTLTNLGAPAAFKYFDTTIDRKRYTS
TKEVLDATLIHQSITGLYETRIDLSQLGGDGGSGSETPGTSESAT
PESGGSGCFLIPVVTNTRKTREVRCKRNPHNLRSIHVSTISQLSL
SVGLWNCQSAVNKADFITSIATYSDYNLMALTETWLRPEDTATHA
TLSANFSFSHTPRQTGRGGGTGLLISKEWKFTLIPSLPTISSFEF
HAVTIIHPFYINVVVIYRPPGKLGHFLDELDVLLSSFSNFDTPLL
VLGDFNIYVDKPQAADFQTLLASFDLKRAPTSATHKSGNQLDLIY
TRHCFTDQTIVTPLQISDHFLLSLNIHITPEPPHTPTLVTFRRNL
RSLSPNRLSTIVSDSLPPSRKLTALDSNSATNTLCSTLASCLDRL
CPLASRPARASPPAPWLSDALREHRSKLRAAERIWRKTKNPAHLL
TYQTLLSSFSAEVTSAKQTYYRLKINNATNPRLLFKTFSSLLYPP
PPPASSTLTTDDFATFFCTKTAKISAQFAAPTTNTQDTTPTPHTL
TSFSQLSESEVSKLVLSSHATTCPLDPIPSHLLQAISPAVIPTLT
HIINTSLDSGLFPTTFKQARVTPLLKKPNLDHTLLENYRPVSLLP
FMAKILEKVVFNQVLDELTQNNLMDNKQSGFKKGHSTETALLSVV
EDLRLAKADSKSSVLILLDLSAAFDTVNHQILLSTLESLGVAGTV
IQWERSYLSDRSFRVSWRGEVSNLQHLNTGVPQGSVLGPLLFSIY
TSSLGPVIQRHGFSYHCYADDTQLYLSFHPDDPSVPARISACLLD
ISHWMKDHHLQLNLAKTEMLVVSANPTLHHNFSIQMDGATITASK
MVKSLGVTIDDQLNFSDHISRTARSCRFALYNIRKIRPFLSEHAA
QLLVQALVLSKLDYCNSLLALLPANSIKPLQLLQNAAARVVFNEP
KRAHVTPLLVRLHWLPVAARIKFKTLMFAYKVTSGLAPSYLHSLL
QIYVPSRNLRSVNERRLVVPSQRGKKSLSRTLTLNLPSWWNELPN
CIRTAESLAIFKKRLKTQLFSLHFTS

SEQ ID NO: 17 illustrates a PCSK9 homing endonuclease L2-2 D237A fusion. The PCSK9 homing endonuclease portion is shown near the N-terminus of the engineered protein, fused via a linker (shown in bold italics text) to a C-terminal L2-2 protein (shown in underlined text) having a D237A substitution. A nuclear localization sequence (shown in bold underlined lowercase text) is shown at the N-terminus of the engineered protein fused to the PCSK9 homing endonuclease.

(SEQ ID NO: 17)
MApkkkrkvNTKYNKEFLLYLAGFVDGDGSIFARIKPSQRSKFKH
KLHLVFAVYQKTQRRWELDKLVDEIGVGYVLDSGSVSFYSLSEIK
PLHNFLTQLQPFLKLKQKQANLVLKIIEQLPSAKESPDKFLEVCT
WVDQIAALNDSKTRKTTSETVRAVLDSLPGSVGGLSPSQASSAAS
SASSSPGSGISEALRAGAGSGTGYNKEFLLYLAGFVDGDGSIYAR
IKPVQRAKFKHELVLGFDVTQKTQRRWFLDKLVDEIGVGYVYDKG
SVSAYRLSQIKPLHNFLTQLQPFLKLKQKQANLVLKIIEQLPSAK
ESPDKFLEVCTWVDQIAALNDSKIRKTTSETVRAVLDSLSEKKKS
SPSGSETPGTSESATPECFLIPVVTNTRKTREVRCKRNPHNLRSI
HVSTISQLSLSVGLWNCQSAVNKADFITSIATYSDYNLMALTETW
LRPEDTATHATLSANFSFSHTPRQTGRGGGTGLLISKEWKFTLIP
SLPTISSFEFHAVTIIHPFYINVVVIYRPPGKLGHFLDELDVLLS
SFSNFDTPLLVLGDFNIYVDKPQAADFQTLLASFDLKRAPTSATH
KSGNQLDLIYTRHCFTDQTIVTPLQISAHFLLSLNIHITPEPPHT
PTLVTERRNLRSLSPNRLSTIVSDSLPPSRKLTALDSNSATNTLC
STLASCLDRLCPLASRPARASPPAPWLSDALREHRSKLRAAERIW
RKTKNPAHLLTYQTLLSSFSAEVTSAKQTYYRLKINNATNPRLLF
KTFSSLLYPPPPPASSTLTTDDFATFFCTKTAKISAQFAAPTINT
QDTTPTPHTLTSFSQLSESEVSKLVLSSHATTCPLDPIPSHLLQA
ISPAVIPTLTHIINTSLDSGLFPTTFKQARVTPLLKKPNLDHTLL
ENYRPVSLLPFMAKILEKVVENQVLDELTQNNLMDNKQSGFKKGH
STETALLSVVEDLRLAKADSKSSVLILLDLSAAFDTVNHQILLST
LESLGVAGTVIQWERSYLSDRSFRVSWRGEVSNLQHLNTGVPQGS
VLGPLLFSIYTSSLGPVIQRHGFSYHCYADDTQLYLSFHPDDPSV
PARISACLLDISHWMKDHHLQLNLAKTEMLVVSANPTLHHNFSIQ
MDGATITASKMVKSLGVTIDDQLNFSDHISRTARSCRFALYNIRK
IRPFLSEHAAQLLVQALVLSKLDYCNSLLALLPANSIKPLQLLQN
AAARVVFNEPKRAHVTPLLVRLHWLPVAARIKFKTLMFAYKVTSG
LAPSYLHSLLQIYVPSRNLRSVNERRLVVPSQRGKKSLSRTLTLN
LPSWWNELPNCIRTAESLAIFKKRLKTQLFSLHFTS

SEQ ID NO: 18 illustrates a PCSK9 homing endonuclease L2-2 endonuclease deleted fusion. The PCSK9 homing endonuclease portion is shown near the N-terminus of the engineered protein, fused via a linker (shown in bold italics text) to a C-terminal L2-2 protein (shown in underlined text) from which the endonuclease domain has been deleted. A nuclear localization sequence (shown in bold underlined lowercase text) is shown at the N-terminus fused the PCSK9 homing endonuclease.

(SEQ ID NO: 18)
MApkkkrkvNTKYNKEFLLYLAGFVDGDGSIFARIKPSQRSKFKH
KLHLVFAVYQKTQRRWFLDKLVDEIGVGYVLDSGSVSFYSLSEIK
PLHNFLTQLQPFLKLKQKQANLVLKIIEQLPSAKESPDKFLEVCT
WVDQIAALNDSKTRKTTSETVRAVLDSLPGSVGGLSPSQASSAAS
SASSSPGSGISEALRAGAGSGTGYNKEFLLYLAGFVDGDGSIYAR
IKPVQRAKFKHELVLGFDVTQKTQRRWFLDKLVDEIGVGYVYDKG
SVSAYRLSQIKPLHNFLTQLQPFLKLKQKQANLVLKIIEQLPSAK
ESPDKFLEVCTWVDQIAALNDSKIRKTTSETVRAVLDSLSEKKKS
SPSGSETPGTSESATPEPPHTPTLVTFRRNLRSLSPNRLSTIVSD
SLPPSRKLTALDSNSATNTLCSTLASCLDRLCPLASRPARASPPA
PWLSDALREHRSKLRAAERIWRKTKNPAHLLTYQTLLSSFSAEVT
SAKQTYYRLKINNATNPRLLFKTFSSLLYPPPPPASSTLTTDDFA
TFFCTKTAKISAQFAAPTTNTQDTTPTPHTLTSFSQLSESEVSKL
VLSSHATTCPLDPIPSHLLQAISPAVIPTLTHIINTSLDSGLFPT
TFKQARVTPLLKKPNLDHTLLENYRPVSLLPFMAKILEKVVFNQV
LDELTQNNLMDNKQSGFKKGHSTETALLSVVEDLRLAKADSKSSV
LILLDLSAAFDTVNHQILLSTLESLGVAGTVIQWERSYLSDRSFR
VSWRGEVSNLQHLNTGVPQGSVLGPLLFSIYTSSLGPVIQRHGFS
YHCYADDTQLYLSFHPDDPSVPARISACLLDISHWMKDHHLQLNL
AKTEMLVVSANPTLHHNFSIQMDGATITASKMVKSLGVTIDDQLN
FSDHISRTARSCRFALYNIRKIRPFLSEHAAQLLVQALVLSKLDY
CNSLLALLPANSIKPLQLLQNAAARVVFNEPKRAHVTPLLVRLHW
LPVAARIKFKTLMFAYKVTSGLAPSYLHSLLQIYVPSRNLRSVNE
RRLVVPSQRGKKSLSRTLTLNLPSWWNELPNCIRTAESLAIFKKR
LKTQLFSLHETS

SEQ ID NO: 19 illustrates a PCSK9 homing nickase (Q47E) L2-2 D237A fusion. The PCSK9 homing nickase portion is shown near the N-terminus of the engineered protein, fused via a linker (shown in bold italics text) to a C-terminal L2-2 protein having a D237A substitution (shown in underlined text). A nuclear localization sequence (shown in bold underlined lowercase text) is shown at the N-terminus fused to the PCSK9 homing nickase.

(SEQ ID NO: 19)
MApkkkrkvNTKYNKEFLLYLAGFVDGDGSIFARIKPSQRSKFKH
KLHLVFAVYEKTQRRWELDKLVDEIGVGYVLDSGSVSFYSLSEIK
PLHNELTQLQPFLKLKQKQANLVLKIIEQLPSAKESPDKFLEVCT
WVDQIAALNDSKTRKTTSETVRAVLDSLPGSVGGLSPSQASSAAS
SASSSPGSGISEALRAGAGSGTGYNKEFLLYLAGFVDGDGSIYAR
IKPVQRAKFKHELVLGFDVTQKTQRRWFLDKLVDEIGVGYVYDKG
SVSAYRLSQIKPLHNFLTQLQPFLKLKQKQANLVLKIIEQLPSAK
ESPDKFLEVCTWVDQIAALNDSKIRKTTSETVRAVLDSLSEKKKS
SPSGSETPGTSESATPECFLIPVVTNTRKTREVRCKRNPHNLRSI
HVSTISQLSLSVGLWNCQSAVNKADFITSIATYSDYNLMALTETW
LRPEDTATHATLSANFSFSHTPRQTGRGGGTGLLISKEWKFTLIP
SLPTISSFEFHAVTIIHPFYINVVVIYRPPGKLGHFLDELDVLLS
SFSNFDTPLLVLGDFNIYVDKPQAADFQTLLASEDLKRAPTSATH
KSGNQLDLIYTRHCFTDQTIVTPLQISAHFLLSLNIHITPEPPHT
PTLVTERRNLRSLSPNRLSTIVSDSLPPSRKLTALDSNSAINTLC
STLASCLDRLCPLASRPARASPPAPWLSDALREHRSKLRAAERIW
RKTKNPAHLLTYQTLLSSFSAEVTSAKQTYYRLKINNATNPRLLF
KTFSSLLYPPPPPASSTLTTDDFATFFCTKTAKISAQFAAPTTNT
QDTTPTPHTLTSFSQLSESEVSKLVLSSHATTCPLDPIPSHLLQA
ISPAVIPTLTHIINTSLDSGLFPTTFKQARVTPLLKKPNLDHTLL
ENYRPVSLLPFMAKILEKVVENQVLDELTQNNLMDNKQSGFKKGH
STETALLSVVEDLRLAKADSKSSVLILLDLSAAFDTVNHQILLST
LESLGVAGTVIQWERSYLSDRSFRVSWRGEVSNLQHLNTGVPQGS
VLGPLLFSIYTSSLGPVIQRHGFSYHCYADDTQLYLSFHPDDPSV
PARISACLLDISHWMKDHHLQLNLAKTEMLVVSANPTLHHNFSIQ
MDGATITASKMVKSLGVTIDDQLNFSDHISRTARSCRFALYNIRK
IRPFLSEHAAQLLVQALVLSKLDYCNSLLALLPANSIKPLQLLQN
AAARVVFNEPKRAHVTPLLVRLHWLPVAARIKFKTLMFAYKVTSG
LAPSYLHSLLQIYVPSRNLRSVNERRLVVPSQRGKKSLSRTLTLN
LPSWWNELPNCIRTAESLAIFKKRLKTQLFSLHFTS

SEQ ID NO: 20 illustrates a PCSK9 homing nickase (Q47E) L2-2 endonuclease deleted fusion. The PCSK9 homing portion is shown at the N-terminus of the engineered protein, fused via a linker (shown in bold italics text) to a C-terminal L2-2 protein from which the endonuclease domain has been deleted (shown in underlined text). A nuclear localization sequence (shown in bold underlined lowercase text) is shown at the N-terminus fused to the PCSK9 homing nickase.

(SEQ ID NO: 20)
MApkkkrkvNTKYNKEFLLYLAGFVDGDGSIFARIKPSQRSKFKH
KLHLVFAVYEKTQRRWELDKLVDEIGVGYVLDSGSVSFYSLSEIK
PLHNFLTQLQPFLKLKQKQANLVLKIIEQLPSAKESPDKFLEVCT
WVDQIAALNDSKIRKTTSETVRAVLDSLPGSVGGLSPSQASSAAS
SASSSPGSGISEALRAGAGSGTGYNKEFLLYLAGFVDGDGSIYAR
IKPVQRAKFKHELVLGFDVTQKTQRRWFLDKLVDEIGVGYVYDKG
SVSAYRLSQIKPLHNFLTQLQPFLKLKQKQANLVLKIIEQLPSAK
ESPDKFLEVCTWVDQIAALNDSKTRKTTSETVRAVLDSLSEKKKS
SPSGSETPGTSESATPEPPHTPTLVTFRRNLRSLSPNRLSTIVSD
SLPPSRKLTALDSNSATNTLCSTLASCLDRLCPLASRPARASPPA
PWLSDALREHRSKLRAAERIWRKTKNPAHLLTYQTLLSSFSAEVT
SAKQTYYRLKINNAINPRLLFKTFSSLLYPPPPPASSTLTTDDFA
TFFCTKTAKISAQFAAPTTNTQDTTPTPHILISFSQLSESEVSKL
VLSSHATTCPLDPIPSHLLQAISPAVIPTLTHIINTSLDSGLFPT
TFKQARVTPLLKKPNLDHTLLENYRPVSLLPFMAKILEKVVFNQV
LDELTQNNLMDNKQSGFKKGHSTETALLSVVEDLRLAKADSKSSV
LILLDLSAAFDTVNHQILLSTLESLGVAGTVIQWERSYLSDRSFR
VSWRGEVSNLQHLNTGVPQGSVLGPLLFSIYTSSLGPVIQRHGFS
YHCYADDTQLYLSFHPDDPSVPARISACLLDISHWMKDHHLQLNL
AKTEMLVVSANPTLHHNFSIQMDGATITASKMVKSLGVTIDDQLN
FSDHISRTARSCRFALYNIRKIRPFLSEHAAQLLVQALVLSKLDY
CNSLLALLPANSIKPLQLLQNAAARVVFNEPKRAHVTPLLVRLHW
LPVAARIKFKTLMFAYKVTSGLAPSYLHSLLQIYVPSRNLRSVNE
RRLVVPSQRGKKSLSRTLTLNLPSWWNELPNCIRTAESLAIFKKR
LKTQLFSLHFTS

SEQ ID NO: 21 illustrates a Cas9 nickase fused to an endonuclease-deleted L2-2. The Cas9 nickase portion is shown near the N-terminus of the engineered protein, fused via a linker (shown in bold italics text) to a C-terminal L2-2 protein from which the endonuclease domain has been deleted (shown in underlined text). A nuclear localization sequence (shown in bold underlined lowercase text) is shown at the N-terminus fused to the Cas9 nickase.

(SEQ ID NO: 21)
MkrtadgsefespkkkrkvDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KERGHFLIEGDLNPDNSDVDKLFIQLVQTYNQLFEENPINASGVD
AKAILSARLSKSRRLENLIAQLPGEKKNGLFGNLIALSLGLTPNE
KSNFDLAEDAKLQLSKDTYDDDLDNLLAQIGDQYADLFLAAKNLS
DAILLSDILRVNTEITKAPLSASMIKRYDEHHQDLILLKALVRQQ
LPEKYKEIFFDQSKNGYAGYIDGGASQEEFYKFIKPILEKMDGTE
ELLVKLNREDLLRKQRTEDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IEINGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLINLGAPAAFKYFDTTID
RKRYTSTKEVLDATLIHQSITGLYETRIDLSQLGGDSGGSSGGSS
GSETPGTSESATPESSGGSSGGSSPPHTPTLVTFRRNLRSLSPNR
LSTIVSDSLPPSRKLTALDSNSATNTLCSTLASCLDRLCPLASRP
ARASPPAPWLSDALREHRSKLRAAERIWRKTKNPAHLLTYQTLLS
SFSAEVTSAKQTYYRLKINNATNPRLLFKTFSSLLYPPPPPASST
LTTDDFATFFCTKTAKISAQFAAPTTNTQDTTPTPHTLTSFSQLS
ESEVSKLVLSSHATTCPLDPIPSHLLQAISPAVIPTLTHIINTSL
DSGLFPTTFKQARVTPLLKKPNLDHTLLENYRPVSLLPFMAKILE
KVVFNQVLDFLTQNNLMDNKQSGFKKGHSTETALLSVVEDLRLAK
ADSKSSVLILLDLSAAFDTVNHQILLSTLESLGVAGTVIQWERSY
LSDRSFRVSWRGEVSNLQHLNTGVPQGSVLGPLLFSIYTSSLGPV
IQRHGFSYHCYADDTQLYLSFHPDDPSVPARISACLLDISHWMKD
HHLQLNLAKTEMLVVSANPTLHHNFSIQMDGATITASKMVKSLGV
TIDDQLNFSDHISRTARSCRFALYNIRKIRPFLSEHAAQLLVQAL
VLSKLDYCNSLLALLPANSIKPLQLLQNAAARVVFNEPKRAHVTP
LLVRLHWLPVAARIKFKTLMFAYKVTSGLAPSYLHSLLQIYVPSR
NLRSVNERRLVVPSQRGKKSLSRTLTLNLPSWWNELPNCIRTAES
LAIFKKRLKTQLFSLHFTS

SEQ ID NO: 22 illustrates a Cas9 nuclease fused to endonuclease-deleted L2-2. The Cas9 nuclease portion is shown near the N-terminus of the engineered protein, fused via a XTEN linker with additional GS sequences (shown in bold italics text) to a C-terminal L2-2 protein from which the endonuclease domain has been deleted (shown in underlined text). A nuclear localization sequence (shown in bold underlined lowercase text) is shown at the N-terminus fused to the Cas9 nuclease portion.

(SEQ ID NO: 22)
MkrtadgsefespkkkrkvDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESELVEEDKKHERHP
IFGNIVDEVAYHEKYPTIYHLRKKLVDSTDKADLRLIYLALAHMI
KERGHFLIEGDLNPDNSDVDKLFIQLVQTYNQLFEENPINASGVD
AKAILSARLSKSRRLENLIAQLPGEKKNGLFGNLIALSLGLTPNE
KSNEDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLEKTNRKVTVKQLKED
YFKKIECFDSVEISGVEDRFNASLGTYHDLLKIIKDKDELDNEEN
EDILEDIVLTLTLFEDREMIEERLKTYAHLEDDKVMKQLKRRRYT
GWGRLSRKLINGIRDKQSGKTILDELKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDHIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDERKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IEINGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLINLGAPAAFKYFDTTID
RKRYTSTKEVLDATLIHQSITGLYETRIDLSQLGGDSGGSSGGSS
GSETPGTSESATPESSGGSSGGSSPPHTPTLVTFRRNLRSLSPNR
LSTIVSDSLPPSRKLTALDSNSATNTLCSTLASCLDRLCPLASRP
ARASPPAPWLSDALREHRSKLRAAERIWRKTKNPAHLLTYQTLLS
SFSAEVISAKQTYYRLKINNATNPRLLFKTFSSLLYPPPPPASST
LTTDDFATFFCTKTAKISAQFAAPTTNTQDTTPTPHTLTSFSQLS
ESEVSKLVLSSHATTCPLDPIPSHLLQAISPAVIPTLTHIINTSL
DSGLFPTTFKQARVTPLLKKPNLDHTLLENYRPVSLLPFMAKILE
KVVENQVLDELTQNNLMDNKQSGFKKGHSTETALLSVVEDLRLAK
ADSKSSVLILLDLSAAFDTVNHQILLSTLESLGVAGTVIQWERSY
LSDRSFRVSWRGEVSNLQHLNTGVPQGSVLGPLLFSIYTSSLGPV
IQRHGFSYHCYADDTQLYLSFHPDDPSVPARISACLLDISHWMKD
HHLQLNLAKTEMLVVSANPTLHHNFSIQMDGATITASKMVKSLGV
TIDDQLNFSDHISRTARSCRFALYNIRKIRPFLSEHAAQLLVQAL
VLSKLDYCNSLLALLPANSIKPLQLLQNAAARVVFNEPKRAHVTP
LLVRLHWLPVAARIKFKTLMFAYKVTSGLAPSYLHSLLQIYVPSR
NLRSVNERRLVVPSQRGKKSLSRTLTLNLPSWWNELPNCIRTAES
LAIFKKRLKTQLFSLHFTS

Nucleic acids (e.g., RNA or DNA) encoding one or more of these proteins can be provided in cis or in trans along with a nucleic acid encoding a transgene (e.g., flanked by terminal sequences).

### Example 3

Nucleic acids were designed and produced to encode non-limiting examples of engineered fusion proteins comprising a ZFL2-2 protein fused to one or more C-terminal and/or N-terminal heterologous polypeptides. These were tested and evaluated using an experimental retrotransposition assay.

The following descriptions outline the components of several non-limiting examples of engineered proteins (driver) that were evaluated in transposition assays.
a) EX282: illustrates a ZFL2-2 driver protein with a C-terminal fusion of an NBN-derived polypeptide. The NBN-derived polypeptide is shown at the C-terminus fused via a linker (shown in bold italics text) to a N-terminal ZFL2-2 protein (shown in underlined text). A nuclear localization sequence (shown in bold underlined lowercase text) is shown at the C-terminus fused to a N-terminal Nibrin-derived polypeptide. The EX282 driver is encoded by the EX282 driver construct (SEQ ID NO: 28) EX282 amino acid sequence:

(SEQ ID NO: 27)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNAINPRLLFKTFSSLLYPPPPPASS
TLTTDDFATFFCTKTAKISAQFAAPTTNTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSGSGSGSGSKNSTSRNPSGINDDYGQ
LKNFKKFKKVTYGSpkkkrkv

b) EX284: illustrates a ZFL2-2 driver protein with an N-terminal fusion of an MDM2-derived polypeptide. The MDM2-derived polypeptide is shown at the N-terminus fused via a linker (shown in bold italics text) to a C-terminal ZFL2-2 protein (shown in underlined text). A nuclear localization sequence (shown in bold underlined lowercase text) is shown at the N-terminus fused via a linker (shown in bold italics text) to a C-terminal MDM2-derived polypeptide. The EX284 driver is encoded by the EX284 driver construct (SEQ ID NO: 30) EX284 amino acid sequence:

(SEQ ID NO: 29)
MApkkkrkvGRGCNTNMSVPTDGAVTTSQIPASEQETLVRPKPLL
LKLLKSVGAQKDTYTMKEVLFYLGQYIMTKRLYDEKQQHIVYCSN
DLLGDLFGVPSFSVKEHRKIYTMIYRNLVVVNQQESSDSGTSVSE
NSGSETPGTSESATPESCFLIPVVINTRKTREVRCKRNPHNLRSI
HVSTISQLSLSVGLWNCQSAVNKADFITSIATYSDYNLMALTETW
LRPEDTATHATLSANFSFSHTPRQTGRGGGTGLLISKEWKFTLIP
SLPTISSFEFHAVTIIHPFYINVVVIYRPPGKLGHFLDELDVLLS
SFSNFDTPLLVLGDFNIYVDKPQAADFQTLLASFDLKRAPTSATH
KSGNQLDLIYTRHCFTDQTIVTPLQISDHFLLSLNIHITPEPPHT
PTLVTFRRNLRSLSPNRLSTIVSDSLPPSRKLTALDSNSAINTLC
STLASCLDRLCPLASRPARASPPAPWLSDALREHRSKLRAAERIW
RKTKNPAHLLTYQTLLSSFSAEVTSAKQTYYRLKINNATNPRLLF
KTESSLLYPPPPPASSTLTTDDFATFFCTKTAKISAQFAAPTTNT
QDTTPTPHTLTSFSQLSESEVSKLVLSSHATTCPLDPIPSHLLQA
ISPAVIPTLTHIINTSLDSGLFPTTFKQARVTPLLKKPNLDHTLL
ENYRPVSLLPFMAKILEKVVFNQVLDELTQNNLMDNKQSGFKKGH
STETALLSVVEDLRLAKADSKSSVLILLDLSAAFDTVNHQILLST
LESLGVAGTVIQWERSYLSDRSFRVSWRGEVSNLQHLNTGVPQGS
VLGPLLFSIYTSSLGPVIQRHGFSYHCYADDTQLYLSFHPDDPSV
PARISACLLDISHWMKDHHLQLNLAKTEMLVVSANPTLHHNFSIQ
MDGATITASKMVKSLGVTIDDQLNFSDHISRTARSCRFALYNIRK
IRPFLSEHAAQLLVQALVLSKLDYCNSLLALLPANSIKPLQLLQN
AAARVVFNEPKRAHVTPLLVRLHWLPVAARIKFKTLMFAYKVTSG
LAPSYLHSLLQIYVPSRNLRSVNERRLVVPSQRGKKSLSRTLTLN
LPSWWNELPNCIRTAESLAIFKKRLKTQLFSLHFTS

c) EX584: illustrates a ZFL2-2 driver protein with a C-terminal fusion of a UL12 polypeptide. The UL12 polypeptide is shown at the C-terminus fused via a linker (shown in bold italics text) to a N-terminal ZFL2-2 protein (shown in underlined text). A nuclear localization sequence (shown in bold underlined lowercase text) is shown at the C-terminus fused via a linker (shown in bold italics text) to a N-terminal UL12 polypeptide. The EX584 driver is encoded by the EX584 driver construct (SEQ ID NO: 32).

### EX584 Amino Acid Sequence:

(SEQ ID NO: 31)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHELDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNATNPRLLFKTFSSLLYPPPPPASS
TLTTDDFATFFCTKTAKISAQFAAPTTNTQDTTPTPHILISFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSSGSETPGTSESATPEGSESTVGPAC
PPGRTVTKRPWALAEDTPRGPDSPPKRPRPNSLPLITTFRPLPPP
PQTTSAVDPSSHSPVNPPRDQHATDTADEKPRAASPALSDASGPP
TPDIPLSPGGTHARDPDADPDSPDLDSGGSGkrtadgsefespkk
krkv

d) EX586: illustrates a ZFL2-2 driver protein with an N647K mutation predicted to improve binding to RNA/DNA and a C-terminal fusion of a UL12 polypeptide. The UL12 polypeptide is shown at the C-terminus fused via a linker (shown in bold italics text) to a N-terminal ZFL2-2 protein (shown in underlined text). A nuclear localization sequence (shown in bold underlined lowercase text) is shown at the C-terminus fused via a linker (shown in bold italics text) to a N-terminal UL12 polypeptide. The EX586 driver is encoded by the EX586 driver construct (SEQ ID NO: 34).

### EX586 Amino Acid Sequence:

(SEQ ID NO: 33)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNATNPRLLFKTESSLLYPPPPPASS
TLTTDDFATFFCTKTAKISAQFAAPTTNTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSKLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLFSLHFTSSGSETPGTSESATPEGSESTVGPAC
PPGRTVTKRPWALAEDTPRGPDSPPKRPRPNSLPLITTFRPLPPP
PQTTSAVDPSSHSPVNPPRDQHATDTADEKPRAASPALSDASGPP
TPDIPLSPGGTHARDPDADPDSPDLDSGGSGkrtadgsefespkk
krkv

e) EX587: illustrates a ZFL2-2 driver protein with a C-terminal fusion of an Sto7D and a UL12 polypeptide. The Sto7D polypeptide is shown at the C-terminus fused via a linker (shown in bold italics text) to a N-terminal ZFL2-2 protein (shown in underlined text). The UL12 polypeptide is shown at the C-terminus fused via a linker (shown in bold italics text) to a N-terminal Sto7D polypeptide. A nuclear localization sequence (shown in bold underlined lowercase text) is shown at the C-terminus fused via a linker (shown in bold italics text) to a N-terminal UL12 polypeptide. The EX587 driver is encoded by the EX587 driver construct (SEQ ID NO: 36).

### EX587 Amino Acid Sequence:

(SEQ ID NO: 35)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNAINPRLLFKTESSLLYPPPPPASS
TLTTDDFATFFCTKTAKISAQFAAPTINTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDFLTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLFSLHFTSSGSETPGTSESATPEGSVTVKFKYK
GEEKEVDISKIKKVWRVGKMISFTYDDNGKTGRGAVSEKDAPKEL
LQMLEKSGSETPGTSESATPEGSESTVGPACPPGRTVTKRPWALA
EDTPRGPDSPPKRPRPNSLPLTTTFRPLPPPPQTTSAVDPSSHSP
VNPPRDQHATDTADEKPRAASPALSDASGPPTPDIPLSPGGTHAR
DPDADPDSPDLDSGGSGkrtadgsefespkkkrkv

f) EX594: illustrates a ZFL2-2 driver protein with a C-terminal fusion of a BRCA2-derived polypeptide. The BRCA2-derived polypeptide is shown at the C-terminus fused via a linker (shown in bold italics text) to a N-terminal ZFL2-2 protein (shown in underlined text). A nuclear localization sequence (shown in bold underlined lowercase text) is shown at the C-terminus fused via a linker (shown in bold italics text) to a N-terminal BRCA2-derived polypeptide. The EX594 driver is encoded by the EX594 driver construct (SEQ ID NO: 38).
g) EX594 amino acid sequence:

(SEQ ID NO: 37)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNATNPRLLFKTFSSLLYPPPPPASS
TLTTDDFATFFCTKTAKISAQFAAPTTNTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVENQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSSGSETPGTSESATPEGSPTLLGFHT
ASGKKVKIAKESLDKVKNLFDEKEQGGSGkrtadgsefespkkkr
kv

h) EX595: illustrates a ZFL2-2 driver protein with an N-terminal fusion of an HMGN1 polypeptide, and a C-terminal fusion of an HMGB1 polypeptide. The HMGN1 polypeptide is shown at the N-terminus fused via a linker (shown in bold italics text) to a C-terminal ZFL2-2 protein (shown in underlined text). The HMGB1 polypeptide is shown at the C-terminus fused via a linker (shown in bold italics text) to a N-terminal ZFL2-2 protein (shown in underlined text). A nuclear localization sequence (shown in bold underlined lowercase text) is shown at the C-terminus fused via a linker (shown in bold italics text) to a N-terminal HMGB1 polypeptide. The EX595 driver is encoded by the EX595 driver construct (SEQ ID NO: 40).

### EX595 Amino Acid Sequence:

(SEQ ID NO: 39)
MPKRKVSSAEGAAKEEPKRRSARLSAKPPAKVEAKPKKAAAKDKS
SDKKVQTKGKRGAKGKQAEVANQETKEDLPAENGETKTEESPASD
EAGEKEAKSDSGSETPGTSESATPESCFLIPVVINTRKTREVRCK
RNPHNLRSIHVSTISQLSLSVGLWNCQSAVNKADFITSIATYSDY
NLMALTETWLRPEDTATHATLSANFSFSHTPRQTGRGGGTGLLIS
KEWKFTLIPSLPTISSFEFHAVTIIHPFYINVVVIYRPPGKLGHF
LDELDVLLSSFSNFDTPLLVLGDENIYVDKPQAADFQTLLASEDL
KRAPTSATHKSGNQLDLIYTRHCFTDQTIVTPLQISDHFLLSLNI
HITPEPPHTPTLVTERRNLRSLSPNRLSTIVSDSLPPSRKLTALD
SNSATNTLCSTLASCLDRLCPLASRPARASPPAPWLSDALREHRS
KLRAAERIWRKTKNPAHLLTYQTLLSSFSAEVTSAKQTYYRLKIN
NATNPRLLFKTESSLLYPPPPPASSTLTTDDFATFFCTKTAKISA
QFAAPTTNTQDTTPTPHTLTSFSQLSESEVSKLVLSSHATTCPLD
PIPSHLLQAISPAVIPTLTHIINTSLDSGLFPTTFKQARVTPLLK
KPNLDHTLLENYRPVSLLPFMAKILEKVVENQVLDELTQNNLMDN
KQSGFKKGHSTETALLSVVEDLRLAKADSKSSVLILLDLSAAFDT
VNHQILLSTLESLGVAGTVIQWERSYLSDRSFRVSWRGEVSNLQH
LNTGVPQGSVLGPLLFSIYTSSLGPVIQRHGFSYHCYADDTQLYL
SFHPDDPSVPARISACLLDISHWMKDHHLQLNLAKTEMLVVSANP
TLHHNFSIQMDGATITASKMVKSLGVTIDDQLNFSDHISRTARSC
RFALYNIRKIRPELSEHAAQLLVQALVLSKLDYCNSLLALLPANS
IKPLQLLQNAAARVVFNEPKRAHVTPLLVRLHWLPVAARIKFKTL
MFAYKVTSGLAPSYLHSLLQIYVPSRNLRSVNERRLVVPSQRGKK
SLSRTLTLNLPSWWNELPNCIRTAESLAIFKKRLKTQLFSLHFTS
SGSETPGTSESATPEGKGDPKKPRGKMSSYAFFVQTCREEHKKKH
PDASVNFSEFSKKCSERWKTMSAKEKGKFEDMAKADKARYEREMK
TYIPPKGEGGSGkrtadgsefespkkkrkv

i) EX596: illustrates a ZFL2-2 driver protein with an N-terminal fusion of an HMGN1 polypeptide, a C-terminal fusion of an HMGB1 polypeptide, and a C-terminal fusion of a UL12 polypeptide. The HMGN1 polypeptide is shown at the N-terminus fused via a linker (shown in bold italics text) to a C-terminal ZFL2-2 protein (shown in underlined text). The UL12 polypeptide is shown at the C-terminus fused via a linker (shown in bold italics text) to a N-terminal ZFL2-2 protein (shown in underlined text). The HMGB1 polypeptide is shown at the C-terminus fused via a linker (shown in bold italics text) to a C-terminal UL12 polypeptide. A nuclear localization sequence (shown in bold underlined lowercase text) is shown at the C-terminus fused via a linker (shown in bold italics text) to a N-terminal HMGB1 polypeptide. The EX596 driver is encoded by the EX596 driver construct (SEQ ID NO: 42).

### EX596 Amino Acid Sequence:

(SEQ ID NO: 41)
MPKRKVSSAEGAAKEEPKRRSARLSAKPPAKVEAKPKKAAAKDKS
SDKKVQTKGKRGAKGKQAEVANQETKEDLPAENGETKTEESPASD
EAGEKEAKSDSGSETPGTSESATPESCFLIPVVTNTRKTREVRCK
RNPHNLRSIHVSTISQLSLSVGLWNCQSAVNKADFITSIATYSDY
NLMALTETWLRPEDTATHATLSANFSFSHTPRQTGRGGGTGLLIS
KEWKFTLIPSLPTISSFEFHAVTIIHPFYINVVVIYRPPGKLGHF
LDELDVLLSSFSNFDTPLLVLGDENIYVDKPQAADFQTLLASEDL
KRAPTSATHKSGNQLDLIYTRHCFTDQTIVTPLQISDHFLLSLNI
HITPEPPHTPTLVTERRNLRSLSPNRLSTIVSDSLPPSRKLTALD
SNSAINTLCSTLASCLDRLCPLASRPARASPPAPWLSDALREHRS
KLRAAERIWRKTKNPAHLLTYQTLLSSFSAEVTSAKQTYYRLKIN
NATNPRLLFKTFSSLLYPPPPPASSTLTTDDFATFFCTKTAKISA
QFAAPTTNTQDTTPTPHTLTSFSQLSESEVSKLVLSSHATTCPLD
PIPSHLLQAISPAVIPTLTHIINTSLDSGLFPTTFKQARVTPLLK
KPNLDHTLLENYRPVSLLPFMAKILEKVVFNQVLDELTQNNLMDN
KQSGFKKGHSTETALLSVVEDLRLAKADSKSSVLILLDLSAAFDT
VNHQILLSTLESLGVAGTVIQWERSYLSDRSFRVSWRGEVSNLQH
LNTGVPQGSVLGPLLFSIYTSSLGPVIQRHGFSYHCYADDTQLYL
SFHPDDPSVPARISACLLDISHWMKDHHLQLNLAKTEMLVVSANP
TLHHNFSIQMDGATITASKMVKSLGVTIDDQLNFSDHISRTARSC
RFALYNIRKIRPFLSEHAAQLLVQALVLSKLDYCNSLLALLPANS
IKPLQLLQNAAARVVFNEPKRAHVTPLLVRLHWLPVAARIKFKTL
MFAYKVTSGLAPSYLHSLLQIYVPSRNLRSVNERRLVVPSQRGKK
SLSRTLTLNLPSWWNELPNCIRTAESLAIFKKRLKTQLFSLHFTS
SGSETPGTSESATPEGSESTVGPACPPGRTVTKRPWALAEDTPRG
PDSPPKRPRPNSLPLITTERPLPPPPQTTSAVDPSSHSPVNPPRD
QHATDTADEKPRAASPALSDASGPPTPDIPLSPGGTHARDPDADP
DSPDLDSSGSETPGTSESATPEGKGDPKKPRGKMSSYAFFVQTCR
EEHKKKHPDASVNFSEFSKKCSERWKTMSAKEKGKFEDMAKADKA
RYEREMKTYIPPKGEGGSGkrtadgsefespkkkrkv

j) EX597: illustrates a ZFL2-2 protein with a C-terminal fusion of a UL12 polypeptide followed by a Sto7D polypeptide. The UL12 polypeptide is shown at the C-terminus fused via a linker (shown in bold italics text) to a N-terminal ZFL2-2 protein (shown in underlined text). The Sto7D polypeptide is shown at the C-terminus fused via a linker (shown in bold italics text) to a N-terminal UL12 polypeptide. A nuclear localization sequence (shown in bold underlined lowercase text) is shown at the C-terminus fused via a linker (shown in bold italics text) to a N-terminal Sto7D polypeptide.

### EX597 Amino Acid Sequence:

(SEQ ID NO: 43)
MCFLIPVVINTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNFDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNAINPRLLFKTFSSLLYPPPPPASS
TLTTDDFATFFCTKTAKISAQFAAPTINTQDTTPTPHTLTSFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVENQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTSSGSETPGTSESATPEGSESTVGPAC
PPGRTVTKRPWALAEDTPRGPDSPPKRPRPNSLPLITTFRPLPPP
PQTTSAVDPSSHSPVNPPRDQHATDTADEKPRAASPALSDASGPP
TPDIPLSPGGTHARDPDADPDSPDLDSSGSETPGTSESATPEGSV
TVKFKYKGEEKEVDISKIKKVWRVGKMISFTYDDNGKTGRGAVSE
KDAPKELLQMLEKGGSGkrtadgsefespkkkrkv

k) EX588: illustrates a ZFL2-2 protein (expressed from an RNA with a Kozak consensus sequence).

### EX588 Amino Acid Sequence:

(SEQ ID NO: 45)
MCFLIPVVINTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSFSNEDTPLLVLGDEN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNAINPRLLFKTFSSLLYPPPPPASS
TLTTDDFATFFCTKTAKISAQFAAPTINTQDITPTPHILISFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDFLTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLFSLHFTS

l) EX666: illustrates a ZFL2-2 protein with a N-terminal Nhp6a derived polypeptide. The Nhp6a derived polypeptide is shown at the N-terminus fused via a linker (shown in bold italics text) to a C-terminal ZFL2-2 protein.

### EX666 Amino Acid Sequence:

(SEQ ID NO: 47)
MAAPREPKKRTTRKKKGSSGCFLIPVVINTRKTREVRCKRNPHNL
RSIHVSTISQLSLSVGLWNCQSAVNKADFITSIATYSDYNLMALT
ETWLRPEDTATHATLSANFSFSHTPRQTGRGGGTGLLISKEWKFT
LIPSLPTISSFEFHAVTIIHPFYINVVVIYRPPGKLGHELDELDV
LLSSFSNEDTPLLVLGDFNIYVDKPQAADFQTLLASFDLKRAPTS
ATHKSGNQLDLIYTRHCFTDQTIVTPLQISDHFLLSLNIHITPEP
PHTPTLVTERRNLRSLSPNRLSTIVSDSLPPSRKLTALDSNSAIN
TLCSTLASCLDRLCPLASRPARASPPAPWLSDALREHRSKLRAAE
RIWRKTKNPAHLLTYQTLLSSFSAEVTSAKQTYYRLKINNATNPR
LLFKTFSSLLYPPPPPASSTLTTDDFATFFCTKTAKISAQFAAPT
TNTQDTTPTPHTLTSFSQLSESEVSKLVLSSHATTCPLDPIPSHL
LQAISPAVIPTLTHIINTSLDSGLFPTTFKQARVTPLLKKPNLDH
TLLENYRPVSLLPFMAKILEKVVENQVLDELTQNNLMDNKQSGFK
KGHSTETALLSVVEDLRLAKADSKSSVLILLDLSAAFDTVNHQIL
LSTLESLGVAGTVIQWFRSYLSDRSFRVSWRGEVSNLQHLNTGVP
QGSVLGPLLFSIYTSSLGPVIQRHGFSYHCYADDTQLYLSFHPDD
PSVPARISACLLDISHWMKDHHLQLNLAKTEMLVVSANPTLHHNF
SIQMDGATITASKMVKSLGVTIDDQLNFSDHISRTARSCRFALYN
IRKIRPFLSEHAAQLLVQALVLSKLDYCNSLLALLPANSIKPLQL
LQNAAARVVFNEPKRAHVTPLLVRLHWLPVAARIKFKTLMFAYKV
TSGLAPSYLHSLLQIYVPSRNLRSVNERRLVVPSQRGKKSLSRTL
TLNLPSWWNELPNCIRTAESLAIFKKRLKTQLFSLHFTS

m) SM002: illustrates a ZFL2-2 driver protein comprising a wild-type ZFL2-2 protein.

### SM002 Amino Acid Sequence:

(SEQ ID NO: 51)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGTGLLISKEWKFTLIPSLPTISSFEFHAVTII
HPFYINVVVIYRPPGKLGHFLDELDVLLSSESNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSAINTLCSTLASCLDRLCPLASR
PARASPPAPWLSDALREHRSKLRAAERIWRKTKNPAHLLTYQTLL
SSFSAEVTSAKQTYYRLKINNAINPRLLFKTFSSLLYPPPPPASS
TLTTDDFATFFCTKTAKISAQFAAPTTNTQDTTPTPHILISFSQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVENQVLDELTQNNLMDNKQSGFKKGHSTETALLSVVEDLRLA
KADSKSSVLILLDLSAAFDTVNHQILLSTLESLGVAGTVIQWERS
YLSDRSFRVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTS

### SM002 DNA Sequence:

(SEQ ID NO: 52)
TGCAGGGTCGACTAATACGACTCACTATAGGGAGAGATATCCCTA
GCTAGTTCACCGCGGCAGCGGTCGCGGCAGCCTCGTGTGAAGACC
GACGAGGGTAAAGACCATCGACTCTACCTGCGCGACTCCACCGAG
CAAAGACACCGACAAAGCACTTGAGTACTTTACTTTATTGTTTTA
CTTTACACTTATTTTTTGTTGTCAGTGCACTTTTATTATGTGTTT
TCTAATTCCTGTTGTTACTAACACTCGCAAAACACGGGAGGTACG
CTGCAAGCGTAATCCTCACAACCTTCGTTCAATACATGTATCTAC
TATTTCACAACTCTCTCTCTCCGTGGGCCTCTGGAATTGTCAATC
AGCTGTTAACAAGGCTGATTTTATTACCTCCATAGCTACATATTC
TGACTATAATCTCATGGCTCTAACTGAGACCTGGTTGAGGCCGGA
GGACACTGCTACACATGCTACTCTTTCTGCTAATTTCTCTTTTTC
CCACACTCCTCGTCAGACAGGGAGAGGGGGTGGGACTGGACTACT
AATTTCCAAAGAATGGAAATTTACTCTGATACCGTCCCTGCCAAC
AATCAGCTCCTTTGAATTCCATGCAGTCACCATTATCCACCCCTT
CTACATAAATGTGGTTGTCATCTACCGCCCACCAGGTAAATTAGG
TCACTTCCTAGATGAACTGGATGTTCTTCTCTCATCTTTTTCTAA
TTTTGACACTCCCTTATTGGTGCTAGGIGACTTCAACATTTACGT
TGACAAACCGCAAGCTGCAGACTTTCAGACTTTGCTTGCCTCTTT
TGACCTAAAAAGAGCACCTACTTCTGCTACCCACAAATCAGGTAA
TCAGCTAGACCTTATTTACACACGACACTGCTTCACTGATCAAAC
AATAGTAACTCCACTACAAATATCTGATCATTTCCTTCTGTCTCT
CAACATCCACATTACTCCTGAGCCGCCACACACTCCTACACTGGT
TACCTTTCGCAGAAACCTACGATCTCTCTCACCCAATAGACTATC
CACCATTGTTTCAGACTCTCTTCCTCCATCTCGCAAACTCACTGC
ACTTGATTCGAACAGTGCCACTAATACACTCTGCTCCACACTAGC
ATCATGTCTAGACCGATTATGTCCTCTTGCATCCAGGCCAGCCCG
TGCCAGTCCTCCTGCACCCTGGCTCTCGGATGCTCTCCGTGAGCA
TCGCTCAAAACTTCGGGCTGCGGAGAGAATTTGGCGGAAAACTAA
AAATCCTGCACATCTCTTAACATACCAAACTCTTCTGTCCTCTTT
CTCAGCTGAGGTTACTTCTGCAAAGCAGACGTATTACCGTCTGAA
AATCAACAATGCCACTAATCCTCGCCTACTTTTTAAAACATTTTC
CTCCCTCCTATATCCTCCTCCTCCACCCGCATCCTCCACACTTAC
TACTGATGACTTTGCTACATTCTTCTGCACCAAAACTGCAAAAAT
CAGTGCTCAATTTGCTGCACCTACAACAAACACGCAAGATACAAC
ACCAACACCACACACACTCACCTCTTTTTCTCAGCTCTCTGAGTC
TGAGGTGTCCAAACTTGTGCTATCTAGCCATGCAACCACCTGTCC
ACTCGATCCCATTCCCTCTCATCTCTTGCAAGCCATCTCTCCTGC
AGTCATACCAACACTGACTCACATAATTAACACATCTCTTGACTC
TGGTTTATTCCCCACTACATTTAAGCAGGCTAGGGTAACCCCACT
GCTAAAGAAACCCAACCTGGACCATACGCTACTTGAAAACTACAG
ACCAGTATCCCTGCTTCCATTCATGGCCAAGATTCTGGAGAAAGT
AGTGTTCAATCAAGTCCTGGACTTTCTTACTCAAAACAATCTCAT
GGACAACAAGCAATCCGGCTTTAAGAAAGGCCACTCAACTGAGAC
TGCCCTGCTCTCGGTCGTGGAGGATCTCAGACTGGCTAAAGCAGA
CTCTAAATCATCAGTCCTCATTTTGCTGGACTTGTCAGCTGCTTT
TGACACTGTCAACCACCAGATCCTGCTATCTACGCTTGAGTCACT
GGGCGTTGCGGGCACTGTTATACAATGGTTTAGATCTTACCTCTC
TGACAGGTCATTCAGGGTGTCTTGGAGGGGAGAGGTGTCCAACCT
ACAGCATCTAAACACTGGGGTACCTCAAGGCTCTGTTCTTGGGCC
ACTTCTCTTCTCCATCTACACATCATCTCTAGGACCAGTCATCCA
GAGACATGGATTCTCCTACCACTGCTATGCTGATGACACCCAGCT
ATACCTCTCTTTTCATCCTGATGATCCCTCGGTTCCAGCTCGTAT
CTCAGCCTGCCTGTTGGATATTICACACTGGATGAAAGATCATCA
TCTTCAGCTGAACCTCGCAAAAACGGAAATGCTTGTAGTTTCTGC
CAACCCGACTCTACACCATAACTTTTCAATCCAGATGGATGGGGC
AACCATTACTGCATCCAAAATGGTGAAAAGCCTTGGAGTAACGAT
TGATGACCAACTAAACTTCTCTGACCACATTTCTAGAACTGCTCG
ATCGTGCAGATTTGCACTCTATAACATCAGAAAGATCCGACCCTT
CTTATCTGAACATGCAGCTCAACTCCTTGTTCAAGCTCTTGTTCT
CTCCAAACTGGATTACTGCAACTCTCTACTAGCTTTGCTTCCAGC
TAACTCTATCAAGCCTCTTCAACTGCTCCAGAATGCAGCAGCACG
AGTTGTCTTCAATGAACCTAAACGAGCACATGTCACTCCGCTGCT
AGTCCGTTTGCACTGGCTGCCAGITGCTGCTCGCATCAAATTCAA
AACTCTGATGTTTGCCTACAAAGTGACTTCTGGCCTAGCACCTTC
TTATCTGCACTCACTTCTGCAGATCTATGTGCCCTCCAGAAACTT
GCGTTCTGTGAATGAACGTCGCCTCGTGGTTCCATCCCAAAGAGG
GAAAAAATCACTTTCGCGAACGCTCACGCTCAATCTGCCCAGTTG
GTGGAATGAACTCCCTAACTGCATCAGAACAGCAGAGTCACTCGC
TATTTTCAAGAAACGACTAAAAACTCAACTATTTAGTCTCCACTT
CACTTCCTAAGCTGCAATTGCCTCTTTGAATATCACACTAATTGT
ACAAAAAAAAAAAAAAAAAAAAAAAAAACTACTAACACTTCCCTT
CTTAGACTTTACAGACCGCGGCCTACTCGGATCCGCGATGATGAT
CAGACATGATAAGATACATTGATGAGTTTGGACAAACCACAACTA
GAATGCAGTGAAAAAAATGCTTTATTTGTGAAATTTGTGATGCTA
TTGCTTTATTTGTAACCATTATAAGCTGCAATAAACAAGTTAACA
ACAACAAAAAAAAAAAAAAAAAAAAATTTAAATGCGCGCATC

a) SM003: illustrates an EGFP reporter gene delivery construct, e.g., for use in trans with a ZFL2-2 driver (e.g. the various ZFL2-2 drivers listed above).

### SM003 DNA Sequence:

(SEQ ID NO: 53)
TGCAGGGTCGACTAATACGACTCACTATAGGGAGAGATAATTGCC
TCTTTGAATATCACACTAATTGTACAAAAAAAAAAAAAAAAAAAA
AAAAAACTACTAACACTTCCCTTCTTAGACTTTACAGACCGCGGC
CTACTCGACGGATCGATCCGAACAAACGACCCAACACCCGTGCGT
TTTATTCTGTCTTTTTATTGCCGATCCCCTCAGAAGAACTCGTCA
AGAAGGCGATAGAAGGCGATGCGCTGCGAATCGGGAGCGGCGATA
CCGTAAAGCACGAGGAAGCGGTCAGCCCATTCGCCGCCAAGCTCT
TCAGCAATATCACGGGTAGCCAACGCTATGTCCTGATAGCGGTCG
GCCGCTTTACTTGTACAGCTCGTCCATGCCGAGAGTGATCCCGGC
GGCGGTCACGAACTCCAGCAGGACCATGTGATCGCGCTTCTCGTT
GGGGTCTTTGCTCAGGGCGGACTGGGTGCTCAGGTAGTGGTTGTC
GGGCAGCAGCACGGGGCCGTCGCCGATGGGGGTGTTCTGCTGGTA
GTGGTCGGCCAGCTGCACGCTGCCGTCCTCGATGTTGTGGCGGAT
CTTGAAGTTCACCTTGATGCCGTTCTTCTGCTTGTCGGCCATGAT
ATAGACGTTGTGGCTGTTGTAGTTGTACTCCAGCTTGTGCCCCAG
GATGTTGCCGTCCTCCTTGAAGTCGATGCCCTTCAGCTCGATGCG
GTTCACCAGGGTGTCGCCCTCGAACTTCACCTCGGCGCGGGTCTT
GTAGTTGCCGTCGTCCTTGAAGAAGATGGTGCGCTCCTGGACGTA
GCCTTCGGGCATGGCGGACTTGAAGAAGTCGTGCTGCTTCATGTG
GTCGGGGTAGCGGCTGAAGCACTGCACGCCGTAGGTCAGGGTGGT
CACGAGGGTGGGCCAGGGCACGGGCAGCTTGCCGGTGGTGCAGAT
GAACTTCAGGGTCAGCTTGCCGTAGGTGGCATCGCCCTCGCCCTC
GCCGGACACGCTGAACTTGTGGCCGTTTACGTCGCCGTCCAGCTC
GACCAGGATGGGCACCACCCCGGTGAACAGCTCCTCGCCCTTGCT
CACCATGGTGGCGAATTCGAAGCTTGAGCTCGAGATCTGAGTCCG
GTAGCGCTAGCGGATCTGACGGTTCACTAAACCAGCTCTGCTTAT
ATAGACCTCCCACCGTACACGCCTACCGCCCATTTGCGTCAATGG
GGCGGAGTTGTTACGACATTTTGGAAAGTCCCGTTGATTTTGGTG
CCAAAACAAACTCCCATTGACGTCAATGGGGTGGAGACTTGGAAA
TCCCCGTGAGTCAAACCGCTATCCACGCCCATTGATGTACTGCCA
AAACCGCATCACCATGGTAATAGCGATGACTAATACGTAGATGTA
CTGCCAAGTAGGAAAGTCCCATAAGGTCATGTACTGGGCATAATG
CCAGGCGGGCCATTTACCGTCATTGACGTCAATAGGGGGCGTACT
TGGCATATGATACACTTGATGTACTGCCAAGTGGGCAGTTTACCG
TAAATACTCCACCCATTGACGTCAATGGAAAGTCCCTATTGGCGT
TACTATGGGAACATACGTCATTATTGACGTCAATGGGCGGGGGTC
GTTGGGCGGTCAGCCAGGCGGGCCATTTACCGTAAGTTATGTAAC
GCGGAACTCCATATATGGGCTATGAACTAATGACCCCGTAATTGA
TTACTATTAAATTCCTGCAGGTTTGGGTGAAACTTGCCTTTAGTA
CTTATTCATTGTTGCTCTTAGTIGTGTAAATTGCTTCCTTGTCCT
CATTTGTAAGTCGCTTTGGATAAAAGCGTCTGCTAAATGACTAAA
TGTAAATGTAAATGTAAAGGATCCGCGATGATGATCAGACATGAT
AAGATACATTGATGAGTTTGGACAAACCACAACTAGAATGCAGTG
AAAAAAATGCTTTATTTGTGAAATTTGTGATGCTATTGCTTTATT
TGTAACCATTATAAGCTGCAATAAACAAGTTAACAACAACAAAAA
AAAAAAAAAAAAAAAATTTAAATGCGCGCATCATC

### Integration Assay:

An integration assay was performed to evaluate the percentage of cells in which a stable reporter (e.g., GFP) encoded as a transgene in a gene delivery construct was integrated by retrotransposition. A GFP reporter gene delivery construct (“reporter construct”) was evaluated in combination with different engineered driver constructs in trans. The reporter construct contains an antisense expression cassette for GFP (driven by a CMV promoter and containing a polyadenylation signal from the thymidine kinase gene from the herpes simplex virus) and the 3′ UTR regulatory sequence of a zebrafish ZFL2-2 retrotransposon, which contains 3 copies of a microsatellite sequence. The trans driver construct contains the single ORF encoded by a zebrafish ZFL2-2 retrotransposon and a polyadenylated SV40 sequence ending in A30-N10-A70 (wherein the N10 are 10 non-adenosine containing nucleotides). The reporter and driver constructs were configured with T7 promoters and a unique Type IIS restriction site at the 3′ end. Upon restriction, these DNAs were used in in vitro transcription (IVT) reactions using NEB HiScribe™ T7 High Yield RNA Synthesis Kit.

Briefly, the plasmid was first dissolved and let stand at room temperature. Then, to assess the concentration of the plasmid, a nanodrop was used to measure the absorbance of the sample. The plasmid was then restricted with a restriction enzyme and cleaned using AMPure XP beads mixed into the solution. Using a magnetic tube rack the solution was aspirated with 70% ethanol added and incubated at room temperature three times. The ethanol was then removed, and the beads dried. The plasmids were then resuspended using an elution buffer, dried and resuspended in water. Once resuspended the plasmid concentration was measured.

Next, for IVT production the NEB HiScribe™ T7 High Yield RNA Synthesis Kit was used. Then for the DNase phase, the TURBO™ DNase kit AM2238 was utilized. The RNA transcript was then purified using Monarch RNA cleanup Kit T2050.

To check the quality of the sample first, quantification is done using the Nanodrop. Then using the Agilent TapStation device and the Controller software the quality and uniformity are measured.

Trans-retrotransposition assays were conducted in U2OS cells. First, cells were seeded 24 hours prior to transfection. Briefly, transfection was done using MessengerMAX™. The mRNA master mix was then prepared and mixed well. The mRNA master mix was then added to the diluted MessengerMAX™ reagent and incubated. The new RNA-lipid complex is then added to the cells and incubated overnight. Reporter integration was then checked by measuring the percent of GFP expressing cells after 24 hours using FACS analyses and fluorescent microscopy.

### Results:

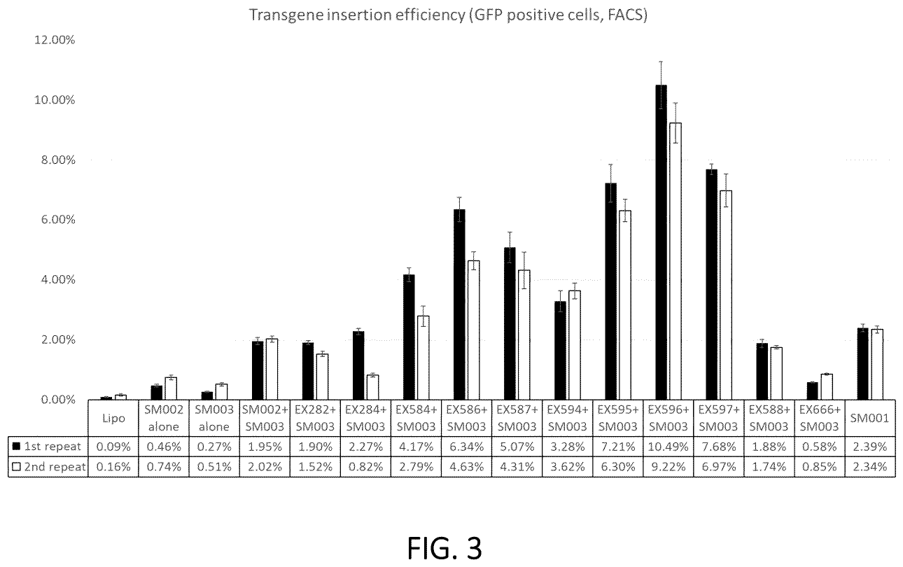

FIG. 3 shows results of integration assays using ZFL2-2 drivers comprising heterologous HDR and chromatin opening domains along with p53 inhibition.

In this experiment, different driver constructs were used with the same reporter construct, GFP reporter gene delivery construct SM003 (SEQ ID NO: 52). The drivers were fusion, and cassette engineered constructs described above. IVT of different RTE drivers and reporters was carried out as described above. U20S cells were used in 24-well plates, at 100K cells/well. 1000 ng RNA was transfected with 1.2 uL Lipofectamine. Integration was assessed based on the percentage of GFP positive cells (% GFP positive cells) after 24 h, with a higher percentage of GFP positive cells being indicative of higher levels of integration. % GFP positive cells was assessed by FACS following 24 hours after transfection with RNA.

C-terminal fusion of UL12 to ZFL2-2 (EX584) increased GFP positive cells approximately two-fold compared to wild-type ZFL2-2 (SM002). C-terminal fusion of BRCA2-derived peptide to ZFL2-2 (EX594) increased GFP positive cells approximately two-fold compared to wild-type ZFL2-2 (SM002). N-terminal fusion of HMGN1 and C-terminal fusion of HMGB1 to ZFL2-2 (EX595) increased GFP positive cells approximately three-fold compared to wild-type ZFL2-2 driver (SM002). Combining these two modifications (EX596) increased GFP positive cells approximately five-fold compared to wild-type. Combining C-terminal UL12 fusion to ZFL2-2 with an N647K mutation (EX586) increased GFP positive cells approximately three-fold relative to wild-type. Combining ZFL2-2 C-terminal UL12 fusion with C-terminal Sto7D fusion increased GFP positive cells relative to wild-type ZFL2-2 by approximately two-fold when Sto7D was positioned between ZFL2-2 and UL12 (EX587), and by approximately four-fold when Sto7D was positioned after ZFL2-2 and UL12 (EX597). In these experiments, not all modifications improved integration efficiency, in particular, C-terminal fusion of NBN-derived peptide (EX282) and N-terminal fusion of Nhp6a (EX666) did not improve integration efficiency.

Altogether, these results illustrate several aspects of the application. It was found that fusing one or more polypeptides that promote HDR to a retroelement-derived enzyme domain (e.g., fusing a retroelement-derived enzyme to UL12 and/or other polypeptide that promotes HDR) improves retrotransposition (e.g., increase retrotransposition frequency relative to using a retroelement-derived enzyme that is not fused to any other polypeptide). It was also found that fusing one or more polypeptides that promote chromatin accessibility to a retroelement-derived enzyme domain (e.g., fusing a retroelement-derived enzyme to one or more HMG proteins and/or other polypeptides that promote chromatin accessibility) improves retrotransposition. It was also found that fusing one or more polypeptides that promote DNA interactions (e.g., that promote DNA binding) to a retroelement-derived enzyme domain (e.g., fusing a retroelement-derived enzyme to one or more Sto7D polypeptides and/or or other polypeptides that promote DNA interactions) improves retrotransposition. It was also found that one or more amino acid substitutions that promote Reverse Transcriptase interactions with RNA and/or DNA (e.g., including one or more amino acid modifications such as N647K substitution in LINE 2-2 and/or other amino acid modifications that promote retroelement-derived protein interactions with RNA and/or DNA) improves retrotransposition. It was also found that combining one or more of these modifications further enhances the rate of retrotransposition. It was also found that a combination of two or more modifications exhibit location-dependent effects. For example, C-terminal fusions of Sto7D followed by UL12 were less active than C-terminal fusions of UL12 followed by Sto7D.

### Example 4

In order to test the effect of mutations in potential RNA-binding regions of the driver, it was hypothesized that improving the electrostatic or structural stability of the RNA-binding domains may improve interaction with template RNA, thereby improving integration efficiency.

Nucleic acids were designed and produced to encode non-limiting examples of engineered proteins comprising a ZFL2-2-derived protein with one or more point mutations in the RNA binding domain and the reverse transcriptase domain. These were tested and evaluated using an experimental retrotransposition assay described in Example 3. Non-limiting examples of the heterologous polypeptides include:
EX120: ZFL2-2 with I343K mutation (SEQ ID NO: 341)
EX121: ZFL2-2 with Q372K mutation (SEQ ID NO: 342)
EX122: ZFL2-2 with E366N mutation (SEQ ID NO: 343)
EX123: ZFL2-2 with L354N mutation (SEQ ID NO: 344)
EX124: ZFL2-2 with D588A mutation (SEQ ID NO: 345)
EX125: ZFL2-2 with E616R+S617K mutation (SEQ ID NO: 346)
EX126: ZFL2-2 with N647K mutation (SEQ ID NO: 347)
EX132: ZFL2-2 with D550T mutation (SEQ ID NO: 348)
EX134: ZFL2-2 with D770H mutation (SEQ ID NO: 349)
EX135: ZFL2-2 with I625L mutation (SEQ ID NO: 350)
EX136: ZFL2-2 with H521P mutation (SEQ ID NO: 351)
EX137: ZFL2-2 with S737P mutation (SEQ ID NO: 352)
EX138: ZFL2-2 with P705A mutation (SEQ ID NO: 353)
EX139: ZFL2-2 with M558L mutation (SEQ ID NO: 354)
EX140: ZFL2-2 with M733L mutation (SEQ ID NO: 355)
EX141: ZFL2-2 with M760S mutation (SEQ ID NO: 356)
EX142: ZFL2-2 with M750L mutation (SEQ ID NO: 357)
EX143: ZFL2-2 with A757P mutation (SEQ ID NO: 358)
EX144: ZFL2-2 with H717A mutation (SEQ ID NO: 359)
EX146: ZFL2-2 with H717K mutation (SEQ ID NO: 360)
EX147: ZFL2-2 with D497S mutation (SEQ ID NO: 361)
EX148: ZFL2-2 with I625H mutation (SEQ ID NO: 362)

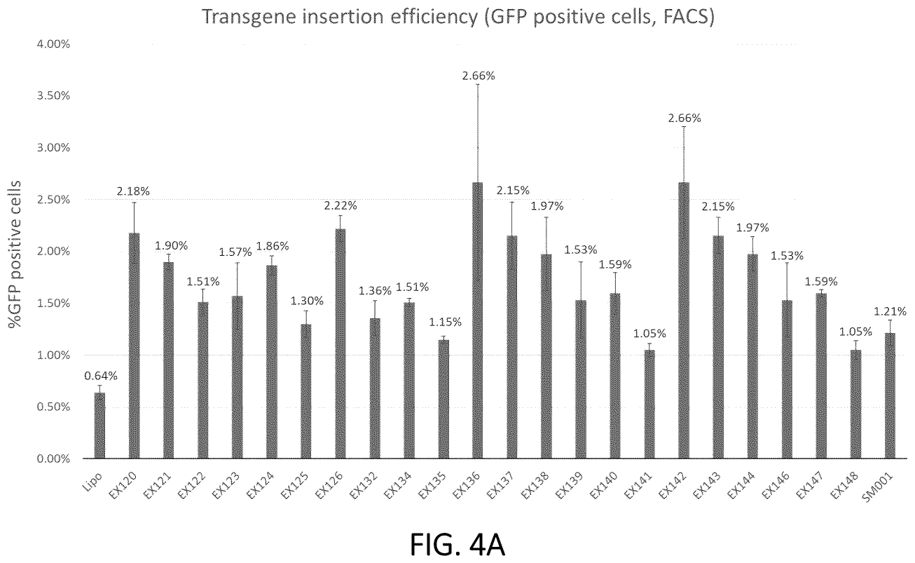

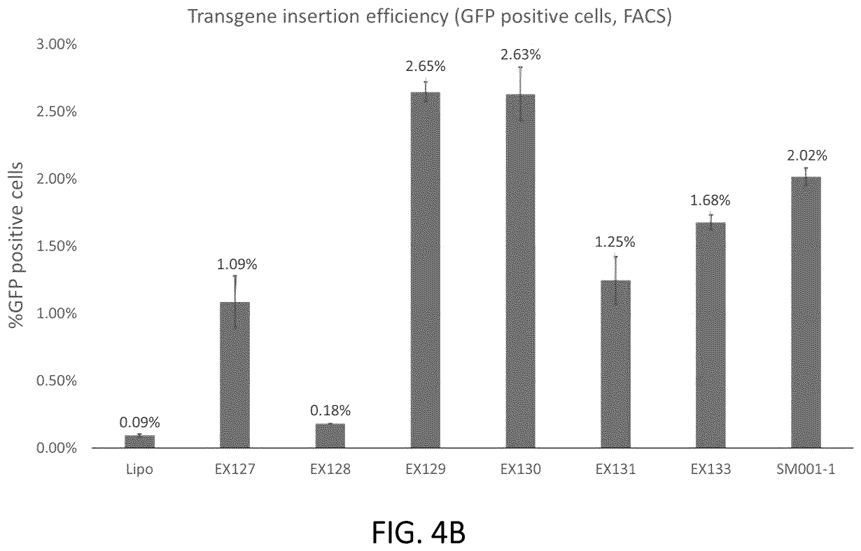

FIG. 4A shows results of integration assays using the above drivers with point mutations. In this experiment, different retrotransposable element constructs were used in the cis configuration (both driver and GFP reporter encoded by the same RNA). As such, the results of the integration assay with the above mutations were compared against a control cis driver/reporter system SM001, in which the SM001 plasmid encodes, inter alia, a ZFL2-2 driver comprising a wild type ZFL2-2 as well as a GFP reporter in a cis configuration. Aside from the mutations noted above, all constructs were identical in sequence to wild-type ZFL2-2 cis driver/reporter system (SM001).

### Amino Acid Sequence of Wild-Type FZL2-2 Driver Encoded in SM001 Plasmid:

(SEQ ID NO: 49)
MCFLIPVVTNTRKTREVRCKRNPHNLRSIHVSTISQLSLSVGLWN
CQSAVNKADFITSIATYSDYNLMALTETWLRPEDTATHATLSANF
SFSHTPRQTGRGGGIGLLISKEWKFTLIPSLPTISSFEFHAVTII
HPFYINVVVIYRPPGKLGHFLDELDVLLSSESNEDTPLLVLGDFN
IYVDKPQAADFQTLLASFDLKRAPTSATHKSGNQLDLIYTRHCFT
DQTIVTPLQISDHFLLSLNIHITPEPPHTPTLVTFRRNLRSLSPN
RLSTIVSDSLPPSRKLTALDSNSATNTLCSTLASCLDRLCPLASR
PARASPPAPWLSDALREHRSKLRAAERIWRKTKNPAHLLTYQTLL
SSFSAEVTSAKQTYYRLKINNAINPRLLFKTFSSLLYPPPPPASS
TLTTDDFATFFCTKTAKISAQFAAPTINTQDTTPTPHTLISESQL
SESEVSKLVLSSHATTCPLDPIPSHLLQAISPAVIPTLTHIINTS
LDSGLFPTTFKQARVTPLLKKPNLDHTLLENYRPVSLLPFMAKIL
EKVVFNQVLDELTQNNLMDNKQSGFKKGHSTETALLSVVEDLRLA
KADSKSSVLILLDLSAAFDTVNHQILLSTLESLGVAGTVIQWFRS
YLSDRSERVSWRGEVSNLQHLNTGVPQGSVLGPLLFSIYTSSLGP
VIQRHGFSYHCYADDTQLYLSFHPDDPSVPARISACLLDISHWMK
DHHLQLNLAKTEMLVVSANPTLHHNFSIQMDGATITASKMVKSLG
VTIDDQLNFSDHISRTARSCRFALYNIRKIRPFLSEHAAQLLVQA
LVLSKLDYCNSLLALLPANSIKPLQLLQNAAARVVFNEPKRAHVT
PLLVRLHWLPVAARIKFKTLMFAYKVTSGLAPSYLHSLLQIYVPS
RNLRSVNERRLVVPSQRGKKSLSRTLTLNLPSWWNELPNCIRTAE
SLAIFKKRLKTQLESLHFTS

### SM001 Plasmid DNA Sequence:

(SEQ ID NO: 50)
tgcaGGGTCGACTAATACGACTCACTATAGGGAGAGATATCCctagcTAGTTCACCGCGGCAGC
GGTCGCGGCAGCCTCGTGTGAAGACCGACGAGGGTAAAGACCATCGACTCTACCTGCGCGACTC
CACCGAGCAAAGACACCGACAAAGCACTTGAGTACTTTACTTTATTGTTTTACTTTACACTTAT
TTTTTGTTGTCAGTGCACTTTTATTatgTGTTTTCTAATTCCTGTTGTTACTAACACTCGCAAA
ACACGGGAGGTACGCTGCAAGCGTAATCCTCACAACCTTCGTTCAATACATGTATCTACTATTT
CACAACTCTCTCTCTCCGTGGGCCTCTGGAATTGTCAATCAGCTGTTAACAAGGCTGATTTTAT
TACCTCCATAGCTACATATTCTGACTATAATCTCATGGCTCTAACTGAGACCTGGTTGAGGCCG
GAGGACACTGCTACACATGCTACTCTTTCTGCTAATTTCTCTTTTTCCCACACTCCTCGTCAGA
CAGGGAGAGGGGGTGGGACTGGACTACTAATTTCCAAAGAATGGAAATTTACTCTGATACCGTC
CCTGCCAACAATCAGCTCCTTTGAATTCCATGCAGTCACCATTATCCACCCCTTCTACATAAAT
GTGGTTGTCATCTACCGCCCACCAGGTAAATTAGGTCACTTCCTAGATGAACTGGATGTTCTTC
TCTCATCTTTTTCTAATTTTGACACTCCCTTATTGGTGCTAGGTGACTTCAACATTTACGTTGA
CAAACCGCAAGCTGCAGACTTTCAGACTTTGCTTGCCTCTTTTGACCTAAAAAGAGCACCTACT
TCTGCTACCCACAAATCAGGTAATCAGCTAGACCTTATTTACACACGACACTGCTTCACTGATC
AAACAATAGTAACTCCACTACAAATATCTGATCATTTCCTTCTGTCTCTCAACATCCACATTAC
TCCTGAGCCGCCACACACTCCTACACTGGTTACCTTTCGCAGAAACCTACGATCTCTCTCACCC
AATAGACTATCCACCATTGTTTCAGACTCTCTTCCTCCATCTCGCAAACTCACTGCACTTGATT
CGAACAGTGCCACTAATACACTCTGCTCCACACTAGCATCATGTCTAGACCGATTATGTCCTCT
TGCATCCAGGCCAGCCCGTGCCAGTCCTCCTGCACCCTGGCTCTCGGATGCTCTCCGTGAGCAT
CGCTCAAAACTTCGGGCTGCGGAGAGAATTTGGCGGAAAACTAAAAATCCTGCACATCTCTTAA
CATACCAAACTCTTCTGTCCTCTTTCTCAGCTGAGGTTACTTCTGCAAAGCAGACGTATTACCG
TCTGAAAATCAACAATGCCACTAATCCTCGCCTACTTTTTAAAACATTTTCCTCCCTCCTATAT
CCTCCTCCTCCACCCGCATCCTCCACACTTACTACTGATGACTTTGCTACATTCTTCTGCACCA
AAACTGCAAAAATCAGTGCTCAATTTGCTGCACCTACAACAAACACGCAAGATACAACACCAAC
ACCACACACACTCACCTCTTTTTCTCAGCTCTCTGAGTCTGAGGTGTCCAAACTTGTGCTATCT
AGCCATGCAACCACCTGTCCACTCGATCCCATTCCCTCTCATCTCTTGCAAGCCATCTCTCCTG
CAGTCATACCAACACTGACTCACATAATTAACACATCTCTTGACTCTGGTTTATTCCCCACTAC
ATTTAAGCAGGCTAGGGTAACCCCACTGCTAAAGAAACCCAACCTGGACCATACGCTACTTGAA
AACTACAGACCAGTATCCCTGCTTCCATTCATGGCCAAGATTCTGGAGAAAGTAGTGTTCAATC
AAGTCCTGGACTTTCTTACTCAAAACAATCTCATGGACAACAAGCAATCCGGCTTTAAGAAAGG
CCACTCAACTGAGACTGCCCTGCTCTCGGTCGTGGAGGATCTCAGACTGGCTAAAGCAGACTCT
AAATCATCAGTCCTCATTTTGCTGGACTTGTCAGCTGCTTTTGACACTGTCAACCACCAGATCC
TGCTATCTACGCTTGAGTCACTGGGCGTTGCGGGCACTGTTATACAATGGTTTAGATCTTACCT
CTCTGACAGGTCATTCAGGGTGTCTTGGAGGGGAGAGGTGTCCAACCTACAGCATCTAAACACT
GGGGTACCTCAAGGCTCTGTTCTTGGGCCACTTCTCTTCTCCATCTACACATCATCTCTAGGAC
CAGTCATCCAGAGACATGGATTCTCCTACCACTGCTATGCTGATGACACCCAGCTATACCTCTC
TTTTCATCCTGATGATCCCTCGGTTCCAGCTCGTATCTCAGCCTGCCTGTTGGATATTTCACAC
TGGATGAAAGATCATCATCTTCAGCTGAACCTCGCAAAAACGGAAATGCTTGTAGTTTCTGCCA
ACCCGACTCTACACCATAACTTTTCAATCCAGATGGATGGGGCAACCATTACTGCATCCAAAAT
GGTGAAAAGCCTTGGAGTAACGATTGATGACCAACTAAACTTCTCTGACCACATTTCTAGAACT
GCTCGATCGTGCAGATTTGCACTCTATAACATCAGAAAGATCCGACCCTTCTTATCTGAACATG
CAGCTCAACTCCTTGTTCAAGCTCTTGTTCTCTCCAAACTGGATTACTGCAACTCTCTACTAGC
TTTGCTTCCAGCTAACTCTATCAAGCCTCTTCAACTGCTCCAGAATGCAGCAGCACGAGTTGTC
TTCAATGAACCTAAACGAGCACATGTCACTCCGCTGCTAGTCCGTTTGCACTGGCTGCCAGTTG
CTGCTCGCATCAAATTCAAAACTCTGATGTTTGCCTACAAAGTGACTTCTGGCCTAGCACCTTC
TTATCTGCACTCACTTCTGCAGATCTATGTGCCCTCCAGAAACTTGCGTTCTGTGAATGAACGT
CGCCTCGTGGTTCCATCCCAAAGAGGGAAAAAATCACTTTCGCGAACGCTCACGCTCAATCTGC
CCAGTTGGTGGAATGAACTCCCTAACTGCATCAGAACAGCAGAGTCACTCGCTATTTTCAAGAA
ACGACTAAAAACTCAACTATTTAGTCTCCACTTCACTTCCtaaGCTGCAATTGCCTCTTTGAAT
ATCACACTAATTGTACAAAAAAAAAAAAAAAAAAAAAAAAAACTACTAACACTTCCCTTCTTAG
ACTTTACAGACCgcggcctacTCGACGGATcgatccgaacaaacgACCCAACACCCGTGCGTTT
TATTCTGTCTTTTTATTGCCGATCCCCTCAGAAGAACTCGTCAAGAAGGCGATAGAAGGCGATG
CGCTGCGAATCGGGAGCGGCGATACCGTAAAGCACGAGGAAGCGGTCAGCCCATTCGCCGCCAA
GCTCTTCAGCAATATCACGGGTAGCCAACGCTATGTCCTGATAGCGGTCGGCCGCTttaCTTGT
ACAGCTCGTCCATGCCGAGAGTGATCCCGGCGGCGGTCACGAACTCCAGCAGGACCATGTGATC
GCGCTTCTCGTTGGGGTCTTTGCTCAGGGCGGACTGGGTGCTCAGGTAGTGGTTGTCGGGCAGC
AGCACGGGGCCGTCGCCGATGGGGGTGTTCTGCTGGTAGTGGTCGGCCAGCTGCACGCTGCCGT
CCTCGATGTTGTGGCGGATCTTGAAGTTCACCTTGATGCCGTTCTTCTGCTTGTCGGCCATGAT
ATAGACGTTGTGGCTGTTGTAGTTGTACTCCAGCTTGTGCCCCAGGATGTTGCCGTCCTCCTTG
AAGTCGATGCCCTTCAGCTCGATGCGGTTCACCAGGGTGTCGCCCTCGAACTTCACCTCGGCGC
GGGTCTTGTAGITGCCGTCGTCCTTGAAGAAGATGGTGCGCTCCTGGACGTAGCCTTCGGGCAT
GGCGGACTTGAAGAAGTCGTGCTGCTTCATGTGGTCGGGGTAGCGGCTGAAGCACTGCACGCCG
TAGGTCAGGGTGGTCACGAGGGTGGGCCAGGGCACGGGCAGCTTGCCGGTGGTGCAGATGAACT
TCAGGGTCAGCTTGCCGTAGGTGGCATCGCCCTCGCCCTCGCCGGACACGCTGAACTTGTGGCC
GTTTACGTCGCCGTCCAGCTCGACCAGGATGGGCACCACCCCGGTGAACAGCTCCTCGCCCTTG
CTCACcatGGTGGCGAATTCGAAGCTTGAGCTCGAGATCTGAGTCCGGTAGCGCTAGCGGATCT
GACGGTTCACTAAACCAGCTCTGCTTATATAGACCTCCCACCGTACACGCCTACCGCCCATTTG
CGTCAATGGGGCGGAGTTGTTACGACATTTTGGAAAGTCCCGTTGATTTTGGTGCCAAAACAAA
CTCCCATTGACGTCAATGGGGTGGAGACTTGGAAATCCCCGTGAGTCAAACCGCTATCCACGCC
CATTGATGTACTGCCAAAACCGCATCACCATGGTAATAGCGATGACTAATACGTAGATGTACTG
CCAAGTAGGAAAGTCCCATAAGGTCATGTACTGGGCATAATGCCAGGCGGGCCATTTACCGTCA
TTGACGTCAATAGGGGGCGTACTTGGCATATGATACACTTGATGTACTGCCAAGTGGGCAGTTT
ACCGTAAATACTCCACCCATTGACGTCAATGGAAAGTCCCTATTGGCGTTACTATGGGAACATA
CGTCATTATTGACGTCAATGGGCGGGGGTCGTTGGGCGGTCAGCCAGGCGGGCCATTTACCGTA
AGTTATGTAACGCGGAACTCCATATATGGGCTATGAACTAATGACCCcgtaattgattactatt
aaattcctgcaggtttgggTGAAACTTGCCTTTAGTACTTATTCATTGTTGCTCTTAGTTGTGT
AAATTGCTTCCTTGTCCTCATTTGTAAGTCGCTTTGGATAAAAGCGTCTGCTAAATGACTAAAT
GTAAATGTAAATGTAAAggatccGCGATGATGATCagacatgataagatacattgatgagtttg
gacaaaccacaactagaatgcagtgaaaaaaatgctttatttgtgaaatttgtgatgctattgc
tttatttgtaaccattataagctgcaataaacaagttaacaacaacaaaaaaaaaaaaaaaaaa
aaATTTAAATgcgcgcatc

IVT of different RTE constructs was carried out as described above. U20S cells were used in 96-well plate, at 15K cells/well. 500 ng RNA was transfected with 0.45 uL Lipofectamine. Integration was assessed based on the percentage of GFP positive cells (% GFP positive cells) after 24 h, with a higher percentage of GFP positive cells being indicative of higher levels of integration. % of GFP expressing cells was assessed by FACS 24 hours after transfection with RNA.

I343K mutation in ZFL2-2 (EX120) increased GFP positive cells approximately two-fold compared to wild-type ZFL2-2 (SM001). Q372K mutation in ZFL2-2 (EX121) increased GFP positive cells approximately 50% compared to wild-type ZFL2-2 (SM001). D588A mutation in ZFL2-2 (EX124) increased GFP positive cells approximately 50% compared to wild-type ZFL2-2 (SM001). N647K mutation in ZFL2-2 (EX126) increased GFP positive cells approximately two-fold compared to wild-type ZFL2-2 (SM001). H521P mutation in ZFL2-2 (EX136) increased GFP positive cells by over two-fold compared to wild-type ZFL2-2 (SM001). S737P mutation in ZFL2-2 (EX137) increased GFP positive cells by approximately two-fold compared to wild-type ZFL2-2 (SM001). P705A mutation in ZFL2-2 (EX138) increased GFP positive cells by approximately 50% compared to wild-type ZFL2-2 (SM001). M750L mutation in ZFL2-2 (EX142) increased GFP positive cells by over two-fold compared to wild-type ZFL2-2 (SM001). A757P mutation in ZFL2-2 (EX143) increased GFP positive cells by over 50% compared to wild-type ZFL2-2 (SM001). H717A mutation in ZFL2-2 (EX144) increased GFP positive cells by over 50% compared to wild-type ZFL2-2 (SM001).

These results illustrate the general principle that mutations in the RNA binding domain and reverse transcriptase domains can improve integration efficiency of retrotransposable elements. The mechanism of improved integration may be related to improved interaction of RNA binding domain with the RNA, due to altered electrostatic interactions, for example adding a positive charge (e.g., Q372K). The mechanism of improved integration may also be related to improved interaction of the reverse transcriptase domain with the RNA that is being reverse transcribed, due to altered electrostatic interactions, for example adding a positive charge (e.g., N647K). Alternatively, structural stability of the reverse transcriptase domain can be enhanced, for example by mutations that stabilize loop regions, for example adding a proline (e.g., H521P).

### Example 5

In order to test the effect of endonuclease domain and other mutations on integration efficiency, it was hypothesized that improving electrostatic interactions with genomic DNA may improve cleavage efficiency and thereby improve observed % integrations.

Nucleic acids were designed and produced to encode non-limiting examples of engineered proteins comprising a ZFL2-2 protein with one or more-point mutations in the endonuclease domain. Two mutations in the endonuclease domain (D64K and Y139K) and other mutations were tested and evaluated using an experimental transposition assay described in Example 3. Non-limiting examples of the heterologous polypeptides include:
EX127: ZFL2-2 with A688V mutation (SEQ ID NO: 348)
EX128: ZFL2-2 with A688I mutation (SEQ ID NO: 349)
EX129: ZFL2-2 with Y139K mutation (SEQ ID NO: 350)
EX130: ZFL2-2 with D64K mutation (SEQ ID NO: 351)
EX131: ZFL2-2 with S960R mutation (SEQ ID NO: 352)
EX133: ZFL2-2 with L444F mutation (SEQ ID NO: 354)

FIG. 4B shows results of integration assays using drivers with point mutations. In this experiment, different retrotransposable element constructs were used in the cis configuration (both driver and GFP reporter encoded by the same RNA). Aside from the mutations listed, all ZFL2-2-derived proteins used were identical in sequence to-type ZFL2-2 driver (SM001). IVT of different RTE constructs was carried out as described above. U20S cells were used in 24-well plate, at 100K cells/well. 1000 ng RNA was transfected with 1.2 uL Lipofectamine. Integration was assessed based on the percentage of GFP positive cells (% GFP positive cells) after 24 h, with a higher percentage of GFP positive cells being indicative of higher levels of integration. % GFP positive cells was assessed by FACS following 24 hours after transfection with RNA.

A688V mutation in ZFL2-2 (EX127) decreased GFP positive cells compared to wild-type ZFL2-2 (SM001). A688I mutation in ZFL2-2 (EX121) significantly decreased GFP positive cells compared to wild-type ZFL2-2 (SM001). Y139K mutation in ZFL2-2 (EX129) increased GFP positive cells approximately two-fold compared to wild-type ZFL2-2 (SM001). D64K mutation in ZFL2-2 (EX130) increased GFP positive cells approximately two-fold compared to wild-type ZFL2-2 (SM001). S960R mutation in ZFL2-2 (EX131) decreased GFP positive cells compared to wild-type ZFL2-2 (SM001). L444K mutation in ZFL2-2 (EX133) decreased GFP positive cells slightly compared to wild-type ZFL2-2 (SM001).

These results illustrate the general principle that mutations in the endonuclease domain of retrotransposable elements can improve integration efficiency. Without wishing to be bound to theory, the mechanism of improved integration may be related to improved interaction of the endonuclease domain with the DNA, due to altered electrostatic interactions (e.g., Y139K adds a positive charge). The results also show the effect on activity of making mutations in the active site of the reverse transcriptase: increasing the volume of active site amino acids (e.g., A688V mutation increases the amino acid volume) can decrease integration activity.

### Example 6

In order to test the effect of combinations of domain additions and mutations on integration efficiency with the ZFL2-2 system, it was hypothesized that a combination of modifications may allow additive or synergistic improvements to integration efficiency.

Nucleic acids were designed and produced to encode non-limiting examples of engineered proteins comprising a ZFL2-2 protein with one or more-point mutations in the endonuclease domain and one or more polypeptide fusions. Mutations in the endonuclease domain (D64K), RNA binding domain (I343K), and reverse transcriptase domain (N647K, L825G) and polypeptide fusions at the N- and C-terminus (HMG and UL12 peptides) were tested and evaluated using an experimental transposition assay described in Example 3. Non-limiting examples of the constructs tested include:
EX2107: Plasmid encoding ZFL2-2-driven GFP reporter. mRNA cassette (bold) contains Clean Cap-compatible T7 RNA promoter (TriLink Biotechnologies) and A30N10A70 polyA tail. [SEQ ID NO:319]

(SEQ ID NO: 319)
TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATATGCGGT
GTGAAATACCGCACAGATGCGTAAGGAGAAAATACCGCATCAGGCGCCATTCGCCATTCAGGCT
GCGCAACTGTTGGGAAGGGCGATCGGTGCGGGCCTCTTCGCTATTACGCCAGCTGGCGAAAGGG
GGATGTGCTGCAAGGCGATTAAGTTGGGTAACGCCAGGGTTTTCCCAGTCACGACGTTGTAAAA
CGACGGCCAGTGAATTGGAGATCGGTACTTCGCGAATGCGTCGAGATTGCAGGGTCGACTAATA
CGACTCACTATAAGGGCGGCCTACTCGACGGATCGATCCGAACAAACGACCCAACACCCGTGCG
TTTTATTCTGTCTTTTTATTGCCGATCCCCTCAGAAGAACTCGTCAAGAAGGCGATAGAAGGCG
ATGCGCTGCGAATCGGGAGCGGCGATACCGTAAAGCACGAGGAAGCGGTCAGCCCATTCGCCGC
CAAGCTCTTCAGCAATATCACGGGTAGCCAACGCTATGTCCTGATAGCGGTCGGCCGCTTTACT
TGTACAGCTCGTCCATGCCGAGAGTGATCCCGGCGGCGGTCACGAACTCCAGCAGGACCATGTG
ATCGCGCTTCTCGTTGGGGTCTTTGCTCAGGGCGGACTGGGTGCTCAGGTAGTGGTTGTCGGGC
AGCAGCACGGGGCCGTCGCCGATGGGGGTGTTCTGCTGGTAGTGGTCGGCCAGCTGCACGCTGC
CGTCCTCGATGTTGTGGCGGATCTTGAAGTTCACCTTGATGCCGTTCTTCTGCTTGTCGGCCAT
GATATAGACGTTGTGGCTGTTGTAGTTGTACTCCAGCTTGTGCCCCAGGATGTTGCCGTCCTCC
TTGAAGTCGATGCCCTTCAGCTCGATGCGGTTCACCAGGGTGTCGCCCTCGAACTTCACCTCGG
CGCGGGTCTTGTAGTTGCCGTCGTCCTTGAAGAAGATGGTGCGCTCCTGGACGTAGCCTTCGGG
CATGGCGGACTTGAAGAAGTCGTGCTGCTTCATGTGGTCGGGGTAGCGGCTGAAGCACTGCACG
CCGTAGGTCAGGGTGGTCACGAGGGTGGGCCAGGGCACGGGCAGCTTGCCGGTGGTGCAGATGA
ACTTCAGGGTCAGCTTGCCGTAGGTGGCATCGCCCTCGCCCTCGCCGGACACGCTGAACTTGTG
GCCGTTTACGTCGCCGTCCAGCTCGACCAGGATGGGCACCACCCCGGTGAACAGCTCCTCGCCC
TTGCTCACCATGGTGGCGAATTCGAAGCTTGAGCTCGAGATCTGAGTCCGGTAGCGCTAGCGGA
TCTGACGGTTCACTAAACCAGCTCTGCTTATATAGACCTCCCACCGTACACGCCTACCGCCCAT
TTGCGTCAATGGGGCGGAGTTGTTACGACATTTTGGAAAGTCCCGTTGATTTTGGTGCCAAAAC
AAACTCCCATTGACGTCAATGGGGTGGAGACTTGGAAATCCCCGTGAGTCAAACCGCTATCCAC
GCCCATTGATGTACTGCCAAAACCGCATCACCATGGTAATAGCGATGACTAATACGTAGATGTA
CTGCCAAGTAGGAAAGTCCCATAAGGTCATGTACTGGGCATAATGCCAGGCGGGCCATTTACCG
TCATTGACGTCAATAGGGGGCGTACTTGGCATATGATACACTTGATGTACTGCCAAGTGGGCAG
TTTACCGTAAATACTCCACCCATTGACGTCAATGGAAAGTCCCTATTGGCGTTACTATGGGAAC
ATACGTCATTATTGACGTCAATGGGCGGGGGTCGTTGGGCGGTCAGCCAGGCGGGCCATTTACC
GTAAGTTATGTAACGCGGAACTCCATATATGGGCTATGAACTAATGACCCCGTAATTGATTACT
ATTAAATTCCTGCAGGTTTGGGTGAAACTTGCCTTTAGTACTTATTCATTGTTGCTCTTAGTTG
TGTAAATTGCTTCCTTGTCCTCATTTGTAAGTCGCTTTGGATAAAAGCGTCTGCTAAATGACTA
AATGTAAATGTAAATGTAAAGGATCCAAAAAAAAAAAAAAAAAAAAAAAAAAAAAAaacgttGA
CTAAAAAAAAAAAAAAAAAAAAAAAAAAAAAAAAAAAAAAAAAAAAAAAAAAAAAAAAAAAAAA
AAAAAAAAaaaagcGTCTTCGCGCGCATCATCGGATGCCGGGACCGACGAGTGCAGAGGCGTGC
AAGCGAGCTTGGCGTAATCATGGTCATAGCTGTTTCCTGTGTGAAATTGTTATCCGCTCACAAT
TCCACACAACATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAGCTCACTCAAAGGCG
GTAATACGGTTATCCACAGAATCAGGGGATAACGCAGGAAAGAACATGTGAGCAAAAGGCCAGC
AAAAGGCCAGGAACCGTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTACAGAGTTCTT
GAAGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCTGGTAGCG
GTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCT
AAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATA
CGGGAGGGCTTACCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTGGTCCTGCAACTTT
ATCCGCCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGGTATGG
CTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATGTAACCCA
CTCGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATACTC
TTCCTTTTTCAATATTATTGAAGCATTTATCAGGGTTATTGTCTCATGAGCGGATACATATTTG
AATGTATTTAGAAAAATAAACAAATAGGGGTTCCGCGCACATTTCCCCGAAAAGTGCCACCTGA
CGTCTAAGAAACCATTATTATCATGACATTAACCTATAAAAATAGGCGTATCACGAGGCCCTTT
CGTC

EX2561: Plasmid encoding ZFL2-2 driver. mRNA cassette (bold) contains Clean Cap-compatible T7 RNA promoter (TriLink Biotechnologies) and A30N10A70 polyA tail. [SEQ ID: 320]

(SEQ ID NO: 320)
TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATATGCGGT
GTGAAATACCGCACAGATGCGTAAGGAGAAAATACCGCATCAGGCGCCATTCGCCATTCAGGCT
GCGCAACTGTTGGGAAGGGCGATCGGTGCGGGCCTCTTCGCTATTACGCCAGCTGGCGAAAGGG
GGATGTGCTGCAAGGCGATTAAGTTGGGTAACGCCAGGGTTTTCCCAGTCACGACGTTGTAAAA
CGACGGCCAGTGAATTGGAGATCGGTACTTCGCGAATGCGTCGAGATTGCAGGGTCGACTAATA
CGACTCACTATAAGGAGAGATATCCCTAGCTAGTTCACCGCGGCAGCGGTCGCGGCAGCCTCGT
GTGAgGACCGACGAGGGTAAAGACCATCGACTCTACCTGCGCGACTCCACCGAGCAAAGACACC
GACAAAGCACTTGAGTACTTTACTTTATTGTTTTACTTTACACTTATTTTTTGTTGTCAGTGCA
CTTTTATTATGTGTTTTCTAATTCCTGTTGTTACTAACACTCGCAAAACACGGGAGGTACGCTG
CAAGCGTAATCCTCACAACCTTCGTTCAATACATGTATCTACTATTTCACAACTCTCTCTCTCC
GTGGGCCTCTGGAATTGTCAATCAGCTGTTAACAAGGCTGATTTTATTACCTCCATAGCTACAT
ATTCTGACTATAATCTCATGGCTCTAACTGAGACCTGGTTGAGGCCGGAGGACACTGCTACACA
TGCTACTCTTTCTGCTAATTTCTCTTTTTCCCACACTCCTCGTCAGACAGGGAGAGGGGGTGGG
ACTGGACTACTAATTTCCAAAGAATGGAAATTTACTCTGATACCGTCCCTGCCAACAATCAGCT
CCTTTGAATTCCATGCAGTCACCATTATCCACCCCTTCTACATAAATGTGGTTGTCATCTACCG
CCCACCAGGTAAATTAGGTCACTTCCTAGATGAACTGGATGTTCTTCTCTCATCTTTTTCTAAT
TTTGACACTCCCTTATTGGTGCTAGGTGACTTCAACATTTACGTTGACAAACCGCAAGCTGCAG
ACTTTCAGACTTTGCTTGCCTCTTTTGACCTAAAAAGAGCACCTACTTCTGCTACCCACAAATC
AGGTAATCAGCTAGACCTTATTTACACACGACACTGCTTCACTGATCAAACAATAGTAACTCCA
CTACAAATATCTGATCATTTCCTTCTGTCTCTCAACATCCACATTACTCCTGAGCCGCCACACA
CTCCTACACTGGTTACCTTTCGCAGAAACCTACGATCTCTCTCACCCAATAGACTATCCACCAT
TGTTTCAGACTCTCTTCCTCCATCTCGCAAACTCACTGCACTTGATTCGAACAGTGCCACTAAT
ACACTCTGCTCCACACTAGCATCATGTCTAGACCGATTATGTCCTCTTGCATCCAGGCCAGCCC
GTGCCAGTCCTCCTGCACCCTGGCTCTCGGATGCTCTCCGTGAGCATCGCTCAAAACTTCGGGC
TGCGGAGAGAATTTGGCGGAAAACTAAAAATCCTGCACATCTCTTAACATACCAAACTCTTCTG
TCCTCTTTCTCAGCTGAGGTTACTTCTGCAAAGCAGACGTATTACCGTCTGAAAATCAACAATG
CCACTAATCCTCGCCTACTTTTTAAAACATTTTCCTCCCTCCTATATCCTCCTCCTCCACCCGC
ATCCTCCACACTTACTACTGATGACTTTGCTACATTCTTCTGCACCAAAACTGCAAAAATCAGT
GCTCAATTTGCTGCACCTACAACAAACACGCAAGATACAACACCAACACCACACACACTCACCT
CTTTTTCTCAGCTCTCTGAGTCTGAGGTGTCCAAACTTGTGCTATCTAGCCATGCAACCACCTG
TCCACTCGATCCCATTCCCTCTCATCTCTTGCAAGCCATCTCTCCTGCAGTCATACCAACACTG
ACTCACATAATTAACACATCTCTTGACTCTGGTTTATTCCCCACTACATTTAAGCAGGCTAGGG
TAACCCCACTGCTAAAGAAACCCAACCTGGACCATACGCTACTTGAAAACTACAGACCAGTATC
CCTGCTTCCATTCATGGCCAAGATTCTGGAGAAAGTAGTGTTCAATCAAGTCCTGGACTTTCTT
ACTCAAAACAATCTCATGGACAACAAGCAATCCGGCTTTAAGAAAGGCCACTCAACTGAGACTG
CCCTGCTCTCGGTCGTGGAGGATCTCAGACTGGCTAAAGCAGACTCTAAATCATCAGTCCTCAT
TTTGCTGGACTTGTCAGCTGCTTTTGACACTGTCAACCACCAGATCCTGCTATCTACGCTTGAG
TCACTGGGCGTTGCGGGCACTGTTATACAATGGTTTAGATCTTACCTCTCTGACAGGTCATTCA
GGGTGTCTTGGAGGGGAGAGGTGTCCAACCTACAGCATCTAAACACTGGGGTACCTCAAGGCTC
TGTTCTTGGGCCACTTCTCTTCTCCATCTACACATCATCTCTAGGACCAGTCATCCAGAGACAT
GGATTCTCCTACCACTGCTATGCTGATGACACCCAGCTATACCTCTCTTTTCATCCTGATGATC
CCTCGGTTCCAGCTCGTATCTCAGCCTGCCTGTTGGATATTTCACACTGGATGAAAGATCATCA
TCTTCAGCTGAACCTCGCAAAAACGGAAATGCTTGTAGTTTCTGCCAACCCGACTCTACACCAT
AACTTTTCAATCCAGATGGATGGGGCAACCATTACTGCATCCAAAATGGTGAAAAGCCTTGGAG
TAACGATTGATGACCAACTAAACTTCTCTGACCACATTTCTAGAACTGCTCGATCGTGCAGATT
TGCACTCTATAACATCAGAAAGATCCGACCCTTCTTATCTGAACATGCAGCTCAACTCCTTGTT
CAAGCTCTTGTTCTCTCCAAACTGGATTACTGCAACTCTCTACTAGCTTTGCTTCCAGCTAACT
CTATCAAGCCTCTTCAACTGCTCCAGAATGCAGCAGCACGAGTTGTgTTCAATGAACCTAAACG
AGCACATGTCACTCCGCTGCTAGTCCGTTTGCACTGGCTGCCAGTTGCTGCTCGCATCAAATTC
AAAACTCTGATGTTTGCCTACAAAGTGACTTCTGGCCTAGCACCTTCTTATCTGCACTCACTTC
TGCAGATCTATGTGCCCTCCAGAAACTTGCGTTCTGTGAATGAACGTCGCCTCGTGGTTCCATC
CCAAAGAGGGAAAAAATCACTTTCGCGAACGCTCACGCTCAATCTGCCCAGTTGGTGGAATGAA
CTCCCTAACTGCATCAGAACAGCAGAGTCACTCGCTATTTTCAAGAAACGACTAAAAACTCAAC
TATTTAGTCTCCACTTCACTTCCtgaTAAtagGCTGCAATTGCCTCTTTGAATATCACACTAAT
TGTACAAAAAAAAAAAAAAAAAAAAAAAAAACTACTAACACTTCCCTTCTTAGACTTTACAGAC
CGCGGCCTACTCGGATCCAAAAAAAAAAAAAAAAAAAAAAAAAAAAAAaacgttGACTAAAAAA
AAAAAAAAAAAAAAAAAAAAAAAAAAAAAAAAAAAAAAAAAAAAAAAAAAAAAAAAAAAAAAAA
aaaagcGTCTTCGCGCGCATCATCGGATGCCGGGACCGACGAGTGCAGAGGCGTGCAAGCGAGC
TTGGCGTAATCATGGTCATAGCTGTTTCCTGTGTGAAATTGTTATCCGCTCACAATTCCACACA
ACATACGAGCCGGAAGCATAAAGTGTAAAGCCTGGGGTGCCTAATGAGTGAGCTAACTCACATT
AATTGCGTTGCGCTCACTGCCCGCTTTCCAGTCGGGAAACCTGTCGTGCCAGCTGCATTAATGA
ATCGGCCAACGCGCGGGGAGAGGCGGTTTGCGTATTGGGCGCTCTTCCGCTTCCTCGCTCACTG
ACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCT
GTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTA
TCAGCAATAAACCAGCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTICATTC
AGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCAA
TACGGGATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGC
AAAATGCCGCAAAAAAGGGAATAAGGGCGACACGGAAATGTTGAATACTCATACTCTTCCTTTT
TCAATATTATTGAAGCATTTATCAGGGTTATTGTCTCATGAGCGGATACATATTTGAATGTATT
TAGAAAAATAAACAAATAGGGGTTCCGCGCACATTTCCCCGAAAAGTGCCACCTGACGTCTAAG
AAACCATTATTATCATGACATTAACCTATAAAAATAGGCGTATCACGAGGCCCTTTCGTC

EX2556: Plasmid encoding ZFL2-2 driver with N-terminal HMGN1, N647K mutation, C-terminal UL12 fusion followed by C-terminal HMGB1 fusion. mRNA cassette (bold) contains Clean Cap-compatible T7 RNA promoter (TriLink Biotechnologies) and A30N10A70 polyA tail. [SEQ ID NO: 321]

(SEQ ID NO: 321)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAATGCGTCGAGATTGCAGGGTCGACTAATACGACTCACTATAAGGAGAGATATCCCTAGCTAGTT
CACCGCGGCAGCGGTCGCGGCAGCCTCGTGTGAgGACCGACGAGGGTAAAGACCATCGACTCTACCTGC
GCGACTCCACCGAGCAAAGACACCGACAAAGCACTTGAGTACTTTACTTTATTGTTTTACTTTACACTTAT
TTTTTGTTGTCAGTGCACTTTTATTATGCCTAAGAGAAAGGTGTCCAGCGCCGAGGGCGCTGCCAAGGAA
GAGCCTAAACGGAGAAGCGCCAGACTGAGCGCCAAGCCCCCCGCCAAGGTGGAAGCCAAGCCTAAGAA
GGCCGCCGCTAAGGACAAGAGCAGCGATAAGAAAGTCCAGACCAAGGGCAAGCGGGGCGCCAAAGGC
AAACAGGCCGAGGTGGCCAACCAGGAGACAAAGGAAGATCTGCCTGCTGAGAACGGCGAAACCAAGAC
CGAGGAATCTCCAGCTTCTGACGAGGCCGGAGAGAAGGAGGCCAAAAGCGACAGCGGCTCTGAGACAC
CTGGAACAAGCGAGAGCGCCACCCCGGAATCCTGTTTTCTAATTCCTGTTGTTACTAACACTCGCAAAAC
ACGGGAGGTACGCTGCAAGCGTAATCCTCACAACCTTCGTTCAATACATGTATCTACTATTTCACAACTCT
CTCTCTCCGTGGGCCTCTGGAATTGTCAATCAGCTGTTAACAAGGCTGATTTTATTACCTCCATAGCTACA
TATTCTGACTATAATCTCATGGCTCTAACTGAGACCTGGTTGAGGCCGGAGGACACTGCTACACATGCTA
CTCTTTCTGCTAATTTCTCTTTTTCCCACACTCCTCGTCAGACAGGGAGAGGGGGTGGGACTGGACTACTA
ATTTCCAAAGAATGGAAATTTACTCTGATACCGTCCCTGCCAACAATCAGCTCCTTTGAATTCCATGCAGT
CACCATTATCCACCCCTTCTACATAAATGTGGTTGTCATCTACCGCCCACCAGGTAAATTAGGTCACTTCCT
AGATGAACTGGATGTTCTTCTCTCATCTTTTTCTAATTTTGACACTCCCTTATTGGTGCTAGGTGACTTCAA
CATTTACGTTGACAAACCGCAAGCTGCAGACTTTCAGACTTTGCTTGCCTCTTTTGACCTAAAAAGAGCAC
CTACTTCTGCTACCCACAAATCAGGTAATCAGCTAGACCTTATTTACACACGACACTGCTTCACTGATCAA
ACAATAGTAACTCCACTACAAATATCTGATCATTTCCTTCTGTCTCTCAACATCCACATTACTCCTGAGCCG
CCACACACTCCTACACTGGTTACCTTTCGCAGAAACCTACGATCTCTCTCACCCAATAGACTATCCACCATT
GTTTCAGACTCTCTTCCTCCATCTCGCAAACTCACTGCACTTGATTCGAACAGTGCCACTAATACACTCTGC
TCCACACTAGCATCATGTCTAGACCGATTATGTCCTCTTGCATCCAGGCCAGCCCGTGCCAGTCCTCCTGC
ACCCTGGCTCTCGGATGCTCTCCGTGAGCATCGCTCAAAACTTCGGGCTGCGGAGAGAATTTGGCGGAA
AACTAAAAATCCTGCACATCTCTTAACATACCAAACTCTTCTGTCCTCTTTCTCAGCTGAGGTTACTTCTGC
AAAGCAGACGTATTACCGTCTGAAAATCAACAATGCCACTAATCCTCGCCTACTTTTTAAAACATTTTCCTC
CCTCCTATATCCTCCTCCTCCACCCGCATCCTCCACACTTACTACTGATGACTTTGCTACATTCTTCTGCACC
AAAACTGCAAAAATCAGTGCTCAATTTGCTGCACCTACAACAAACACGCAAGATACAACACCAACACCAC
ACACACTCACCTCTTTTTCTCAGCTCTCTGAGTCTGAGGTGTCCAAACTTGTGCTATCTAGCCATGCAACC
ACCTGTCCACTCGATCCCATTCCCTCTCATCTCTTGCAAGCCATCTCTCCTGCAGTCATACCAACACTGACT
CACATAATTAACACATCTCTTGACTCTGGTTTATTCCCCACTACATTTAAGCAGGCTAGGGTAACCCCACT
GCTAAAGAAACCCAACCTGGACCATACGCTACTTGAAAACTACAGACCAGTATCCCTGCTTCCATTCATG
GCCAAGATTCTGGAGAAAGTAGTGTTCAATCAAGTCCTGGACTTTCTTACTCAAAACAATCTCATGGACA
ACAAGCAATCCGGCTTTAAGAAAGGCCACTCAACTGAGACTGCCCTGCTCTCGGTCGTGGAGGATCTCA
GACTGGCTAAAGCAGACTCTAAATCATCAGTCCTCATTTTGCTGGACTTGTCAGCTGCTTTTGACACTGTC
AACCACCAGATCCTGCTATCTACGCTTGAGTCACTGGGCGTTGCGGGCACTGTTATACAATGGTTTAGAT
CTTACCTCTCTGACAGGTCATTCAGGGTGTCTTGGAGGGGAGAGGTGTCCaagCTACAGCATCTAAACAC
TGGGGTACCTCAAGGCTCTGTTCTTGGGCCACTTCTCTTCTCCATCTACACATCATCTCTAGGACCAGTCA
TCCAGAGACATGGATTCTCCTACCACTGCTATGCTGATGACACCCAGCTATACCTCTCTTTTCATCCTGAT
GATCCCTCGGTTCCAGCTCGTATCTCAGCCTGCCTGTTGGATATTTCACACTGGATGAAAGATCATCATCT
TCAGCTGAACCTCGCAAAAACGGAAATGCTTGTAGTTTCTGCCAACCCGACTCTACACCATAACTTTTCAA
TCCAGATGGATGGGGCAACCATTACTGCATCCAAAATGGTGAAAAGCCTTGGAGTAACGATTGATGACC
AACTAAACTTCTCTGACCACATTTCTAGAACTGCTCGATCGTGCAGATTTGCACTCTATAACATCAGAAAG
ATCCGACCCTTCTTATCTGAACATGCAGCTCAACTCCTTGTTCAAGCTCTTGTTCTCTCCAAACTGGATTAC
TGCAACTCTCTACTAGCTTTGCTTCCAGCTAACTCTATCAAGCCTCTTCAACTGCTCCAGAATGCAGCAGC
ACGAGTTGTgTTCAATGAACCTAAACGAGCACATGTCACTCCGCTGCTAGTCCGTTTGCACTGGCTGCCA
GTTGCTGCTCGCATCAAATTCAAAACTCTGATGTTTGCCTACAAAGTGACTTCTGGCCTAGCACCTTCTTA
TCTGCACTCACTTCTGCAGATCTATGTGCCCTCCAGAAACTTGCGTTCTGTGAATGAACGTCGCCTCGTGG
TTCCATCCCAAAGAGGGAAAAAATCACTTTCGCGAACGCTCACGCTCAATCTGCCCAGTTGGTGGAATGA
ACTCCCTAACTGCATCAGAACAGCAGAGTCACTCGCTATTTTCAAGAAACGACTAAAAACTCAACTATTTA
GTCTCCACTTCACTTCCTCCGGCTCCGAGACTCCTGGAACAAGCGAGAGCGCTACACCTGAGGGCAGCG
AAAGCACAGTCGGCCCTGCTTGCCCCCCAGGAAGAACCGTGACCAAGCGGCCCTGGGCCCTGGCCGAG
GACACCCCTAGGGGCCCCGATAGCCCCCCCAAAAGACCCCGGCCTAACAGCCTGCCTCTGACCACAACAT
TCAGACCTCTGCCTCCTCCACCTCAGACCACCAGCGCCGTGGACCCTAGCAGCCACTCCCCTGTGAACCC
CCCTCGGGACCAGCACGCCACAGATACCGCCGACGAGAAGCCCAGAGCCGCTTCTCCAGCCCTGAGCGA
CGCCAGCGGACCTCCTACCCCTGACATCCCCCTGTCTCCTGGCGGCACCCACGCCAGAGATCCTGATGCT
GATCCTGACTCTCCAGACCTGGACAGCTCCGGCTCCGAGACTCCTGGAACAAGCGAGAGCGCTACACCT
GAGGGAAAAGGCGACCCCAAGAAACCTAGAGGCAAGATGAGCAGCTACGCCTTTTTCGTGCAGACCTGT
CGGGAAGAGCACAAGAAAAAGCACCCTGACGCCAGCGTGAACTTCTCTGAGTTCAGCAAGAAGTGCAG
CGAGAGATGGAAaACAATGTCCGCCAAGGAAAAGGGCAAGTTCGAGGACATGGCCAAGGCTGATAAGG
CCAGATACGAGCGGGAAATGAAAACCTACATCCCACCTAAGGGCGAGGGCGGATCTGGCAAAAGAACC
GCTGATGGCAGCGAGTTCGAGAGCCCCAAGAAAAAGAGAAAGGTGtgaTAAtagGCTGCAATTGCCTCTT
TGAATATCACACTAATTGTACAAAAAAAAAAAAAAAAAAAAAAAAAACTACTAACACTTCCCTTCTTAGAC
TTTACAGACCGCGGCCTACTCGGATCCAAAAAAAAAAAAAAAAAAAAAAAAAAAAAAaacgttGACTAAAA
AAAAAAAAAAAAAAAAAAAAAAAAAAAAAAAAAAAAAAAAAAAAAAAAAAAAAAAAAAAAAAAAAAaaa
agcGTCTTCGCGCGCATCATCGGATGCCGGGACCGACGAGTGCAGAGGCGTGCAAGCGAGCTTGGCGTA
ATCATGGTCATAGCTGTTTCCTGTGTGAAATTGTTATCCGCTCACAATTCCACACAACATACGAGCCGGAA
GCATAAAGTGTAAAGCCTGGGGTGCCTAATGAGTGAGCTAACTCACATTAATTGCGTTGCGCTCACTGCC
CGCTTTCCAGTCGGGAAACCTGTCGTGCCAGCTGCATTAATGAATCGGCCAACGCGCGGGGAGAGGCG
GTTTGCGTATTGGGCGCTCTTCCGCTTCCTCGCTCACTGACTCGCTGCGCTCGGTCGTTCGGCTGCGGCG
AGCGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGTTCAGCCCGACCGCTGCGCCT
TATCCGGTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAACTACGG
CTACACTAGAAGAACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTCGTGTAGATAACTA
CGATACGGGAGGGCTTACCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATAGTTTGCGCAA
CGTTGTTGCCATTGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCTGAGAATAGTGT
ATGCGGCGACCGAGTTGCTCTTGCCCGGCGTCAATACGGGATAATACCGCGCCACATAGCAGAACTTTA
AAAGTGCTCATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTTTCTGGGTGA
GCAAAAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTTGAATG
TATTTAGAAAAATAAACAAATAGGGGTTCCGCGCACATTTCCCCGAAAAGTGCCACCTGACGTCTAAGAA
ACCATTATTATCATGACATTAACCTATAAAAATAGGCGTATCACGAGGCCCTTTCGTC

EX2195: Plasmid encoding ZFL2-2 driver with N-terminal HMGN1, N647K and I343K mutations, C-terminal UL12 fusion followed by C-terminal HMGB1 fusion. mRNA cassette (bold) contains Clean Cap-compatible T7 RNA promoter (TriLink Biotechnologies) and A30N10A70 polyA tail. [SEQ ID NO:322]

(SEQ ID NO: 322)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAATGCGTCGAGATTGCAGGGTCGACTAATACGACTCACTATAAGGAGAGATATCCCTAGCTAGTT
CACCGCGGCAGCGGTCGCGGCAGCCTCGTGTGAgGACCGACGAGGGTAAAGACCATCGACTCTACCTGC
GCGACTCCACCGAGCAAAGACACCGACAAAGCACTTGAGTACTTTACTTTATTGTTTTACTTTACACTTAT
TTTTTGTTGTCAGTGCACTTTTATTATGCCTAAGAGAAAGGTGTCCAGCGCCGAGGGCGCTGCCAAGGAA
GAGCCTAAACGGAGAAGCGCCAGACTGAGCGCCAAGCCCCCCGCCAAGGTGGAAGCCAAGCCTAAGAA
GGCCGCCGCTAAGGACAAGAGCAGCGATAAGAAAGTCCAGACCAAGGGCAAGCGGGGCGCCAAAGGC
AAACAGGCCGAGGTGGCCAACCAGGAGACAAAGGAAGATCTGCCTGCTGAGAACGGCGAAACCAAGAC
CGAGGAATCTCCAGCTTCTGACGAGGCCGGAGAGAAGGAGGCCAAAAGCGACAGCGGCTCTGAGACAC
CTGGAACAAGCGAGAGCGCCACCCCGGAATCCTGTTTTCTAATTCCTGTTGTTACTAACACTCGCAAAAC
ACGGGAGGTACGCTGCAAGCGTAATCCTCACAACCTTCGTTCAATACATGTATCTACTATTTCACAACTCT
CTCTCTCCGTGGGCCTCTGGAATTGTCAATCAGCTGTTAACAAGGCTGATTTTATTACCTCCATAGCTACA
TATTCTGACTATAATCTCATGGCTCTAACTGAGACCTGGTTGAGGCCGGAGGACACTGCTACACATGCTA
CTCTTTCTGCTAATTTCTCTTTTTCCCACACTCCTCGTCAGACAGGGAGAGGGGGTGGGACTGGACTACTA
ATTTCCAAAGAATGGAAATTTACTCTGATACCGTCCCTGCCAACAATCAGCTCCTTTGAATTCCATGCAGT
CACCATTATCCACCCCTTCTACATAAATGTGGTTGTCATCTACCGCCCACCAGGTAAATTAGGTCACTTCCT
AGATGAACTGGATGTTCTTCTCTCATCTTTTTCTAATTTTGACACTCCCTTATTGGTGCTAGGTGACTTCAA
CATTTACGTTGACAAACCGCAAGCTGCAGACTTTCAGACTTTGCTTGCCTCTTTTGACCTAAAAAGAGCAC
CTACTTCTGCTACCCACAAATCAGGTAATCAGCTAGACCTTATTTACACACGACACTGCTTCACTGATCAA
ACAATAGTAACTCCACTACAAATATCTGATCATTTCCTTCTGTCTCTCAACATCCACATTACTCCTGAGCCG
CCACACACTCCTACACTGGTTACCTTTCGCAGAAACCTACGATCTCTCTCACCCAATAGACTATCCACCATT
GTTTCAGACTCTCTTCCTCCATCTCGCAAACTCACTGCACTTGATTCGAACAGTGCCACTAATACACTCTGC
TCCACACTAGCATCATGTCTAGACCGATTATGTCCTCTTGCATCCAGGCCAGCCCGTGCCAGTCCTCCTGC
ACCCTGGCTCTCGGATGCTCTCCGTGAGCATCGCTCAAAACTTCGGGCTGCGGAGAGAaagTGGCGGAA
AACTAAAAATCCTGCACATCTCTTAACATACCAAACTCTTCTGTCCTCTTTCTCAGCTGAGGTTACTTCTGC
AAAGCAGACGTATTACCGTCTGAAAATCAACAATGCCACTAATCCTCGCCTACTTTTTAAAACATTTTCCTC
CCTCCTATATCCTCCTCCTCCACCCGCATCCTCCACACTTACTACTGATGACTTTGCTACATTCTTCTGCACC
AAAACTGCAAAAATCAGTGCTCAATTTGCTGCACCTACAACAAACACGCAAGATACAACACCAACACCAC
ACACACTCACCTCTTTTTCTCAGCTCTCTGAGTCTGAGGTGTCCAAACTTGTGCTATCTAGCCATGCAACC
ACCTGTCCACTCGATCCCATTCCCTCTCATCTCTTGCAAGCCATCTCTCCTGCAGTCATACCAACACTGACT
CACATAATTAACACATCTCTTGACTCTGGTTTATTCCCCACTACATTTAAGCAGGCTAGGGTAACCCCACT
GCTAAAGAAACCCAACCTGGACCATACGCTACTTGAAAACTACAGACCAGTATCCCTGCTTCCATTCATG
GCCAAGATTCTGGAGAAAGTAGTGTTCAATCAAGTCCTGGACTTTCTTACTCAAAACAATCTCATGGACA
ACAAGCAATCCGGCTTTAAGAAAGGCCACTCAACTGAGACTGCCCTGCTCTCGGTCGTGGAGGATCTCA
GACTGGCTAAAGCAGACTCTAAATCATCAGTCCTCATTTTGCTGGACTTGTCAGCTGCTTTTGACACTGTC
AACCACCAGATCCTGCTATCTACGCTTGAGTCACTGGGCGTTGCGGGCACTGTTATACAATGGTTTAGAT
CTTACCTCTCTGACAGGTCATTCAGGGTGTCTTGGAGGGGAGAGGTGTCCaagCTACAGCATCTAAACAC
TGGGGTACCTCAAGGCTCTGTTCTTGGGCCACTTCTCTTCTCCATCTACACATCATCTCTAGGACCAGTCA
TCCAGAGACATGGATTCTCCTACCACTGCTATGCTGATGACACCCAGCTATACCTCTCTTTTCATCCTGAT
GATCCCTCGGTTCCAGCTCGTATCTCAGCCTGCCTGTTGGATATTTCACACTGGATGAAAGATCATCATCT
TCAGCTGAACCTCGCAAAAACGGAAATGCTTGTAGTTTCTGCCAACCCGACTCTACACCATAACTTTTCAA
TCCAGATGGATGGGGCAACCATTACTGCATCCAAAATGGTGAAAAGCCTTGGAGTAACGATTGATGACC
AACTAAACTTCTCTGACCACATTTCTAGAACTGCTCGATCGTGCAGATTTGCACTCTATAACATCAGAAAG
ATCCGACCCTTCTTATCTGAACATGCAGCTCAACTCCTTGTTCAAGCTCTTGTTCTCTCCAAACTGGATTAC
TGCAACTCTCTACTAGCTTTGCTTCCAGCTAACTCTATCAAGCCTCTTCAACTGCTCCAGAATGCAGCAGC
ACGAGTTGTgTTCAATGAACCTAAACGAGCACATGTCACTCCGCTGCTAGTCCGTTTGCACTGGCTGCCA
GTTGCTGCTCGCATCAAATTCAAAACTCTGATGTTTGCCTACAAAGTGACTTCTGGCCTAGCACCTTCTTA
TCTGCACTCACTTCTGCAGATCTATGTGCCCTCCAGAAACTTGCGTTCTGTGAATGAACGTCGCCTCGTGG
TTCCATCCCAAAGAGGGAAAAAATCACTTTCGCGAACGCTCACGCTCAATCTGCCCAGTTGGTGGAATGA
ACTCCCTAACTGCATCAGAACAGCAGAGTCACTCGCTATTTTCAAGAAACGACTAAAAACTCAACTATTTA
GTCTCCACTTCACTTCCTCCGGCTCCGAGACTCCTGGAACAAGCGAGAGCGCTACACCTGAGGGCAGCG
AAAGCACAGTCGGCCCTGCTTGCCCCCCAGGAAGAACCGTGACCAAGCGGCCCTGGGCCCTGGCCGAG
GACACCCCTAGGGGCCCCGATAGCCCCCCCAAAAGACCCCGGCCTAACAGCCTGCCTCTGACCACAACAT
TCAGACCTCTGCCTCCTCCACCTCAGACCACCAGCGCCGTGGACCCTAGCAGCCACTCCCCTGTGAACCC
CCCTCGGGACCAGCACGCCACAGATACCGCCGACGAGAAGCCCAGAGCCGCTTCTCCAGCCCTGAGCGA
CGCCAGCGGACCTCCTACCCCTGACATCCCCCTGTCTCCTGGCGGCACCCACGCCAGAGATCCTGATGCT
GATCCTGACTCTCCAGACCTGGACAGCTCCGGCTCCGAGACTCCTGGAACAAGCGAGAGCGCTACACCT
GAGGGAAAAGGCGACCCCAAGAAACCTAGAGGCAAGATGAGCAGCTACGCCTTTTTCGTGCAGACCTGT
CGGGAAGAGCACAAGAAAAAGCACCCTGACGCCAGCGTGAACTTCTCTGAGTTCAGCAAGAAGTGCAG
CGAGAGATGGAAaACAATGTCCGCCAAGGAAAAGGGCAAGTTCGAGGACATGGCCAAGGCTGATAAGG
CCAGATACGAGCGGGAAATGAAAACCTACATCCCACCTAAGGGCGAGGGCGGATCTGGCAAAAGAACC
GCTGATGGCAGCGAGTTCGAGAGCCCCAAGAAAAAGAGAAAGGTGtaaGCTGCAATTGCCTCTTTGAAT
ATCACACTAATTGTACAAAAAAAAAAAAAAAAAAAAAAAAAACTACTAACACTTCCCTTCTTAGACTTTAC
AGACCGCGGCCTACTCGGATCCAAAAAAAAAAAAAAAAAAAAAAAAAAAAAAaacgttGACTAAAAAAAA
AAAAAAAAAAAAAAAAAAAAAAAAAAAAAAAAAAAAAAAAAAAAAAAAAAAAAAAAAAAAAAaaaagcGT
CTTCGCGCGCATCATCGGATGCCGGGACCGACGAGTGCAGAGGCGTGCAAGCGAGCTTGGCGTAATCA
TGGTCATAGCTGTTTCCTGTGTGAAATTGTTATCCGCTCACAATTCCACACAACATACGAGCCGGAAGCAT
AAAGTGTAAAGCCTGGGGTGCCTAATGAGTGAGCTAACTCACATTAATTGCGTTGCGCTCACTGCCCGCT
TTCCAGTCGGGAAACCTGTCGTGCCAGCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTCGGCTGCGGCGAGCGG
TATCAGCTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCATAGGCTCC
GCCCCCCTGACGAGCATCACAAAAATCGACGCTCAAGTCAGAGGTGGCGAAACCCGACAGGACTATAAA
GATACCAGGCGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTACGGCTACA
CTAGAAGAACAGTATTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGCAGATTACGCGCAGA
AAAAAAGGATCTCAAGAAGATCCTTTGATCTTTTCTACGGGGTCTGACGCTCAGTGGAACGAAAACTCAC
GTTAAGGGATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGTGAGGCACC
TATCTCAGCGATCTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCACCGGCTCCAGATT
TATCAGCAATAAACCAGCCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGAATAGTGTATGCG
GCGACCGAGTTGCTCTTGCCCGGCGTCAATACGGGATAATACCGCGCCACATAGCAGAACTTTAAAAGT
GCTCATCATTGGAAAACGTTCTTCGGGGCGAAAACTCTCAAGGATCTTACCGCTGTTGAGATCCAGTTCG
ATGTAACCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACGTCTAAGAAACCATT
ATTATCATGACATTAACCTATAAAAATAGGCGTATCACGAGGCCCTTTCGTC

EX2196: Plasmid encoding ZFL2-2 driver with N-terminal HMGN1, D64K, N647K, and I343K mutations, C-terminal UL12 fusion followed by C-terminal HMGB1 fusion. mRNA cassette (bold) contains Clean Cap-compatible T7 RNA promoter (TriLink Biotechnologies) and A30N10A70 polyA tail. [SEQ ID NO: 323]

(SEQ ID NO: 323)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAATGCGTCGAGATTGCAGGGTCGACTAATACGACTCACTATAAGGAGAGATATCCCTAGCTAGTT
CACCGCGGCAGCGGTCGCGGCAGCCTCGTGTGAgGACCGACGAGGGTAAAGACCATCGACTCTACCTGC
GCGACTCCACCGAGCAAAGACACCGACAAAGCACTTGAGTACTTTACTTTATTGTTTTACTTTACACTTAT
TTTTTGTTGTCAGTGCACTTTTATTATGCCTAAGAGAAAGGTGTCCAGCGCCGAGGGCGCTGCCAAGGAA
GAGCCTAAACGGAGAAGCGCCAGACTGAGCGCCAAGCCCCCCGCCAAGGTGGAAGCCAAGCCTAAGAA
GGCCGCCGCTAAGGACAAGAGCAGCGATAAGAAAGTCCAGACCAAGGGCAAGCGGGGCGCCAAAGGC
AAACAGGCCGAGGTGGCCAACCAGGAGACAAAGGAAGATCTGCCTGCTGAGAACGGCGAAACCAAGAC
CGAGGAATCTCCAGCTTCTGACGAGGCCGGAGAGAAGGAGGCCAAAAGCGACAGCGGCTCTGAGACAC
CTGGAACAAGCGAGAGCGCCACCCCGGAATCCTGTTTTCTAATTCCTGTTGTTACTAACACTCGCAAAAC
ACGGGAGGTACGCTGCAAGCGTAATCCTCACAACCTTCGTTCAATACATGTATCTACTATTTCACAACTCT
CTCTCTCCGTGGGCCTCTGGAATTGTCAATCAGCTGTTAACAAGGCTGATTTTATTACCTCCATAGCTACA
TATTCTaagTATAATCTCATGGCTCTAACTGAGACCTGGTTGAGGCCGGAGGACACTGCTACACATGCTAC
TCTTTCTGCTAATTTCTCTTTTTCCCACACTCCTCGTCAGACAGGGAGAGGGGGTGGGACTGGACTACTAA
TTTCCAAAGAATGGAAATTTACTCTGATACCGTCCCTGCCAACAATCAGCTCCTTTGAATTCCATGCAGTC
ACCATTATCCACCCCTTCTACATAAATGTGGTTGTCATCTACCGCCCACCAGGTAAATTAGGTCACTTCCTA
GATGAACTGGATGTTCTTCTCTCATCTTTTTCTAATTTTGACACTCCCTTATTGGTGCTAGGTGACTTCAAC
ATTTACGTTGACAAACCGCAAGCTGCAGACTTTCAGACTTTGCTTGCCTCTTTTGACCTAAAAAGAGCACC
TACTTCTGCTACCCACAAATCAGGTAATCAGCTAGACCTTATTTACACACGACACTGCTTCACTGATCAAA
CAATAGTAACTCCACTACAAATATCTGATCATTTCCTTCTGTCTCTCAACATCCACATTACTCCTGAGCCGC
CACACACTCCTACACTGGTTACCTTTCGCAGAAACCTACGATCTCTCTCACCCAATAGACTATCCACCATTG
TTTCAGACTCTCTTCCTCCATCTCGCAAACTCACTGCACTTGATTCGAACAGTGCCACTAATACACTCTGCT
CCACACTAGCATCATGTCTAGACCGATTATGTCCTCTTGCATCCAGGCCAGCCCGTGCCAGTCCTCCTGCA
CCCTGGCTCTCGGATGCTCTCCGTGAGCATCGCTCAAAACTTCGGGCTGCGGAGAGAaagTGGCGGAAA
ACTAAAAATCCTGCACATCTCTTAACATACCAAACTCTTCTGTCCTCTTTCTCAGCTGAGGTTACTTCTGCA
AAGCAGACGTATTACCGTCTGAAAATCAACAATGCCACTAATCCTCGCCTACTTTTTAAAACATTTTCCTCC
CTCCTATATCCTCCTCCTCCACCCGCATCCTCCACACTTACTACTGATGACTTTGCTACATTCTTCTGCACCA
AAACTGCAAAAATCAGTGCTCAATTTGCTGCACCTACAACAAACACGCAAGATACAACACCAACACCACA
CACACTCACCTCTTTTTCTCAGCTCTCTGAGTCTGAGGTGTCCAAACTTGTGCTATCTAGCCATGCAACCAC
CTGTCCACTCGATCCCATTCCCTCTCATCTCTTGCAAGCCATCTCTCCTGCAGTCATACCAACACTGACTCA
CATAATTAACACATCTCTTGACTCTGGTTTATTCCCCACTACATTTAAGCAGGCTAGGGTAACCCCACTGC
TAAAGAAACCCAACCTGGACCATACGCTACTTGAAAACTACAGACCAGTATCCCTGCTTCCATTCATGGCC
AAGATTCTGGAGAAAGTAGTGTTCAATCAAGTCCTGGACTTTCTTACTCAAAACAATCTCATGGACAACA
AGCAATCCGGCTTTAAGAAAGGCCACTCAACTGAGACTGCCCTGCTCTCGGTCGTGGAGGATCTCAGACT
GGCTAAAGCAGACTCTAAATCATCAGTCCTCATTTTGCTGGACTTGTCAGCTGCTTTTGACACTGTCAACC
ACCAGATCCTGCTATCTACGCTTGAGTCACTGGGCGTTGCGGGCACTGTTATACAATGGTTTAGATCTTA
CCTCTCTGACAGGTCATTCAGGGTGTCTTGGAGGGGAGAGGTGTCCaagCTACAGCATCTAAACACTGGG
GTACCTCAAGGCTCTGTTCTTGGGCCACTTCTCTTCTCCATCTACACATCATCTCTAGGACCAGTCATCCAG
AGACATGGATTCTCCTACCACTGCTATGCTGATGACACCCAGCTATACCTCTCTTTTCATCCTGATGATCCC
TCGGTTCCAGCTCGTATCTCAGCCTGCCTGTTGGATATTTCACACTGGATGAAAGATCATCATCTTCAGCT
GAACCTCGCAAAAACGGAAATGCTTGTAGTTTCTGCCAACCCGACTCTACACCATAACTTTTCAATCCAGA
TGGATGGGGCAACCATTACTGCATCCAAAATGGTGAAAAGCCTTGGAGTAACGATTGATGACCAACTAA
ACTTCTCTGACCACATTTCTAGAACTGCTCGATCGTGCAGATTTGCACTCTATAACATCAGAAAGATCCGA
CCCTTCTTATCTGAACATGCAGCTCAACTCCTTGTTCAAGCTCTTGTTCTCTCCAAACTGGATTACTGCAAC
TCTCTACTAGCTTTGCTTCCAGCTAACTCTATCAAGCCTCTTCAACTGCTCCAGAATGCAGCAGCACGAGT
TGTgTTCAATGAACCTAAACGAGCACATGTCACTCCGCTGCTAGTCCGTTTGCACTGGCTGCCAGTTGCTG
CTCGCATCAAATTCAAAACTCTGATGTTTGCCTACAAAGTGACTTCTGGCCTAGCACCTTCTTATCTGCACT
CACTTCTGCAGATCTATGTGCCCTCCAGAAACTTGCGTTCTGTGAATGAACGTCGCCTCGTGGTTCCATCC
CAAAGAGGGAAAAAATCACTTTCGCGAACGCTCACGCTCAATCTGCCCAGTTGGTGGAATGAACTCCCTA
ACTGCATCAGAACAGCAGAGTCACTCGCTATTTTCAAGAAACGACTAAAAACTCAACTATTTAGTCTCCAC
TTCACTTCCTCCGGCTCCGAGACTCCTGGAACAAGCGAGAGCGCTACACCTGAGGGCAGCGAAAGCACA
GTCGGCCCTGCTTGCCCCCCAGGAAGAACCGTGACCAAGCGGCCCTGGGCCCTGGCCGAGGACACCCCT
AGGGGCCCCGATAGCCCCCCCAAAAGACCCCGGCCTAACAGCCTGCCTCTGACCACAACATTCAGACCTC
TGCCTCCTCCACCTCAGACCACCAGCGCCGTGGACCCTAGCAGCCACTCCCCTGTGAACCCCCCTCGGGA
CCAGCACGCCACAGATACCGCCGACGAGAAGCCCAGAGCCGCTTCTCCAGCCCTGAGCGACGCCAGCG
GACCTCCTACCCCTGACATCCCCCTGTCTCCTGGCGGCACCCACGCCAGAGATCCTGATGCTGATCCTGA
CTCTCCAGACCTGGACAGCTCCGGCTCCGAGACTCCTGGAACAAGCGAGAGCGCTACACCTGAGGGAAA
AGGCGACCCCAAGAAACCTAGAGGCAAGATGAGCAGCTACGCCTTTTTCGTGCAGACCTGTCGGGAAGA
GCACAAGAAAAAGCACCCTGACGCCAGCGTGAACTTCTCTGAGTTCAGCAAGAAGTGCAGCGAGAGATG
GAAaACAATGTCCGCCAAGGAAAAGGGCAAGTTCGAGGACATGGCCAAGGCTGATAAGGCCAGATACG
AGCGGGAAATGAAAACCTACATCCCACCTAAGGGCGAGGGCGGATCTGGCAAAAGAACCGCTGATGGC
AGCGAGTTCGAGAGCCCCAAGAAAAAGAGAAAGGTGtaaGCTGCAATTGCCTCTTTGAATATCACACTAA
TTGTACAAAAAAAAAAAAAAAAAAAAAAAAAACTACTAACACTTCCCTTCTTAGACTTTACAGACCGCGG
CCTACTCGGATCCAAAAAAAAAAAAAAAAAAAAAAAAAAAAAAaacgttGACTAAAAAAAAAAAAAAAAAA
AAAAAAAAAAAAAAAAAAAAAAAAAAAAAAAAAAAAAAAAAAAAAAAAAAAAaaaagcGTCTTCGCGCGC
ATCATCGGATGCCGGGACCGACGAGTGCAGAGGCGTGCAAGCGAGCTTGGCGTAATCATGGTCATAGC
TGTTTCCTGTGTGAAATTGTTATCCGCTCACAATTCCACACAACATACGAGCCGGAAGCATAAAGTGTAAA
GCCTGGGGTGCCTAATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTTGTCA
GAAGTAAGTTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTT
CAATATTATTGAAGCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGACGTCTAAGAAACCATTATTATC
ATGACATTAACCTATAAAAATAGGCGTATCACGAGGCCCTTTCGTC

EX2199: Plasmid encoding ZFL2-2 driver with N-terminal HMGN1, D64K, N647K, L825G, and I343K mutations, C-terminal UL12 fusion followed by C-terminal HMGB1 fusion. mRNA cassette (bold) contains Clean Cap-compatible T7 RNA promoter (TriLink Biotechnologies) and A30N10A70 polyA tail. [SEQ ID NO: 324]

(SEQ ID NO: 324)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAATGCGTCGAGATTGCAGGGTCGACTAATACGACTCACTATAAGGAGAGATATCCCTAGCTAGTT
CACCGCGGCAGCGGTCGCGGCAGCCTCGTGTGAgGACCGACGAGGGTAAAGACCATCGACTCTACCTGC
GCGACTCCACCGAGCAAAGACACCGACAAAGCACTTGAGTACTTTACTTTATTGTTTTACTTTACACTTAT
TTTTTGTTGTCAGTGCACTTTTATTATGCCTAAGAGAAAGGTGTCCAGCGCCGAGGGCGCTGCCAAGGAA
GAGCCTAAACGGAGAAGCGCCAGACTGAGCGCCAAGCCCCCCGCCAAGGTGGAAGCCAAGCCTAAGAA
GGCCGCCGCTAAGGACAAGAGCAGCGATAAGAAAGTCCAGACCAAGGGCAAGCGGGGCGCCAAAGGC
AAACAGGCCGAGGTGGCCAACCAGGAGACAAAGGAAGATCTGCCTGCTGAGAACGGCGAAACCAAGAC
CGAGGAATCTCCAGCTTCTGACGAGGCCGGAGAGAAGGAGGCCAAAAGCGACAGCGGCTCTGAGACAC
CTGGAACAAGCGAGAGCGCCACCCCGGAATCCTGTTTTCTAATTCCTGTTGTTACTAACACTCGCAAAAC
ACGGGAGGTACGCTGCAAGCGTAATCCTCACAACCTTCGTTCAATACATGTATCTACTATTTCACAACTCT
CTCTCTCCGTGGGCCTCTGGAATTGTCAATCAGCTGTTAACAAGGCTGATTTTATTACCTCCATAGCTACA
TATTCTaagTATAATCTCATGGCTCTAACTGAGACCTGGTTGAGGCCGGAGGACACTGCTACACATGCTAC
TCTTTCTGCTAATTTCTCTTTTTCCCACACTCCTCGTCAGACAGGGAGAGGGGGTGGGACTGGACTACTAA
TTTCCAAAGAATGGAAATTTACTCTGATACCGTCCCTGCCAACAATCAGCTCCTTTGAATTCCATGCAGTC
ACCATTATCCACCCCTTCTACATAAATGTGGTTGTCATCTACCGCCCACCAGGTAAATTAGGTCACTTCCTA
GATGAACTGGATGTTCTTCTCTCATCTTTTTCTAATTTTGACACTCCCTTATTGGTGCTAGGTGACTTCAAC
ATTTACGTTGACAAACCGCAAGCTGCAGACTTTCAGACTTTGCTTGCCTCTTTTGACCTAAAAAGAGCACC
TACTTCTGCTACCCACAAATCAGGTAATCAGCTAGACCTTATTTACACACGACACTGCTTCACTGATCAAA
CAATAGTAACTCCACTACAAATATCTGATCATTTCCTTCTGTCTCTCAACATCCACATTACTCCTGAGCCGC
CACACACTCCTACACTGGTTACCTTTCGCAGAAACCTACGATCTCTCTCACCCAATAGACTATCCACCATTG
TTTCAGACTCTCTTCCTCCATCTCGCAAACTCACTGCACTTGATTCGAACAGTGCCACTAATACACTCTGCT
CCACACTAGCATCATGTCTAGACCGATTATGTCCTCTTGCATCCAGGCCAGCCCGTGCCAGTCCTCCTGCA
CCCTGGCTCTCGGATGCTCTCCGTGAGCATCGCTCAAAACTTCGGGCTGCGGAGAGAaagTGGCGGAAA
ACTAAAAATCCTGCACATCTCTTAACATACCAAACTCTTCTGTCCTCTTTCTCAGCTGAGGTTACTTCTGCA
AAGCAGACGTATTACCGTCTGAAAATCAACAATGCCACTAATCCTCGCCTACTTTTTAAAACATTTTCCTCC
CTCCTATATCCTCCTCCTCCACCCGCATCCTCCACACTTACTACTGATGACTTTGCTACATTCTTCTGCACCA
AAACTGCAAAAATCAGTGCTCAATTTGCTGCACCTACAACAAACACGCAAGATACAACACCAACACCACA
CACACTCACCTCTTTTTCTCAGCTCTCTGAGTCTGAGGTGTCCAAACTTGTGCTATCTAGCCATGCAACCAC
CTGTCCACTCGATCCCATTCCCTCTCATCTCTTGCAAGCCATCTCTCCTGCAGTCATACCAACACTGACTCA
CATAATTAACACATCTCTTGACTCTGGTTTATTCCCCACTACATTTAAGCAGGCTAGGGTAACCCCACTGC
TAAAGAAACCCAACCTGGACCATACGCTACTTGAAAACTACAGACCAGTATCCCTGCTTCCATTCATGGCC
AAGATTCTGGAGAAAGTAGTGTTCAATCAAGTCCTGGACTTTCTTACTCAAAACAATCTCATGGACAACA
AGCAATCCGGCTTTAAGAAAGGCCACTCAACTGAGACTGCCCTGCTCTCGGTCGTGGAGGATCTCAGACT
GGCTAAAGCAGACTCTAAATCATCAGTCCTCATTTTGCTGGACTTGTCAGCTGCTTTTGACACTGTCAACC
ACCAGATCCTGCTATCTACGCTTGAGTCACTGGGCGTTGCGGGCACTGTTATACAATGGTTTAGATCTTA
CCTCTCTGACAGGTCATTCAGGGTGTCTTGGAGGGGAGAGGTGTCCaagCTACAGCATCTAAACACTGGG
GTACCTCAAGGCTCTGTTCTTGGGCCACTTCTCTTCTCCATCTACACATCATCTCTAGGACCAGTCATCCAG
AGACATGGATTCTCCTACCACTGCTATGCTGATGACACCCAGCTATACCTCTCTTTTCATCCTGATGATCCC
TCGGTTCCAGCTCGTATCTCAGCCTGCCTGTTGGATATTTCACACTGGATGAAAGATCATCATCTTCAGCT
GAACCTCGCAAAAACGGAAATGCTTGTAGTTTCTGCCAACCCGACTCTACACCATAACTTTTCAATCCAGA
TGGATGGGGCAACCATTACTGCATCCAAAATGGTGAAAAGCCTTGGAGTAACGATTGATGACCAACTAA
ACTTCTCTGACCACATTTCTAGAACTGCTCGATCGTGCAGATTTGCACTCTATAACATCAGAAAGATCCGA
CCCTTCTTATCTGAACATGCAGCTCAACTCCTTGTTCAAGCTCTTGTTCTCTCCAAACTGGATTACTGCAAC
TCTCTACTAGCTggcCTTCCAGCTAACTCTATCAAGCCTCTTCAACTGCTCCAGAATGCAGCAGCACGAGTT
GTgTTCAATGAACCTAAACGAGCACATGTCACTCCGCTGCTAGTCCGTTTGCACTGGCTGCCAGTTGCTGC
TCGCATCAAATTCAAAACTCTGATGTTTGCCTACAAAGTGACTTCTGGCCTAGCACCTTCTTATCTGCACTC
ACTTCTGCAGATCTATGTGCCCTCCAGAAACTTGCGTTCTGTGAATGAACGTCGCCTCGTGGTTCCATCCC
AAAGAGGGAAAAAATCACTTTCGCGAACGCTCACGCTCAATCTGCCCAGTTGGTGGAATGAACTCCCTAA
CTGCATCAGAACAGCAGAGTCACTCGCTATTTTCAAGAAACGACTAAAAACTCAACTATTTAGTCTCCACT
TCACTTCCTCCGGCTCCGAGACTCCTGGAACAAGCGAGAGCGCTACACCTGAGGGCAGCGAAAGCACAG
TCGGCCCTGCTTGCCCCCCAGGAAGAACCGTGACCAAGCGGCCCTGGGCCCTGGCCGAGGACACCCCTA
GGGGCCCCGATAGCCCCCCCAAAAGACCCCGGCCTAACAGCCTGCCTCTGACCACAACATTCAGACCTCT
GCCTCCTCCACCTCAGACCACCAGCGCCGTGGACCCTAGCAGCCACTCCCCTGTGAACCCCCCTCGGGAC
CAGCACGCCACAGATACCGCCGACGAGAAGCCCAGAGCCGCTTCTCCAGCCCTGAGCGACGCCAGCGG
ACCTCCTACCCCTGACATCCCCCTGTCTCCTGGCGGCACCCACGCCAGAGATCCTGATGCTGATCCTGACT
CTCCAGACCTGGACAGCTCCGGCTCCGAGACTCCTGGAACAAGCGAGAGCGCTACACCTGAGGGAAAA
GGCGACCCCAAGAAACCTAGAGGCAAGATGAGCAGCTACGCCTTTTTCGTGCAGACCTGTCGGGAAGAG
CACAAGAAAAAGCACCCTGACGCCAGCGTGAACTTCTCTGAGTTCAGCAAGAAGTGCAGCGAGAGATGG
AAaACAATGTCCGCCAAGGAAAAGGGCAAGTTCGAGGACATGGCCAAGGCTGATAAGGCCAGATACGA
GCGGGAAATGAAAACCTACATCCCACCTAAGGGCGAGGGCGGATCTGGCAAAAGAACCGCTGATGGCA
GCGAGTTCGAGAGCCCCAAGAAAAAGAGAAAGGTGtaaGCTGCAATTGCCTCTTTGAATATCACACTAAT
TGTACAAAAAAAAAAAAAAAAAAAAAAAAAACTACTAACACTTCCCTTCTTAGACTTTACAGACCGCGGC
CTACTCGGATCCAAAAAAAAAAAAAAAAAAAAAAAAAAAAAAaacgttGACTAAAAAAAAAAAAAAAAAAA
AAAAAAAAAAAAAAAAAAAAAAAAAAAAAAAAAAAAAAAAAAAAAAAAAAAaaaagcGTCTTCGCGCGCA
TCATCGGATGCCGGGACCGACGAGTGCAGAGGCGTGCAAGCGAGCTTGGCGTAATCATGGTCATAGCT
GTTTCCTGTGTGAAATTGTTATCCGCTCACAATTCCACACAACATACGAGCCGGAAGCATAAAGTGTAAA
GCCTGGGGTGCCTAATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTTGTCA
GAAGTAAGTTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTT
CAATATTATTGAAGCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGACGTCTAAGAAACCATTATTATC
ATGACATTAACCTATAAAAATAGGCGTATCACGAGGCCCTTTCGTC

EX2200: Plasmid encoding ZFL2-2 driver with N-terminal HMGN1, D64K, N647K, M750L, and I343K mutations, C-terminal UL12 fusion followed by C-terminal HMGB1 fusion. mRNA cassette (bold) contains Clean Cap-compatible T7 RNA promoter (TriLink Biotechnologies) and A30N10A70 polyA tail.

(SEQ ID NO: 325)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAATGCGTCGAGATTGCAGGGTCGACTAATACGACTCACTATAAGGAGAGATATCCCTAGCTAGTT
CACCGCGGCAGCGGTCGCGGCAGCCTCGTGTGAgGACCGACGAGGGTAAAGACCATCGACTCTACCTGC
GCGACTCCACCGAGCAAAGACACCGACAAAGCACTTGAGTACTTTACTTTATTGTTTTACTTTACACTTAT
TTTTTGTTGTCAGTGCACTTTTATTATGCCTAAGAGAAAGGTGTCCAGCGCCGAGGGCGCTGCCAAGGAA
GAGCCTAAACGGAGAAGCGCCAGACTGAGCGCCAAGCCCCCCGCCAAGGTGGAAGCCAAGCCTAAGAA
GGCCGCCGCTAAGGACAAGAGCAGCGATAAGAAAGTCCAGACCAAGGGCAAGCGGGGCGCCAAAGGC
AAACAGGCCGAGGTGGCCAACCAGGAGACAAAGGAAGATCTGCCTGCTGAGAACGGCGAAACCAAGAC
CGAGGAATCTCCAGCTTCTGACGAGGCCGGAGAGAAGGAGGCCAAAAGCGACAGCGGCTCTGAGACAC
CTGGAACAAGCGAGAGCGCCACCCCGGAATCCTGTTTTCTAATTCCTGTTGTTACTAACACTCGCAAAAC
ACGGGAGGTACGCTGCAAGCGTAATCCTCACAACCTTCGTTCAATACATGTATCTACTATTTCACAACTCT
CTCTCTCCGTGGGCCTCTGGAATTGTCAATCAGCTGTTAACAAGGCTGATTTTATTACCTCCATAGCTACA
TATTCTaagTATAATCTCATGGCTCTAACTGAGACCTGGTTGAGGCCGGAGGACACTGCTACACATGCTAC
TCTTTCTGCTAATTTCTCTTTTTCCCACACTCCTCGTCAGACAGGGAGAGGGGGTGGGACTGGACTACTAA
TTTCCAAAGAATGGAAATTTACTCTGATACCGTCCCTGCCAACAATCAGCTCCTTTGAATTCCATGCAGTC
ACCATTATCCACCCCTTCTACATAAATGTGGTTGTCATCTACCGCCCACCAGGTAAATTAGGTCACTTCCTA
GATGAACTGGATGTTCTTCTCTCATCTTTTTCTAATTTTGACACTCCCTTATTGGTGCTAGGTGACTTCAAC
ATTTACGTTGACAAACCGCAAGCTGCAGACTTTCAGACTTTGCTTGCCTCTTTTGACCTAAAAAGAGCACC
TACTTCTGCTACCCACAAATCAGGTAATCAGCTAGACCTTATTTACACACGACACTGCTTCACTGATCAAA
CAATAGTAACTCCACTACAAATATCTGATCATTTCCTTCTGTCTCTCAACATCCACATTACTCCTGAGCCGC
CACACACTCCTACACTGGTTACCTTTCGCAGAAACCTACGATCTCTCTCACCCAATAGACTATCCACCATTG
TTTCAGACTCTCTTCCTCCATCTCGCAAACTCACTGCACTTGATTCGAACAGTGCCACTAATACACTCTGCT
CCACACTAGCATCATGTCTAGACCGATTATGTCCTCTTGCATCCAGGCCAGCCCGTGCCAGTCCTCCTGCA
CCCTGGCTCTCGGATGCTCTCCGTGAGCATCGCTCAAAACTTCGGGCTGCGGAGAGAaagTGGCGGAAA
ACTAAAAATCCTGCACATCTCTTAACATACCAAACTCTTCTGTCCTCTTTCTCAGCTGAGGTTACTTCTGCA
AAGCAGACGTATTACCGTCTGAAAATCAACAATGCCACTAATCCTCGCCTACTTTTTAAAACATTTTCCTCC
CTCCTATATCCTCCTCCTCCACCCGCATCCTCCACACTTACTACTGATGACTTTGCTACATTCTTCTGCACCA
AAACTGCAAAAATCAGTGCTCAATTTGCTGCACCTACAACAAACACGCAAGATACAACACCAACACCACA
CACACTCACCTCTTTTTCTCAGCTCTCTGAGTCTGAGGTGTCCAAACTTGTGCTATCTAGCCATGCAACCAC
CTGTCCACTCGATCCCATTCCCTCTCATCTCTTGCAAGCCATCTCTCCTGCAGTCATACCAACACTGACTCA
CATAATTAACACATCTCTTGACTCTGGTTTATTCCCCACTACATTTAAGCAGGCTAGGGTAACCCCACTGC
TAAAGAAACCCAACCTGGACCATACGCTACTTGAAAACTACAGACCAGTATCCCTGCTTCCATTCATGGCC
AAGATTCTGGAGAAAGTAGTGTTCAATCAAGTCCTGGACTTTCTTACTCAAAACAATCTCATGGACAACA
AGCAATCCGGCTTTAAGAAAGGCCACTCAACTGAGACTGCCCTGCTCTCGGTCGTGGAGGATCTCAGACT
GGCTAAAGCAGACTCTAAATCATCAGTCCTCATTTTGCTGGACTTGTCAGCTGCTTTTGACACTGTCAACC
ACCAGATCCTGCTATCTACGCTTGAGTCACTGGGCGTTGCGGGCACTGTTATACAATGGTTTAGATCTTA
CCTCTCTGACAGGTCATTCAGGGTGTCTTGGAGGGGAGAGGTGTCCaagCTACAGCATCTAAACACTGGG
GTACCTCAAGGCTCTGTTCTTGGGCCACTTCTCTTCTCCATCTACACATCATCTCTAGGACCAGTCATCCAG
AGACATGGATTCTCCTACCACTGCTATGCTGATGACACCCAGCTATACCTCTCTTTTCATCCTGATGATCCC
TCGGTTCCAGCTCGTATCTCAGCCTGCCTGTTGGATATTTCACACTGGATGAAAGATCATCATCTTCAGCT
GAACCTCGCAAAAACGGAAATGCTTGTAGTTTCTGCCAACCCGACTCTACACCATAACTTTTCAATCCAGc
tgGATGGGGCAACCATTACTGCATCCAAAATGGTGAAAAGCCTTGGAGTAACGATTGATGACCAACTAAA
CTTCTCTGACCACATTTCTAGAACTGCTCGATCGTGCAGATTTGCACTCTATAACATCAGAAAGATCCGAC
CCTTCTTATCTGAACATGCAGCTCAACTCCTTGTTCAAGCTCTTGTTCTCTCCAAACTGGATTACTGCAACT
CTCTACTAGCTTTGCTTCCAGCTAACTCTATCAAGCCTCTTCAACTGCTCCAGAATGCAGCAGCACGAGTT
GTgTTCAATGAACCTAAACGAGCACATGTCACTCCGCTGCTAGTCCGTTTGCACTGGCTGCCAGTTGCTGC
TCGCATCAAATTCAAAACTCTGATGTTTGCCTACAAAGTGACTTCTGGCCTAGCACCTTCTTATCTGCACTC
ACTTCTGCAGATCTATGTGCCCTCCAGAAACTTGCGTTCTGTGAATGAACGTCGCCTCGTGGTTCCATCCC
AAAGAGGGAAAAAATCACTTTCGCGAACGCTCACGCTCAATCTGCCCAGTTGGTGGAATGAACTCCCTAA
CTGCATCAGAACAGCAGAGTCACTCGCTATTTTCAAGAAACGACTAAAAACTCAACTATTTAGTCTCCACT
TCACTTCCTCCGGCTCCGAGACTCCTGGAACAAGCGAGAGCGCTACACCTGAGGGCAGCGAAAGCACAG
TCGGCCCTGCTTGCCCCCCAGGAAGAACCGTGACCAAGCGGCCCTGGGCCCTGGCCGAGGACACCCCTA
GGGGCCCCGATAGCCCCCCCAAAAGACCCCGGCCTAACAGCCTGCCTCTGACCACAACATTCAGACCTCT
GCCTCCTCCACCTCAGACCACCAGCGCCGTGGACCCTAGCAGCCACTCCCCTGTGAACCCCCCTCGGGAC
CAGCACGCCACAGATACCGCCGACGAGAAGCCCAGAGCCGCTTCTCCAGCCCTGAGCGACGCCAGCGG
ACCTCCTACCCCTGACATCCCCCTGTCTCCTGGCGGCACCCACGCCAGAGATCCTGATGCTGATCCTGACT
CTCCAGACCTGGACAGCTCCGGCTCCGAGACTCCTGGAACAAGCGAGAGCGCTACACCTGAGGGAAAA
GGCGACCCCAAGAAACCTAGAGGCAAGATGAGCAGCTACGCCTTTTTCGTGCAGACCTGTCGGGAAGAG
CACAAGAAAAAGCACCCTGACGCCAGCGTGAACTTCTCTGAGTTCAGCAAGAAGTGCAGCGAGAGATGG
AAaACAATGTCCGCCAAGGAAAAGGGCAAGTTCGAGGACATGGCCAAGGCTGATAAGGCCAGATACGA
GCGGGAAATGAAAACCTACATCCCACCTAAGGGCGAGGGCGGATCTGGCAAAAGAACCGCTGATGGCA
GCGAGTTCGAGAGCCCCAAGAAAAAGAGAAAGGTGtaaGCTGCAATTGCCTCTTTGAATATCACACTAAT
TGTACAAAAAAAAAAAAAAAAAAAAAAAAAACTACTAACACTTCCCTTCTTAGACTTTACAGACCGCGGC
CTACTCGGATCCAAAAAAAAAAAAAAAAAAAAAAAAAAAAAAaacgttGACTAAAAAAAAAAAAAAAAAAA
AAAAAAAAAAAAAAAAAAAAAAAAAAAAAAAAAAAAAAAAAAAAAAAAAAAaaaagcGTCTTCGCGCGCA
TCATCGGATGCCGGGACCGACGAGTGCAGAGGCGTGCAAGCGAGCTTGGCGTAATCATGGTCATAGCT
GTTTCCTGTGTGAAATTGTTATCCGCTCACAATTCCACACAACATACGAGCCGGAAGCATAAAGTGTAAA
GCCTGGGGTGCCTAATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTTGTCA
GAAGTAAGTTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTT
CAATATTATTGAAGCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGACGTCTAAGAAACCATTATTATC
ATGACATTAACCTATAAAAATAGGCGTATCACGAGGCCCTTTCGTC

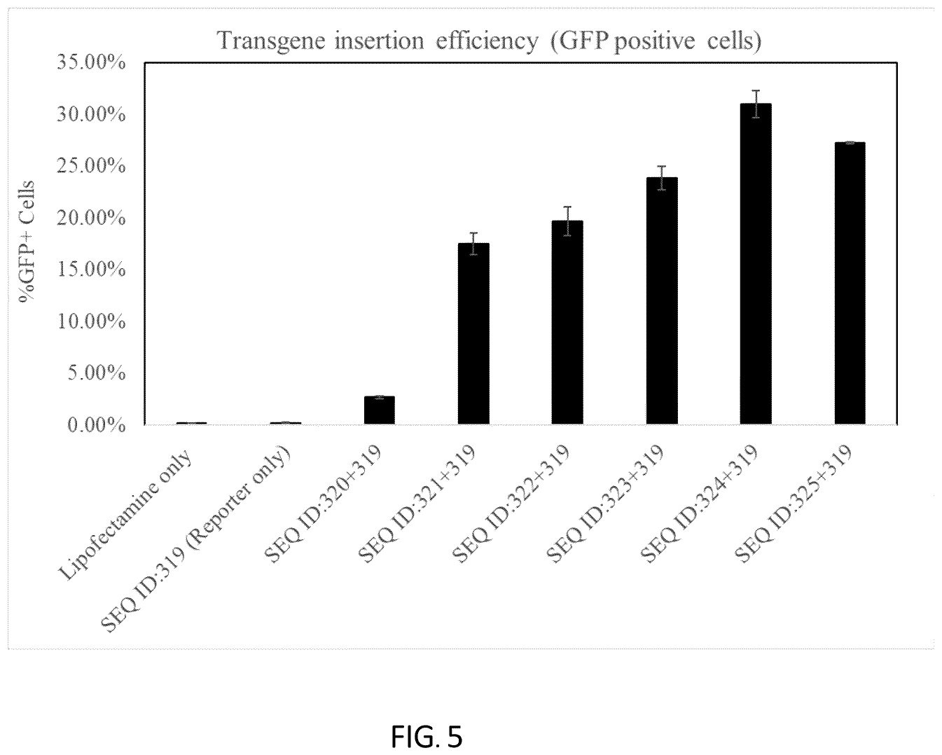

FIG. 5 shows results of integration assays using drivers with combinations of domain fusions and point mutations. In this experiment, different retrotransposable element constructs were used in the trans configuration (driver and GFP reporter encoded by different RNA). A common gene delivery construct encoding the GFP reporter (EX2107; SEQ ID NO: 319) was used for all driver constructs tested. Aside from the mutations and fusions listed, all constructs were identical in sequence to SM002. IVT of different RTE constructs was carried out as described above. U20S cells were used in 24-well plate, at 120K cells/well. 1000 ng RNA was transfected with 1.2 uL Lipofectamine. Integration was assessed based on the percentage of GFP positive cells (% GFP positive cells) after 24 h, with a higher percentage of GFP positive cells being indicative of higher levels of integration. % GFP positive cells was assessed by FACS following 24 hours after transfection with RNA.

It was observed that by combining multiple different modifications targeting different retrotransposable element functions, significant improvements in integration is possible. Combining UL12 (hypothesized to interact with cellular DNA repair), with HMG domains (hypothesized to improve chromatin binding and/or accessibility), with N647K mutation (hypothesized to improve reverse transcriptase domain activity/stability) (SEQ ID:321) lead to approximately 7-fold improvement in integration activity. Further adding I343K mutation (hypothesized to improve RNA binding) (SEQ ID:322) to this construct improved integration activity slightly by almost another fold. Further adding D64K mutation (hypothesized to improve endonuclease binding to DNA) to this construct (SEQ ID:323) improved activity by another fold (EX2196). Further adding L825G mutation (hypothesized to improve reverse transcriptase stability/activity) to this construct (SEQ ID:324) improved activity by almost another 3-fold. Adding M750L (hypothesized to improve reverse transcriptase stability/activity) instead of L825G (SEQ ID:325) also improved activity but only by ˜1-fold.

Altogether, approximately 12-fold improvement was demonstrated in integration activity of a non-LTR retrotransposable element through combination of domain fusions and mutations.

### Example 7

In order to test the effect of domain additions and mutations on improving integration efficiency of another LINE element, known as Vingi. Vingi-1 was taken from the genome of Anolis carolinensis. The driver and GFP reporter were configured in trans, transcribed as mRNA from plasmid DNA.

An exemplary Vingi-1 driver comprising Wild-type Vingi-1_Acar retrotransposon is encoded by EX2985 plasmid (SEQ ID NO: 326), with mRNA cassette containing Clean Cap-compatible T7 RNA promoter (TriLink Biotechnologies, bold) and A30N10A70 polyA tail (underlined). Vingi-1_Acar protein ORF is underlined and in italics. In between the T7 RNA promoter and ORF is the 5′UTR of Vingi-1 retrotransposon.

### EX2985:

(SEQ ID NO: 326)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAGTTTAAACTAATACGACTCACTATAAGGGGGGGACACGGAAAGAGCCTCCCCGAAGATTGAGT
gAATTCAGTCGGGCGTCCCCTGGGCAACGTTTCTTGTAAGCGGCCGATCTTTCCAcCCCAAAAGCATTGG
ATGaATGGATGAATACCAAAGGTCTTTATCAAGACCATTGCTAACGATTATGTCTATTAATATAGAAGGTT
TGTCACTTGCTAAGGAAGAACTATTAGCCAAAATGTCTGAGGACATcTCgTGTGACATCCTATGTATACAG
GAAACACACAGAGACATCACAATGAGgAGACCAAAAATTCTTGGAATGCAaCTGGCAGTGGAACGACCTC
ACAGaCAATATGGCAGTGCCATTTTTGTACGATCTGGTGTAGCAATCTCTGCAAcTTCCCTCACAGAAGtG
AACAACATTGAAATCTTATCTGTGGAACTTGATaGTTGCACCGTATCATCACTCTATAAACCACCTGGGGCT
GATTTCTATTTTACaCCCCCAACCAgTTGCCACAATCATGAAGCCCATTTTGTTGTGGGAGATTTCAATAGC
CACAGCTGTGTCTGGGGCTATGACGAAGATGATAGAAATGGCgAAGCAGTTCTAACGTGGGCcGACAAT
AGTAGAATGAGCCTCCTTCATGACAGTAAATTACCACCATCATTTAATAGCGGCCGATGGAAGCGTGGTT
ATAACCCTGATCTGATTTTTGTAAAGGAAAGCATAAGCCACCAATGCACCAAAAGGGTATTAAACCCaATA
CCTAACACACAACACAGACCAATATGCTGCGTAGCATATGCAGCTGTAAGACCgAAAAGTGTCCCATTCCG
CAGAAGATttaacttcaATAAAGCTAACTGGACAAAGTTTACAGAGACCttGGaAGCTGCTATTTCTGATATA
GAACCTTCTATAGAAAATTATGACCTGTTcGTAGAAGCTGTGAAAAGATCCTCAAGGCTCTCAATCCCTAG
AGGCTGTCGCACAAGCTaCCTACCAGGCCTAAACGAAGAATCaCTAAATCAGCTACAaGAATATCTCAGAt
TATTTCAAGAGAAcCCATACAGTGATGGGACTATAGCAGCAGGCCAAAAACTATCTACAGCCTTAGCTAAT
GCTAAGAAAGACCGTTGGATAGAGCTGCTTGAGAACCTgGACATGTCCAAGAGTAGCCGAAAAGCCTGG
CAAtTGCTGAGAcGCCTGGATAGTGACCCTCTGGTCAACCcTGGACACGCgAACGTGACACCAGAtCAGAT
AGCTCACCAGCTAATTCAGAATGGGAAAACCAACTGCAGCAGAATAAAGATGAAAATCAACAGGGTGCC
AGAACTTGAAACCCACCAGTTGTCcTCTCCTCTAAACCTGAAAGAACTCAGAGAAGCCATCAAGCGATGTA
AGACTGGTAAAGCACCTGGCCTAGATGACCTGATGATGGAGCAAATCAAACACTTGGGgcCCAAAGCTGA
AAACTGGCTTTTGAAATTCTACAATCAATGCcTGGCACACAAACAGATTCCCAGAGCATGGAGGAAAACT
AAGATaATTGCCATcTTAAAACCTGGTAAAGATGCCTCCAATGCCAGGAACTAtCGACCAATCTCCCTCTTA
TGTCATCTATATAAAGTCTATGAGAGGATGCTATTAAATCGACTAGGACCTGTTATCGAACCCAAGCTTAT
TGCACAACAAGCAGGTTTCAGACCAGGGAAAAACTGTACAGGTCAAATTCTTCATCTGACTGAACATATC
GAGGAAGGCTAtGAGAAAGGCTGCATtACGGGAACAGTcTTTGTGGACCTTACGGCAGCCTATGACACGG
TGCAACATAGAAAAATGCTGCATAAAGTCTACCATATCACCCGGGACTTTGACTTTACAAAAACTGTCCAG
ACCCTCtTAGAAAACCgCAGCTTCTATGTGGAGTTTCAGGGCCAGAAAAGCAGATGGAGGAGGCAAAAG
AATGGTTTACCCCAAGGCAGCGTTCTTGCACCaACCTTATTTAAcATCTTCACGAACGATCAGCCACAACCA
CCACTCACAAAGAGCTTTATATATGCTGATGACCTTGGCCTTACAACACAAGCAAAAGATTTTGAAACAGT
TGAAAAGCAACTCACCAATGCCTTGAAAGAtCTCTCCAGCTACTACAAAGAGAACCACCTGAAGCCtAACC
CTGCCAAGACACAAGTGTGTGCTTTCCACCTACGTAACCGCGAAGCCAACAGGaAACTGAAAGTTACTTG
GGAAGGCCAAGAGCTCGAACACTGTTTCCATCCTAAATACCTTGGTGTCACCTTAGACCGAACACTAACAT
ATAGGAAACACTGCATGAACACCAAGCACAAAGTAGCTGCACGCAATAACATCCTGCGGAAACTGACTG
GCAGCGCATGGGGaGCAGACCCACAAGTAATAAGAACATCAGCCCTGGCCTTGTCTTTCTCAACTGCAGA
GTATGCCTGTCCTGTTTGGCACAAGTCTGCCCATGCaAAGCAGGTGGACATAGCACTGAATGAAACATGC
AGAATcATCACgGGATGCCTTAAACCTACACCTGTTGATAAACTCTACAAGTTAGCTGGCATTGCCCCTCCT
GACGTGCGACGGGAAGTTGCTGCTAACgGTGAGAGAAAaAAGGTcGAACATTGTGAAAGCCACCCACTG
CATGgCTATCAcCCTCCTCCCACCAGACTCAAATCAAGGAAGGGCTTCATGAGAACCACCACTCCTCTTGAT
GTTCCTCCAGCAgCAGCAAGGGTGTCcCTCTGGGCAGCTAAACCTGGCAATTCTAACTGGATGGCCCCCC
AaGAGGGaCTTCCTCCAGGGGCAAACCAaGAATGGGCAACTTGGAAGTCCCTGAACAGACTCAGAAGtG
GAGTGGGCAGATCAAAAGACAACTTGGCAAGGTGGCACTACCTGGAGGAATCCTCCACCTTGTGTGACT
GTGGAGCtGAACAAACAACTCaGCATATGTATGCTTGCCCACAATGcCCTGCCTCATGtACGGAGGAGGA
GTTGTTTAAAGCTACaGACAATGCGGTTGCTGTTGCCCGCTTTTGGTCCAAAACTATTTAGGTCGACgctag
cACCGGTAAAAAAAAAAAAAAAAAAAAAAAAAAAAAAaacgttGACTAAAAAAAAAAAAAAAAAAAAAAA
AAAAAAAAAAAAAAAAAAAAAAAAAAAAAAAAAAAAAAAAAAAAAAAaaaaaaGTCTTCGCGCGCATCAT
CGGATGCCGGGACCGACGAGTGCAGAGGCGTGCAAGCGAGCTTGGCGTAATCATGGTCATAGCTGTTT
CCTGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGAAA
CCTGTCGTGCCAGCTGCATTAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTGATCCGGCA
AACAAACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAAGGGAT
TTTGGTCATGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGATACGGGAGGGCTT
ACCATCTGGCCCCAGTGCTGCAATGATACCGCGGGACCCACGCTCACCGGCTCCAGATTTATCAGCAATA
AACCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAAAAGTGCTCATCATTGG
AAAACGTTCTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAACAGGAAGGC
AAAATGCCGCAAAAAAGGGAATAAGGGCGACACGGAAATGTTGAATACTCATACTCTTCCTTTTTCAATA
TTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCATTATTATCATGAC
ATTAACCTATAAAAATAGGCGTATCACGAGGCCCTTTCGTC

An exemplary wild-type Vingi-1 driver encoded by EX2985 plasmid (SEQ ID NO: 326) has the following amino acid sequence (SEQ ID NO: 327):

(SEQ ID NO: 327)
MDEYQRSLSRPLLTIMSINIEGLSLAKEELLAKMSEDISCDILCIQET
HRDITMRRPKILGMQLAVERPHRQYGSAIFVRSGVAISATSLTEVNNI
EILSVELDSCTVSSLYKPPGADFYFTPPTSCHNHEAHFVVGDFNSHSC
VWGYDEDDRNGEAVLTWADNSRMSLLHDSKLPPSFNSGRWKRGYNPDL
IFVKESISHQCTKRVLNPIPNTQHRPICCVAYAAVRPKSVPFRRRFNF
NKANWTKFTETLEAAISDIEPSIENYDLFVEAVKRSSRLSIPRGCRTS
YLPGLNEESLNQLQEYLRLFQENPYSDGTIAAGQKLSTALANAKKDRW
IELLENLDMSKSSRKAWQLLRRLDSDPLVNPGHANVTPDQIAHQLIQN
GKTNCSRIKMKINRVPELETHQLSSPLNLKELREAIKRCKTGKAPGLD
DLMMEQIKHLGPKAENWLLKFYNQCLAHKQIPRAWRKTKIIAILKPGK
DASNARNYRPISLLCHLYKVYERMLLNRLGPVIEPKLIAQQAGFRPGK
NCTGQILHLTEHIEEGYEKGCITGTVFVDLTAAYDTVQHRKMLHKVYH
ITRDFDFTKTVQTLLENRSFYVEFQGQKSRWRRQKNGLPQGSVLAPTL
FNIFTNDQPQPPLTKSFIYADDLGLTTQAKDFETVEKQLTNALKDLSS
YYKENHLKPNPAKTQVCAFHLRNREANRKLKVTWEGQELEHCFHPKYL
GVTLDRTLTYRKHCMNTKHKVAARNNILRKLTGSAWGADPQVIRTSAL
ALSFSTAEYACPVWHKSAHAKQVDIALNETCRIITGCLKPTPVDKLYK
LAGIAPPDVRREVAANGERKKVEHCESHPLHGYHPPPTRLKSRKGFMR
TTTPLDVPPAAARVSLWAAKPGNSNWMAPQEGLPPGANQEWATWKSLN
RLRSGVGRSKDNLARWHYLEESSTLCDCGAEQTTQHMYACPQCPASCT
EEELFKATDNAVAVARFWSKTI

An exemplary Vingi-1 GFP reporter is encoded by gene delivery construct EX2988 (SEQ ID NO: 328) for use with a Vingi-1 driver, is shown below. The gene delivery construct comprises mRNA cassette containing Clean Cap-compatible T7 RNA promoter (TriLink Biotechnologies, bold) and A30N10A70 polyA tail (underlined). GFP cassette including MNDopt promoter, GFP ORF, and synthetic polyadenylation signal is in anti-sense and in italics. In between the T7 promoter and GFP cassette is the 5′UTR from Vingi-1 element, and in between the GFP cassette and polyA tail is the 3′UTR from Vingi-1 element.

(SEQ ID NO: 328)
TCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGTGTGAAATACCGCAC
AGATGCGTAAGGAGAAAATACCGCATCAGGCGCCATTCGCCATTCAGGCTGCGCAACTGTTGGGAAGGG
CGATCGGTGCGGGCCTCTTCGCTATTACGCCAGCTGGCGAAAGGGGGATGTGCTGCAAGGCGATTAAGT
TGGGTAACGCCAGGGTTTTCCCAGTCACGACGTTGTAAAACGACGGCCAGTGAATTGGAGATCGGTACT
TCGCGAGTTTAAACTAATACGACTCACTATAAGGGGGGGACACGGAAAGAGCCTCCCCGAAGATTGAGT
gAATTCAGTCGGGCGTCCCCTGGGCAACGTTTCTTGTAAGCGGCCGATCTTTCCAcCCCAAAAGCATTGG
ATGaGTCGACGCGGCCTACTCGACGGATCGATCCGAACAAACGACCCAACACCCGTGCGTTTTATTCTGTC
TTTTTATTGCCGATCCCCTCAGAAGAACTCGTCAAGAAGGCGATAGAAGGCGATGCGCTGCGAATCGGG
AGCGGCGATACCGTAAAGCACGAGGAAGCGGTCAGCCCATTCGCCGCCAAGCTCTTCAGCAATATCACG
GGTAGCCAACGCTATGTCCTGATAGCGGTCGGCCGCTTTACTTGTACAGCTCGTCCATGCCGAGAGTGAT
CCCGGCGGCGGTCACGAACTCCAGCAGGACCATGTGATCGCGCTTCTCGTTGGGGTCTTTGCTCAGGGC
GGACTGGGTGCTCAGGTAGTGGTTGTCGGGCAGCAGCACGGGGCCGTCGCCGATGGGGGTGTTCTGCT
GGTAGTGGTCGGCCAGCTGCACGCTGCCGTCCTCGATGTTGTGGCGGATCTTGAAGTTCACCTTGATGCC
GTTCTTCTGCTTGTCGGCCATGATATAGACGTTGTGGCTGTTGTAGTTGTACTCCAGCTTGTGCCCCAGGA
TGTTGCCGTCCTCCTTGAAGTCGATGCCCTTCAGCTCGATGCGGTTCACCAGGGTGTCGCCCTCGAACTTC
ACCTCGGCGCGGGTCTTGTAGTTGCCGTCGTCCTTGAAGAAGATGGTGCGCTCCTGGACGTAGCCTTCGG
GCATGGCGGACTTGAAGAAGTCGTGCTGCTTCATGTGGTCGGGGTAGCGGCTGAAGCACTGCACGCCGT
AGGTCAGGGTGGTCACGAGGGTGGGCCAGGGCACGGGCAGCTTGCCGGTGGTGCAGATGAACTTCAGG
GTCAGCTTGCCGTAGGTGGCATCGCCCTCGCCCTCGCCGGACACGCTGAACTTGTGGCCGTTTACGTCGC
CGTCCAGCTCGACCAGGATGGGCACCACCCCGGTGAACAGCTCCTCGCCCTTGCTCACCATGGTGGCtcga
gaactagatcgcgccgagtgagggttgtgggctcttttattgagctcggggagcagaagcgcgcgaacagaagcgagaagcgaa
ctgattggttagttcaaataaggcacagggtcatttcaggtccttggggcaccctggaaacatctgatggttctctagaaactgctga
gggcgggaccgcatctggggaccatctgttcttggccctgagccggggcaggaactgcttaccacagatatcctgtttggcccatatt
ctgctgttccaactgttcttggccctgagccggggcaggaactgcttaccacagatatcctgtttggcccatattctgctgtctctctgttc
ctaaccttgatcctagcttgccaaacctacaggtggggtctttcattcccccctttttctggagactaaataaaatcttttattctatctat
ggctcgtactctataggcttcagctggtgatattgttgagtcaaaactagagcctggaccactgatatcctgtctttaacaaattggac
taatcgaattcgaagcttTTGCTTGTGATTTCTTTTCTTTTtTaTTTTATTTCCATTATTTGAAATGTATTTGcTGTAc
CAATGCTTTTGACACGAAATAAATAAAgctagcACCGGTAAAAAAAAAAAAAAAAAAAAAAAAAAAAAAaa
cgttGACTAAAAAAAAAAAAAAAAAAAAAAAAAAAAAAAAAAAAAAAAAAAAAAAAAAAAAAAAAAAAAA
AAAAAAAAaaaaaaGTCTTCGCGCGCATCATCGGATGCCGGGACCGACGAGTGCAGAGGCGTGCAAGCG
AGCTTGGCGTAATCATGGTCATAGCTGTTTCCTGTGTGAAATTGTTATCCGCTCACAATTCCACACAACAT
ACGAGCCGGAAGCATAAAGTGTAAAGCCTGGGGTGCCTAATGAGTGAGCTAACTCACATTAATTGCGTT
GCGCTCACTGCCCGCTTTCCAGTCGGGAAACCTGTCGTGCCAGCTGCATTAATGAATCGGCCAACGCGCG
GGGAGAGGCGGTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGCCGCGTTGCTGGCG
TTTTTCCATAGGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCTTGAAGTGGTG
GCCTAACTACGGCTACACTAGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCGCGGGACCCACG
CTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTCATGGTTATGGCAGCACTGCA
TAATTCTCTTACTGTCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATACCGCGCCACATAG
CAGAACTTTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCATGAGCGGATACAT
ATTTGAATGTATTTAGAAAAATAAACAAATAGGGGTTCCGCGCACATTTCCCCGAAAAGTGCCACCTGAC
GTCTAAGAAACCATTATTATCATGACATTAACCTATAAAAATAGGCGTATCACGAGGCCCTTTCGTC

Nucleic acids were designed and produced to encode non-limiting examples of engineered proteins comprising a Vingi-1 driver protein (SEQ ID:327) with one or more-point mutations and/or domain fusions. Mutations were made in the endonuclease domain (residues 40-234), RNA binding domain (residues 235-340), and reverse transcriptase domain (residues 341-982) and polypeptide fusions were made at the N- and C-terminus.

In some examples, the following HMGN1 polypeptide was incorporated (e.g., as an N-terminal fusion):

(SEQ ID NO: 23)
MPKRKVSSAEGAAKEEPKRRSARLSAKPPAKVEAKPKKAAAKDKSSD
KKVQTKGKRGAKGKQAEVANQETKEDLPAENGETKTEESPASDEAGE
KEAKSD

In some examples, the following HMGB1 polypeptide was incorporated (e.g. as a C-terminal fusion):

(SEQ ID NO: 24)
GKGDPKKPRGKMSSYAFFVQTCREEHKKKHPDASVNFSEFSKKCSER
WKTMSAKEKGKFEDMAKADKARYEREMKTYIPPKGE

In some examples, the following UL12 polypeptide was incorporated (e.g., as a C-terminal fusion):

(SEQ ID NO: 25)
ESTVGPACPPGRTVTKRPWALAEDTPRGPDSPPKRPRPNSLPLTTTF
RPLPPPPQTTSAVDPSSHSPVNPPRDQHATDTADEKPRAASPALSDA
SGPPTPDIPLSPGGTHARDPDADPDSPDLDS

In some examples, the following Sto7d polypeptide was incorporated (e.g., as a C-terminal fusion):

(SEQ ID NO: 26)
VTVKFKYKGEEKEVDISKIKKVWRVGKMISFTYDDNGKTGRGAVSEK
DAPKELLQMLEK

In some examples, the following Sso7d polypeptide was incorporated (e.g., as a C-terminal fusion):

(SEQ ID NO: 377)
VTVKFKYKGEEKEVDISKIKKVWRVGKMISFTYDDNGKTGRGAVSEK
DAPKELLQMLEK

In some examples, the following GP45 protein from T4 phage was incorporated (e.g. as a C-terminal fusion):

(SEQ ID NO: 329)
KLSKDTTALLKNFATINSGIMLKSGQFIMTRAVNGTTYAEANISDVI
DFDVAIYDLNGFLGILSLVNDDAEISQSEDGNIKIADARSTIFWPAA
DPSTVVAPNKPIPFPVASAVTEIKAEDLQQLLRVSRGLQIDTIAITV
KEGKIVINGFNKVEDSALTRVKYSLTLGDYDGENTFNFIINMANMKM
QPGNYKLLLWAKGKQGAAKFEGEHANYVVALEADSTHDF

In some examples, peptides (SEQ ID NOs: 379, 380) derived from HIV Viral Infectivity Factor (VIF) (SEQ ID NO: 378) were incorporated.

In some examples, the following RecT polypeptide from Pseudomonas aeruginosa (paRecT, SEQ ID NO: 381) were incorporated:

MGTALTPLLTKFATRYEMGTTPEEVANTLKQTCFKGQVNDSQMVALLIVA
DQYKLNPFTKELYAFPDKNNGIVPVVGVDGWARIINENPQFDGMEFSMDQ
QGTECTCKIYRKDRSHAISATEYMAECKRNTQPWQSHPRRMLRHKAMIQC
ARLAFGFAGIYDQDEAERIVERDVTPAEQYEDVSEAICLIKDSPTMEDLQ
AAFSNAWKAYKTKGARDQLTAAKDQRKKELLDAPIDVEFEETGDDRAA

In some examples, the following i53 peptide was incorporated (SEQ ID NO: 382):

AASLNGAPLIKDPMLIFVKTLTGKTITLEVEPSDTIENVKAKIQDKEGIP
PDQQRLAFAGKSLEDGRTLSDYNILKDSKLHPLLRLR

In some examples, the following NLS peptide from PARP1 was incorporated (SEQ ID NO: 384): KKKSKK

In some examples, the following NLS peptide from TOPB1 was incorporated (SEQ ID NO: 383): PSQQKRK

In some examples, the following PolD3 polypeptide was incorporated (SEQ ID NO: 385):

ADQLYLENIDEFVTDQNKIVTYKWLSYTLGVHVNQAKQMLYDYVERKRKE
NSGAQLHVTYLVSGSLIQNGHSCHKVAVVREDKLEAVKSKLAVTASIHVY
SIQKAMLKDSGPLFNTDYDILKSNLQNCSKFSAIQCAAAVPRAPAESSSS
SKKFEQSHLHMSSETQANNELTTNGHGPPASKQVSQQPKGIMGMFASKAA
AKTQETNKETKTEAKEVTNASAAGNKAPGKGNMMSNFFGKAAMNKFKVNL
DSEQAVKEEKIVEQPTVSVTEPKLATPAGLKKSSKKAEPVKVLQKEKKRG
KRVALSDDETKETENMRKKRRRIKLPESDSSEDEVFPDSPGAYEAESPSP
PPPPSPPLEPVPKTEPEPPSVKSSSGENKRKRKRVLKSKTYLDGEGCIVT
EKVYESESCTDSEEELNMKTSSVHRPPAMTVKKEPREERKGPKKGTAALG
KANRQVSITGFFQRK

In some examples, the following RAD17 polypedite was incorporated (SEQ ID NO: 386):

LVEPEEVVEMSHMPGDLFNLYLHQNYIDFFMEIDDIVRASEFLSFADILS
GDWNTRSLLREYSTSIATRGVMHSNKARGYAHCQGGGSSFRPLHKPQWFL
INKKYRENCLAAKALFPDFCLPALCLQTQLLPYLALLTIPMRNQAQISFI
QDIGRLPLKRHFGRLKMEALTDREHGMIDPDSGDEAQLNGGHSAEESLGE
PTQATVPETWSLPLSQNSASELPASQPQPFSAQGDMEENIIIEDYESDGT

In some examples, the following SCML1 polypeptide was incorporated (SEQ ID NO: 387):

WSVEAVVLFLKQTDPLALCPLVDLFRSHEIDGKALLLLTSDVLLKHLGVK
LGTAVKLCYYIDRLKQGK

In some examples, the following MDC1-derived polypeptide was incorporated (SEQ ID NO: 388):

EDTQAIDWDVEEEEETEQSSESLRCNVEPVGRLHIFSGAHGPEKDFPLHL
GKNVVGRMPDCSVALPFPSISKQHAEIEILAWDKAPILRDCGSLNGTQIL
RPPKVLSPGVSHRLRDQELILFADLLCQYHRLDVSLPFVSRGPLTVEETP
RVQGETQPQRLLLAEDSEEEVDFLSERRMVKKSRTTSSSVIVPESDEEGH
SPVLGGLGPPFAFNLNSDT

In some examples, the following CDKN2a polypeptide was incorporated (SEQ ID NO: 389):

MVRRFLVTLRIRRACGPPRVRVFVVHIPRLTGEWAAPGAPAAVALVLMLL
RSQRLGQQPLPRRP

In some examples, the following MDM2 NLS was incorporated (SEQ ID NO: 390):

RQRKRHK

In some examples, the following PCNA interaction motif from CHAF1A was incorporated (SEQ ID NO: 391):

MLEELECGAPGARGAATAMDCKDRPAFPVKKLIQARLPFKRLNLVPKGK

In some examples, the following MSH4 polypeptide was incorporated (SEQ ID NO: 392):

MLRPEISSTSPSAPAVSPSSGETRSPQGPRYNFGLQETPQSRPSVQVVSA
STCPGTSGAAGDRSSSSSSLPCPAPNSRPAQGS

In some examples, the following WPRE 3′UTR was incorporated (SEQ ID NO: 393):

AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCcTCTTCGCCTTCGCC
CTCAGACGAGTCGGATCTCCCTTTGGGCCGCCTCCCCGC

In some examples, the following FEN1 PCNA interaction motif was incorporated (SEQ ID NO: 394):

STQGRLDDFFKVTGSL

In some examples, the following P21 PCNA interaction motif was incorporated (SEQ ID NO: 395):

RKRRQTSMTDFYHSKRRLIFSKRKP

In some examples, the following ANKRD28-derived polypeptide was incorporated (SEQ ID NO: 396):

EKRTPLHAAAYLGDAEIIELLILSGARVNA

Constructs were evaluated using an experimental transposition assay described in Example 3. Non-limiting examples of the constructs tested include:

Single mutations or their combinations, made in a retroelement-derived polypeptide derived from a wild-type Vingi-1_Acar retrotransposon (EX2985):
H929G (SEQ ID: 70), Q634L (SEQ ID: 71), A684S (SEQ ID: 72), F977Y (SEQ ID: 73), H850Q (SEQ ID: 74), F238Y (SEQ ID: 75), A875T (SEQ ID: 76), 145L (SEQ ID: 77), L434I (SEQ ID: 78), I439L (SEQ ID: 79), T470A (SEQ ID: 80), Y673W (SEQ ID: 81), Y950M (SEQ ID: 82), A901H (SEQ ID: 83), G833I (SEQ ID: 84), G833S (SEQ ID: 85), R350K (SEQ ID: 86), S35C (SEQ ID: 87), L111V (SEQ ID: 88), M16I (SEQ ID: 89), A87S (SEQ ID: 90), N311D (SEQ ID: 91), 152S (SEQ ID: 92), Y313F (SEQ ID: 93), 152P (SEQ ID: 94), S109T (SEQ ID: 95), Q215D (SEQ ID: 96), R468K (SEQ ID: 97), C495S (SEQ ID: 98), N529S (SEQ ID: 99), L476R (SEQ ID: 100), I473R (SEQ ID: 101), L493R (SEQ ID: 102), W353R (SEQ ID: 103), M345K (SEQ ID: 104), I475R (SEQ ID: 105), L25Q (SEQ ID: 106), S39K (SEQ ID: 107), I52E (SEQ ID: 108), Q63T (SEQ ID: 109), S89Q (SEQ ID: 110), G116N (SEQ ID: 111), A132T (SEQ ID: 112), V145S (SEQ ID: 113), K196W (SEQ ID: 114), N299K (SEQ ID: 115), Q302K (SEQ ID: 116), A329S (SEQ ID: 117), E933R (SEQ ID: 118), K703R (SEQ ID: 119), K480Q (SEQ ID: 120), K675R (SEQ ID: 121), K789R (SEQ ID: 122), H787R (SEQ ID: 123), 1793R (SEQ ID: 124), P808K (SEQ ID: 125), D792K (SEQ ID: 126), 1793K (SEQ ID: 127), E797R (SEQ ID: 128), D792M (SEQ ID: 129), P808R (SEQ ID: 130), M735R (SEQ ID: 131), A742K (SEQ ID: 132), L693K (SEQ ID: 133), N745K (SEQ ID: 134), Q354K (SEQ ID: 135), R357K (SEQ ID: 136), D362R (SEQ ID: 137), N412E (SEQ ID: 138), K424R (SEQ ID: 139), M435Y (SEQ ID: 140), E447Q (SEQ ID: 141), R486K (SEQ ID: 142), P511S (SEQ ID: 143), P515E (SEQ ID: 144), R568K (SEQ ID: 145), H576K (SEQ ID: 146), S595R (SEQ ID: 147), E676R (SEQ ID: 148), A684E (SEQ ID: 149), A874E (SEQ ID: 150), M570L (SEQ ID: 151), V574L (SEQ ID: 152), L590F (SEQ ID: 153), A621S (SEQ ID: 154), Y950I (SEQ ID: 155), M735E (SEQ ID: 156), G886P (SEQ ID: 157), Q300L (SEQ ID: 158), A519P (SEQ ID: 159), G833V (SEQ ID: 160), K784R (SEQ ID: 161), E514A (SEQ ID: 162), C938D (SEQ ID: 163), P515K (SEQ ID: 164), P780A (SEQ ID: 165), K807A (SEQ ID: 166), K414R (SEQ ID: 167), K966R (SEQ ID: 168), Y562F (SEQ ID: 169), A742M (SEQ ID: 170), H460R (SEQ ID: 171), E418R (SEQ ID: 172), D334E (SEQ ID: 173), D191A (SEQ ID: 174), R609K (SEQ ID: 175), K611T (SEQ ID: 176), N665K (SEQ ID: 177), N695R (SEQ ID: 178), R696H (SEQ ID: 179), A742T (SEQ ID: 180), K705R (SEQ ID: 181), A755K (SEQ ID: 182), A786E (SEQ ID: 183), K807R (SEQ ID: 184), P808T (SEQ ID: 185), C841D (SEQ ID: 186), E842P (SEQ ID: 187), T854Q (SEQ ID: 188), T867R (SEQ ID: 189), Q947E (SEQ ID: 190), A951V (SEQ ID: 191), Q954K (SEQ ID: 192), N330E (SEQ ID: 193), L60P (SEQ ID: 194), G833K (SEQ ID: 195), G861S (SEQ ID: 196), Y950V (SEQ ID: 197), G833L (SEQ ID: 198), A226T (SEQ ID: 199), K604R (SEQ ID: 200), S35A (SEQ ID: 201), C144T (SEQ ID: 202), G316E (SEQ ID: 203), N330K (SEQ ID: 204), D360A (SEQ ID: 205), M167L (SEQ ID: 206), T214S (SEQ ID: 207), Y950L (SEQ ID: 208), V838Q (SEQ ID: 209), D375N (SEQ ID: 210), H783A (SEQ ID: 211), P511K (SEQ ID: 212), P515S (SEQ ID: 213), C779A (SEQ ID: 214), P808W (SEQ ID: 215), S754Y (SEQ ID: 216), 1793N (SEQ ID: 217), P478W (SEQ ID: 218), I491R (SEQ ID: 219), H201N (SEQ ID: 220), A223V (SEQ ID: 221), Q309K (SEQ ID: 222), K333R (SEQ ID: 223), R465K (SEQ ID: 224), R489Q (SEQ ID: 225), H840T (SEQ ID: 226), S858R (SEQ ID: 227), A892H (SEQ ID: 228), E904H (SEQ ID: 229), F715E (SEQ ID: 230), K661R (SEQ ID: 231), K611Q (SEQ ID: 232), D792R (SEQ ID: 233), G426R (SEQ ID: 234), R606K (SEQ ID: 235), H739K (SEQ ID: 236), S771C (SEQ ID: 237), H171N (SEQ ID: 238), H929R (SEQ ID: 239), C127I (SEQ ID: 240), L637N (SEQ ID: 241), R731K (SEQ ID: 242), S754T (SEQ ID: 243), Q790K (SEQ ID: 244), R927K (SEQ ID: 245).
   Peptide/Domain fusions: N-terminal HMGN1 fusion (SEQ ID: 246), C-terminal HMGB1 (SEQ ID: 247), UL12 (SEQ ID: 249), PCNA interaction motif from FEN1 (SEQ ID: 250), PCNA interaction motif from P21 (SEQ ID: 251), Gp45 (SEQ ID: 252), Sso7D (SEQ ID: 253), Vif motifs (SEQ ID: 254), Sto7D (SEQ ID: 255), RAD51 (SEQ ID: 256), Dead Cas9 (SEQ ID: 259), I53 (SEQ ID: 263), MDC1 (SEQ ID: 264), BRCA2-derived peptide (SEQ ID: 265), PolD3 fusion (SEQ ID: 266), i53 (SEQ ID: 267), TOPBP1 NLS (SEQ ID: 268), PARP1 NLS (SEQ ID: 269), ANKRD28 (SEQ ID: 270), RAD17 (SEQ ID: 271), SCML1 (SEQ ID: 272), CDKN2a (SEQ ID: 273), PCNA interaction motif from CHAF1A (SEQ ID: 274), paRecT (SEQ ID: 275), MSH4 (SEQ ID: 276), Mdm2 NLS (SEQ ID: 277).
   Peptide deletions: RNASEH deletion (SEQ ID: 248), endonuclease domain deletion (SEQ ID: 257), Zinc finger domain deletion (SEQ ID: 285),
   Heterologous UTRs may also be added to the mRNA: WPRE3 3′ UTR (SEQ ID: 260), hag 3′ UTR (SEQ ID: 261), human alpha globin 5′ UTR (SEQ ID: 262).

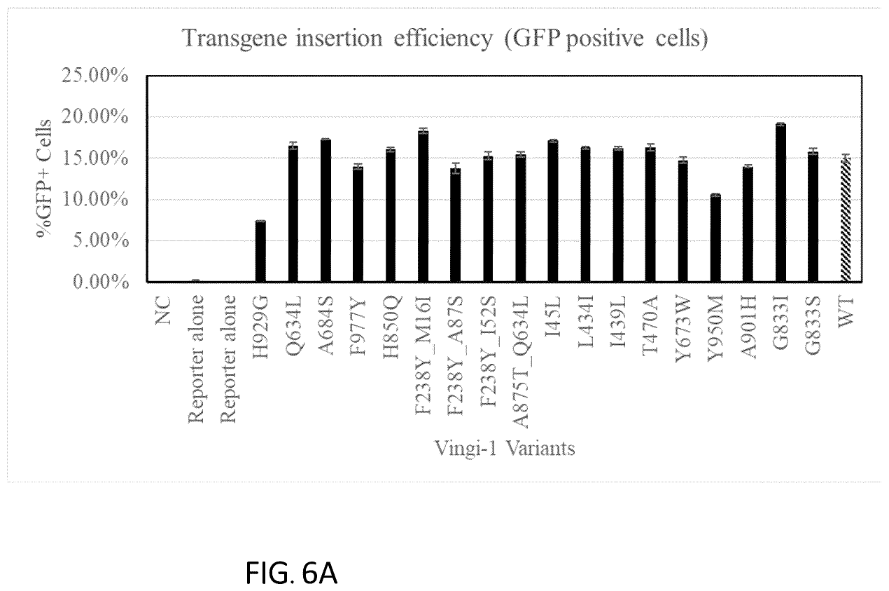

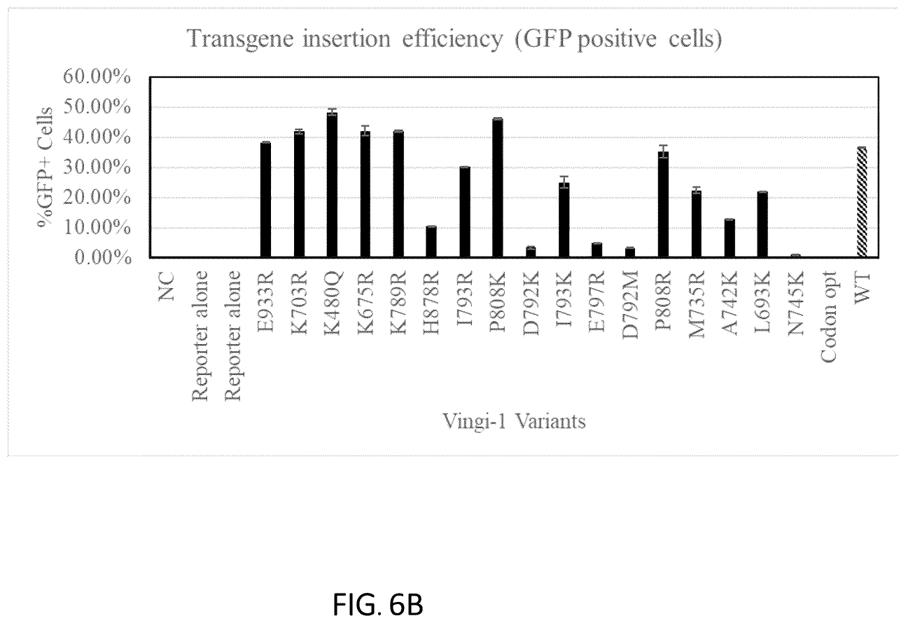

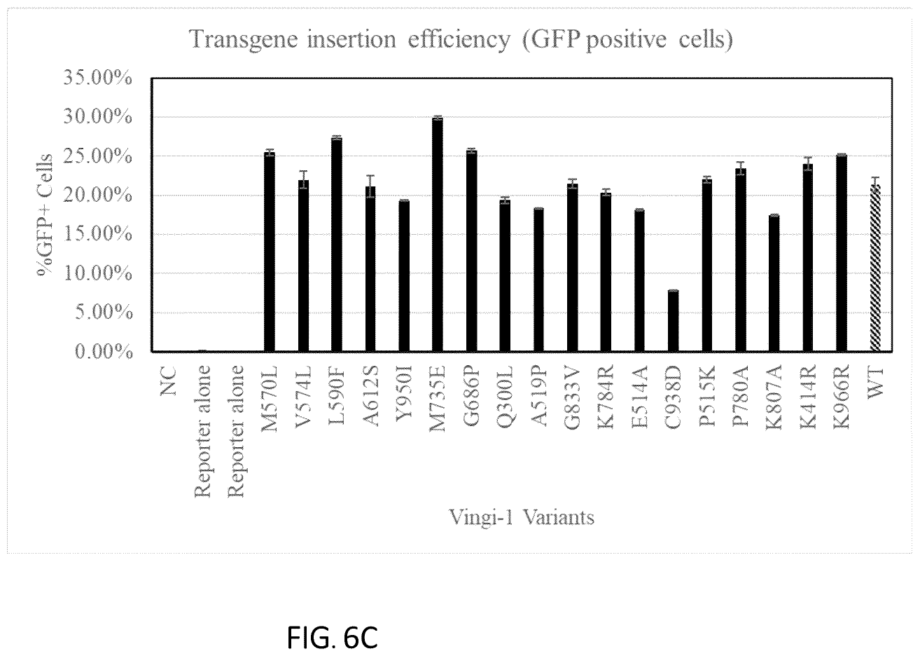

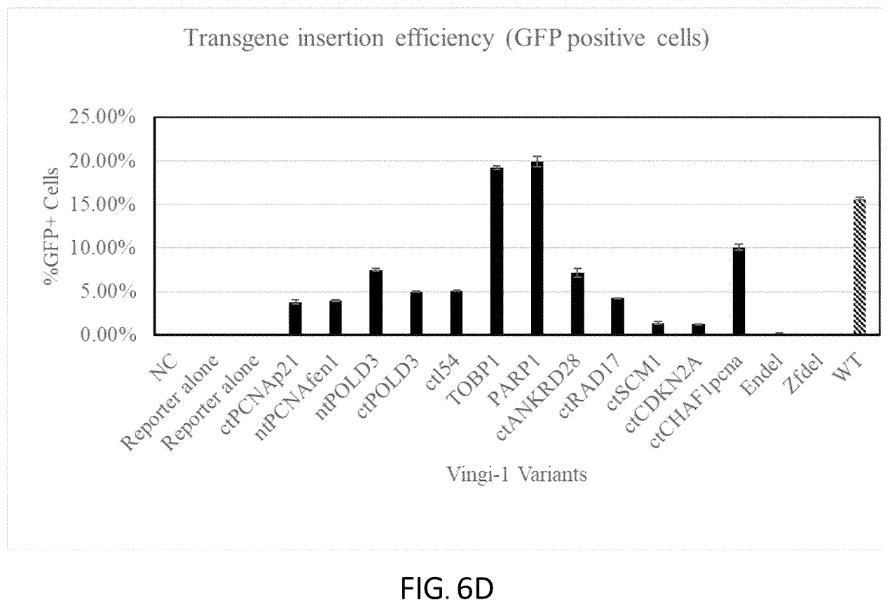

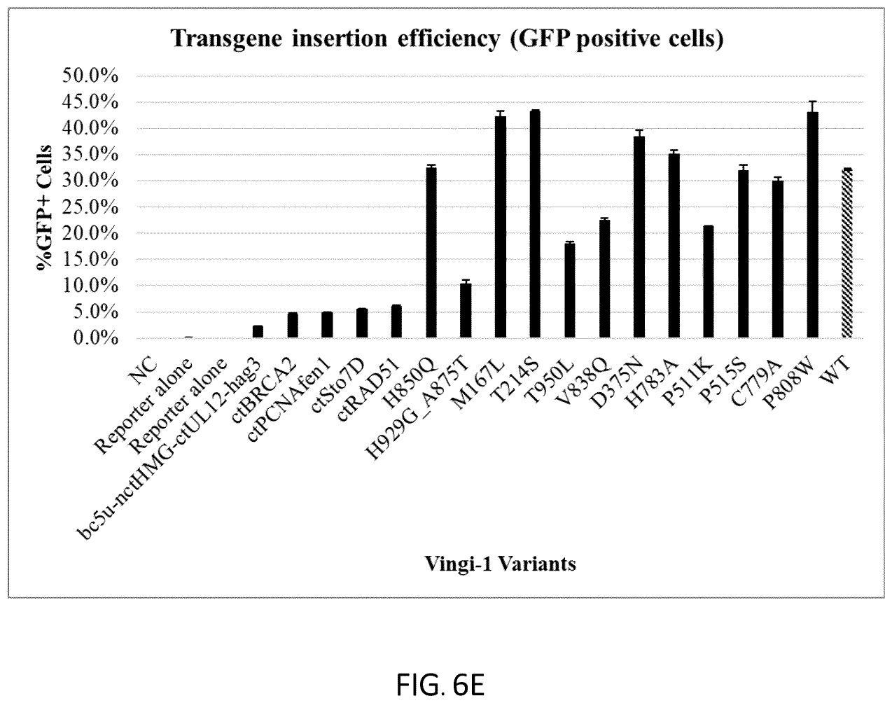

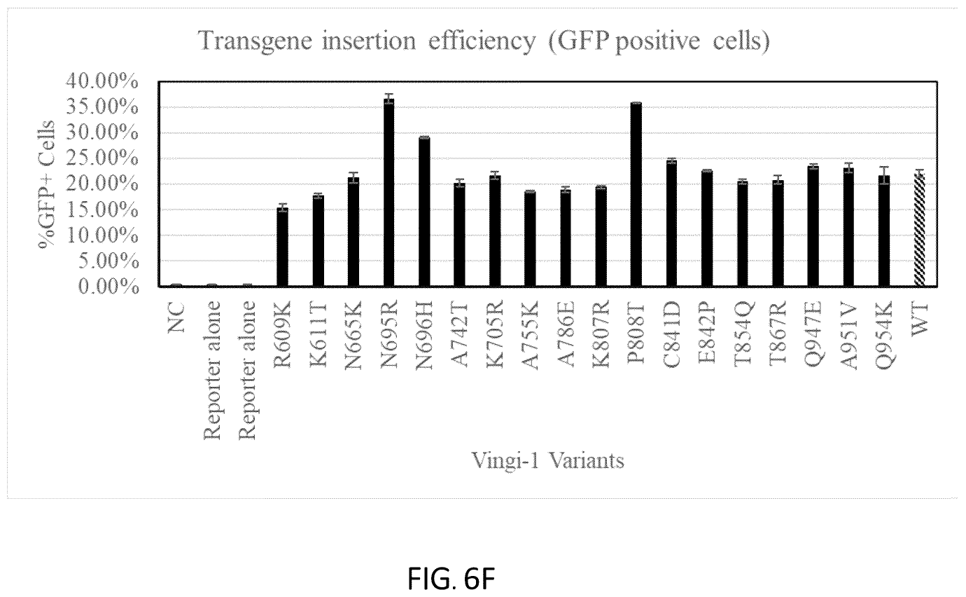

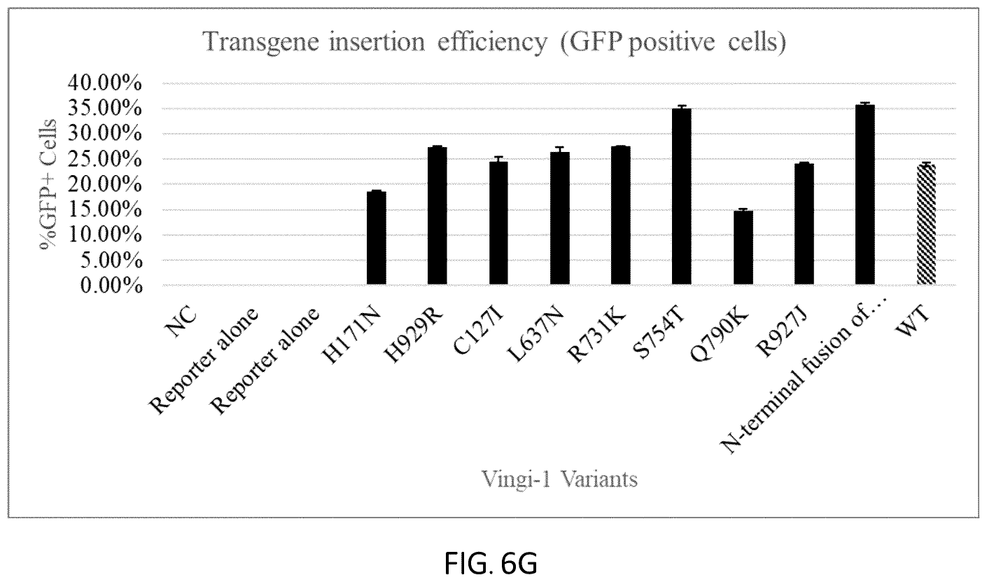

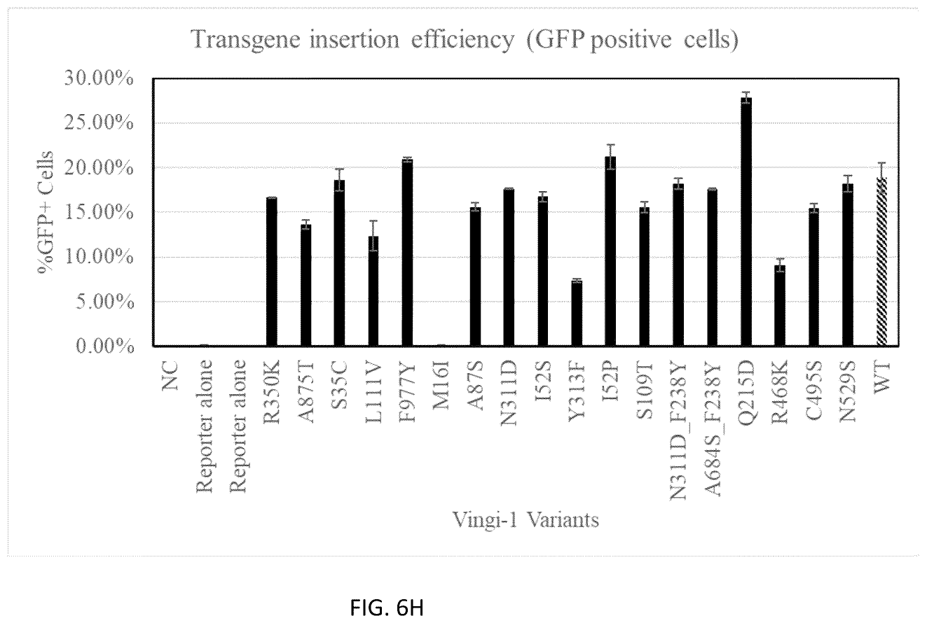

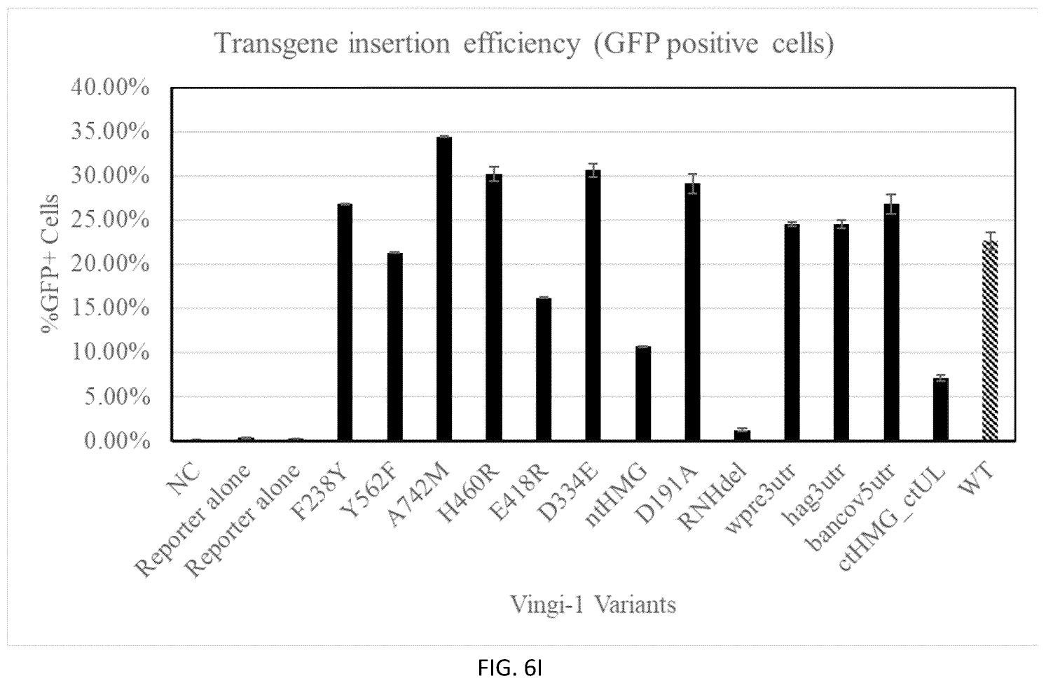

FIGS. 6A-6I shows integration assays results using Vingi-1 drivers with combinations of domain fusions and point mutations. In these experiment, different retrotransposable element (driver) constructs were used in the trans configuration (driver and GFP reporter encoded by different RNA). A common gene delivery construct encoding the GFP reporter (EX2988; SEQ ID NO: 328) was used for all driver constructs tested. Aside from the mutations and fusions listed, all constructs were identical in sequence to EX2985 (WT, marked with pattern). IVT of different RTE constructs was carried out as described above. U20S cells were used in 24-well plate, at 120K cells/well. 1000 ng RNA was transfected with 1.2 uL Lipofectamine. Integration was assessed based on the percentage of GFP positive cells (% GFP positive cells) after 24 h, with a higher percentage of GFP positive cells being indicative of higher levels of integration. % GFP positive cells was assessed by FACS following 24 hours after transfection with RNA.

As shown in FIG. 6A, Vingi-1 with the mutation—G833I improved GFP signal by 5% compared to the WT driver. Both Isoleucine and Glycine are hydrophobic residues, but Glycine can interrupt an alpha helix or a beta sheet secondary structure, thus changing the protein conformation. In FIG. 6B, Vingi-1 with the mutation P808K improved the GFP signal by 10% compared to the WT driver. As lysine is a positively charged residue and Proline isn't, this mutation can increase the affinity of the driver towards the RNA template in the RT domain. In FIG. 6C, Vingi-1 with the mutation M735E, improved the GFP signal by 10% compared to the WT driver. Methionine is a large bulky hydrophobic residue that can cause steric interference in the structure of the protein. Also, Glutamate is a negatively charged amino acid that can form hydrogen bonds. Thus, removal of Methionine in position 735, can either stabilize the protein conformation, or forming stabilizing intramolecular H-bonds. In FIG. 6D, two different NLS peptides Vingi-1 fusions, PARP1 and TOBP1, improved the GFP signal up to 5% compared to the WT. In FIG. 6E, the mutation Vingi-1 with the mutation T214S, improved the GFP signal by 8-10% compared to the WT. A substitution of a Threonine amino acid with Serine, which is very similar in properties but is different by size, can alter the protein conformation, leading to a more stable protein structure. In FIG. 6F, the introduction of a positively charged residue to the Vingi-1 driver, N695R, improved the GFP signal in more than 10% compared to the WT, probably due to increased RNA or DNA binding affinity of the reverse transcriptase domain. In FIG. 6G, N-terminal Vingi-1 fusion of paRecT improved the GFP signal by 10% compared to the WT, possibly as a result of induced activation of the recombination pathway by the RecT protein fusion. In FIG. 6H, Vingi-1 with the mutation F977Y, had a higher GFP signal of about 5% compared to the WT. As both Phenylalanine and Tyrosine have aromatic ring, the addition a hydroxyl group on the Tyrosine can form additional intramolecular or intermolecular hydrogen bonds, such as intermolecular H-bonds. In addition, Vingi-1 with the mutation Q215D, improved the activity by almost 2-fold than the WT. Position 215 may serve as the catalytic residue in the endonuclease domain, and by mutation from Glutamine to Aspartate can increase the catalytic efficiency, leading to higher activation. In FIG. 6I, Vingi-1 with the mutation A742M improved the activity by about 12%, Both of Alanine and Methionine are hydrophobic amino acids, but Methionine is bigger and can have a larger hydrophobic core packing effect on the protein, thus, increase the protein stability.

It was observed that numerous mutations and domains fusions were found to improve Vingi-1 driver activity, while others had negative effects. As shown for ZFL2-2, by combining multiple different modifications targeting different retrotransposable element functions, significant improvements in integration are possible. Overall, with the tested mutations and domains fusions introduced to Vingi-1 driver modifications have shown up to 2-fold improvement in activity compared to WT.

### Example 8

In order to test the effect of activity-improving modifications on improving integration efficiency of retrotransposable elements in human cells, Vingi-1_Acer mutants were tested for its ability to deliver transgenes to human T-cells. Vingi-1_Acar is a Vingi LINE element taken from the genome of Anolis carolinensis (green anole lizard). The driver and GFP reporter were configured in trans (with separate driver construct and gene delivery construct, respectively), transcribed as mRNA from plasmid DNA as described in previous examples.

### Materials:

Peripheral blood mononuclear cells (PBMCs)—Cell Generation, Cyropreserved (cat #1010025)
Medium components:
Thawing medium—complete RPMI1640
Activation and culturing medium—ImmunoCult™-XF T cell Expansion Medium (Cat #10981, StemCell™ Technologies)
Penicillin—Streptomycin (“penstrep”) Solution 100×—(Cat #L0022)
Fetal bovine serum (“FBS”) qualified, heat inactivated, Brazil. (Cat #10500064)
IL2—Human IL-2 IS (improved sequence), premium grade, 1000 ug (Cat #130-097-748, Miltenyi) suspended in deionized water to 1×10⁵ U/ml

### Reagents

T Cell TransAct™, human (Cat #130-111-160, Miltenyi Biotec)
ApoE 0.1 mg/ml
LNPs—prepared in house

### Flow Cytometry

DAPI—(Cat #) suspended to 1 mg/ml in DW for live/dead discrimination
Cytoflex Plus—4 Lasers (V, B, Y, R)

### Labware

T75 flask
T25 flask
96-well flat-bottom microplates, TC treated, clear
96-well round-bottom microplates, TC treated, clear
PCR Strips

### Preparation of Mediums/Reagents:

Complete RPMI 1640 (“cRPMI”) thawing medium preparation.
   Prepare by adding 1% penstrep and 10% FBS by adding 5 ml penstrep and 50 ml FBS to 500 ml of RPMI 1640 medium.
“Complete T cell medium” preparation
   Re-constitute 1000 μg of lyophilized human IL2 IS premium grade (Miltenyi) to a concentration of 1×10⁵ U/ml (>100 μg/ml) in deionized water under sterile conditions.
   Divide to aliquots of 100 μl and store at −80 C. Before use thaw and dilute 1:10 by adding 900 μl ImmunoCult™-XF T cell Expansion Medium. Store up to 10 days at 4 C.
   Prepare 0.5% penstrep ImmunoCult™-XF T cell Expansion Medium by adding 2.5 ml penstrep to 500 ml of medium.
   Prepare the complete T cell medium by adding 50 μL of ×1000 IL-2 to every 50 mL of 0.5% penstrep ImmunoCult™-XF T cell Expansion Medium (Final IL2 concentration of 0.1p g/ml for Gibco or 100 U/ml for Miltenyi)

### Procedure:

Day −2 (relative to transfection, 0 days post thaw)—[insert date]
   PBMCs Thawing
      Thaw the PBMCs vial in a 37° C. water bath. Thawing should be rapid (approximately 2 minutes).
      Remove the vial from the water bath as soon as the contents are thawed and decontaminate by dipping in or spraying with 70% ethanol.
      Gently add 1 ml of 100% FBS to thawed vial of PBMCs
      Transfer the vial contents to a centrifuge tube containing 10.0 mL RPMI complete medium and spin at 300 g for 10 minutes.
      Resuspend the cell pellet with 10 ml complete T cells medium.
      Count the cells using an automatic cell counter and Trypan Blue for live/dead discrimination
      Bring PBMCs to final concentration of 2×10⁶ viable cells/mL and dispense into a T75 flask
   T cell activation
      Activate T cells by adding 100 ul TransAct per 10 ml T cells complete medium.
      Incubate at 37 C, 5% CO² for 2 days
      Monitor activation by observing density and clumps under microscope
Day 0 (relative to transfection, 2 days post thaw)
   Lipid nanoparticles are produced according to manufacturer's instructions (Ignite Nano Assembler, Precision Nanosystems).

### LNP Treatment:

Count activated T cells
Take the appropriate number of cells for the LNP treatment
Centrifuge cells at 300 g for 5 min at RT
Aspirate the supernatant and discard.
Bring cells to 1×10⁶ cells/ml by resuspend cell's pellet with T cells complete medium
supplemented with 2 ug/ml ApoE (0.1 mg/ml stock).
Seed 100 μl per well in 96-well plate
Dropwise pipette the LNP treatments
Incubate at 37C, 5% CO2 for 1 day

### Transfection:

Day 1 (relative to transfection, 3 days post thaw)
   LNP residues removal
      After 24 h centrifuge 96-well plates at 300 g for 5 min at RT.
      Carefully discard most of the medium using a multichannel pipette
      Resuspend cells with 100 μl fresh complete T cells medium—Without ApoE.
Day 2 (relative to transfection, 4 days post thaw)
   Split cells
      Before splitting cells use EVOS to observe GFP expression
      Split cells 1: by taking μl into μl (total of 200 μl) on new 96-well plate
Day 5 (relative to transfection, 7 days post thaw)—[insert date]
   Detection of transgene expression (FACS) and copies (dPCR)—5 day time point
      For dPCR—transfer 80 μl from each sample into PCR strips.
      a. Centrifuge at 2100 g for 10 min at 4 C
      b. Discard supernatant and freeze pellet at −80 C
      For FACS—transfer 80 μl from each sample into PCR strips.
      c. Prepare PBS+DAPI×2 solution by diluting 1 mg/ml stock 1:1000 in PBS−/−
      d. Place 80 μl of DAPI×2 solution into wells of a 96-well round bottom plate
      e. Transfer 80 μl of cells into the wells containing the 2×DAPI and mix
      f. Run on CytoFlex flow cytometer using the following settings and with stopping gate on 15000 live cells:

TABLE 13

| Parameter (channel) | gain |
|---|---|
| Threshold FSC-H | 30k |
| FSC | 64 (beads 49) |
| SSC | 57 (beads 23) |
| FITC (GFP) | 70 |
| APC (CD19-CAR) | 800 |
| PB450 (Dapi) | 48 |

For further culture
Split cells 1:5 every 2-3 days or 1:10 every 4 days with fresh complete T cell medium
Day x (relative to transfection, x+2 days post thaw)
Detection of transgene expression (FACS) and copies (dPCR)—
Repeat steps detailed on day 5 FACS and dPCR timepoint

### Results:

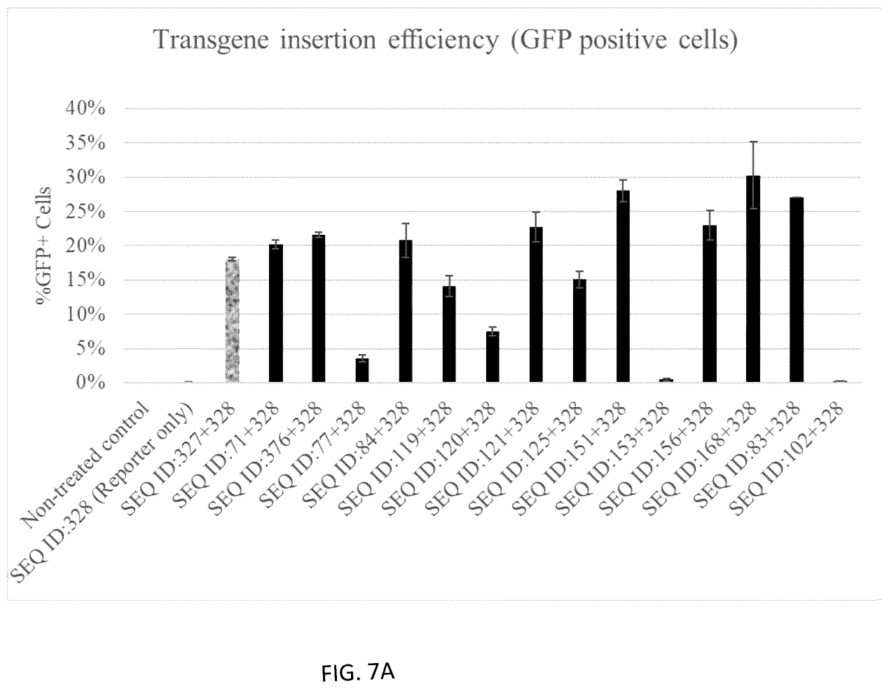

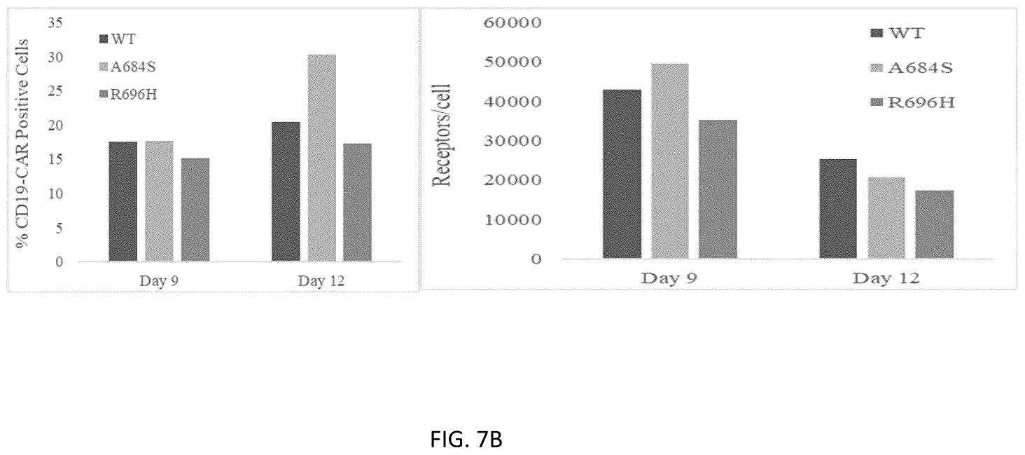

FIG. 7A shows results of integration assays using Vingi-1 drivers with point mutations. In this experiment, different retrotransposable element constructs were used in the trans configuration (driver and GFP reporter encoded by different RNA). A common gene delivery construct encoding the GFP reporter (EX2988; SEQ ID NO: 328) was used for all driver constructs tested. The following mutations in Vingi-1 were tested in this experiment: Q634L (SEQ ID NO: 71), F238Y+M16I (SEQ ID NO:376), I45L (SEQ ID NO: 77), G833I (SEQ ID NO: 84), K703R (SEQ ID NO: 119), K480Q (SEQ ID NO: 120), K675R (SEQ ID NO: 121), P808K (SEQ ID NO: 125), M570L (SEQ ID NO: 151), L590F (SEQ ID NO: 153), M735E (SEQ ID NO: 156), K966R (SEQ ID NO: 168), A901H (SEQ ID NO: 83), L493R (SEQ ID NO: 102). The wild-type Vingi-1 driver is shown in grey (SEQ ID NO:327).

Aside from the mutations listed, all Vingi-1 driver constructs were identical in sequence to SEQ ID NO:327. IVT of different RTE constructs was carried out as described above. Human T-cells were used in 96-well plate, at 10K cells/well. 400 ng mRNA was delivered with LNP. Integration was assessed based on the percentage of GFP positive cells (% GFP positive cells) after 5d, with a higher percentage of GFP positive cells being indicative of higher levels of integration. % GFP positive cells was assessed by FACS following 5d hours after LNP delivery of mRNA.

These results indicate that the described retrotransposable element system can deliver transgenes to primary human cells in all-mRNA LNP compositions and that point mutations can significantly improve insertion efficiency (>50% with single mutations K966R (SEQ ID NO: 168), A901H (SEQ ID NO: 83), M570L (SEQ ID NO: 151)).

In order to test the effect of activity-improving modifications on improving integration efficiency of retrotransposable elements in human cells, Vingi-1 mutants were tested for its ability to deliver a chimeric antigen receptor (CAR) transgene to human T-cells. The chimeric antigen receptor comprises anti-CD19 scfv, CD8 hinge, CD8 transmembrane, 4-1BB co-stimulatory domain, and CD3zeta cytoplasmic domain.

Vingi-1 CAR reporter is encoded by plasmid SEQ ID NO: 398, with mRNA cassette containing Clean Cap-compatible T7 RNA promoter (TriLink Biotechnologies, bold) and A30N10A70 polyA tail (underlined). CAR cassette including EF1-A promoter (italics), CAR ORF (bold italics), and synthetic polyadenylation signal is in anti-sense. In between the T7 promoter and CAR cassette is the 5′UTR from Vingi-1 element (italics underlined), and in between the GFP cassette and polyA tail is the 3′UTR from Vingi-1 element (bold underlined).

(SEQ ID NO: 398)
TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATATGCGGT
GTGAAATACCGCACAGATGCGTAAGGAGAAAATACCGCATCAGGCGCCATTCGCCATTCAGGCT
GCGCAACTGTTGGGAAGGGCGATCGGTGCGGGCCTCTTCGCTATTACGCCAGCTGGCGAAAGGG
GGATGTGCTGCAAGGCGATTAAGTTGGGTAACGCCAGGGTTTTCCCAGTCACGACGTTGTAAAA
CGACGGCCAGTGAATTGGAGATCGGTACTTCGCGAGTTTAAACTAATACGACTCACTATAAGGG
GGGGACACGGAAAGAGCCTCCCCGAAGATTGAGTgAATTCAGTCGGGCGTCCCCTGGGCAACGT
TTCTTGTAAGCGGCCGATCTTTCCACCCCAAAAGCATTGGATGaGTCGACGCGGCCTACTCGAC
GGATCGATCCGAACAAACGACCCAACACCCGTGCGTTTTATTCTGTCTTTTTATTGCCGATCCC
CTCAGAAGAACTCGTCAAGAAGGCGATAGAAGGCGATGCGCTGCGAATCGGGAGCGGCGATACC
GTAAAGCACGAGGAAGCGGTCAGCCCATTCGCCGCCAAGCTCTTCAGCAATATCACGGGTAGCC
AACGCTATGTCCTGATAGCGGTCGGCCGCTTTAGCGAGGGGGCAGGGCCTGCATGTGAAGGGCG
TCGTAGGTGTCCTTGGTGGCTGTACTCAGACCCTGGTAAAGGCCATCGTGCCCCTTGCCCCTCC
GGCGCTCGCCTTTCATCCCAATCTCACTGTAGGCCTCCGCCATCTTATCTTTCTGCAGTTCATT
GTACAGGCCTTCCTGAGGGTTCTTCCTTCTCGGCTTTCCCCCCATCTCAGGGTCCCGGCCACGT
CTCTTGTCCAAAACATCGTACTCCTCTCTTCGTCCTAGATTGAGCTCGTTATAGAGCTGGTTCT
GGCCCTGCTTGTACGCGGGGGCGTCTGCGCTCCTGCTGAACTTCACTCTCAGTTCACATCCTCC
TTCTTCTTCTTCTGGAAATCGGCAGCTACAGCCATCTTCCTCTTGAGTAGTTTGTACTGGCCTC
ATAAATGGTTGTTTGAATATATACAGGAGTTTCTTTCTGCCCCGTTTGCAGTAAAGGGTGATAA
CCAGTGACAGGAGAAGGACCCCACAAGTCCCGGCCAAGGGCGCCCAGATGTAGATATCACAGGC
GAAGTCCAGCCCCCTCGTGTGCACTGCGCCCCCCGCCGCTGGCCGGCACGCCTCTGGGCGCAGG
GACAGGGGCTGCGACGCGATGGTGGGCGCCGGTGTTGGTGGTCGCGGCGCTGGCGTCGTGGTTG
AGGAGACGGTGACTGAGGTTCCTTGGCCCCAGTAGTCCATAGCATAGCTACCACCGTAGTAATA
ATGTTTGGCACAGTAGTAAATGGCTGTGTCATCAGTTTGCAGACTGTTCATTTTTAAGAAAACT
TGGCTCTTGGAGTTGTCCTTGATGATGGTCAGTCTGGATTTGAGAGCTGAATTATAGTATGTGG
TTTCACTACCCCATATTACTCCCAGCCACTCCAGACCCTTTCGTGGAGGCTGGCGAATCCAGCT
TACACCATAGTCGGGTAATGAGACGCCTGAGACAGTGCATGTGACGGACAGGCTCTGTGAGGGC
GCCACCAGGCCAGGTCCTGACTCCTGCAGTTTCACCTCAGATCCGCCGCCACCCGACCCACCAC
CGCCCGAGCCACCGCCACCTGTGATCTCCAGCTTGGTCCCCCCTCCGAACGTGTACGGAAGCGT
ATTACCCTGTTGGCAAAAGTAAGTGGCAATATCTTCTTGCTCCAGGTTGCTAATGGTGAGAGAA
TAATCTGTTCCAGACCCACTGCCACTGAACCTTGATGGGACTCCTGAGTGTAATCTTGATGTAT
GGTAGATCAGGAGTTTAACAGTTCCATCTGGTTTCTGCTGATACCAATTTAAATATTTACTAAT
GTCCTGACTTGCCCTGCAACTGATGGTGACTCTGTCTCCCAGAGAGGCAGACAGGGAGGATGTA
GTCTGTGTCATCTGGATGTCCGGCCTGGCGGCGTGGAGCAGCAAGGCCAGCGGCAGGAGCAAGG
CGGTCACTGGTAAGGCCATGGTGGCTCACGACACCTGAAATGGAAGAAAAAAACTTTGAACCAC
TGTCTGAGGCTTGAGAATGAACCAAGATCCAAACTCAAAAAGGGCAAATTCCAAGGAGAATTAC
ATCAAGTGCCAAGCTGGCCTAACTTCAGTCTCCACCCACTCAGTGTGGGGAAACTCCATCGCAT
AAAACCCCTCCCCCCAACCTAAAGACGACGTACTCCAAAAGCTCGAGAACTAATCGAGGTGCCT
GGACGGCGCCCGGTACTCCGTGGAGTCACATGAAGCGACGGCTGAGGACGGAAAGGCCCTTTTC
CTTTGTGTGGGTGACTCACCCGCCCGCTCTCCCGAGCGCCGCGTCCTCCATTTTGAGCTCCCTG
CAGCAGGGCCGGGAAGCGGCCATCTTTCCGCTCACGCAACTGGTGCCGACCGGGCCAGCCTTGC
CGCCCAGGGGGGGGCGATACACGGCGGCGCGAGGCCAGGCACCAGAGCAGGCCGGCCAGCTTGA
GACTACCCCCGTCCGATTCTCGGTGGCCGCGCTCGCAGGCCCCGCCTCGCCGAACATGTGCGCT
GGGACGCACGGGCCCCGTCGCCGCCCGCGGCCCCAAAAACCGAAATACCAGTGTGCAGATCTTG
GCCCGCATTTACAAGACTATCTTGCCAGAAAAAAAGCGTCGCAGCAGGTCATCAAAAATTTTAA
ATGGCTAGAGACTTATCGAAAGCAGCGAGACAGGCGCGAAGGTGCCACCAGATTCGCACGCGGC
GGCCCCAGCGCCCAGGCCAGGCCTCAACTCAAGCACGAGGCGAAGGGGCTCCTTAAGCGCAAGG
CCTCGAACTCTCCCACCCACTTCCAACCCGAAGCTCGGGATCAAGAATCACGTACTGCAGCCAG
GTGGAAGTAATTCAAGGCACGCAAGGGCCATAACCCGTAAAGAGGCCAGGCCCGCGGGAACCAC
ACACGGCACTTACCTGTGTTCTGGCGGCAAACCCGTTGCGAAAAAGAACGTTCACGGCGACTAC
TGCACTTATATACGGTTCTCCCCCACCCTCGGGAAAAAGGCGGAGCCAGTACACGACATCACTT
TCCCAGTTTACCCCGCGCCACCTTCTCTAGGCACCGGTTCAATTGCCGACCCCTCCCCCCAACT
TCTCGGGGACTGTGGGCGATGTGCGCTCTGCCCACTGACGGGCACCGGAGCCgaattcgaagct
tTTGCTTGTGATTTCTTTTCTTTTtTTTTTATTTCCATTATTTGAAATGTATTTGCTGTACCA
ATGCTTTTGACACGAAATAAATAAAgctagcACCGGTAAAAAAAAAAAAAAAAAAAAAAAAAAA
AAAaacgttGACTAAAAAAAAAAAAAAAAAAAAAAAAAAAAAAAAAAAAAAAAAAAAAAAAAAA
AAAAAAAAAAAAAAAAAAAaaaaaaGTCTTCGCGCGCATCATCGGATGCCGGGACCGACGAGTG
CAGAGGCGTGCAAGCGAGCTTGGCGTAATCATGGTCATAGCTGTTTCCTGTGTGAAATTGTTAT
CCGCTCACAATTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAACCCGACAGGAC
TATAAAGATACCAGGCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTTCTCATAGCTCACGC
TGTAGGTATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCT
ACAGAGTTCTTGAAGTGGTGGCCTAACTACGGCTACACTAGAAGAACAGTATTTGGTATCTGCG
CTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGGGACCCACG
CTCACCGGCTCCAGATTTATCAGCAATAAACCAGCCAGCCGGAAGGGCCGAGCGCAGAAGTGGT
CCTGCAACTTTATCCGCCTCCATCCAGTCTATTAATTGTTGCCGGGAAGCTAGAGTAAGTAGTT
CGCCAGTTAATAGTTTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAA
GTGCCACCTGACGTCTAAGAAACCATTATTATCATGACATTAACCTATAAAAATAGGCGTATCA
CGAGGCCCTTTCGTC

The above protocol was modified with the following after Day 2 post activation:
Day 2 (day of transfection)
   LNP Treatment
      Count activated T cells
      Take the appropriate number of cells for the LNP treatment
      Centrifuge cells at 300 g for 5 min at RT
      Aspirate the supernatant and discard.
      Bring cells to 1×10⁶ cells/ml by resuspend cell's pellet with 3 ml T cells complete
      medium supplemented with 2 ug/ml ApoE (0.1 mg/ml stock).
      Seed 100 μl per well in 96-well plate, according to the request
      Dropwise pipette the LNP treatments (see sample list—transfection table)
      Incubate at 37 C, 5% CO2 for 1 day
Day 3 (1 day post transfection)
   LNP residues removal
      After 24 h centrifuge 96-well plates at 300 g for 5 min at RT.
      Carefully discard most of the medium using a multichannel pipette
      Resuspend cells with 100 μl fresh complete T cells medium—Without ApoE.
      Transfer the cells into 24w G-Rex (both duplicates) and add up to 7 ml T cell medium with IL-2
Day 4+ (2+ days post transfection)
   Every 2-3 days replace medium (if needed) or add fresh IL-2
Day 5, 9 and 12 post transfection
   Detection of transgene expression (FACS) and copies (dPCR)
      Mix each well thoroughly.
      For dPCR—transfer from each sample into PCR strips (make 2 technical samples).
         Centrifuge at 2100 g for 10 min at 4 C
         Discard supernatant and freeze pellet at −80C
      For FACS—transfer from each sample into U shaped 96 well for staining.
         Mark the samples wells on the bottom of the plate
         Centrifuge the plate at 300 g for 5 min at room temperature (RT)
         Discard supernatant by flipping the plate once
         Prepare staining mix by diluting 1:50 the antibody into FACS buffer Add 50 ul of the staining mix and cover the plate with aluminum foil
         Incubate for 15 min at RT
         Stop staining by adding FACS buffer to each well
         Centrifuge the plate at 300 g for 5 min at RT
         Discard supernatant by flipping the plate once
         Prepare PBS+DAPI×1 solution by diluting 1 mg/ml stock 1:2000 in PBS−/−
         Place 150-200 μl of DAPI×1 solution into the appropriate wells
         Run on CytoFlex flow cytometer using the following settings and with stopping gate on 15000 live cells:

TABLE 14

| Parameter (channel) | gain |
|---|---|
| Threshold FSC-H | 30k |
| FSC | 64 (beads 49) |
| SSC | 57 (beads 23) |
| FITC (GFP) | 70 |
| APC (CD19-CAR) | 800 |
| PB450 (Dapi) | 48 |

Export from each sample the following data:
a. % CAR positive cells
b. MFI (median and geometric) of CAR (APC-A)
c. events of singlets
d. events of CAR positive cells
Receptor quantification (MESF quick Qal)—
Prepare 2 tubes with 400 ul of FACS buffer each
Mark Blank or Beads and add one drop of each bottle to the designated tube (blank to Blank, and number 1-4 to Beads)
Mix thoroughly and transfer 250 ul to a 96 well plate
Run by FACS and read 5000 events of each population at the same acquisition settings as the experiment (except for FSC SSC—see Gain).
Analyze and calculate receptor/cell

### Results:

FIG. 7B shows results of integration assays using Vingi-1 drivers with point mutations. In this experiment, different retrotransposable element constructs were used in the trans configuration (driver and CAR reporter encoded by different RNA). The same CAR reporter was used for all constructs (SEQ ID NO: 398). The following mutations in Vingi-1 were tested in this experiment: A684S (SEQ ID NO: 72), R696H (SEQ ID NO: 179).

Aside from the mutations listed, all Vingi-1 driver constructs were identical in sequence to SEQ ID NO:327. IVT of different RTE constructs was carried out as described above. Human T-cells were used in 96-well plate, at 10K cells/well. 400 ng mRNA was delivered with LNP. Integration was assessed based on the percentage of CD19-CAR positive cells after 9-12d, with a higher percentage of CAR positive cells being indicative of higher levels of integration. Receptors/cell was assessed by FACS following 9-12d hours after LNP delivery of mRNA.

A sequence description table with a brief description of the sequences disclosed herein is provided below:

TABLE 15

| SEQ ID NO: | EX# | Protein/DNA | Category | Description |
|---|---|---|---|---|
| 1 | EX154 | PROTEIN | ZFL2-2 | ZFL2-2 driver having N-terminal CtIP fragment |
| | | | | ZFL2-2 fusion |
| 2 | EX155 | PROTEIN | ZFL2-2 | ZFL2-2 driver having N-terminal RAD51 |
| | | | | ZFL2-2 fusion |
| 3 | EX156 | PROTEIN | ZFL2-2 | ZFL2-2 driver having N-terminal UL12 ZFL2-2 |
| | | | | fusion |
| 4 | EX157 | PROTEIN | ZFL2-2 | ZFL2-2 driver having N-terminal BRCA2 |
| | | | | fragment ZFL2-2 fusion |
| 5 | EX158 | PROTEIN | ZFL2-2 | ZFL2-2 driver having N-terminal DSS1 peptide |
| | | | | ZFL2-2 fusion |
| 6 | EX170 | PROTEIN | ZFL2-2 | ZFL2-2 driver having N-terminal HMGN1 |
| | | | | ZFL2-2 fusion |
| 7 | EX171 | PROTEIN | ZFL2-2 | ZFL2-2 driver having C-terminal HMGB1 |
| | | | | ZFL2-2 fusion |
| 8 | EX153 | PROTEIN | ZFL2-2 | ZFL2-2 driver having C-terminal Sto7D ZFL2- |
| | | | | 2 fusion |
| 9 | EX282 | PROTEIN | ZFL2-2 | ZFL2-2 driver having C-terminal Nibrin |
| | | | | MRE11 recruitment peptide ZFL2-2 fusion |
| 10 | EX300 | PROTEIN | ZFL2-2 | ZFL2-2 driver having C-terminal MDM2 |
| | | | | ZFL2-2 fusion |
| 11 | EX301 | PROTEIN | ZFL2-2 | ZFL2-2 driver having C-terminal p53 inhibiting |
| | | | | peptide to ZFL2-2 fusion |
| 12 | EX302 | PROTEIN | ZFL2-2 | ZFL2-2 driver having C-terminal Nanog |
| | | | | derived peptide ZFL2-2 fusion |
| 13 | EX298 | PROTEIN | ZFL2-2 | ZFL2-2 driver having C-terminal E. coli |
| | | | | RNAseH1 ZFL2-2 fusion |
| 14 | EX169 | PROTEIN | ZFL2-2 | ZFL2-2 driver having C-terminal RNAseH1 |
| | | | | ZFL2-2 fusion |
| 15 | EX272 | PROTEIN | ZFL2-2 | ZFL2-2 driver having N-terminal AAVS1 Zinc |
| | | | | finger ZFL2-2 fusion |
| 16 | EX274 | PROTEIN | ZFL2-2 | ZFL2-2 driver having N-terminal dead Cas9 |
| | | | | (D10A, H840A) ZFL2-2 fusion |
| 17 | EX277 | PROTEIN | ZFL2-2 | ZFL2-2 driver having N-terminal PCSK9 |
| | | | | homing endonuclease ZFL2-2 (endonuclease |
| | | | | mutant D237A) fusion |
| 18 | EX278 | PROTEIN | ZFL2-2 | ZFL2-2 driver having N-terminal PCSK9 |
| | | | | homing endonuclease ZFL2-2 (endonuclease |
| | | | | deleted) fusion |
| 19 | EX294 | PROTEIN | ZFL2-2 | ZFL2-2 driver having N-terminal PCSK9 |
| | | | | homing nickase Q47E ZFL2-2 (endonuclease |
| | | | | deleted) fusion |
| 20 | EX295 | PROTEIN | ZFL2-2 | ZFL2-2 driver having N-terminal PCSK9 |
| | | | | homing nickase Q47E ZFL2-2 (endonuclease |
| | | | | domain mutant D237A) fusion |
| 21 | EX283 | PROTEIN | ZFL2-2 | ZFL2-2 driver having N-terminal nickase Cas9 |
| | | | | (H840A) ZFL2-2 (endonuclease domain |
| | | | | deleted) fusion |
| 22 | EX312 | PROTEIN | ZFL2-2 | ZFL2-2 driver having SpCas9 fusion to ZFL2-2 |
| | | | | Reverse Transcriptase domain |
| 23 | | PROTEIN | Heterologous | human HMGN1 protein |
| 24 | | PROTEIN | Heterologous | human HMGB1 protein |
| 25 | | PROTEIN | Heterologous | UL12 protein |
| 26 | | PROTEIN | Heterologous | Sulfolobus tokodaii Sto7D protein |
| 27 | EX282 | PROTEIN | ZFL2-2 | ZFL2-2 driver having C-terminal NBN peptide |
| | | | | ZFL2-2 fusion |
| 28 | EX282 | DNA | ZFL2-2 | Plasmid encoding ZFL2-2 driver having C- |
| | | | | terminal NBN peptide ZFL2-2 fusion |
| 29 | EX284 | PROTEIN | ZFL2-2 | ZFL2-2 driver having N-terminal MDM2 |
| | | | | peptide ZFL2-2 fusion |
| 30 | EX284 | DNA | ZFL2-2 | Plasmid encoding ZFL2-2 driver having N- |
| | | | | terminal MDM2 peptide ZFL2-2 fusion |
| 31 | EX584 | PROTEIN | ZFL2-2 | ZFL2-2 driver having C-terminal UL12 ZFL2-2 |
| | | | | fusion |
| 32 | EX584 | DNA | ZFL2-2 | Plasmid encoding ZFL2-2 driver having C- |
| | | | | terminal UL12 ZFL2-2 fusion |
| 33 | EX586 | PROTEIN | ZFL2-2 | ZFL2-2 driver having C-terminal UL12 fused |
| | | | | to ZFL2-2, N647K mutation |
| 34 | EX586 | DNA | ZFL2-2 | Plasmid encoding ZFL2-2 driver having C- |
| | | | | terminal UL12 fused to ZFL2-2, N647K |
| | | | | mutation |
| 35 | EX587 | PROTEIN | ZFL2-2 | ZFL2-2 driver having C-terminal Sto7D and |
| | | | | UL12 ZFL2-2 fusion |
| 36 | EX587 | DNA | ZFL2-2 | Plasmid encoding ZFL2-2 driver having C- |
| | | | | terminal Sto7D and UL12 ZFL2-2 fusion |
| 37 | EX594 | PROTEIN | ZFL2-2 | ZFL2-2 driver having C-terminal BRCA2 |
| | | | | peptide ZFL2-2 fusion |
| 38 | EX594 | DNA | ZFL2-2 | Plasmid encoding ZFL2-2 driver having C- |
| | | | | terminal BRCA2 peptide ZFL2-2 fusion |
| 39 | EX595 | PROTEIN | ZFL2-2 | ZFL2-2 driver having N-terminal HMGN1, C- |
| | | | | terminal HMGB1 ZFL2-2 fusion |
| 40 | EX595 | DNA | ZFL2-2 | Plasmid encoding ZFL2-2 driver having N- |
| | | | | terminal HMGN1, C-terminal HMGB1 ZFL2-2 |
| | | | | fusion |
| 41 | EX596 | PROTEIN | ZFL2-2 | ZFL2-2 driver having C-terminal HMGN1 |
| | | | | UL12 ZFL2-2 fusion |
| 42 | EX596 | DNA | ZFL2-2 | Plasmid encoding ZFL2-2 driver having C- |
| | | | | terminal HMGN1 UL12 ZFL2-2 fusion |
| 43 | EX597 | PROTEIN | ZFL2-2 | ZFL2-2 driver having C-terminal UL12 Sto7D |
| | | | | ZFL2-2 fusion |
| 44 | EX597 | DNA | ZFL2-2 | Plasmid encoding ZFL2-2 driver having C- |
| | | | | terminal UL12 Sto7D ZFL2-2 fusion |
| 45 | EX588 | PROTEIN | ZFL2-2 | ZFL2-2 driver having ZFL2-2 fusion encoded |
| | | | | by SEQ ID NO: 46 |
| 46 | EX588 | DNA | ZFL2-2 | Plasmid encoding ZFL2-2 mRNA with Human |
| | | | | beta globin 5′ UTR |
| 47 | EX666 | PROTEIN | ZFL2-2 | ZFL2-2 driver having N-terminal Nhp6a ZFL2- |
| | | | | 2 fusion |
| 48 | EX666 | DNA | ZFL2-2 | Plasmid encoding ZFL2-2 driver having N- |
| | | | | terminal Nhp6a ZFL2-2 fusion |
| 49 | SM001 | PROTEIN | ZFL2-2 | ZFL2-2 protein (as encoded in SM001 plasmid) |
| 50 | SM001 | DNA | ZFL2-2 | L2-2 cis driver and GFP reporter encoding |
| | | | | plasmid |
| 51 | SM002 | PROTEIN | ZFL2-2 | Danio rerio (Zebrafish) ZFL2-2 protein |
| 52 | SM002 | DNA | ZFL2-2 | ZFL2-2 protein encoding plasmid |
| 53 | SM003 | DNA | ZFL2-2 | ZFL2-2 GFP transgene reporter gene delivery |
| | | | | construct |
| 54 | | PROTEIN | Heterologous | SV40 NLS |
| 55 | | PROTEIN | Heterologous | Nucleoplasmin NLS |
| 56 | | PROTEIN | Heterologous | Bipartite SV40 NLS |
| 57 | | PROTEIN | Heterologous | PNRC Nucleolar localization signal |
| 58 | | PROTEIN | Heterologous | PolyR sequence |
| 59 | | PROTEIN | Heterologous | H2B NLS |
| 60 | | DNA | ZFL2-2 | ZFL2-2 3′ UTR |
| 61 | | DNA | ZFL2-1 | ZFL2-1 3′UTR |
| 62 | | DNA | UnaL | UnaL 3′UTR |
| 63 | | DNA | Vingi-1 | Vingi-1 3′UTR |
| 64 | | DNA | ZFL2-2 | ZFL2-2 5′ UTR |
| 65 | | DNA | ZFL2-1 | ZFL2-1 5′UTR |
| 66 | | DNA | UnaL | UnaL 5′UTR |
| 67 | | DNA | Vingi-1 | Vingi-1 5′UTR |
| 68 | | DNA | Heterologous | human beta globin 3′UTR |
| 69 | | DNA | Heterologous | human alpha globin 3′UTR |
| 70 | EX3310 | PROTEIN | Vingi-1 | Vingi-1 driver H929G mutant |
| 71 | EX3311 | PROTEIN | Vingi-1 | Vingi-1 driver Q634L mutant |
| 72 | EX3320 | PROTEIN | Vingi-1 | Vingi-1 driver A684S mutant |
| 73 | EX3314 | PROTEIN | Vingi-1 | Vingi-1 driver F977Y mutant |
| 74 | EX3315 | PROTEIN | Vingi-1 | Vingi-1 driver H850Q mutant |
| 75 | EX3308 | PROTEIN | Vingi-1 | Vingi-1 driver F238Y mutant |
| 76 | EX3309 | PROTEIN | Vingi-1 | Vingi-1 driver A875T mutant |
| 77 | EX3342 | PROTEIN | Vingi-1 | Vingi-1 driver I45L mutant |
| 78 | EX3347 | PROTEIN | Vingi-1 | Vingi-1 driver L434I mutant |
| 79 | EX3348 | PROTEIN | Vingi-1 | Vingi-1 driver I439L mutant |
| 80 | EX3350 | PROTEIN | Vingi-1 | Vingi-1 driver T470A mutant |
| 81 | EX3358 | PROTEIN | Vingi-1 | Vingi-1 driver Y673W mutant |
| 82 | EX3364 | PROTEIN | Vingi-1 | Vingi-1 driver Y950M mutant |
| 83 | EX3366 | PROTEIN | Vingi-1 | Vingi-1 driver A901H mutant |
| 84 | EX3370 | PROTEIN | Vingi-1 | Vingi-1 driver G833I mutant |
| 85 | EX3371 | PROTEIN | Vingi-1 | Vingi-1 driver G833S mutant |
| 86 | EX3463 | PROTEIN | Vingi-1 | Vingi-1 driver R350K mutant |
| 87 | EX3312 | PROTEIN | Vingi-1 | Vingi-1 driver S35C mutant |
| 88 | EX3313 | PROTEIN | Vingi-1 | Vingi-1 driver L111V mutant |
| 89 | EX3316 | PROTEIN | Vingi-1 | Vingi-1 driver M16I mutant |
| 90 | EX3317 | PROTEIN | Vingi-1 | Vingi-1 driver A87S mutant |
| 91 | EX3318 | PROTEIN | Vingi-1 | Vingi-1 driver N311D mutant |
| 92 | EX3319 | PROTEIN | Vingi-1 | Vingi-1 driver I52S mutant |
| 93 | EX3321 | PROTEIN | Vingi-1 | Vingi-1 driver Y313F mutant |
| 94 | EX3322 | PROTEIN | Vingi-1 | Vingi-1 driver I52P mutant |
| 95 | EX3323 | PROTEIN | Vingi-1 | Vingi-1 driver S109T mutant |
| 96 | EX3346 | PROTEIN | Vingi-1 | Vingi-1 driver Q215D mutant |
| 97 | EX3349 | PROTEIN | Vingi-1 | Vingi-1 driver R468K mutant |
| 98 | EX3351 | PROTEIN | Vingi-1 | Vingi-1 driver C495S mutant |
| 99 | EX3352 | PROTEIN | Vingi-1 | Vingi-1 driver N529S mutant |
| 100 | EX3431 | PROTEIN | Vingi-1 | Vingi-1 driver L476R mutant |
| 101 | EX3432 | PROTEIN | Vingi-1 | Vingi-1 driver I473R mutant |
| 102 | EX3433 | PROTEIN | Vingi-1 | Vingi-1 driver L493R mutant |
| 103 | EX3434 | PROTEIN | Vingi-1 | Vingi-1 driver W353R mutant |
| 104 | EX3435 | PROTEIN | Vingi-1 | Vingi-1 driver M345K mutant |
| 105 | EX3438 | PROTEIN | Vingi-1 | Vingi-1 driver I475R mutant |
| 106 | EX3439 | PROTEIN | Vingi-1 | Vingi-1 driver L25Q mutant |
| 107 | EX3441 | PROTEIN | Vingi-1 | Vingi-1 driver S39K mutant |
| 108 | EX3442 | PROTEIN | Vingi-1 | Vingi-1 driver I52E mutant |
| 109 | EX3443 | PROTEIN | Vingi-1 | Vingi-1 driver Q63T mutant |
| 110 | EX3444 | PROTEIN | Vingi-1 | Vingi-1 driver S89Q mutant |
| 111 | EX3445 | PROTEIN | Vingi-1 | Vingi-1 driver G116N mutant |
| 112 | EX3447 | PROTEIN | Vingi-1 | Vingi-1 driver A132T mutant |
| 113 | EX3449 | PROTEIN | Vingi-1 | Vingi-1 driver V145S mutant |
| 114 | EX3452 | PROTEIN | Vingi-1 | Vingi-1 driver K196W mutant |
| 115 | EX3455 | PROTEIN | Vingi-1 | Vingi-1 driver N299K mutant |
| 116 | EX3456 | PROTEIN | Vingi-1 | Vingi-1 driver Q302K mutant |
| 117 | EX3459 | PROTEIN | Vingi-1 | Vingi-1 driver A329S mutant |
| 118 | EX3391 | PROTEIN | Vingi-1 | Vingi-1 driver E933R mutant |
| 119 | EX3392 | PROTEIN | Vingi-1 | Vingi-1 driver K703R mutant |
| 120 | EX3393 | PROTEIN | Vingi-1 | Vingi-1 driver K480Q mutant |
| 121 | EX3394 | PROTEIN | Vingi-1 | Vingi-1 driver K675R mutant |
| 122 | EX3395 | PROTEIN | Vingi-1 | Vingi-1 driver K789R mutant |
| 123 | EX3398 | PROTEIN | Vingi-1 | Vingi-1 driver H787R mutant |
| 124 | EX3399 | PROTEIN | Vingi-1 | Vingi-1 driver I793R mutant |
| 125 | EX3401 | PROTEIN | Vingi-1 | Vingi-1 driver P808K mutant |
| 126 | EX3402 | PROTEIN | Vingi-1 | Vingi-1 driver D792K mutant |
| 127 | EX3403 | PROTEIN | Vingi-1 | Vingi-1 driver I793K mutant |
| 128 | EX3404 | PROTEIN | Vingi-1 | Vingi-1 driver E797R mutant |
| 129 | EX3406 | PROTEIN | Vingi-1 | Vingi-1 driver D792M mutant |
| 130 | EX3410 | PROTEIN | Vingi-1 | Vingi-1 driver P808R mutant |
| 131 | EX3411 | PROTEIN | Vingi-1 | Vingi-1 driver M735R mutant |
| 132 | EX3412 | PROTEIN | Vingi-1 | Vingi-1 driver A742K mutant |
| 133 | EX3413 | PROTEIN | Vingi-1 | Vingi-1 driver L693K mutant |
| 134 | EX3414 | PROTEIN | Vingi-1 | Vingi-1 driver N745K mutant |
| 135 | EX3464 | PROTEIN | Vingi-1 | Vingi-1 driver Q354K mutant |
| 136 | EX3465 | PROTEIN | Vingi-1 | Vingi-1 driver R357K mutant |
| 137 | EX3467 | PROTEIN | Vingi-1 | Vingi-1 driver D362R mutant |
| 138 | EX3468 | PROTEIN | Vingi-1 | Vingi-1 driver N412E mutant |
| 139 | EX3469 | PROTEIN | Vingi-1 | Vingi-1 driver K424R mutant |
| 140 | EX3470 | PROTEIN | Vingi-1 | Vingi-1 driver M435Y mutant |
| 141 | EX3471 | PROTEIN | Vingi-1 | Vingi-1 driver E447Q mutant |
| 142 | EX3473 | PROTEIN | Vingi-1 | Vingi-1 driver R486K mutant |
| 143 | EX3475 | PROTEIN | Vingi-1 | Vingi-1 driver P511S mutant |
| 144 | EX3476 | PROTEIN | Vingi-1 | Vingi-1 driver P515E mutant |
| 145 | EX3477 | PROTEIN | Vingi-1 | Vingi-1 driver R568K mutant |
| 146 | EX3478 | PROTEIN | Vingi-1 | Vingi-1 driver H576K mutant |
| 147 | EX3479 | PROTEIN | Vingi-1 | Vingi-1 driver S595R mutant |
| 148 | EX3485 | PROTEIN | Vingi-1 | Vingi-1 driver E676R mutant |
| 149 | EX3486 | PROTEIN | Vingi-1 | Vingi-1 driver A684E mutant |
| 150 | EX3507 | PROTEIN | Vingi-1 | Vingi-1 driver A874E mutant |
| 151 | EX3354 | PROTEIN | Vingi-1 | Vingi-1 driver M570L mutant |
| 152 | EX3355 | PROTEIN | Vingi-1 | Vingi-1 driver V574L mutant |
| 153 | EX3356 | PROTEIN | Vingi-1 | Vingi-1 driver L590F mutant |
| 154 | EX3357 | PROTEIN | Vingi-1 | Vingi-1 driver A621S mutant |
| 155 | EX3360 | PROTEIN | Vingi-1 | Vingi-1 driver Y950I mutant |
| 156 | EX3362 | PROTEIN | Vingi-1 | Vingi-1 driver M735E mutant |
| 157 | EX3363 | PROTEIN | Vingi-1 | Vingi-1 driver G886P mutant |
| 158 | EX3369 | PROTEIN | Vingi-1 | Vingi-1 driver Q300L mutant |
| 159 | EX3373 | PROTEIN | Vingi-1 | Vingi-1 driver A519P mutant |
| 160 | EX3374 | PROTEIN | Vingi-1 | Vingi-1 driver G833V mutant |
| 161 | EX3375 | PROTEIN | Vingi-1 | Vingi-1 driver K784R mutant |
| 162 | EX3378 | PROTEIN | Vingi-1 | Vingi-1 driver E514A mutant |
| 163 | EX3379 | PROTEIN | Vingi-1 | Vingi-1 driver C938D mutant |
| 164 | EX3381 | PROTEIN | Vingi-1 | Vingi-1 driver P515K mutant |
| 165 | EX3384 | PROTEIN | Vingi-1 | Vingi-1 driver P780A mutant |
| 166 | EX3385 | PROTEIN | Vingi-1 | Vingi-1 driver K807A mutant |
| 167 | EX3387 | PROTEIN | Vingi-1 | Vingi-1 driver K414R mutant |
| 168 | EX3388 | PROTEIN | Vingi-1 | Vingi-1 driver K966R mutant |
| 169 | EX3353 | PROTEIN | Vingi-1 | Vingi-1 driver Y562F mutant |
| 170 | EX3400 | PROTEIN | Vingi-1 | Vingi-1 driver A742M mutant |
| 171 | EX3389 | PROTEIN | Vingi-1 | Vingi-1 driver H460R mutant |
| 172 | EX3430 | PROTEIN | Vingi-1 | Vingi-1 driver E418R mutant |
| 173 | EX3462 | PROTEIN | Vingi-1 | Vingi-1 driver D334E mutant |
| 174 | EX3219 | PROTEIN | Vingi-1 | Vingi-1 driver D191A mutant |
| 175 | EX3481 | PROTEIN | Vingi-1 | Vingi-1 driver R609K mutant |
| 176 | EX3482 | PROTEIN | Vingi-1 | Vingi-1 driver K611T mutant |
| 177 | EX3484 | PROTEIN | Vingi-1 | Vingi-1 driver N665K mutant |
| 178 | EX3487 | PROTEIN | Vingi-1 | Vingi-1 driver N695R mutant |
| 179 | EX3488 | PROTEIN | Vingi-1 | Vingi-1 driver R696H mutant |
| 180 | EX3491 | PROTEIN | Vingi-1 | Vingi-1 driver A742T mutant |
| 181 | EX3492 | PROTEIN | Vingi-1 | Vingi-1 driver K705R mutant |
| 182 | EX3494 | PROTEIN | Vingi-1 | Vingi-1 driver A755K mutant |
| 183 | EX3497 | PROTEIN | Vingi-1 | Vingi-1 driver A786E mutant |
| 184 | EX3499 | PROTEIN | Vingi-1 | Vingi-1 driver K807R mutant |
| 185 | EX3500 | PROTEIN | Vingi-1 | Vingi-1 driver P808T mutant |
| 186 | EX3502 | PROTEIN | Vingi-1 | Vingi-1 driver C841D mutant |
| 187 | EX3503 | PROTEIN | Vingi-1 | Vingi-1 driver E842P mutant |
| 188 | EX3504 | PROTEIN | Vingi-1 | Vingi-1 driver T854Q mutant |
| 189 | EX3506 | PROTEIN | Vingi-1 | Vingi-1 driver T867R mutant |
| 190 | EX3512 | PROTEIN | Vingi-1 | Vingi-1 driver Q947E mutant |
| 191 | EX3513 | PROTEIN | Vingi-1 | Vingi-1 driver A951V mutant |
| 192 | EX3514 | PROTEIN | Vingi-1 | Vingi-1 driver Q954K mutant |
| 193 | EX3324 | PROTEIN | Vingi-1 | Vingi-1 driver N330E mutant |
| 194 | EX3325 | PROTEIN | Vingi-1 | Vingi-1 driver L60P mutant |
| 195 | EX3326 | PROTEIN | Vingi-1 | Vingi-1 driver G833K mutant |
| 196 | EX3327 | PROTEIN | Vingi-1 | Vingi-1 driver G861S mutant |
| 197 | EX3361 | PROTEIN | Vingi-1 | Vingi-1 driver Y950V mutant |
| 198 | EX3376 | PROTEIN | Vingi-1 | Vingi-1 driver G833L mutant |
| 199 | EX3380 | PROTEIN | Vingi-1 | Vingi-1 driver A226T mutant |
| 200 | EX3396 | PROTEIN | Vingi-1 | Vingi-1 driver K604R mutant |
| 201 | EX3440 | PROTEIN | Vingi-1 | Vingi-1 driver S35A mutant |
| 202 | EX3448 | PROTEIN | Vingi-1 | Vingi-1 driver C144T mutant |
| 203 | EX3458 | PROTEIN | Vingi-1 | Vingi-1 driver G316E mutant |
| 204 | EX3460 | PROTEIN | Vingi-1 | Vingi-1 driver N330K mutant |
| 205 | EX3466 | PROTEIN | Vingi-1 | Vingi-1 driver D360A mutant |
| 206 | EX3343 | PROTEIN | Vingi-1 | Vingi-1 driver M167L mutant |
| 207 | EX3345 | PROTEIN | Vingi-1 | Vingi-1 driver T214S mutant |
| 208 | EX3359 | PROTEIN | Vingi-1 | Vingi-1 driver Y950L mutant |
| 209 | EX3367 | PROTEIN | Vingi-1 | Vingi-1 driver V838Q mutant |
| 210 | EX3368 | PROTEIN | Vingi-1 | Vingi-1 driver D375N mutant |
| 211 | EX3372 | PROTEIN | Vingi-1 | Vingi-1 driver H783A mutant |
| 212 | EX3377 | PROTEIN | Vingi-1 | Vingi-1 driver P511K mutant |
| 213 | EX3382 | PROTEIN | Vingi-1 | Vingi-1 driver P515S mutant |
| 214 | EX3383 | PROTEIN | Vingi-1 | Vingi-1 driver C779A mutant |
| 215 | EX3386 | PROTEIN | Vingi-1 | Vingi-1 driver P808W mutant |
| 216 | EX3407 | PROTEIN | Vingi-1 | Vingi-1 driver S754Y mutant |
| 217 | EX3408 | PROTEIN | Vingi-1 | Vingi-1 driver I793N mutant |
| 218 | EX3428 | PROTEIN | Vingi-1 | Vingi-1 driver P478W mutant |
| 219 | EX3437 | PROTEIN | Vingi-1 | Vingi-1 driver I491R mutant |
| 220 | EX3453 | PROTEIN | Vingi-1 | Vingi-1 driver H201N mutant |
| 221 | EX3454 | PROTEIN | Vingi-1 | Vingi-1 driver A223V mutant |
| 222 | EX3457 | PROTEIN | Vingi-1 | Vingi-1 driver Q309K mutant |
| 223 | EX3461 | PROTEIN | Vingi-1 | Vingi-1 driver K333R mutant |
| 224 | EX3472 | PROTEIN | Vingi-1 | Vingi-1 driver R465K mutant |
| 225 | EX3474 | PROTEIN | Vingi-1 | Vingi-1 driver R489Q mutant |
| 226 | EX3501 | PROTEIN | Vingi-1 | Vingi-1 driver H840T mutant |
| 227 | EX3505 | PROTEIN | Vingi-1 | Vingi-1 driver S858R mutant |
| 228 | EX3508 | PROTEIN | Vingi-1 | Vingi-1 driver A892H mutant |
| 229 | EX3509 | PROTEIN | Vingi-1 | Vingi-1 driver E904H mutant |
| 230 | EX3365 | PROTEIN | Vingi-1 | Vingi-1 driver F715E mutant |
| 231 | EX3390 | PROTEIN | Vingi-1 | Vingi-1 driver K661R mutant |
| 232 | EX3397 | PROTEIN | Vingi-1 | Vingi-1 driver K611Q mutant |
| 233 | EX3405 | PROTEIN | Vingi-1 | Vingi-1 driver D792R mutant |
| 234 | EX3436 | PROTEIN | Vingi-1 | Vingi-1 driver G426R mutant |
| 235 | EX3480 | PROTEIN | Vingi-1 | Vingi-1 driver R606K mutant |
| 236 | EX3490 | PROTEIN | Vingi-1 | Vingi-1 driver H739K mutant |
| 237 | EX3496 | PROTEIN | Vingi-1 | Vingi-1 driver S771C mutant |
| 238 | EX3344 | PROTEIN | Vingi-1 | Vingi-1 driver H171N mutant |
| 239 | EX3409 | PROTEIN | Vingi-1 | Vingi-1 driver H929R mutant |
| 240 | EX3446 | PROTEIN | Vingi-1 | Vingi-1 driver C127I mutant |
| 241 | EX3483 | PROTEIN | Vingi-1 | Vingi-1 driver L637N mutant |
| 242 | EX3489 | PROTEIN | Vingi-1 | Vingi-1 driver R731K mutant |
| 243 | EX3493 | PROTEIN | Vingi-1 | Vingi-1 driver S754T mutant |
| 244 | EX3498 | PROTEIN | Vingi-1 | Vingi-1 driver Q790K mutant |
| 245 | EX3511 | PROTEIN | Vingi-1 | Vingi-1 driver R927K mutant |
| 246 | EX3224 | PROTEIN | Vingi-1 | Vingi-1 driver HMGN1 mutant |
| 247 | EX3565 | DNA | Vingi-1 | Plasmid encoding Vingi-1 driver mRNA with |
| | | | | human alpha globin 5′ and 3′ UTR, N-terminal |
| | | | | HMGN1, C-terminal UL12, and C-termina |
| | | | | HMGB1 fusion |
| 248 | EX3220 | PROTEIN | Vingi-1 | Vingi-1 driver RNASEH deletion mutant |
| 249 | EX3565 | PROTEIN | Vingi-1 | Plasmid encoding Vingi-1 driver mRNA with |
| | | | | human alpha globin 5′ and 3′ UTR, N-terminal |
| | | | | HMGN1, C-terminal UL12, and C-termina |
| | | | | HMGB1 fusion |
| 250 | EX3242 | PROTEIN | Vingi-1 | C-terminal fusion of FEN1 PCNA interaction |
| | | | | motif to Vingi-1 driver |
| 251 | EX3424 | PROTEIN | Vingi-1 | Vingi-1 driver with natural PCNA interaction |
| | | | | motif replaced by PCNA interaction motif from |
| | | | | P21 |
| 252 | EX3425 | PROTEIN | Vingi-1 | C-terminal fusion of T4 phage GP45 to Vingi-1 |
| | | | | protein |
| 253 | EX3426 | PROTEIN | Vingi-1 | C-terminal fusion of Sso7D to Vingi-1 protein |
| 254 | EX3427 | PROTEIN | Vingi-1 | Vingi-1 driver with C-terminal fusion of Vif |
| | | | | derived peptides |
| 255 | EX3421 | PROTEIN | Vingi-1 | C-terminal fusion of Sto7D to Vingi-1 protein |
| 256 | EX3423 | PROTEIN | Vingi-1 | C-terminal fusion of Rad51 to Vingi-1 protein |
| 257 | EX3563 | PROTEIN | Vingi-1 | Vingi-1 driver endonuclease deletion mutant |
| 258 | EX3564 | PROTEIN | Vingi-1 | Vingi-1 driver Zinc fingerdeletion mutant |
| 259 | EX3420 | PROTEIN | Vingi-1 | N-terminal dead Cas9 (D10A, H840A) Vingi-1 |
| | | | | fusion |
| 260 | EX3221 | DNA | Vingi-1 | Plasmid encoding Vingi-1 driver mRNA with |
| | | | | WPRE 3′ UTR |
| 261 | EX3222 | DNA | Vingi-1 | Plasmid encoding Vingi-1 driver mRNA with |
| | | | | Human alpha globin 3′ UTR |
| 262 | EX3223 | DNA | Vingi-1 | Plasmid encoding Vingi-1 driver mRNA with |
| | | | | human alpha globin 5′UTR |
| 263 | EX3257 | PROTEIN | Vingi-1 | N-terminal i53 Vingi-1 fusion |
| 264 | EX3529 | PROTEIN | Vingi-1 | C-terminal MDC1 fusion to Vingi-1 |
| 265 | EX3240 | PROTEIN | Vingi-1 | C-terminal BRCA2-derived peptide Vingi-1 |
| | | | | fusion |
| 266 | EX3255 | PROTEIN | Vingi-1 | N-terminal PolD3 Vingi-1 fusion |
| 267 | EX3258 | PROTEIN | Vingi-1 | C-terminal ctI53tide 15-5 fusion |
| 268 | EX3525 | PROTEIN | Vingi-1 | Vingi-1 driver N-terminal TOPBP1 NLS fusion |
| 269 | EX3527 | PROTEIN | Vingi-1 | Vingi-1 driver N-terminal PARP1 NLS fusion |
| 270 | EX3528 | PROTEIN | Vingi-1 | C-terminal ANKRD28 Vingi-1 fusion |
| 271 | EX3531 | PROTEIN | Vingi-1 | C-terminal RAD17 Vingi-1 fusion |
| 272 | EX3532 | PROTEIN | Vingi-1 | C-terminal SCML1 fusion to Vingi-1 |
| 273 | EX3533 | PROTEIN | Vingi-1 | C-terminal CDKN2a Vingi-1 fusion |
| 274 | EX3534 | PROTEIN | Vingi-1 | C-terminal CHAF1A PCNA-interaction motif |
| | | | | to Vingi-1 driver |
| 275 | EX3518 | PROTEIN | Vingi-1 | N-terminal fusion of paRecT to Vingi-1 driver |
| 276 | EX3530 | PROTEIN | Vingi-1 | C-terminal fusion of MSH4 to Vingi-1 |
| 277 | EX3526 | PROTEIN | Vingi-1 | C-terminal fusion MDM2 NLS to Vingi-1 |
| 293 | EX661 | PROTEIN | ZFL2-2 | N-terminal Cas9 fusion to ZFL2-2 |
| 294 | EX662 | PROTEIN | ZFL2-2 | N-terminal Cas9 nickase (H840A) fusion to |
| | | | | ZFL2-2 endonuclease domain mutant (D216A) |
| 295 | EX274 | PROTEIN | ZFL2-2 | N-terminal dead Cas9 ZFL2-2 fusion |
| 296 | EX3419 | PROTEIN | Vingi-1 | N-terminal nickase Cas9 (H840A) fusion to |
| | | | | Vingi-1 driver with endonuclease domain |
| | | | | mutation (D191A) |
| 297 | EX3420 | PROTEIN | Vingi-1 | N-terminal dead Cas9 fusion to Vingi-1 driver |
| 298 | EX4758 | PROTEIN | Vingi-1 | Vingi-1-Acar-D51A-S6 driver D51A mutant |
| 299 | EX4759 | PROTEIN | Vingi-1 | Vingi-1-Acar-D138A-S6 driver D138A mutant |
| 300 | EX4760 | PROTEIN | Vingi-1 | Vingi-1-Acar-D149A-S6 driver D149A mutant |
| 301 | EX4761 | PROTEIN | Vingi-1 | Vingi-1-Acar-D152A-S6 driver D152A mutant |
| 302 | EX4762 | PROTEIN | Vingi-1 | Vingi-1-Acar-D172A-S6 driver D172A mutant |
| 303 | EX4763 | PROTEIN | Vingi-1 | Vingi-1-Acar-D118A-S6 driver D118A mutant |
| 304 | EX4764 | PROTEIN | Vingi-1 | Vingi-1-Acar-Q215A-S6 driver Q215A mutant |
| 305 | EX291 | PROTEIN | ZFL2-2 | L22 D216A endonuclease domain mutant |
| 306 | EX276 | PROTEIN | ZFL2-2 | L22 D237A endonuclease domain mutant |
| 307 | EX663 | PROTEIN | ZFL2-2 | N-terminal Cas9 L22 endonuclease mutant |
| | | | | fusion |
| 308 | | PROTEIN | Heterologous | BRCA2-derived peptide |
| 309 | | PROTEIN | Heterologous | DSS1-derived peptide |
| 310 | | PROTEIN | Heterologous | CtIP-derived peptide |
| 311 | | PROTEIN | Heterologous | RAD51 protein |
| 312 | | PROTEIN | Heterologous | Nibrin MRE11 recruitment peptide |
| 313 | | PROTEIN | Heterologous | MDM2 p53 inhibitory peptide |
| 314 | | PROTEIN | Heterologous | p53-inhibiting peptide |
| 315 | | PROTEIN | Heterologous | Nanog-derived peptide |
| 316 | | PROTEIN | Heterologous | E. coli RNaseH1 domain |
| 317 | | PROTEIN | Heterologous | human RNase H1 catalytic domain |
| 318 | | PROTEIN | Heterologous | Zinc finger AAVS1 DNA-binding domain |
| 319 | EX2107 | DNA | ZFL2-2 | Plasmid encoding ZFL2-2-drivenGFP reporter. |
| 320 | EX2561 | DNA | ZFL2-2 | Plasmid encoding ZFL2-2 driver |
| 321 | EX2556 | DNA | ZFL2-2 | Plasmid encoding ZFL2-2 driver with N- |
| | | | | terminal HMGN1, N647K mutation, C-terminal |
| | | | | UL12 fusion followed by C-terminal HMGB1 |
| | | | | fusion. |
| 322 | EX2195 | DNA | ZFL2-2 | Plasmid encoding ZFL2-2 driver with N- |
| | | | | terminal HMGN1, N647K and I343K |
| | | | | mutations, C-terminal UL12 fusion followed by |
| | | | | C-terminal HMGB1 fusion |
| 323 | EX2196 | DNA | ZFL2-2 | Plasmid encoding ZFL2-2 driver with N- |
| | | | | terminal HMGN1, D64K, N647K, and I343K |
| | | | | mutations, C-terminal UL12 fusion followed by |
| | | | | C-terminal HMGB1 fusion |
| 324 | EX2199 | DNA | ZFL2-2 | Plasmid encoding ZFL2-2 driver with N- |
| | | | | terminal HMGN1, D64K, N647K, L825G, and |
| | | | | I343K mutations, C-terminal UL12 fusion |
| | | | | followed by C-terminal HMGB1 fusion. |
| 325 | EX2200 | DNA | ZFL2-2 | Plasmid encoding ZFL2-2 driver with N- |
| | | | | terminal HMGN1, D64K, N647K, M750L, and |
| | | | | I343K mutations, C-terminal UL12 fusion |
| | | | | followed by C-terminal HMGB1 fusion |
| 326 | EX2985 | DNA | Vingi-1 | Plasmid encoding Vingi-1 driver |
| 327 | EX2985 | PROTEIN | Vingi-1 | Vingi-1 driver protein |
| 328 | EX2988 | DNA | Vingi-1 | Vingi-1 GFP reporter gene delivery construct |
| 329 | | PROTEIN | Heterologous | GP45 protein from T4 phage |
| 330 | | PROTEIN | Heterologous | dead Cas9 (D10A H840A) |
| 331 | | PROTEIN | Heterologous | PCSK9 homing endonuclease |
| 332 | | PROTEIN | Heterologous | PCSK9 homing nickase (Q47E) |
| 333 | | PROTEIN | Heterologous | Cas9 nickase (H840A) |
| 334 | | PROTEIN | Heterologous | Rigid linker |
| 335 | | PROTEIN | Heterologous | GS linker 1 |
| 336 | | PROTEIN | Heterologous | GS linker 2 |
| 337 | | PROTEIN | Heterologous | GS linker 3 |
| 338 | | PROTEIN | Heterologous | GS linker 4 |
| 339 | | PROTEIN | Heterologous | GS linker 5 |
| 340 | | PROTEIN | Heterologous | GS linker 6 |
| 341 | EX120 | PROTEIN | ZFL2-2 | L2-2 with I343K mutation |
| 342 | EX121 | PROTEIN | ZFL2-2 | L2-2 with Q372 mutation |
| 343 | EX122 | PROTEIN | ZFL2-2 | L2-2 with E366N mutation |
| 344 | EX123 | PROTEIN | ZFL2-2 | L2-2 with L354N mutation |
| 345 | EX124 | PROTEIN | ZFL2-2 | L2-2 with D588A mutation |
| 346 | EX125 | PROTEIN | ZFL2-2 | L2-2 with E616R and S617K mutation |
| 347 | EX126 | PROTEIN | ZFL2-2 | L2-2 with N647K mutation |
| 348 | EX127 | PROTEIN | ZFL2-2 | L2-2 with A688V mutation |
| 349 | EX128 | PROTEIN | ZFL2-2 | L2-2 with A688I mutation |
| 350 | EX129 | PROTEIN | ZFL2-2 | L2-2 with Y139K mutation |
| 351 | EX130 | PROTEIN | ZFL2-2 | L2-2 with D64K mutation |
| 352 | EX131 | PROTEIN | ZFL2-2 | L2-2 with S960R mutation |
| 353 | EX132 | PROTEIN | ZFL2-2 | L2-2 with D550T mutation |
| 354 | EX133 | PROTEIN | ZFL2-2 | L2-2 with L444F mutation |
| 355 | EX134 | PROTEIN | ZFL2-2 | L2-2 with D770H mutation |
| 356 | EX135 | PROTEIN | ZFL2-2 | L2-2 with I625L mutation |
| 357 | EX136 | PROTEIN | ZFL2-2 | L2-2 with H521P mutation |
| 358 | EX137 | PROTEIN | ZFL2-2 | L2-2 with S737P mutation |
| 359 | EX138 | PROTEIN | ZFL2-2 | L2-2 with P705A mutation |
| 360 | EX139 | PROTEIN | ZFL2-2 | L2-2 with M558L mutation |
| 361 | EX140 | PROTEIN | ZFL2-2 | L2-2 with M733L mutation |
| 362 | EX141 | PROTEIN | ZFL2-2 | L2-2 with M760S mutation |
| 363 | EX142 | PROTEIN | ZFL2-2 | L2-2 with M750L mutation |
| 364 | EX143 | PROTEIN | ZFL2-2 | L2-2 with A757P mutation |
| 365 | EX144 | PROTEIN | ZFL2-2 | L2-2 with H717A mutation |
| 366 | EX145 | PROTEIN | ZFL2-2 | L2-2 with H717K mutation |
| 367 | EX146 | PROTEIN | ZFL2-2 | L2-2 with D497S mutation |
| 368 | EX147 | PROTEIN | ZFL2-2 | L2-2 with I625H mutation |
| 372 | | DNA | Vingi-1 | Vingi-1 RNA stem loop |
| 373 | | DNA | Vingi-1 | Vingi-1 RNA microsattelite |
| 374 | | DNA | ZFL2-2 | L22 RNA stem loop |
| 375 | | DNA | ZFL2-2 | L22 RNA microsattelite |
| 376 | EX3335 | PROTEIN | Vingi-1 | Vingi-1 driver with F238Y + M16I mutations |
| 377 | | PROTEIN | Heterologous | Sso7D protein from Saccharolobus solfataricus |
| 378 | | PROTEIN | Heterologous | Vif protein from HIV |
| 379 | | PROTEIN | Heterologous | Vif-derived peptide |
| 380 | | PROTEIN | Heterologous | Vif-derived peptide |
| 381 | | PROTEIN | Heterologous | paRecT from Pseudomonas aeruginosa |
| 382 | | PROTEIN | Heterologous | i53 |
| 383 | | PROTEIN | Heterologous | TOPBP1 NLS |
| 384 | | PROTEIN | Heterologous | PARP1 NLS |
| 385 | | PROTEIN | Heterologous | human PolD3 |
| 386 | | PROTEIN | Heterologous | Rad17 protein fragment |
| 387 | | PROTEIN | Heterologous | SCML1 protein fragment |
| 388 | | PROTEIN | Heterologous | MDC1 protein fragment |
| 389 | | PROTEIN | Heterologous | CDKN2a protein fragment |
| 390 | | PROTEIN | Heterologous | MDM2 NLS |
| 391 | | PROTEIN | Heterologous | FEN1 PCNA interaction motif |
| 392 | | PROTEIN | Heterologous | MSH4 protein fragment |
| 393 | | DNA | Heterologous | WPRE 3′ UTR |
| 394 | | PROTEIN | Heterologous | FEN1 PCNA interaction motif |
| 395 | | PROTEIN | Heterologous | P21 PCNA interaction motif |
| 396 | | PROTEIN | Heterologous | ANKRD28 protein fragment |
| 397 | EX3415 | DNA | Heterolog | Plasmid encoding mRNA for Vingi-1 with |
| | | | | altered codons |
| 398 | EX2996 | DNA | Heterolog | Plasmid encoding Vingi-1 reporter gene |
| | | | | delivery construct with Kymriah transgene |
| | | | | under EF-1a promoter |
| 400 | EX174 | PROTEIN | Heterologous | SpCas9 nuclease |
| 401 | | PROTEIN | Heterologous | TALE AAVS1 DNA binding domain |
| 402 | | PROTEIN | Heterologous | StkC DNA binding protein |

Mutant A684S showed a large increase in integration efficiency of CAR transgene after 12 days (approximately 50% increased relative to wild type). Robust cellular expression of CAR transgene was observed for all constructs.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “any or all” of the elements conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A nucleic acid encoding an engineered protein comprising a retroelement-derived polypeptide and at least one heterologous polypeptide-;
wherein the retroelement-derived polypeptide is derived from a non-long terminal repeat (non-LTR) retrotransposon, wherein the non-LTR retrotransposon is an apurinic/apyrimidinic endonucleases (APE)-type retrotransposon selected from a ZFL2-2 retrotransposon, a Vingi-1_Acar retrotransposon, a Vingi-2_Acar retrotransposon, a L2-18_Acar retrotransposon, or a CR1-1_Acar retrotransposon;
wherein the at least one heterologous polypeptide comprises an RNA/DNA processing polypeptide or domain thereof, an RNA/DNA repair polypeptide or domain thereof, a nucleic acid binding polypeptide or domain thereof, or a nucleosome binding polypeptide or domain thereof, and
wherein the engineered protein exhibits at least one improved integration characteristic, as compared to a retroelement-derived polypeptide not fused to the at least one heterologous polypeptide.

2.-3. (canceled)

4. The nucleic acid of claim 1, wherein the at least one heterologous polypeptide comprises an RNA/DNA processing polypeptide or domain thereof.

5. (canceled)

6. The nucleic acid of claim 4, wherein the RNA/DNA processing polypeptide is a Rad51 polypeptide, an RNAseH domain, or a DNA polymerase.

7. (canceled)

8. The nucleic acid of claim 1, wherein the at least one heterologous polypeptide comprises an RNA/DNA repair polypeptide or domain thereof.

9. (canceled)

10. The nucleic acid of claim 8, wherein the RNA/DNA repair polypeptide is a CtIP-derived polypeptide, a RecT-derived polypeptide, an HSV-1 alkaline nuclease-derived polypeptide, a BRCA2-derived polypeptide, a DSS1-derived polypeptide, a nanog-derived polypeptide, an NBN-derived polypeptide, a RAD17-derived polypeptide, an ANKRD28-derived polypeptide, a PCNA interaction motif polypeptide, a MDC1-derived polypeptide, a MSH4-derived polypeptide, a SCML1-derived polypeptide, a CDKN2A-derived polypeptide, a 53BP1 inhibitor, or a p53 inhibitor.

11.-21. (canceled)

22. The nucleic acid of claim 1, wherein the at least one heterologous polypeptide comprises a nucleic acid binding polypeptide or domain thereof.

23. The nucleic acid of claim 22, wherein the nucleic acid binding polypeptide comprises a non-sequence specific DNA binding polypeptide or domain thereof.

24. The nucleic acid of claim 23, wherein the non-sequence specific DNA binding polypeptide comprises a Sto7d DNA binding domain having an amino acid sequence that is at least 70% identical to the sequence as set forth in SEQ ID NO: 26, or an Sso7d DNA binding domain having an amino acid sequence that is at least 70% identical to the sequence as set forth in SEQ ID NO: 377.

25.-26. (canceled)

27. The nucleic acid of claim 1, wherein at least one heterologous polypeptide comprises a nucleosome binding polypeptide or domain thereof.

28. The nucleic acid of claim 27, wherein the nucleosome binding polypeptide comprises;
(a) an HMGN1 polypeptide having an amino acid sequence that is at least 70% identical to the sequence as set forth in SEQ ID NO:23;
(b) an HMGB1 polypeptide having an amino acid sequence that is at least 70% identical to the sequence as set forth in SEQ ID NO:24; or
(c) an StkC DNA binding domain having an amino acid sequence that is at least 70% identical to the sequence as set forth in SEQ ID NO:402.

29.-30. (canceled)

31. The nucleic acid of claim 1, wherein the engineered protein comprises the at least one heterologous polypeptide fused to the N-terminus of the retroelement-derived polypeptide, to the C-terminus of the retroelement-derived polypeptide and/or internally within the retroelement-derived polypeptide.

32.-35. (canceled)

36. The nucleic acid of claim 1, wherein engineered protein comprises a plurality of heterologous polypeptides.

37.-54. (canceled)

55. The nucleic acid of claim 1, wherein the retroelement-derived polypeptide is a retroelement-derived polypeptide variant comprising an amino acid substitution, an amino acid deletion, an amino acid truncation, or a combination thereof, when compared to a wild type retroelement-derived polypeptide.

56. The nucleic acid of claim 1, wherein the retroelement-derived polypeptide comprises a reverse transcriptase domain, an endonuclease domain, an integrase domain, and/or an RNA binding domain.

57.-126. (canceled)

127. The nucleic acid of claim 1 comprising a codon optimized sequence, wherein the codon optimized sequence is optimized for expression in human cells.

128.-130. (canceled)

131. An engineered protein encoded by the nucleic acid of claim 1.

132. A composition comprising:
a) the first nucleic acid of claim 1; and
b) a second nucleic acid comprising a polynucleotide encoding a gene of interest.

133. The composition of claim 132, wherein the first and second nucleic acids are separate DNA molecules or separate RNA molecules, or wherein one of the first and second nucleic acids is a DNA molecule and one of the first and second nucleic acids is an RNA molecule.

134.-160. (canceled)

161. The composition of claim 132, wherein the first and second nucleic acids are comprised within a plurality of LNP particles.

162. A method of modifying a polynucleotide, comprising contacting a polynucleotide with the nucleic acid of claim 1.

163. (canceled)

164. A method of treating a subject in need thereof comprising administering to the subject the nucleic acid of claim 1.

165.-166. (canceled)
